(19) United States
(12) Patent Application Publication
(10) Pub. No.: US 2026/0248784 A1
(43) Pub. Date: Aug. 27, 2026

(54) BTK Degrader and Use Thereof

(71) Applicant: JIANGSU VCARE PHARMATECH CO., LTD., Nanjing, Jiangsu (CN)

(72) Inventors: Yong WU, Nanjing, Jiangsu (CN); Yanchun GONG, Nanjing, Jiangsu (CN); Yongqiang LIU, Nanjing, Jiangsu (CN)

(21) Appl. No.: 19/156,298

(22) PCT Filed: Feb. 7, 2024
(86) PCT No.: PCT/CN2024/076690

§ 371 Date: Aug. 13, 2025

(30) Foreign Application Priority Data
Feb. 17, 2023 (CN) 202310131483.8
Nov. 27, 2023 (CN) 202311593736.X

(51) Int. Cl. A61K 31/4985; A61K 31/4545; A61K 31/496; A61K 31/5025; A61K 31/513; A61K 31/52; A61K 31/53; A61P 35/00; C07D 401/14; C07D 471/04; C07D 473/34; C07D 487/04; C07D 519/00

(52) U.S. Cl. CPC A61K 31/4985; A61K 31/4545; A61K 31/496; A61K 31/5025; A61K 31/513; A61K 31/52; A61K 31/53; A61P 35/00; C07D 401/14; C07D 471/04; C07D 473/34; C07D 487/04; C07D 519/00

(57) ABSTRACT

A BTK degrader and the use thereof are provided. A compound as shown in general formula (I), an isomer thereof or a pharmaceutically acceptable salt thereof and the use thereof, in particular, the use thereof in the preparation of a drug for treating diseases related to the inhibition or degradation of BTK are provided. Each of the substituents in formula (I) is as defined in the description.

The present invention is a U.S. national stage entry of PCT International Application No. PCT/CN2024/076690, filed on Feb. 7, 2024, which claims the priority of the earlier application filed with the China National Intellectual Property Administration (CNIPA) on Feb. 17, 2023, with patent application number 202310131483.8 and entitled “BTK degrader and use thereof”, the present invention claims the priority of the earlier application filed with the CNIPA on Nov. 27, 2023, with patent application number 202311593736.X and entitled “BTK degrader and use thereof”. The content of each is incorporated herein by reference in its entirety.

### FIELD OF THE INVENTION

The present application belongs to the technical field of medicines, and in particular relates to a BTK degrader as shown in formula (I), an isomer thereof or a pharmaceutically acceptable salt thereof, and the use thereof.

### DESCRIPTION OF RELATED ART

Bruton's tyrosine kinase (BTK) is widely involved in the activation, proliferation, and survival of B cells, and also involved in signaling pathways such as FcR, cytokine, and chemokine. It is a key kinase in B cells and immune regulation. The B cell receptor (BCR) is a transmembrane receptor on the surface of B lymphocytes that plays a key role in B cell development and adaptive immune responses. BTK is an important component of the B cell receptor signaling pathway, transmitting information received by the BCR and regulating B cell proliferation and survival. In the field of tumor treatment, there are many BTK degraders on the market. In recent years, BTK has received widespread attention in the fields of autoimmune diseases, inflammatory diseases, and the like.

Proteolysis-targeting chimeras (PROTACs) represent a novel therapeutic technology that blocks a signaling pathway by degrading a target protein, thereby exerting therapeutic effects. A PROTAC molecule usually consists of three parts: a target protein binding ligand, an E3 ubiquitin ligase ligand and a linker. The PROTAC molecule tightly binds to the target protein at one end of the chimera and binds to the E3 ubiquitin ligase at the other end to form a target protein-PROTAC-E3 ubiquitin ligase ternary complex. The target protein is “marked” with ubiquitination by E3 ubiquitin ligase-mediated ubiquitination; and the ubiquitination-“marked” target protein is then transported to the proteasome and degraded by the proteasome.

Compared with traditional small molecule inhibitors, PROTAC technology has the following advantages: 1. It may require a smaller dosage of administration. The target protein degradation process is similar to a catalytic reaction, in which the PROTAC molecule can be reused, so only a catalytic amount of a drug is needed to inhibit the target protein. 2. It can avoid the feedback upregulation of target protein expression caused by inhibition of the target protein. 3. It can expand the scope of drug targeting. PROTAC technology makes proteins that cannot be directly acted upon by traditional small molecule inhibitors, such as transcription factors, regulatory proteins and other non-enzyme proteins, become regulatable proteins, turning the target from “non-druggable” to “druggable”.

Drugs for targeted inhibition of BTK (such as ibrutinib and zanubrutinib) have demonstrated excellent efficacy against B cell-related cancers.

The present invention has found a class of molecules with good Bruton's tyrosine kinase degradation activity, which has good prospects for therapeutic use.

### SUMMARY OF THE INVENTION

The object of the present invention is to provide a PROTAC compound for Bruton's tyrosine kinase (BTK), an isomer thereof or a pharmaceutically acceptable salt thereof.

Technical solution of the present invention is as follows.

A compound as shown in formula (I), or an isomer or pharmaceutically acceptable salt thereof:

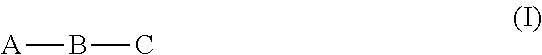

wherein,
A is

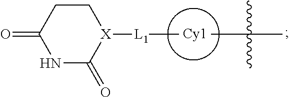

X is selected from N and CH;
Cy1 is selected from

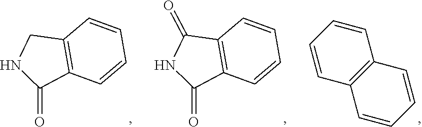

and phenyl, wherein the

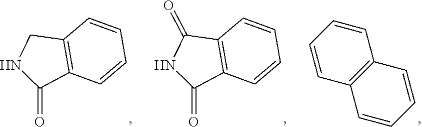

and phenyl are unsubstituted or each independently substituted with 1-3 Rₐ₁;
each Rₐ₁ is independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₄ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
B is -L2-Cy2-L3-(Cy3)p-L4-Cy4-L5-(Cy5)q-;
p and q are independently 0 or 1;
C is -L6-Cy6-L7-Cy7-L8-Cy8;
L1, L2, L3, L4, L5, L6, L7 and L8 are independently selected from a bond, —(CH₂)ₙ—(NH)ₘ—, —O—, —S—, —NR₂, —CR₃₁R₃₂, —C(O)—, —C(O)O—, —C(O)NR₄—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S; Cy3 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₃, a 4-6 membered heterocyclyl group substituted with 1-3 Rₐ₃, a 4-6 membered cycloalkenyl group substituted with 1-3 Rₐ₃, and a 4-6 membered heterocycloalkenyl group substituted with 1-3 Rₐ₃, wherein the heteroatom of the 4-6 membered heterocyclyl group or the 4-6 membered heterocycloalkenyl group is selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
Cy5 is a 4-6 membered cycloalkyl group, a 4-6 membered heterocyclyl group, a 4-6 membered heterocycloalkenyl group, a 4-6 membered cycloalkyl group substituted with 1-3 Rₐ₅, a 4-6 membered heterocyclyl group substituted with 1-3 Rₐ₅, a 4-6 membered heterocycloalkenyl group substituted with 1-3 Rₐ₅, or

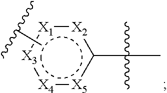

X₁ is N or CR_{b1};
X₂ is N or CR_{b2};
X₃ is N or CR_{b3};
X₄ is N or CR_{b4};
X₅ is N or CR_{b5};
R_{b1}, R_{b2}, R_{b3}, R_{b4} and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₈, wherein the heteroatom of the 5-6 membered heteroaryl group is selected from O, N and S; and
each of Rₐ₂, Rₐ₃, Rₐ₄, Rₐ₅, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

In another embodiment, the compound of formula (I), or the isomer or pharmaceutically acceptable salt thereof, having a structure as shown in general formula (II):

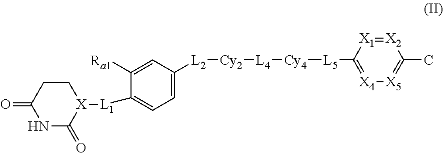

wherein,
X is selected from N and CH;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —CR₃₁R₃₂, —C(O)O—, —C(O)NR₄—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
X₁ is N or CR_{b1};
X₂ is N or CR_{b2};
X₄ is N or CR_{b4};
X₅ is N or CR_{b5};
R_{b1}, R_{b2}, R_{b4}, and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₅, and a 5-6 membered heteroaryl group substituted with 1-3 Rₐ₈, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

In another embodiment, the compound of formula (II), or the isomer or pharmaceutically acceptable salt thereof,
wherein,
X is selected from N and CH;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —CR₃₁R₃₂, —C(O)O—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
X₁ is N or CR_{b1};
X₂ is N or CR_{b2};
X₄ is N or CR_{b4};
X₅ is N or CR_{b5};
R_{b1}, R_{b2}, R_{b4}, and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₅, and a 5-6 membered heteroaryl group substituted with 1-3 R₈, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₄ alkoxy, halogenated C₁₋₆ alkoxy, hydroxy C₁₋₆ alkyl, C₁₋₆ alkylamino. (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

In another embodiment, the compound of formula (II), or the isomer or pharmaceutically acceptable salt thereof.
X is selected from N and CH;
Rₐ₁ is selected from hydrogen, halogen, hydroxy, amino, C₁₋₆ alkyl, halogenated C₁₋₆ alkyl, C₁₋₆ alkoxy, halogenated C₁₋₆ alkoxy, and hydroxy C₁₋₆ alkyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —C(O)O—, —C(O)NR₄—, and —C₁₋₆ alkyl-NR₅—C(O)—;
n and m are independently any integer from zero to three;
R₂, R₄, and R₅ are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 4-12 membered cycloalkyl group substituted with 1-3 Rₐ₄, and a 4-12 membered heterocyclyl group substituted with 1-3 Rₐ₄, wherein the heteroatom of the 4-12 membered heterocyclyl group is selected from O, N and S;
X₁ is N or CR_{b1};
X₂ is N or CR_{b2};
X₄ is N or CR_{b4};
X₅ is N or CR_{b5};
R_{b1}, R_{b2}, R_{b3}, R_{b4}, and R_{b5} are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₅, and a 5-6 membered heteroaryl group substituted with 1-3 Rₐ₅, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

In another embodiment, the compound of formula (II), or the isomer or pharmaceutically acceptable salt thereof, having a structure as shown in general formula (III):

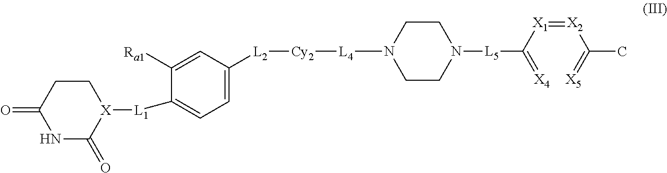

X is selected from N and CH;
Rₐ₁ is selected from hydrogen and methyl;
C is -L6-Cy6-L7-Cy7-L8-Cy8;
L1, L2, L4, L5, L6, L7 and L8 are independently selected from a bond, —CH₂—, and —CH₂—NH—C(O)—;
Cy2 is a 5-6 membered nitrogen-containing heterocyclyl group, preferably

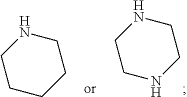 [SMILES: C1CCNCC1 ; C1CNCCN1]

X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen and F;
Cy6 is selected from a 5-6 membered heteroaryl group substituted with 1-3 Rₐ₆, and a 9-10 membered fused heteroaryl group substituted with 1-3 Rₐ₆, wherein the 5-6 membered heteroaryl group and the 9-10 membered fused heteroaryl group are preferably

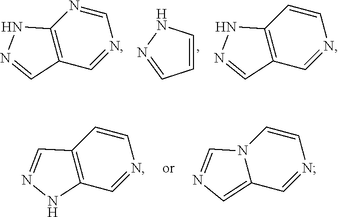

preferably

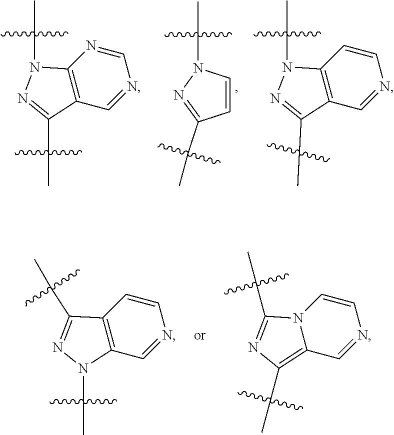 [SMILES: *c1nn(*)c2ncncc12 ; *c1ccn(*)n1 ; *c1nn(*)c2ccncc12 ; *c1nn(*)c2cnccc12 ; *c1nc(*)n2ccncc12]

more preferably

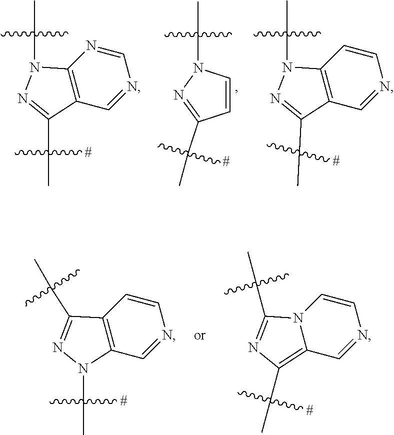 [SMILES: *c1nn(*)c2ncncc12 ; *c1ccn(*)n1 ; *c1nn(*)c2ccncc12 ; *c1nn(*)c2cnccc12 ; *c1nc(*)n2ccncc12]

wherein the * end is connected to L6, and the #end is connected to L7;
each Rₐ₆ is independently selected from hydrogen, amino, and aminocarbonyl;
Cy7 is selected from phenyl and phenyl substituted with 1-3 Rₐ₇;
each Rₐ₇ is independently selected from hydrogen and methyl;
Cy8 is phenyl substituted with 1-3 Rₐ₈; and
each Rₐ₈ is independently selected from hydrogen. F, and methoxy.

In one embodiment, the compound of formula (III), or the isomer or pharmaceutically acceptable salt thereof,
X is selected from N and CH;
Rₐ₁ is selected from hydrogen and methyl;
L1, L2, L4 and L5 are independently selected from a bond and —CH₂—;
Cy2 is a 5-6 membered nitrogen-containing heterocyclyl group, preferably

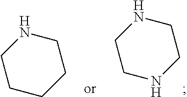 [SMILES: C1CCNCC1 ; C1CNCCN1]

X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen and F;
C is -L6-Cy6-L7-Cy7-L8-Cy8; and
-L6-Cy6-L7-Cy7-L8-Cy8 is

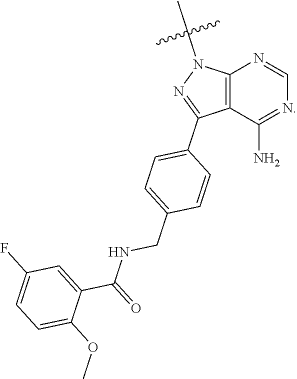 [SMILES: *n1nc(-c2ccc(CNC(=O)c3cc(F)ccc3OC)cc2)c2c(N)ncnc21]

In one embodiment, the compound of formula (III), or the isomer or pharmaceutically acceptable salt thereof,
X is selected from N and CH;
Rₐ₁ is selected from hydrogen and methyl;
L1, L2, L4 and L5 are independently selected from a bond and —CH₂—;

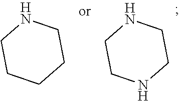

Cy2 is a 5-6 membered nitrogen-containing heterocyclyl group, preferably or H;
X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen and F;
C is -L6-Cy6-L7-Cy7-L8-Cy8; and
-L6-Cy6-L7-Cy7-L8-Cy8 is

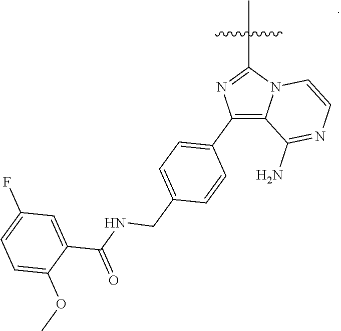 [SMILES: *c1nc(-c2ccc(CNC(=O)c3cc(F)ccc3OC)cc2)c2c(N)nccn12]

In another embodiment, the compound of formula (I), or the isomer or pharmaceutically acceptable salt thereof, having a structure as shown in general formula (IV),

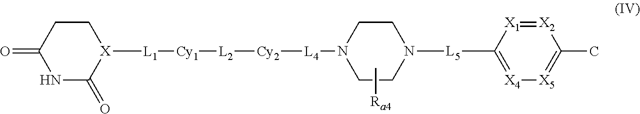

X is selected from N and CH
Cy1 is selected from

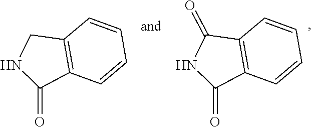 [SMILES: O=C1NCc2ccccc21 ; O=C1NC(=O)c2ccccc21]

wherein the

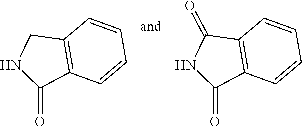 [SMILES: O=C1NCc2ccccc21 ; O=C1NC(=O)c2ccccc21]

are unsubstituted or each independently substituted with 1-3 Rₐ₁;
each Rₐ₁ is independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₄ alkylaminocarbonyl, and (C₁₋₆ alkyl)₂ aminocarbonyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —CR₃₁R₃₂, —C(O)O—, —C(O)NR₄—, and —C₁₋₄ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₄ alkyl, halogenated C₁₋₄ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₄ alkyl, halogenated C₁₋₄ alkyl, C₁₋₄ alkoxy, halogenated C₁₋₆ alkoxy, hydroxy C₁₋₄ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₄ alkyl, C₁₋₆ alkoxy, C₁₋₆ alkoxy C₁₋₄ alkoxy, C₁₋₄ alkylamino, (C₁₋₆ alkyl)₂ amino, C_{1-f} alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₄ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
X₁ is N or CR_{b1};
X₂ is N or CR_{b2};
X₄ is N or CR_{b4};
X₅ is N or CR_{b5};
R_{b1}, R_{b2}, R_{b4}, and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆ wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇ and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₅, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, hydroxy, carboxyl, amino, cyano, nitro, C₁₋₄ alkyl, halogenated C₁₋₆ alkyl, C₁₋₆ alkoxy, halogenated C₁₋₄ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

In another embodiment, the compound of formula (IV), or the isomer or pharmaceutically acceptable salt thereof,
X is selected from N and CH;
Cy1 is selected from

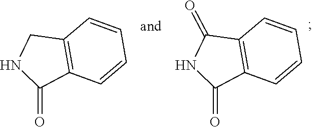 [SMILES: O=C1NCc2ccccc21 ; O=C1NC(=O)c2ccccc21]

C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —C(O)O—, —C(O)NR₄—, and —C₁₋₆ alkyl-NR₅—C(O)—;
n and m are independently any integer from zero to three;
R₂, R₄, and R₅ are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
X₁ is N or CR_{b1};
X₂ is N or CR_{b2};
X₄ is N or CR_{b4};
X₅ is N or CR_{b5};
R_{b1}, R_{b2}, R_{b4}, and R_{b5} are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 R_{b6}, a 5-6 membered heterocycloalkenyl group substituted with 1-3 Rₐ₆, a 9-10 membered fused heterocycloalkenyl group substituted with 1-3 Rₐ₆ and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇ and a 5-6 membered heteroaryl group substituted with 1-3 Rₐ₆, wherein the heteroatom of the 5-6 membered heteroaryl group is selected from O, N and S;
Cy8 is selected from phenyl, a 5-6 membered heteroaryl group, phenyl substituted with 1-3 Rₐ₅, and a 5-6 membered heteroaryl group substituted with 1-3 Rₐ₅, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

In another embodiment, the compound of formula (IV), or the isomer or pharmaceutically acceptable salt thereof:
X is CH;
Cy1 is selected from

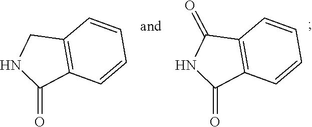 [SMILES: O=C1NCc2ccccc21 ; O=C1NC(=O)c2ccccc21]

C is -Cy6-L7-Cy7-L8-Cy8;
L1, L2, L4, L5, L6, L7 and L8 are independently selected from a bond, —CH₂—, and —CH₂—NH—C(O)—;
Cy2 is a 5-6 membered nitrogen-containing heterocyclyl group, preferably

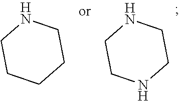

Rₐ₄ is hydrogen;
X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen and F;
Cy6 is selected from a 5-6 membered heteroaryl group substituted with 1-3 R₆, a 9-10 membered fused heterocycloalkenyl group substituted with 1-3 Rₐ₆, and a 9-10 membered fused heteroaryl group substituted with 1-3 Rₐ₆ wherein the 5-6 membered heteroaryl group, the 9-10 membered fused heterocycloalkenyl group, and the 9-10 membered fused heteroaryl group are preferably

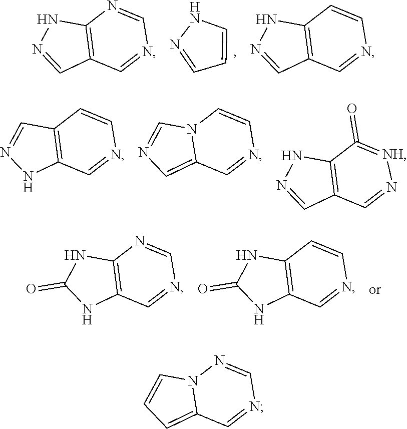

each Rₐ₆ is independently selected from hydrogen, amino, and aminocarbonyl;
Cy7 is selected from phenyl and phenyl substituted with 1-3 Rₐ₇;
each Rₐ₇ is independently selected from hydrogen, methyl, and F;
Cy8 is phenyl substituted with 1-3 Rₐ₈; and
each Rₐ₈ is independently selected from hydrogen, F, and methoxy.

In another embodiment of the present invention, the compound as shown in formula (I), or the isomer or pharmaceutically acceptable salt thereof, having a structure as shown in formula (V),

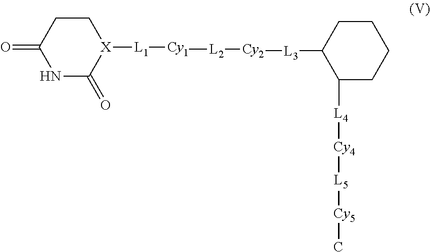

X is selected from N and CH;
Cy1 is selected from

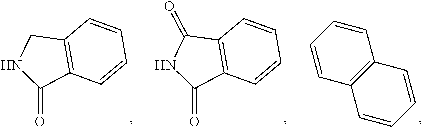

and phenyl, wherein the

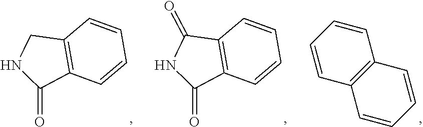

and phenyl are unsubstituted or each independently substituted with 1-3 Rₐ₁;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₄ alkylaminocarbonyl, and (C₁₋₄ alkyl)₂ aminocarbonyl;
C is -L6-Cy6-L7-Cy7-L8-Cy8;
L1, L2, L3, L4, L5, L6, L7 and L8 are independently selected from a bond, —(CH₂)ₙ—(NH)ₘ—, —O—, —S—, —NR₂, —CR₃₁R₃₂, —C(O)—, —C(O)O—, —C(O)NR₄—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C1-6 alkylamino, (C₁₋₆ alkyl)₂ amino, C₂₋₈ alkenyl, C₂₋₈ alkynyl, C₁₋₄ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₄ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₄ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
Cy5 is a 4-6 membered cycloalkyl group, a 4-6 membered heterocyclyl group, a 4-6 membered heterocycloalkenyl group, a 4-6 membered cycloalkyl group substituted with 1-3 Rₐ₅, a 4-6 membered heterocyclyl group substituted with 1-3 Rₐ₅, a 4-6 membered heterocycloalkenyl group substituted with 1-3 Rₐ₅, or

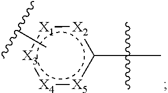

X₁ is N or CR_{b1};
X₂ is N or CR_{b2};
X₃ is N or CR_{b3};
X₄ is N or CR_{b4};
X₅ is N or CR_{b5};
R_{b1}, R_{b2}, R_{b3}, R_{b4} and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₄ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₈, wherein the heteroatom of the 5-6 membered heteroaryl group is selected from O, N and S; and
each of Rₐ₂, Rₐ₃, Rₐ₄, Rₐ₅, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₄ alkyl, C₁₋₆ alkylamino, (C₁₋₄ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₄ alkyl)₂ aminocarbonyl.

In another embodiment of the present invention, the compound as shown in formula (V), or the isomer or pharmaceutically acceptable salt thereof,
X is selected from N and CH;
Cy1 is selected from

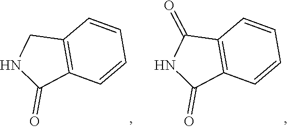

and phenyl, wherein the

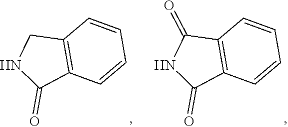

and phenyl are unsubstituted or each independently substituted with 1-3 Rₐ₁;
Rₐ₁ is selected from hydrogen, halogen, hydroxy, amino, C₁₋₆ alkyl, halogenated C₁₋₆ alkyl, C₁₋₆ alkoxy, halogenated C₁₋₆ alkoxy, and hydroxy C₁₋₆ alkyl;
C is -L6-Cy6-L7-Cy7-L8-Cy8;
L1, L2, L3, L4, L5, L6, L7 and L8 are independently selected from a bond, —(CH₂)ₙ—(NH)ₘ—, —O—, —S—, —NR₂, —C(O)—, —C(O)O—, —C(O)NR₄—, and —C₁₋₆ alkyl-NR₅—C(O)—;
n and m are independently any integer from zero to three;
R₂, R₄, and R₅ are independently selected from hydrogen, halogen, hydroxy, amino, C₁₋₄ alkyl, halogenated C₁₋₆ alkyl, C₁₋₆ alkoxy, halogenated C₁₋alkoxy, hydroxy C₁₋₆ alkyl, C₁₋₆ alkylamino, and (C₁₋₆ alkyl)₂ amino;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 4-6 membered cycloalkyl group substituted with 1-12 Rₐ₄, and a 4-12 membered heterocyclyl group substituted with 1-3 Rₐ₄, wherein the heteroatom of the 4-12 membered heterocyclyl group is selected from O, N and S;
Cy5 is a 4-6 membered heterocyclyl group, a 4-6 membered heterocyclyl group substituted with 1-3 Rₐ₈, or

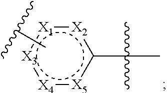

X₁ is N or CR_{b1};
X₂ is N or CR_{b2};
X₁ is N or CR_{b3};
X₄ is N or CR_{b4};
X₅ is N or CR_{b5};
R_{b1}, R_{b2}, R_{b3}, R_{b4}, and R_{b5} are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 as, and a 5-6 membered heteroaryl group substituted with 1-3 as, wherein the heteroatom of the 5-6 membered heteroaryl group is selected from O, N and S; and
each of Rₐ₂, Rₐ₄, Rₐ₅, Rₐ₆, Rₐ₇, and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

In another embodiment of the present invention, the compound as shown in formula (V), or the isomer or pharmaceutically acceptable salt thereof,
X is selected from N and CH;
Cy1 is selected from

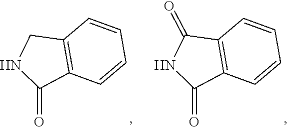

phenyl, and

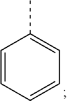

C is -Cy6-L7-Cy7-L8-Cy8;
L1, L2, L3, L4. L5, L6, L7 and L8 are independently selected from a bond, —CH₂—, —O—, and —CH₂—NH—C(O)—;
Cy2 is a 5-6 membered nitrogen-containing heterocyclyl group, preferably

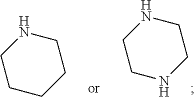 [SMILES: C1CCNCC1 ; C1CNCCN1]

Cy4 is a 4-6 membered nitrogen-containing heterocyclyl group, preferably

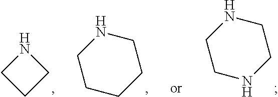

Cy5 is a 5-6 membered nitrogen-containing heterocyclyl group or

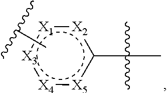

wherein the 5-6 membered nitrogen-containing heterocyclyl group is preferably

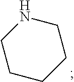

X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen and F;
Cy6 is selected from a 5-6 membered heteroaryl group substituted with 1-3 Rₐ₆, a 9-10 membered fused heterocycloalkenyl group substituted with 1-3 Rₐ₆ and a 9-10 membered fused heteroaryl group substituted with 1-3 Rₐ₆, wherein the 5-6 membered heteroaryl group, the 9-10 membered fused heterocycloalkenyl group, and the 9-10 membered fused heteroaryl group are preferably

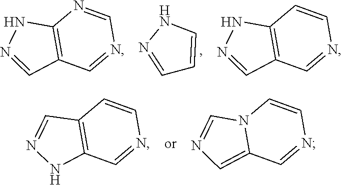

each Rₐ₆ is independently selected from hydrogen, amino, and aminocarbonyl;
Cy7 is phenyl;
Cy8 is selected from phenyl and phenyl substituted with 1-3 Rₐ₈; and
each Rₐ₈ is independently selected from hydrogen, F, and methoxy.

In another embodiment of the present invention, the compound as shown in formula (I), or the isomer or pharmaceutically acceptable salt thereof,
B is selected from

[chemical structure]

wherein the corresponding Cy2 group is unsubstituted or substituted with 1-3 Rₐ₂, the corresponding Cy3 group is unsubstituted or substituted with 1-3 Rₐ₃, the corresponding Cy4 group is unsubstituted or substituted with 1-3 Rₐ₄, and when the corresponding Cy5 group is a 5-6 membered heterocyclyl group, Cy5 is not substituted or substituted with 1-3 Rₐ₅, or Cy5 group is

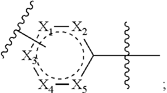

and each of Rₐ₂, Rₐ₃, Rₐ₄, Rₐ₅, R_{b1}, R_{b2}, R_{b3}, R_{b4}, and R_{b5} is at occurrence, independently selected from hydrogen and F.

In another embodiment of the present invention, the compound as shown in formula (II), or the isomer or pharmaceutically acceptable salt thereof,

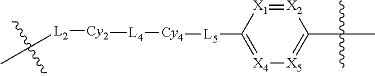

is selected from

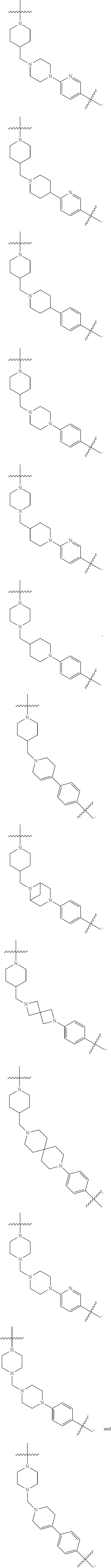

wherein the corresponding Cy2 group is unsubstituted or substituted with 1-3 Rₐ₂, the corresponding Cy4 group is unsubstituted or substituted with 1-3 Rₐ₄, and the corresponding X₁, X₂, X₄, and X₅ are unsubstituted or substituted with the corresponding R_{b1}, R_{b2}, R_{b4}, and R_{b5} respectively; and
each of Rₐ₂, Rₐ₄, R_{b1}, R_{b2}, R_{b4}, and R_{b5} is at occurrence, independently selected from hydrogen and F.

In another embodiment of the present invention, the compound as shown in formula (IV), or the isomer or pharmaceutically acceptable salt thereof,

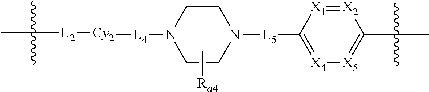

is selected from

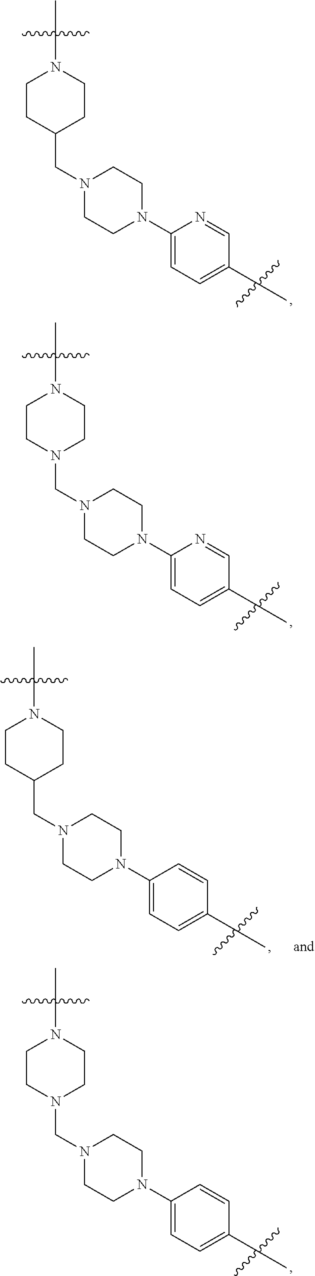 [SMILES: *c1ccc(N2CCN(CC3CCN(*)CC3)CC2)nc1 ; *c1ccc(N2CCN(CN3CCN(*)CC3)CC2)nc1 ; *c1ccc(N2CCN(CC3CCN(*)CC3)CC2)cc1 ; *c1ccc(N2CCN(CN3CCN(*)CC3)CC2)cc1]

wherein the corresponding Cy2 group is unsubstituted or substituted with 1-3 Rₐ₂, and the corresponding X₁, X₂, X₄, and X₅ are unsubstituted or substituted with the corresponding R_{b1}, R_{b2}, R_{b4}, and R_{b5} respectively; and
each of Rₐ₂, Rₐ₄, R_{b1}, R_{b2}, R_{b4}, and R_{b5} is at occurrence, independently selected from hydrogen and F.

In another embodiment of the present invention, the compound as shown in formula (V), or the isomer or pharmaceutically acceptable salt thereof,

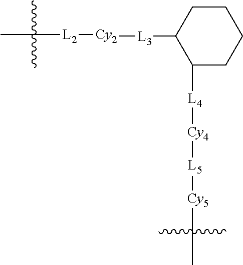

is selected from

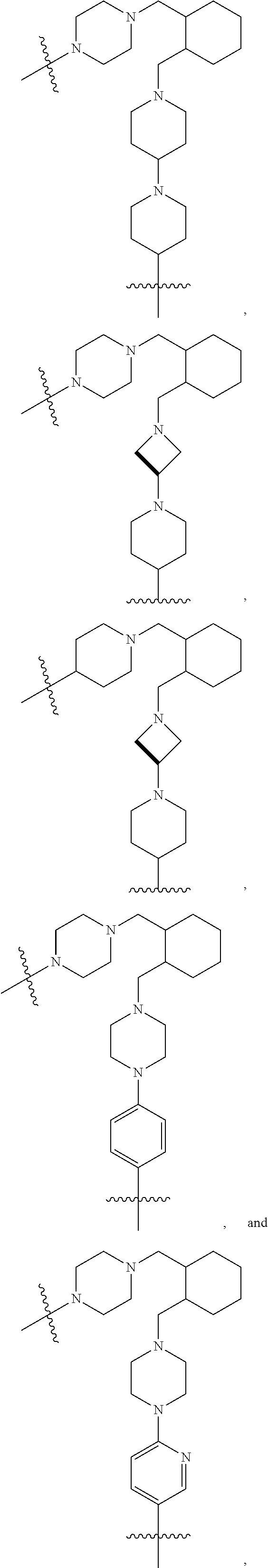

wherein the corresponding Cy2 group is unsubstituted or substituted with 1-3 Rₐ₂, the corresponding Cy4 group is unsubstituted or substituted with 1-3 Rₐ₄, and when the corresponding Cy5 group is a 5-6 membered heterocyclyl group, Cy5 is not substituted or substituted with 1-3 Rₐ₅, or Cy5 group is

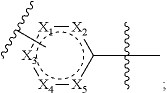

and each of Rₐ₂, Rₐ₃, Rₐ₄, Rₐ₅, R_{b1}, R_{b2}, R_{b3}, R_{b4}, and R_{b5} is at occurrence, independently selected from hydrogen and F.

In another embodiment of the present invention, the compound as shown in formula (I), or the isomer or pharmaceutically acceptable salt thereof,
C is -Cy6-L7-Cy7-L8-Cy8;
Cy6 is selected from a 5-6 membered heteroaryl group substituted with 1-3 Rₐ₆, a 9-10 membered heterocycloalkenyl group substituted with 1-3 Rₐ₆, and a 9-10 membered heteroaryl group substituted with 1-3 Rₐ₆, wherein the 5-6 membered heteroaryl group, the 9-10 membered heterocycloalkenyl group, and the heteroaryl group are preferably

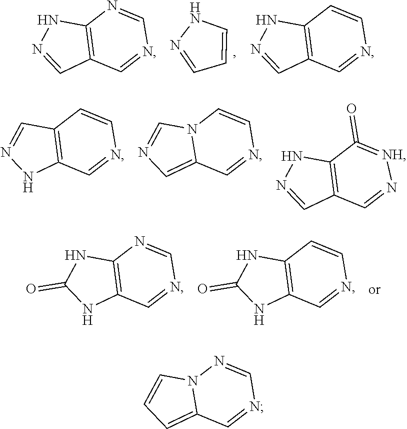

each Rₐ₆ is independently selected from hydrogen, amino, and aminocarbonyl;
-L7-Cy7-L8-Cy8 is selected from

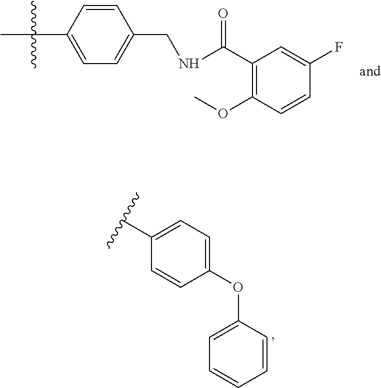 [SMILES: *c1ccc(CNC(=O)c2cc(F)ccc2OC)cc1 ; *c1ccc(Oc2ccccc2)cc1]

wherein the corresponding Cy7 group is unsubstituted or substituted with 1-3 Rₐ₇; and
Rₐ₇ is at each occurrence, independently selected from hydrogen, methyl, and F.

In another embodiment of the present invention, the compound as shown in formula (I), or the isomer or pharmaceutically acceptable salt thereof,
Cy6 is selected from

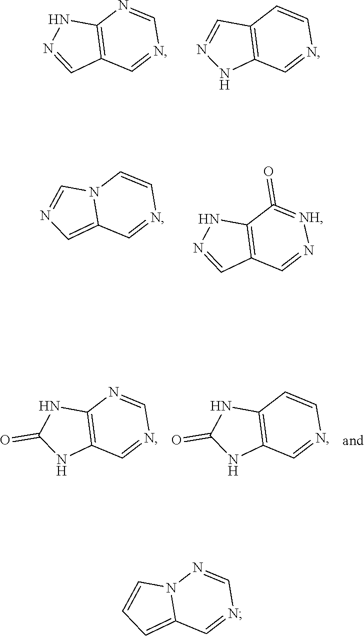

wherein these groups are unsubstituted or substituted with 1-3 Rₐ₆.

In another embodiment of the present invention, the compound as shown in formula (II), or the isomer or pharmaceutically acceptable salt thereof, having a structure as shown in formula (VI),

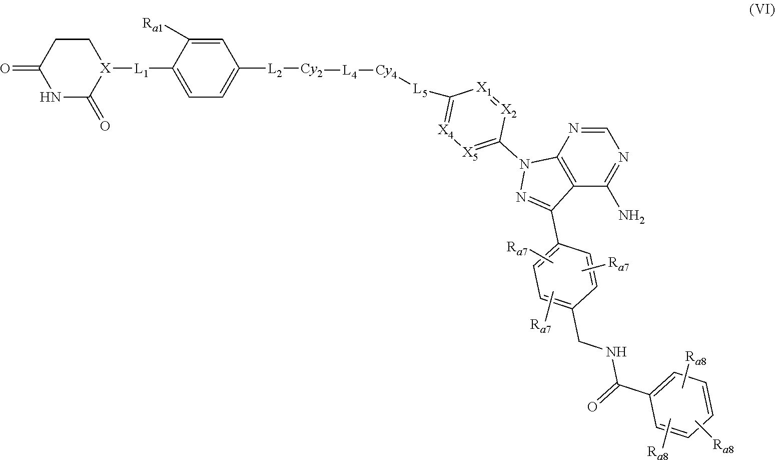

X is selected from N and CH;
L1 is a bond;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
L2, L4 and L5 are independently selected from a bond, —(CH₂)ₙ—(NH)ₘ—, —O—, and —S—,
n and m are independently any integer from zero to three;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 3-8 membered heterocycloalkenyl group, a 4-12 membered cycloalkyl group substituted with 1-3 Rₐ₄, a 4-12 membered heterocyclyl group substituted with 1-3 Rₐ₄, and a 3-8 membered heterocycloalkenyl group substituted with 1-3 Rₐ₄, wherein the heteroatoms of the 4-12 membered heterocyclyl group and the 3-8 membered heterocycloalkenyl group are selected from O, N and S;
each of Rₐ₂ and Rₐ₄ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl;
X₁ is N or CR_{b1};
X₂ is N or CR_{b2};
X₄ is N or CR_{b4};
X₅ is N or CR_{b5};
R_{b1}, R_{b2}, R_{b4} and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
each of Rₐ₇ and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

In another embodiment of the present invention, the compound as shown in formula (VI), or the isomer or pharmaceutically acceptable salt thereof,
X is selected from N and CH;
L1 is a bond;
Rₐ₁ is selected from hydrogen and methyl;
L2, L4 and L5 are independently selected from a bond and —CH₂—;
Cy2 is a 5-6 membered nitrogen-containing heterocyclyl group or a 5-6 membered nitrogen-containing heterocyclyl group substituted with 1-3 Rₐ₂, wherein the 5-6 membered nitrogen-containing heterocyclyl group is preferably

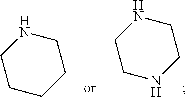 [SMILES: C1CCNCC1 ; C1CNCCN1]

Cy4 is a 5-6 membered nitrogen-containing heterocyclyl group, a 5-6 membered nitrogen-containing heterocycloalkenyl group, a 7-11 membered nitrogen-containing spiro heterocyclyl group or a 6-12 membered nitrogen-containing bridged heterocyclyl group, a 5-6 membered nitrogen-containing heterocyclyl group substituted with 1-3 Rₐ₄, a 5-6 membered nitrogen-containing heterocycloalkenyl group substituted with 1-3 Rₐ₄, a 7-11 membered nitrogen-containing spiro heterocyclyl group substituted with 1-3 Rₐ₄, or a 6-12 membered nitrogen-containing bridged heterocyclyl group substituted with 1-3 Rₐ₄, wherein the 5-6 membered nitrogen-containing heterocyclyl group is preferably

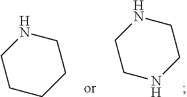 [SMILES: C1CCNCC1 ; C1CNCCN1]

the 5-6 membered nitrogen-containing heterocycloalkenyl group is preferably

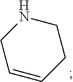

the 6-12 membered nitrogen-containing bridged heterocyclyl group is preferably

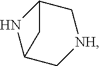

and the 7-11 membered nitrogen-containing spiro heterocyclyl group is preferably

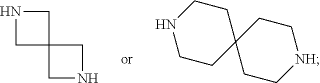 [SMILES: C1NCC12CNC2 ; C1CC2(CCN1)CCNCC2]

each of Rₐ₂ and Rₐ₄ is at occurrence, independently selected from hydrogen and methyl;
X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen, F, ethyl, and methoxy;
each Rₐ₇ is independently selected from hydrogen, methyl, and F; and
each Rₐ₈ is independently selected from hydrogen, F, and methoxy.

In another embodiment of the present invention, the compound as shown in formula (II), or the isomer or pharmaceutically acceptable salt thereof, having a structure as shown in formula (VII).

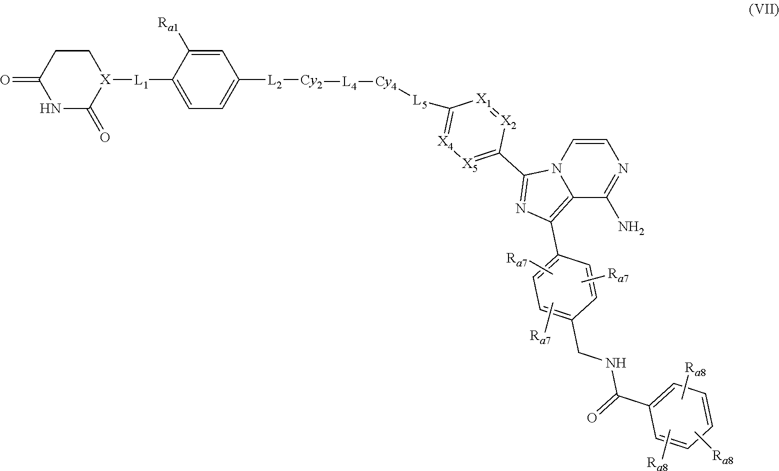

X is selected from N and CH;
L1 is a bond;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
L2, L4 and L5 are independently selected from a bond, —(CH₂)ₙ—(NH)ₘ—, —O—, and —S—;
n and m are independently any integer from zero to three;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 3-8 membered heterocycloalkenyl group, a 4-12 membered cycloalkyl group substituted with 1-3 Rₐ₄, a 4-12 membered heterocyclyl group substituted with 1-3 Rₐ₄, and a 3-8 membered heterocycloalkenyl group substituted with 1-3 Rₐ₄, wherein the heteroatoms of the 4-12 membered heterocyclyl group and the 3-8 membered heterocycloalkenyl group are selected from O, N and S;
each of Rₐ₂ and Rₐ₄ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl;
X₁ is N or CR_{b1};
X₂ is N or CR_{b2};
X₄ is N or CR_{b4};
X₅ is N or CR_{b5};
R_{b1}, R_{b2}, R_{b4}, and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
each of Rₐ₇ and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

In another embodiment of the present invention, the compound as shown in formula (VII), or the isomer or pharmaceutically acceptable salt thereof,
X is selected from N and CH;
L1 is a bond;
Rₐ₁ is selected from hydrogen and methyl;
L2, L4 and L5 are independently selected from a bond and —CH₂—;
Cy2 is a 5-6 membered nitrogen-containing heterocyclyl group or a 5-6 membered nitrogen-containing heterocyclyl group substituted with 1-3 Rₐ₂, wherein the 5-6 membered nitrogen-containing heterocyclyl group is preferably

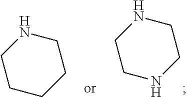 [SMILES: C1CCNCC1 ; C1CNCCN1]

Cy4 is a 5-6 membered nitrogen-containing heterocyclyl group, a 5-6 membered nitrogen-containing heterocycloalkenyl group, a 7-11 membered nitrogen-containing spiro heterocyclyl group or a 6-12 membered nitrogen-containing bridged heterocyclyl group, a 5-6 membered nitrogen-containing heterocyclyl group substituted with 1-3 Rₐ₄, a 5-6 membered nitrogen-containing heterocycloalkenyl group substituted with 1-3 Rₐ₄, a 7-11 membered nitrogen-containing spiro heterocyclyl group substituted with 1-3 Rₐ₄, or a 6-12 membered nitrogen-containing bridged heterocyclyl group substituted with 1-3 Rₐ₄, wherein the 5-6 membered nitrogen-containing heterocyclyl group is preferably

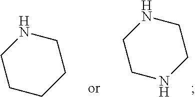 [SMILES: C1CCNCC1 ; C1CNCCN1]

the 5-6 membered nitrogen-containing heterocycloalkenyl group is preferably

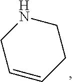

the 6-12 membered nitrogen-containing bridged heterocyclyl group is preferably

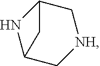

and the 7-11 membered nitrogen-containing spiro heterocyclyl group is preferably

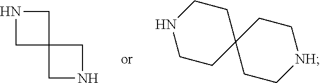 [SMILES: C1NCC12CNC2 ; C1CC2(CCN1)CCNCC2]

each of Rₐ₂ and Rₐ₄ is at occurrence, independently selected from hydrogen and methyl;
X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen, F, ethyl, and methoxy;
each Rₐ₇ is independently selected from hydrogen, methyl, and F; and
each Rₐ₈ is independently selected from hydrogen, F, and methoxy.

The compound of any embodiment of the present invention, or the isomer or pharmaceutically acceptable salt thereof,
C is

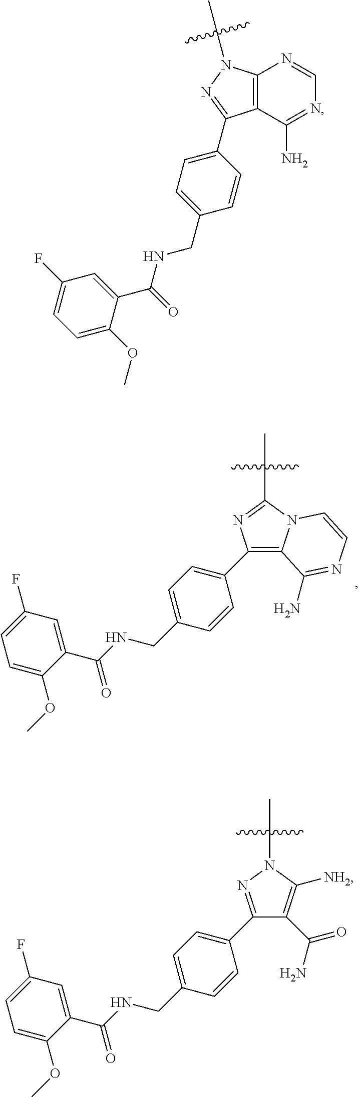 [SMILES: *n1nc(-c2ccc(CNC(=O)c3cc(F)ccc3OC)cc2)c2c(N)ncnc21 ; *c1nc(-c2ccc(CNC(=O)c3cc(F)ccc3OC)cc2)c2c(N)nccn12 ; *n1nc(-c2ccc(CNC(=O)c3cc(F)ccc3OC)cc2)c(C(N)=O)c1N]

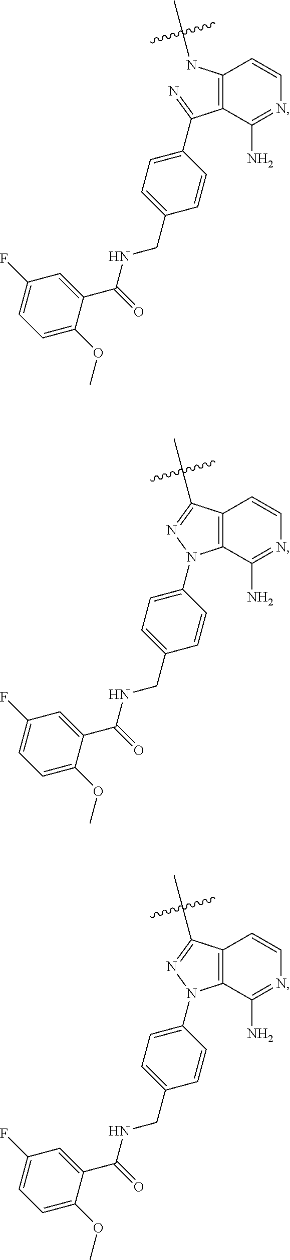 [SMILES: *Nc1ccnc(N)c1C(=N)c1ccc(CNC(=O)c2cc(F)ccc2OC)cc1 ; *c1nn(-c2ccc(CNC(=O)c3cc(F)ccc3OC)cc2)c2c(N)nccc12 ; *c1nn(-c2ccc(CNC(=O)c3cc(F)ccc3OC)cc2)c2c(N)nccc12]

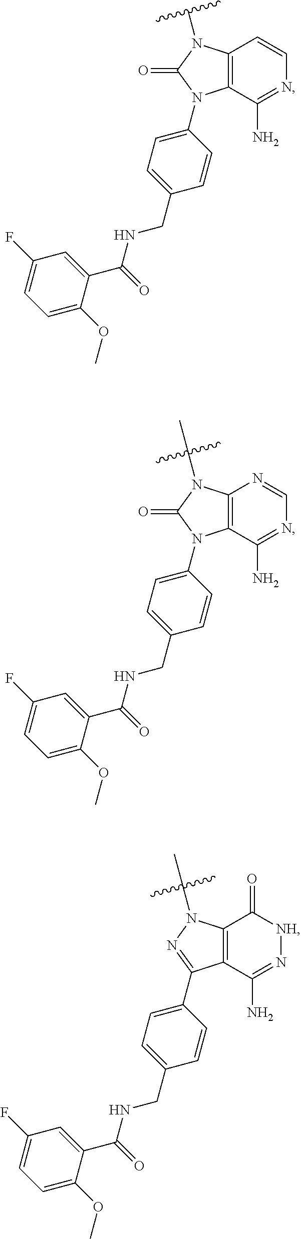 [SMILES: *n1c(=O)n(-c2ccc(CNC(=O)c3cc(F)ccc3OC)cc2)c2c(N)nccc21 ; *n1c(=O)n(-c2ccc(CNC(=O)c3cc(F)ccc3OC)cc2)c2c(N)ncnc21 ; *n1nc(-c2ccc(CNC(=O)c3cc(F)ccc3OC)cc2)c2c(N)n[nH]c(=O)c21]

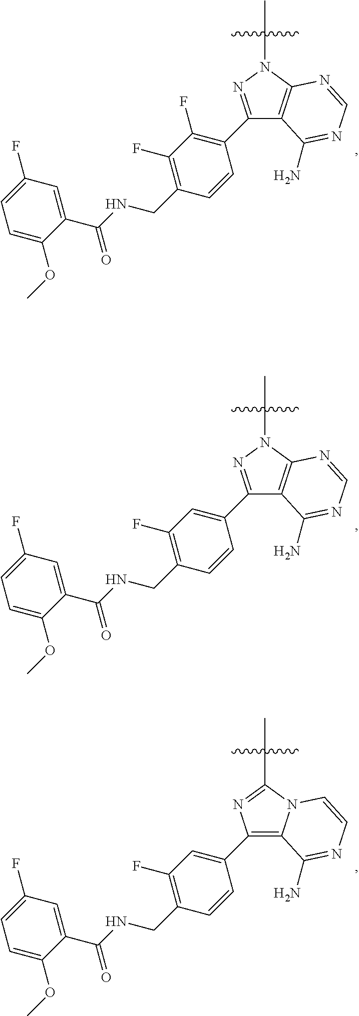 [SMILES: *n1nc(-c2ccc(CNC(=O)c3cc(F)ccc3OC)c(F)c2F)c2c(N)ncnc21 ; *n1nc(-c2ccc(CNC(=O)c3cc(F)ccc3OC)c(F)c2)c2c(N)ncnc21 ; *c1nc(-c2ccc(CNC(=O)c3cc(F)ccc3OC)c(F)c2)c2c(N)nccn12]

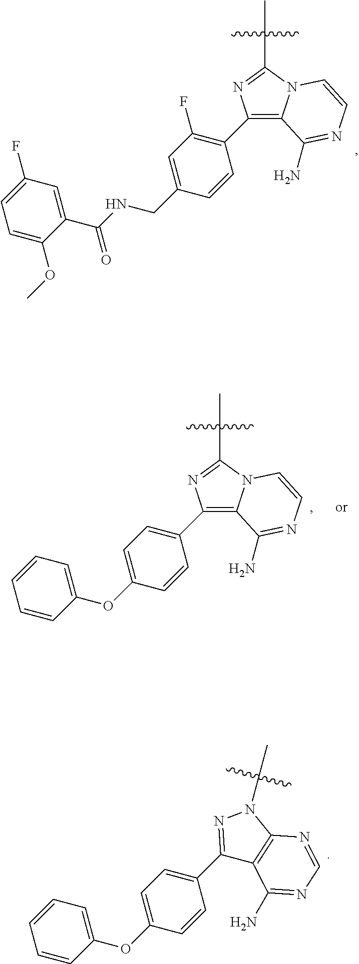 [SMILES: *c1nc(-c2ccc(CNC(=O)c3cc(F)ccc3OC)cc2F)c2c(N)nccn12 ; *c1nc(-c2ccc(Oc3ccccc3)cc2)c2c(N)nccn12 ; *n1nc(-c2ccc(Oc3ccccc3)cc2)c2c(N)ncnc21]

In another embodiment of the present invention, the compound as shown in any one of formula (I), (II) or (V), or the isomer or pharmaceutically acceptable salt thereof,
Cy4 is selected from a 5-6 membered heterocyclyl group, a 5-6 membered heteroalkenyl group, a 7-12 membered bridged heterocyclyl group, a 7-12 membered spiro heterocyclyl group, a 5-6 membered heterocyclyl group substituted with 1-3 Rₐ₄, a 5-6 membered heteroalkenyl group substituted with 1-3 Rₐ₄, a 7-12 membered bridged heterocyclyl group substituted with 1-3 Rₐ₄, and a 7-12 membered spiro heterocyclyl group substituted with 1-3 Rₐ₄.

In another embodiment of the present invention, the compound as shown in any one of formula (I), (II) or (V), or the isomer or pharmaceutically acceptable salt thereof,
Cy4 is selected from a 5-6 membered heterocyclyl group and a 5-6 membered heterocyclyl group substituted with 1-3 Rₐ₄.

In another embodiment of the present invention, the compound as shown in any one of formula (I), (II) or (V), or the isomer or pharmaceutically acceptable salt thereof,
Cy4 is selected from a 5-6 membered heteroalkenyl group, a 7-12 membered bridged heterocyclyl group, a 7-12 membered spiro heterocyclyl group, a 5-6 membered heteroalkenyl group substituted with 1-3 Rₐ₄, a 7-12 membered bridged heterocyclyl group substituted with 1-3 Rₐ₄, and a 7-12 membered spiro heterocyclyl group substituted with 1-3 Rₐ₄.

In another embodiment of the present invention, the compound of any one of formula (I), (II), (III), (IV) or (V), or the isomer or pharmaceutically acceptable salt thereof,
Cy2 is selected from

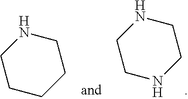 [SMILES: C1CCNCC1 ; C1CNCCN1]

In another embodiment of the present invention, the compound of any one of formula (I), (II) or (V), or the isomer or pharmaceutically acceptable salt thereof,
Cy4 is selected from

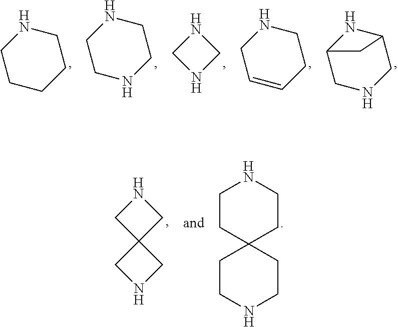

In another embodiment of the present invention, the compound of any one of formula (I), (II), (III), (IV) or (V), or the isomer or pharmaceutically acceptable salt thereof,
Cy5 is selected from

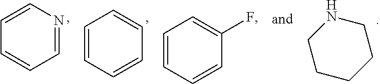

In another embodiment of the present invention, the compound of any one of formula (I), (II), (III), (IV) or (V), or the isomer or pharmaceutically acceptable salt thereof,
L1, L2, L3, L5, L6 and L7 are independently a bond.

In another embodiment of the present invention, the compound of any one of formula (I), (II), (III), (IV) or (V), or the isomer or pharmaceutically acceptable salt thereof.
L4 is —CH₂—.

In another embodiment of the present invention, the compound of any one of formula (I), (II), (III), (IV) or (V), or the isomer or pharmaceutically acceptable salt thereof.
L8 is —O— or —CH₂—NH—C(O)—.

In another embodiment of the present invention, the compound of any one of formula (I), (II), (III), (IV) or (V), or the isomer or pharmaceutically acceptable salt thereof,
Cy6 is selected from

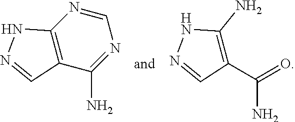 [SMILES: Nc1ncnc2[nH]ncc12 ; NC(=O)c1cn[nH]c1N]

In any embodiment of the present invention, Cy6 is selected from a 5-6 membered heteroaryl group substituted with 1-3 Ra, and a 9-10 membered fused heteroaryl group substituted with 1-3 Rₐ₆, wherein the 5-6 membered heteroaryl group and the 9-10 membered fused heteroaryl group are preferably

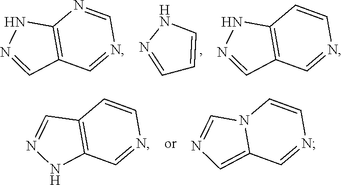

preferably

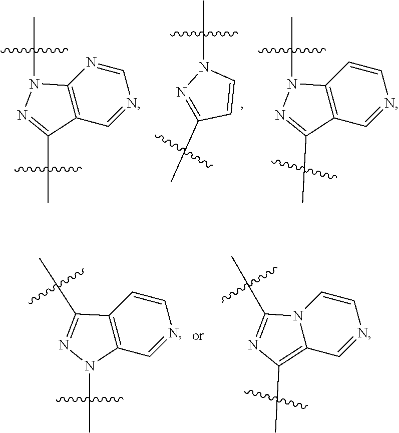 [SMILES: *c1nn(*)c2ncncc12 ; *c1ccn(*)n1 ; *c1nn(*)c2ccncc12 ; *c1nn(*)c2cnccc12 ; *c1nc(*)n2ccncc12]

more preferably

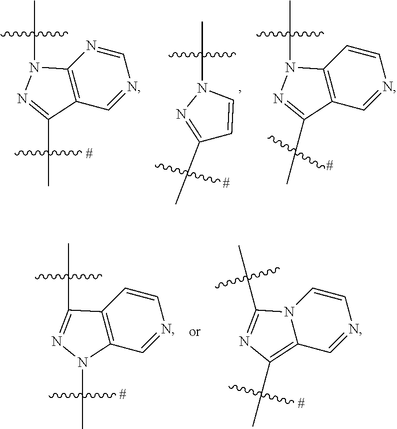

wherein the * end is connected to L6, and the #end is connected to L7; more preferably

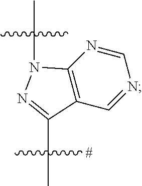 [SMILES: *c1nn(*)c2ncncc12]

and more preferably

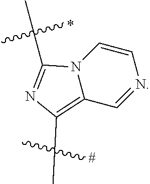 [SMILES: *c1nc(*)n2ccncc12]

In another embodiment of the present invention, the compound as shown in formula (I), or the isomer or pharmaceutically acceptable salt thereof is shown in Table 1-3:

TABLE 1

| Serial No. | Structure |
|---|---|
| I-1 | 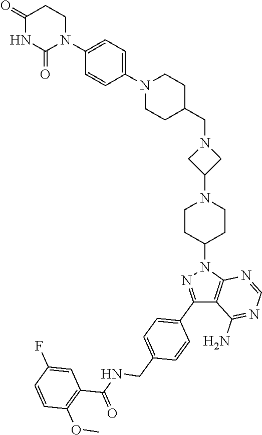 [SMILES: COc1ccc(F)cc1C(=O)NCc1ccc(-c2nn(C3CCN(C4CN(CC5CCN(c6ccc(N7CCC(=O)NC7=O)cc6)CC5)C4)CC3)c3ncnc(N)c23)cc1] |
| I-2 | 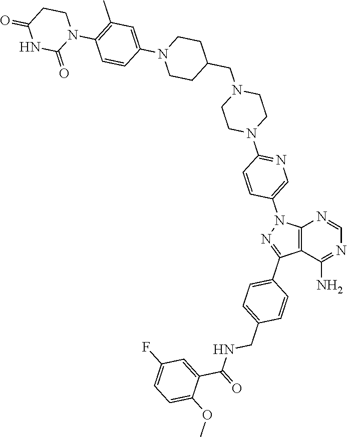 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1] |
| I-3 | 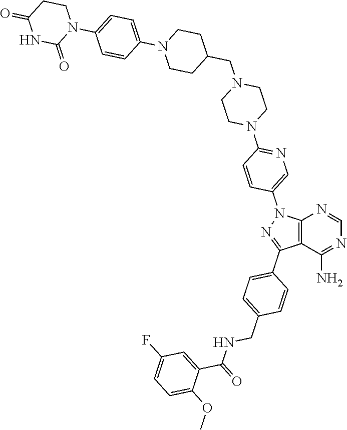 [SMILES: COc1ccc(F)cc1C(=O)NCc1ccc(-c2nn(-c3ccc(N4CCN(CC5CCN(c6ccc(N7CCC(=O)NC7=O)cc6)CC5)CC4)nc3)c3ncnc(N)c23)cc1] |
| I-4 | 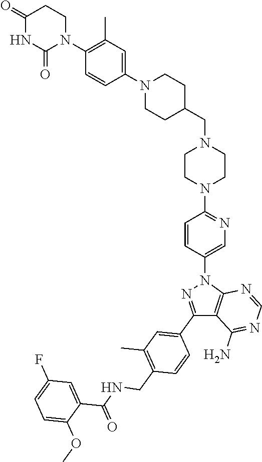 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1C] |
| I-5 | 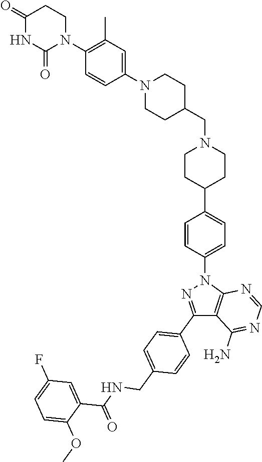 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1] |
| I-6 | 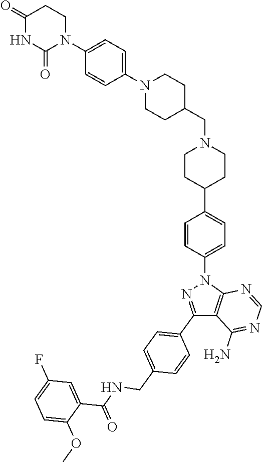 [SMILES: COc1ccc(F)cc1C(=O)NCc1ccc(-c2nn(-c3ccc(C4CCN(CC5CCN(c6ccc(N7CCC(=O)NC7=O)cc6)CC5)CC4)cc3)c3ncnc(N)c23)cc1] |
| I-7 | 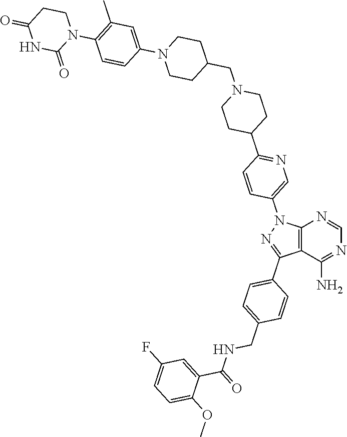 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c3ncnc(N)c23)cc1] |
| I-8 | 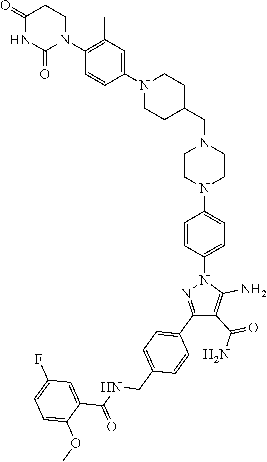 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c(N)c2C(N)=O)cc1] |
| I-9 | 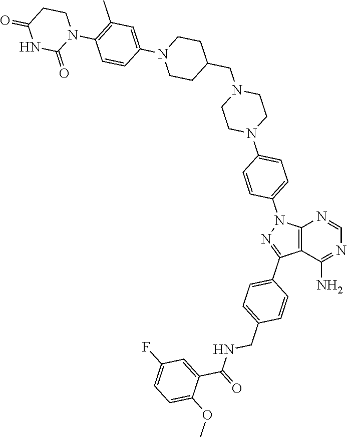 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ncnc(N)c23)cc1] |
| I-10 | 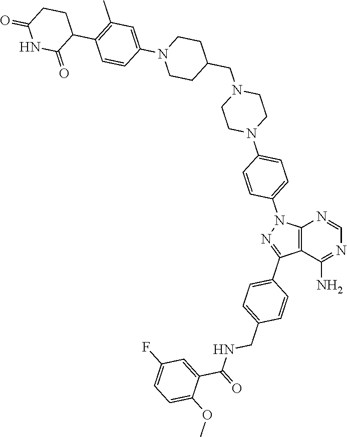 [SMILES: COc1ccc(F)cc1C(=O)NCc1ccc(-c2nn(-c3ccc(N4CCN(CC5CCN(c6ccc(C7CCC(=O)NC7=O)c(C)c6)CC5)CC4)cc3)c3ncnc(N)c23)cc1] |
| I-11 | 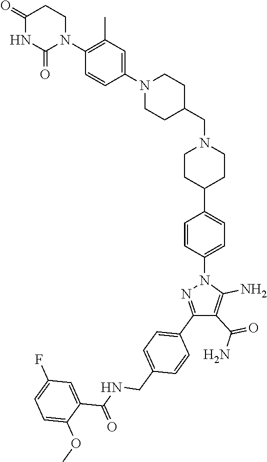 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c(N)c2C(N)=O)cc1] |
| I-12 | 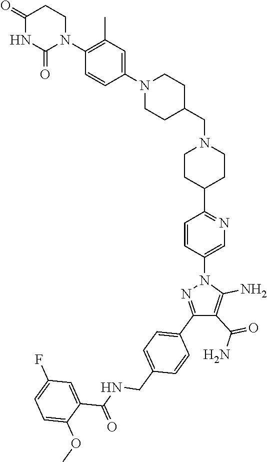 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c(N)c2C(N)=O)cc1] |
| I-13 | 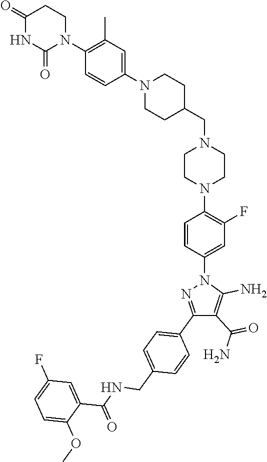 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)c(F)c3)c(N)c2C(N)=O)cc1] |
| I-14 | 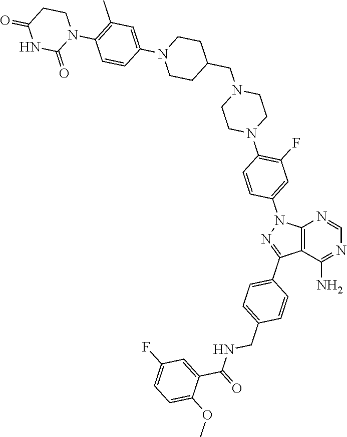 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)c(F)c3)c3ncnc(N)c23)cc1] |
| I-15 | 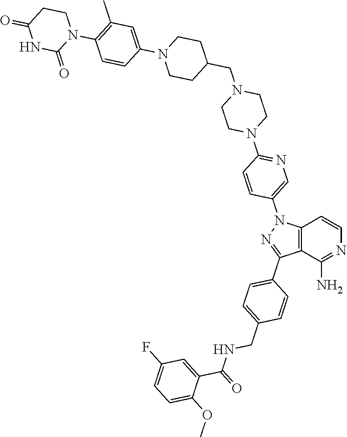 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ccnc(N)c23)cc1] |
| I-16 | 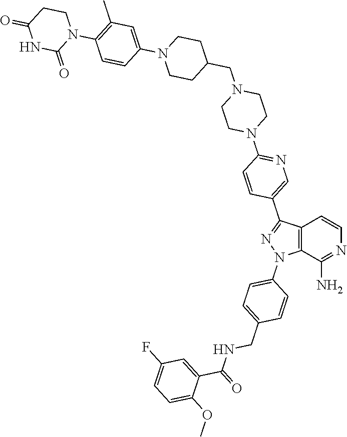 [SMILES: COc1ccc(F)cc1C(=O)NCc1ccc(-n2nc(-c3ccc(N4CCN(CC5CCN(c6ccc(N7CCC(=O)NC7=O)c(C)c6)CC5)CC4)nc3)c3ccnc(N)c32)cc1] |
| I-17 | 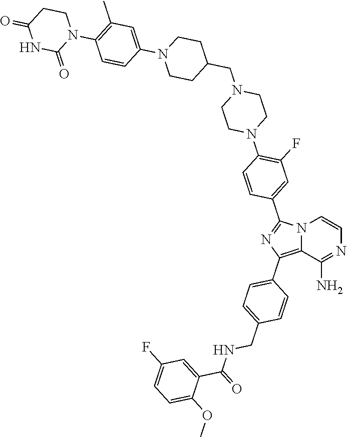 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)c(F)c3)n3ccnc(N)c23)cc1] |
| I-18 | 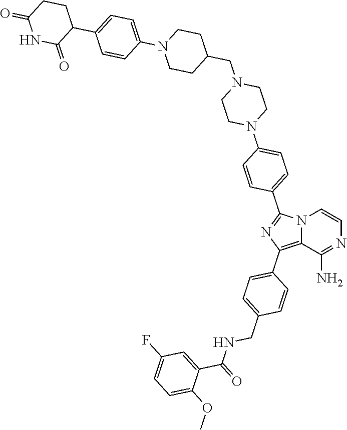 [SMILES: COc1ccc(F)cc1C(=O)NCc1ccc(-c2nc(-c3ccc(N4CCN(CC5CCN(c6ccc(C7CCC(=O)NC7=O)cc6)CC5)CC4)cc3)n3ccnc(N)c23)cc1] |
| I-19 | 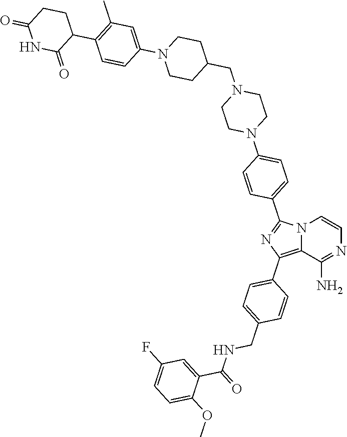 [SMILES: COc1ccc(F)cc1C(=O)NCc1ccc(-c2nc(-c3ccc(N4CCN(CC5CCN(c6ccc(C7CCC(=O)NC7=O)c(C)c6)CC5)CC4)cc3)n3ccnc(N)c23)cc1] |
| I-20 | 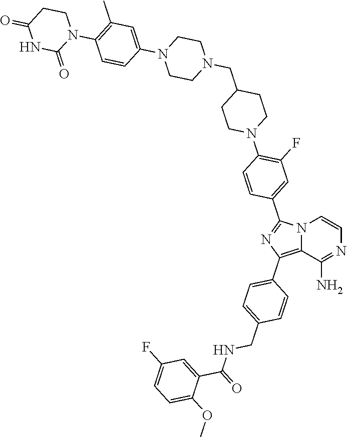 [SMILES: COc1ccc(F)cc1C(=O)NCc1ccc(-c2nc(-c3ccc(N4CCC(CN5CCN(c6ccc(N7CCC(=O)NC7=O)c(C)c6)CC5)CC4)c(F)c3)n3ccnc(N)c23)cc1] |
| I-21 | 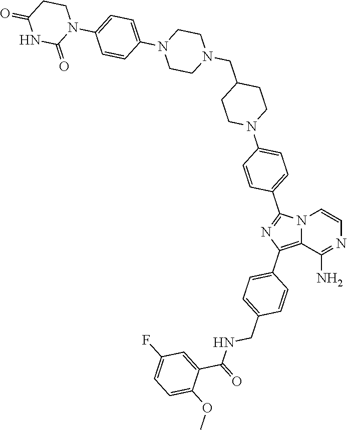 [SMILES: COc1ccc(F)cc1C(=O)NCc1ccc(-c2nc(-c3ccc(N4CCC(CN5CCN(c6ccc(N7CCC(=O)NC7=O)cc6)CC5)CC4)cc3)n3ccnc(N)c23)cc1] |
| I-22 | 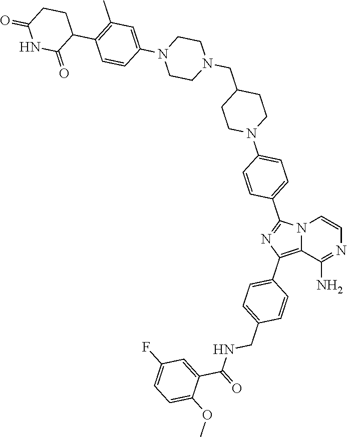 [SMILES: COc1ccc(F)cc1C(=O)NCc1ccc(-c2nc(-c3ccc(N4CCC(CN5CCN(c6ccc(C7CCC(=O)NC7=O)c(C)c6)CC5)CC4)cc3)n3ccnc(N)c23)cc1] |
| I-23 | 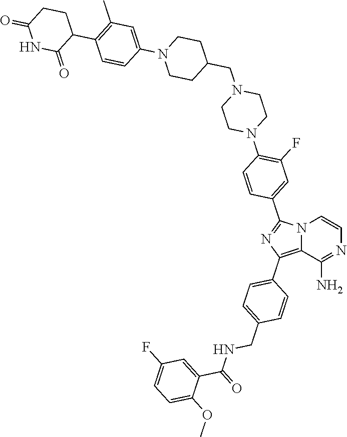 [SMILES: COc1ccc(F)cc1C(=O)NCc1ccc(-c2nc(-c3ccc(N4CCN(CC5CCN(c6ccc(C7CCC(=O)NC7=O)c(C)c6)CC5)CC4)c(F)c3)n3ccnc(N)c23)cc1] |
| I-24 | 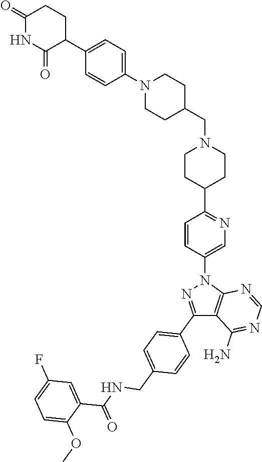 [SMILES: COc1ccc(F)cc1C(=O)NCc1ccc(-c2nn(-c3ccc(C4CCN(CC5CCN(c6ccc(C7CCC(=O)NC7=O)cc6)CC5)CC4)nc3)c3ncnc(N)c23)cc1] |
| I-25 | 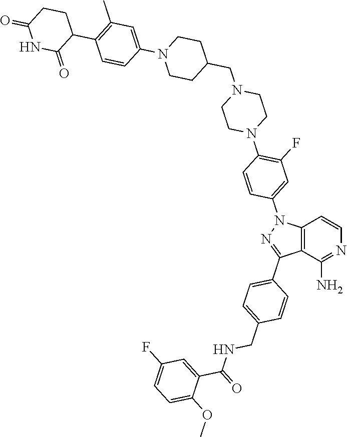 [SMILES: COc1ccc(F)cc1C(=O)NCc1ccc(-c2nn(-c3ccc(N4CCN(CC5CCN(c6ccc(C7CCC(=O)NC7=O)c(C)c6)CC5)CC4)c(F)c3)c3ccnc(N)c23)cc1] |
| I-26 | 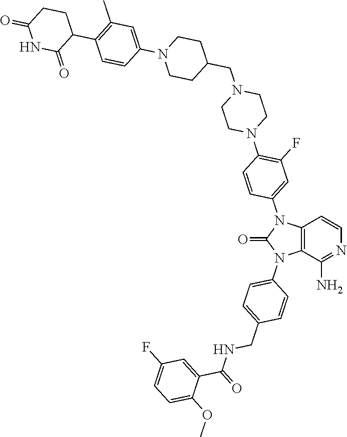 [SMILES: COc1ccc(F)cc1C(=O)NCc1ccc(-n2c(=O)n(-c3ccc(N4CCN(CC5CCN(c6ccc(C7CCC(=O)NC7=O)c(C)c6)CC5)CC4)c(F)c3)c3ccnc(N)c32)cc1] |
| I-27 | 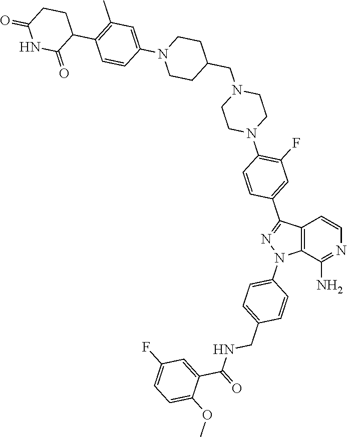 [SMILES: COc1ccc(F)cc1C(=O)NCc1ccc(-n2nc(-c3ccc(N4CCN(CC5CCN(c6ccc(C7CCC(=O)NC7=O)c(C)c6)CC5)CC4)c(F)c3)c3ccnc(N)c32)cc1] |
| I-28 | 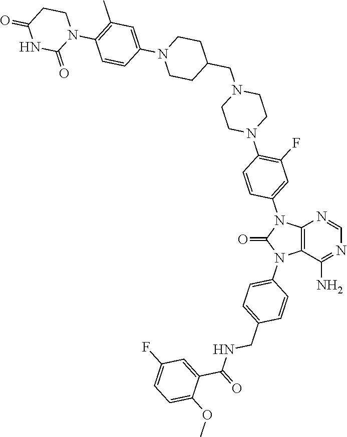 [SMILES: COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)c(F)c3)c3ncnc(N)c32)cc1] |
| I-29 | 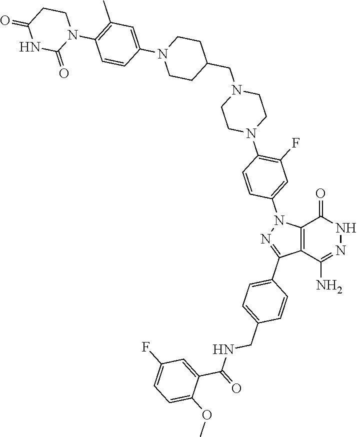 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)c(F)c3)c3c(=O)[nH]nc(N)c23)cc1] |
| I-30 | 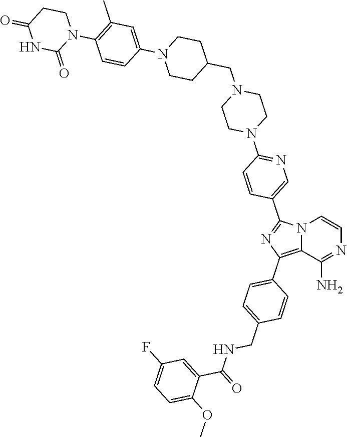 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)nc3)n3ccnc(N)c23)cc1] |
| I-31 | 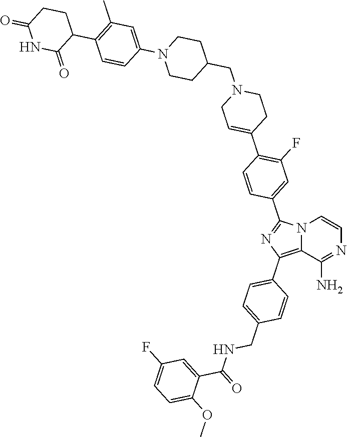 [SMILES: COc1ccc(F)cc1C(=O)NCc1ccc(-c2nc(-c3ccc(C4=CCN(CC5CCN(c6ccc(C7CCC(=O)NC7=O)c(C)c6)CC5)CC4)c(F)c3)n3ccnc(N)c23)cc1] |
| I-32 | 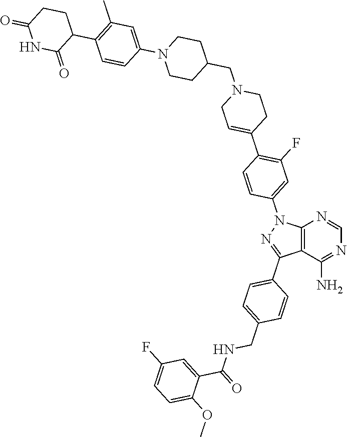 [SMILES: COc1ccc(F)cc1C(=O)NCc1ccc(-c2nn(-c3ccc(C4=CCN(CC5CCN(c6ccc(C7CCC(=O)NC7=O)c(C)c6)CC5)CC4)c(F)c3)c3ncnc(N)c23)cc1] |
| I-33 | 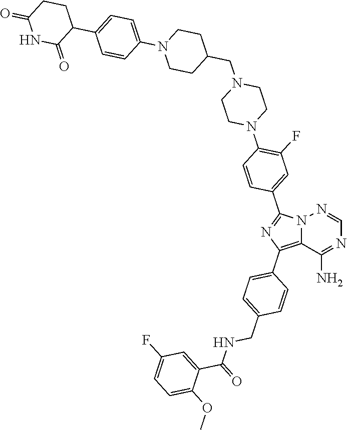 [SMILES: COc1ccc(F)cc1C(=O)NCc1ccc(-c2nc(-c3ccc(N4CCN(CC5CCN(c6ccc(C7CCC(=O)NC7=O)cc6)CC5)CC4)c(F)c3)n3ncnc(N)c23)cc1] |
| I-34 | 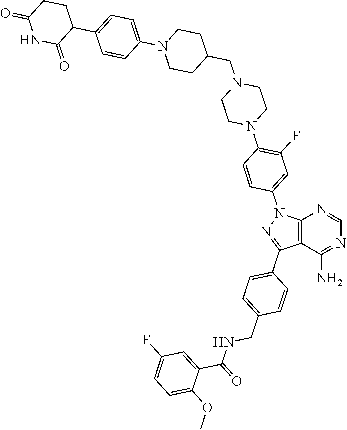 [SMILES: COc1ccc(F)cc1C(=O)NCc1ccc(-c2nn(-c3ccc(N4CCN(CC5CCN(c6ccc(C7CCC(=O)NC7=O)cc6)CC5)CC4)c(F)c3)c3ncnc(N)c23)cc1] |
| I-35 | 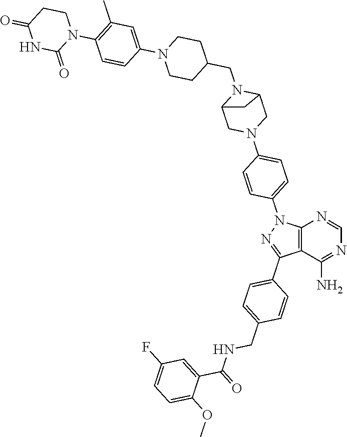 [SMILES: COc1ccc(F)cc1C(=O)NCc1ccc(-c2nn(-c3ccc(N4CC5CC(C4)N5CC4CCN(c5ccc(N6CCC(=O)NC6=O)c(C)c5)CC4)cc3)c3ncnc(N)c23)cc1] |
| I-36 | 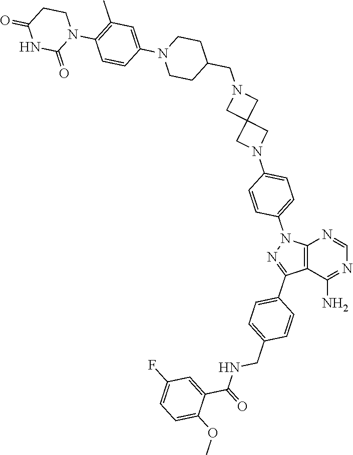 [SMILES: COc1ccc(F)cc1C(=O)NCc1ccc(-c2nn(-c3ccc(N4CC5(CN(CC6CCN(c7ccc(N8CCC(=O)NC8=O)c(C)c7)CC6)C5)C4)cc3)c3ncnc(N)c23)cc1] |
| I-37 | 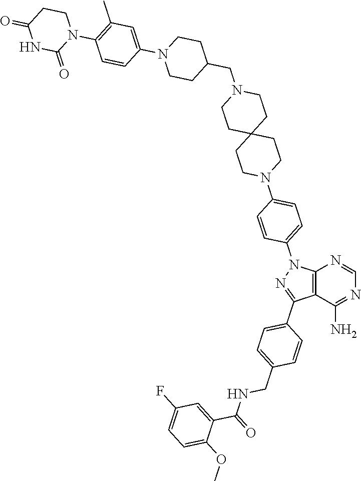 [SMILES: COc1ccc(F)cc1C(=O)NCc1ccc(-c2nn(-c3ccc(N4CCC5(CCN(CC6CCN(c7ccc(N8CCC(=O)NC8=O)c(C)c7)CC6)CC5)CC4)cc3)c3ncnc(N)c23)cc1] |
| I-38 | 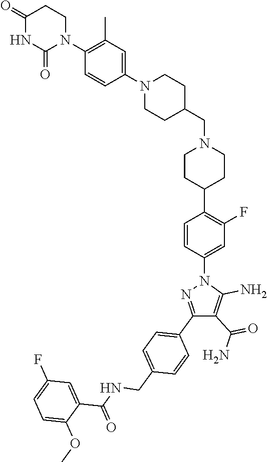 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)c(F)c3)c(N)c2C(N)=O)cc1] |
| I-39 | 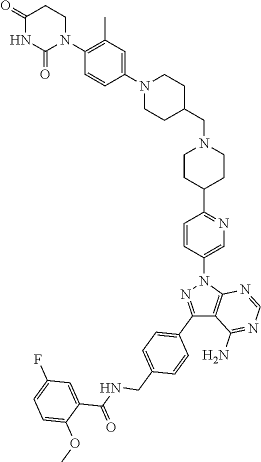 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c3ncnc(N)c23)cc1] |
| I-40 | 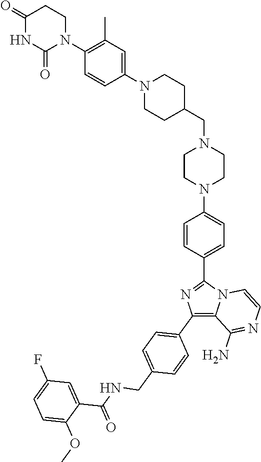 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1] |
| I-41 | 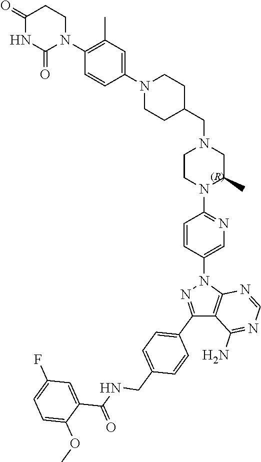 |
| I-42 | 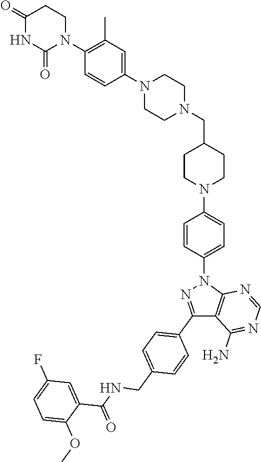 [SMILES: COc1ccc(F)cc1C(=O)NCc1ccc(-c2nn(-c3ccc(N4CCC(CN5CCN(c6ccc(N7CCC(=O)NC7=O)c(C)c6)CC5)CC4)cc3)c3ncnc(N)c23)cc1] |
| I-43 | 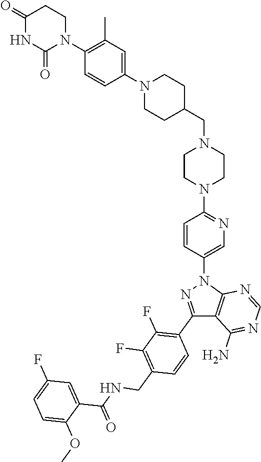 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)c(F)c1F] |
| I-44 | 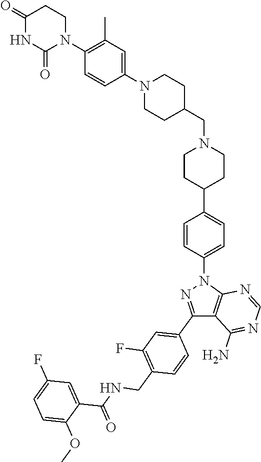 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1F] |
| I-45 | 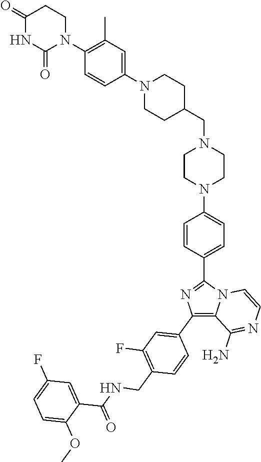 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1F] |
| I-46 | 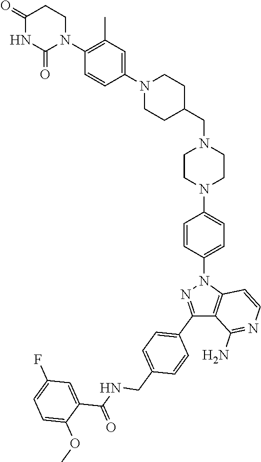 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ccnc(N)c23)cc1] |
| I-47 | 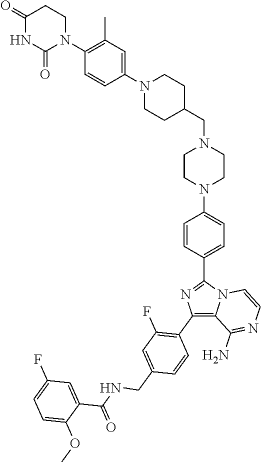 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)c(F)c1] |
| I-48 | 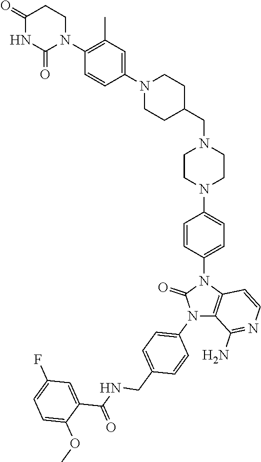 [SMILES: COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)cc3)c3ccnc(N)c32)cc1] |
| I-49 | 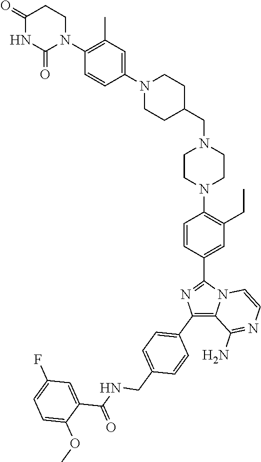 [SMILES: CCc1cc(-c2nc(-c3ccc(CNC(=O)c4cc(F)ccc4OC)cc3)c3c(N)nccn23)ccc1N1CCN(CC2CCN(c3ccc(N4CCC(=O)NC4=O)c(C)c3)CC2)CC1] |
| I-50 | 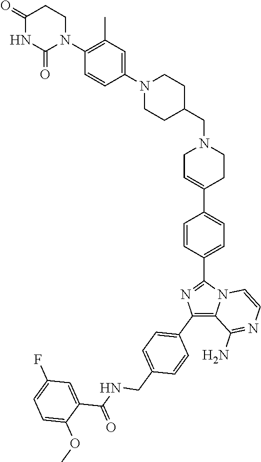 [SMILES: COc1ccc(F)cc1C(=O)NCc1ccc(-c2nc(-c3ccc(C4=CCN(CC5CCN(c6ccc(N7CCC(=O)NC7=O)c(C)c6)CC5)CC4)cc3)n3ccnc(N)c23)cc1] |
| I-51 | 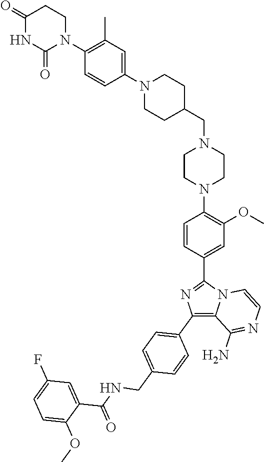 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)c(OC)c3)n3ccnc(N)c23)cc1] |
| I-52 | 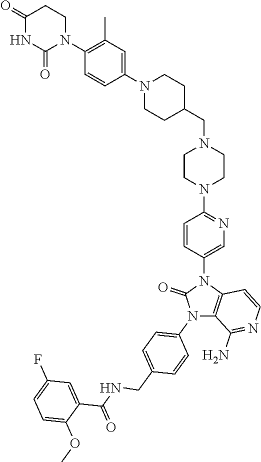 [SMILES: COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)nc3)c3ccnc(N)c32)cc1] |
| I-53 | 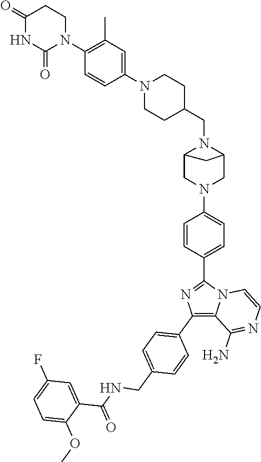 [SMILES: COc1ccc(F)cc1C(=O)NCc1ccc(-c2nc(-c3ccc(N4CC5CC(C4)N5CC4CCN(c5ccc(N6CCC(=O)NC6=O)c(C)c5)CC4)cc3)n3ccnc(N)c23)cc1] |
| I-54 | 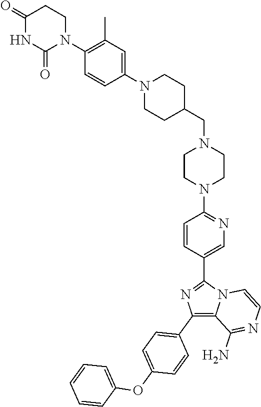 [SMILES: Cc1cc(N2CCC(CN3CCN(c4ccc(-c5nc(-c6ccc(Oc7ccccc7)cc6)c6c(N)nccn56)cn4)CC3)CC2)ccc1N1CCC(=O)NC1=O] |
| I-55 | 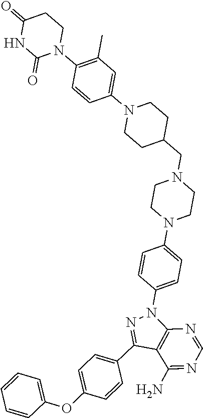 [SMILES: Cc1cc(N2CCC(CN3CCN(c4ccc(-n5nc(-c6ccc(Oc7ccccc7)cc6)c6c(N)ncnc65)cc4)CC3)CC2)ccc1N1CCC(=O)NC1=O] |

TABLE 2

| Serial No. | Structure |
|---|---|
| II-1 | 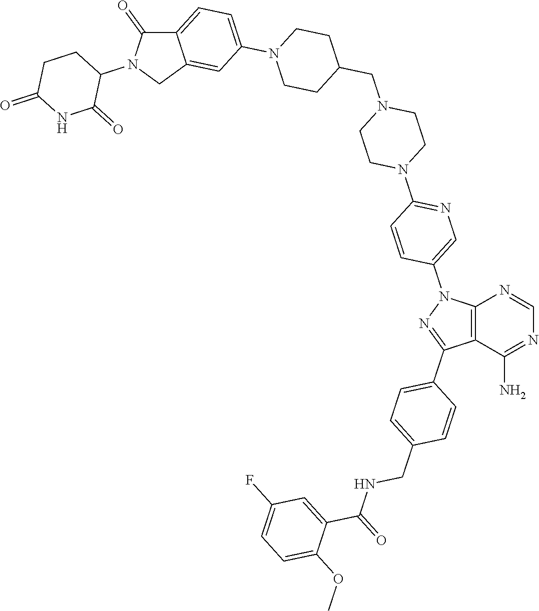 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1] |
| II-2 | 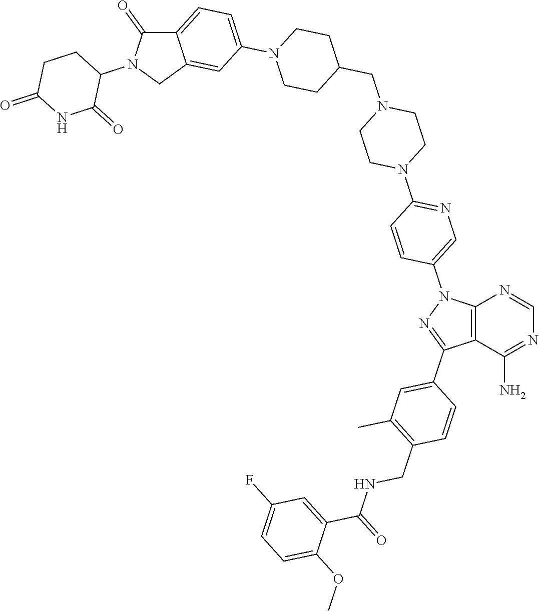 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1C] |
| II-3 | 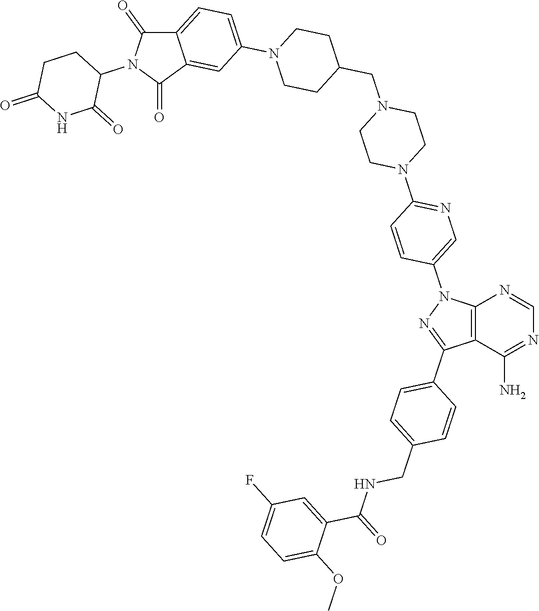 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1] |
| II-4 | 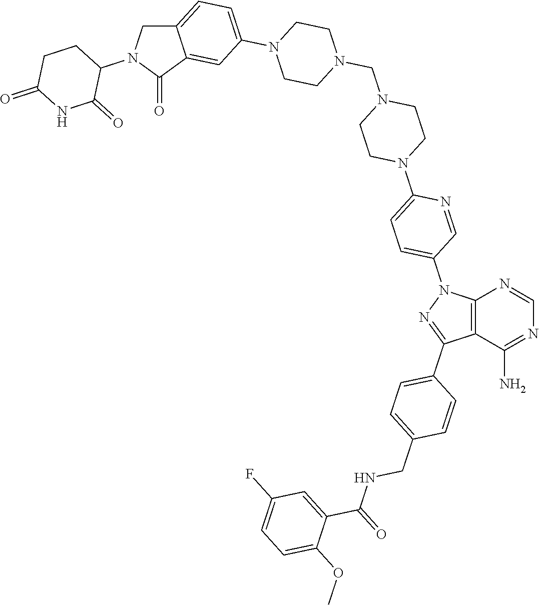 [SMILES: COc1ccc(F)cc1C(=O)NCc1ccc(-c2nn(-c3ccc(N4CCN(CN5CCN(c6ccc7c(c6)C(=O)N(C6CCC(=O)NC6=O)C7)CC5)CC4)nc3)c3ncnc(N)c23)cc1] |
| II-5 | 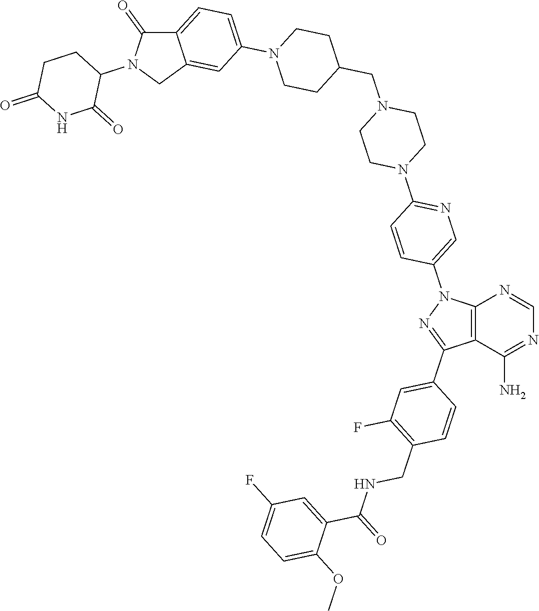 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1F] |
| II-6 | 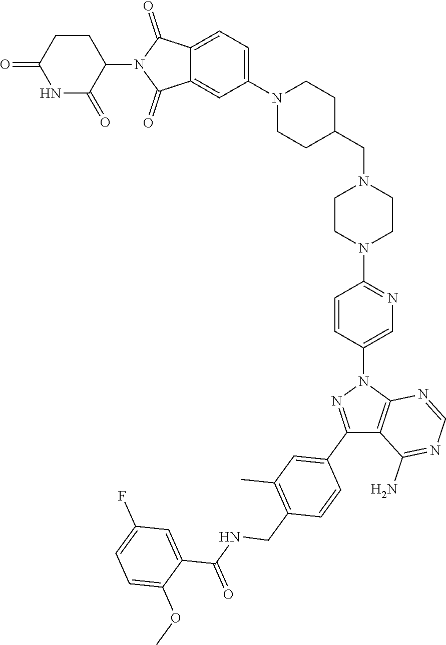 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1C] |
| II-7 | 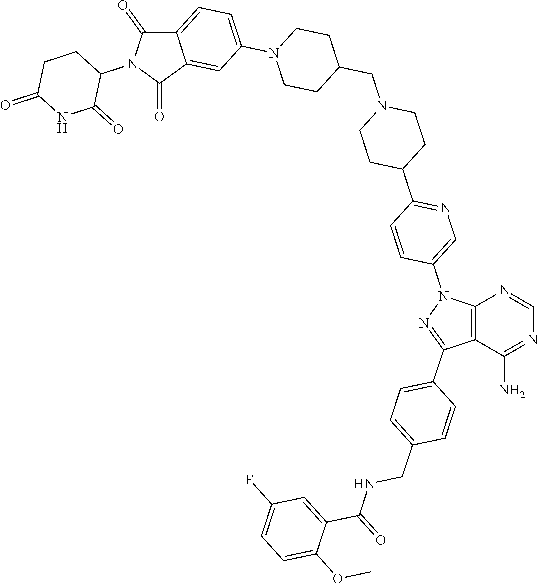 [SMILES: COc1ccc(F)cc1C(=O)NCc1ccc(-c2nn(-c3ccc(C4CCN(CC5CCN(c6ccc7c(c6)C(=O)N(C6CCC(=O)NC6=O)C7=O)CC5)CC4)nc3)c3ncnc(N)c23)cc1] |
| II-8 | 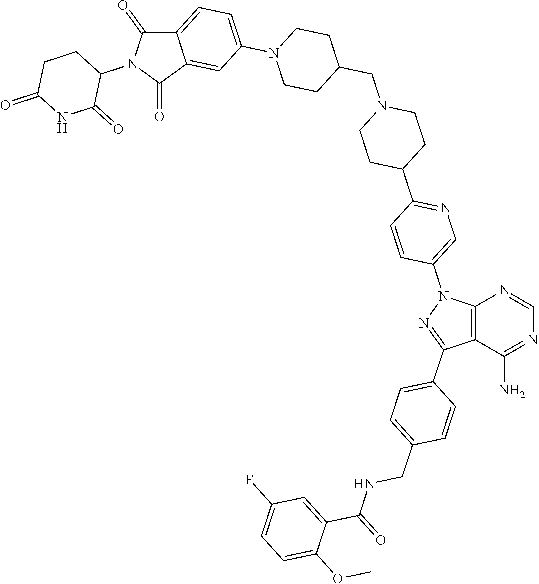 [SMILES: COc1ccc(F)cc1C(=O)NCc1ccc(-c2nn(-c3ccc(C4CCN(CC5CCN(c6ccc7c(c6)C(=O)N(C6CCC(=O)NC6=O)C7=O)CC5)CC4)nc3)c3ncnc(N)c23)cc1] |
| II-9 | 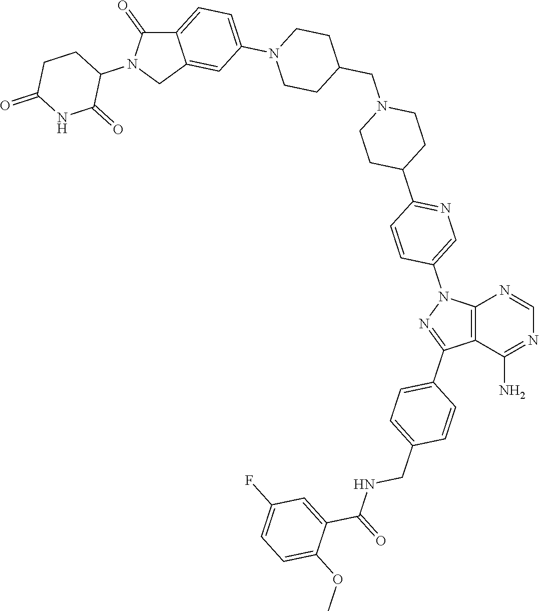 [SMILES: COc1ccc(F)cc1C(=O)NCc1ccc(-c2nn(-c3ccc(C4CCN(CC5CCN(c6ccc7c(c6)CN(C6CCC(=O)NC6=O)C7=O)CC5)CC4)nc3)c3ncnc(N)c23)cc1] |
| II-10 | 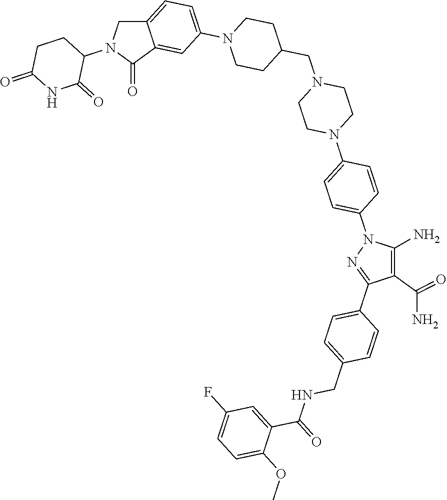 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)cc3)c(N)c2C(N)=O)cc1] |
| II-11 | 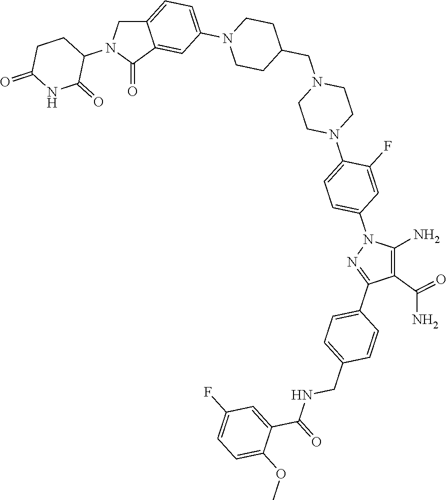 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)c(F)c3)c(N)c2C(N)=O)cc1] |
| II-12 | 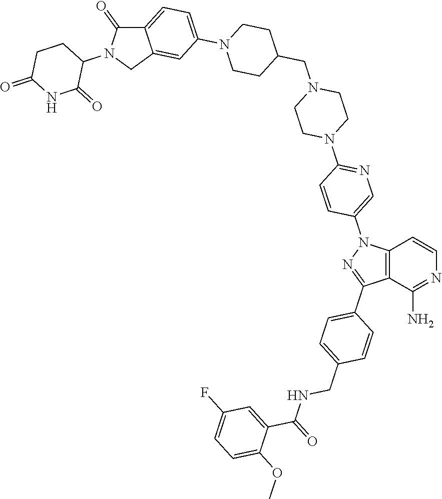 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ccnc(N)c23)cc1] |
| II-13 | 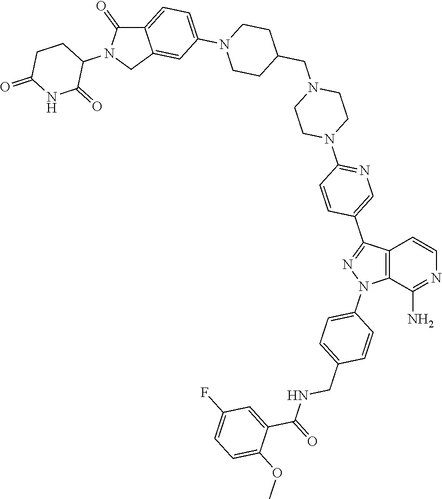 [SMILES: COc1ccc(F)cc1C(=O)NCc1ccc(-n2nc(-c3ccc(N4CCN(CC5CCN(c6ccc7c(c6)CN(C6CCC(=O)NC6=O)C7=O)CC5)CC4)nc3)c3ccnc(N)c32)cc1] |
| II-14 | 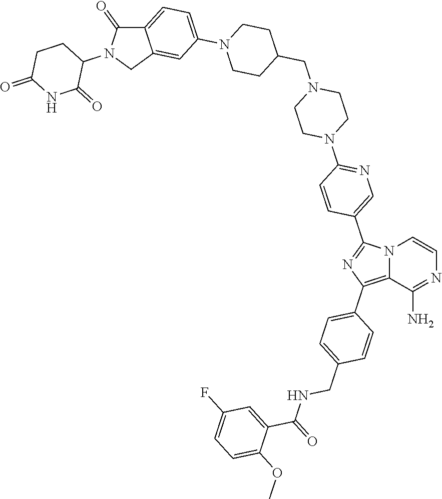 [SMILES: COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)nc3)n3ccnc(N)c23)cc1] |
| II-15 | 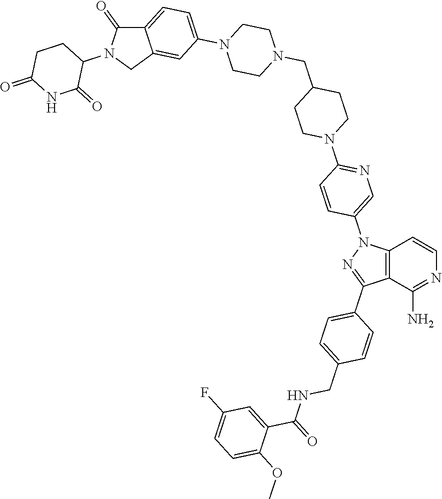 [SMILES: COc1ccc(F)cc1C(=O)NCc1ccc(-c2nn(-c3ccc(N4CCC(CN5CCN(c6ccc7c(c6)CN(C6CCC(=O)NC6=O)C7=O)CC5)CC4)nc3)c3ccnc(N)c23)cc1] |
| II-16 | 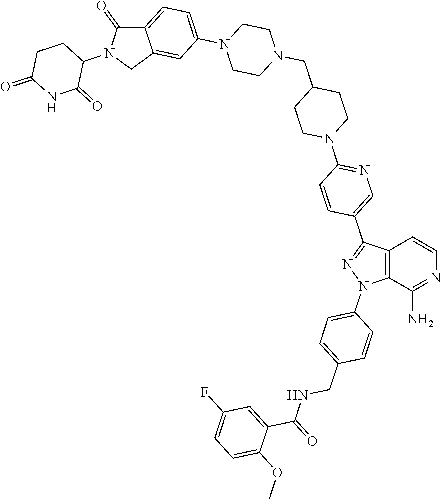 [SMILES: COc1ccc(F)cc1C(=O)NCc1ccc(-n2nc(-c3ccc(N4CCC(CN5CCN(c6ccc7c(c6)CN(C6CCC(=O)NC6=O)C7=O)CC5)CC4)nc3)c3ccnc(N)c32)cc1] |
| II-17 | 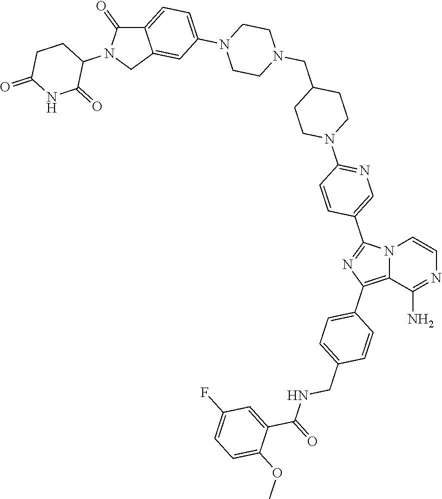 [SMILES: COc1ccc(F)cc1C(=O)NCc1ccc(-c2nc(-c3ccc(N4CCC(CN5CCN(c6ccc7c(c6)CN(C6CCC(=O)NC6=O)C7=O)CC5)CC4)nc3)n3ccnc(N)c23)cc1] |
| II-18 | 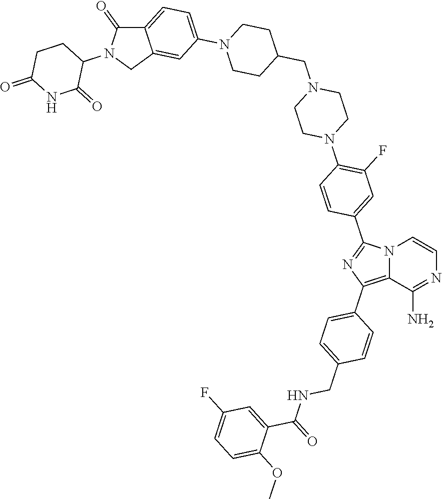 [SMILES: COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)c(F)c3)n3ccnc(N)c23)cc1] |
| II-19 | 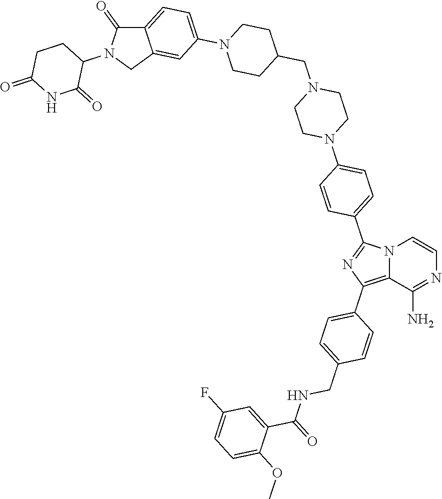 [SMILES: COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)cc3)n3ccnc(N)c23)cc1] |
| II-20 | 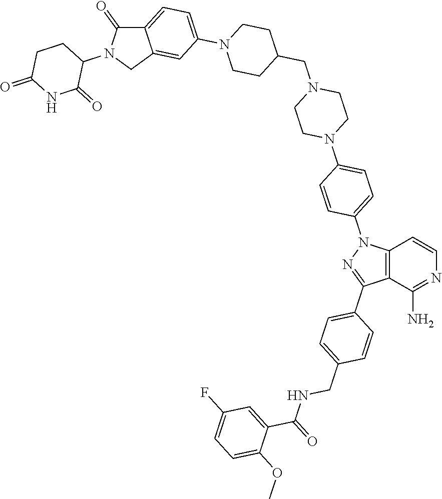 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)cc3)c3ccnc(N)c23)cc1] |
| II-21 | 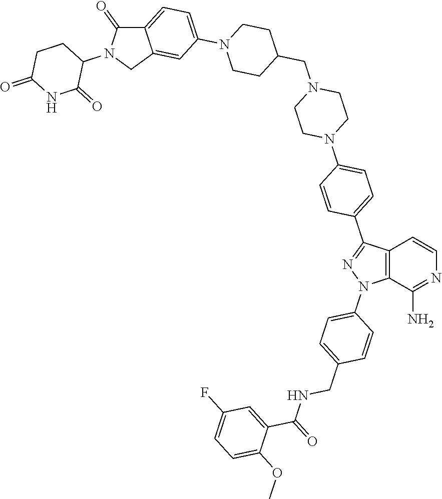 [SMILES: COc1ccc(F)cc1C(=O)NCc1ccc(-n2nc(-c3ccc(N4CCN(CC5CCN(c6ccc7c(c6)CN(C6CCC(=O)NC6=O)C7=O)CC5)CC4)cc3)c3ccnc(N)c32)cc1] |
| II-22 | 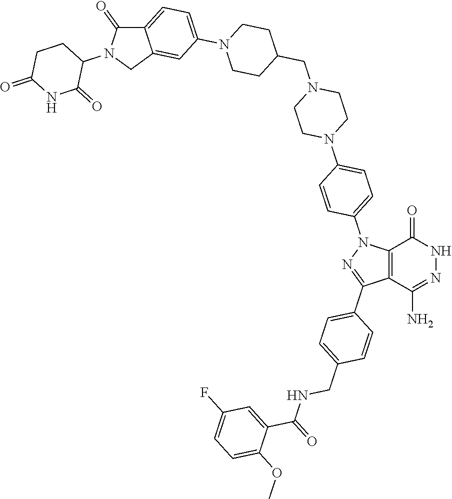 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)cc3)c3c(=O)[nH]nc(N)c23)cc1] |
| II-23 | 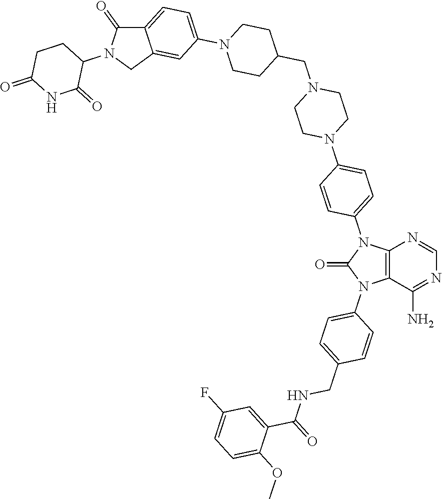 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)cc3)c3ncnc(N)c32)cc1] |
| II-24 | 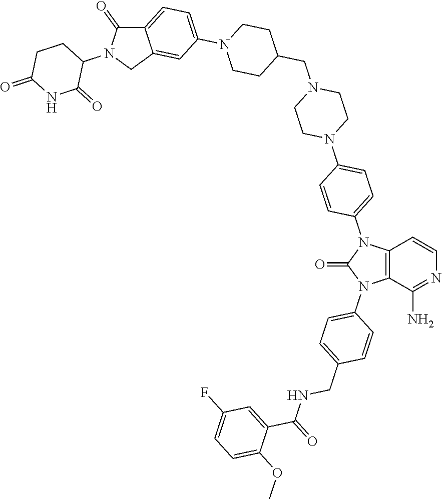 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)cc3)c3ccnc(N)c32)cc1] |
| II-25 | 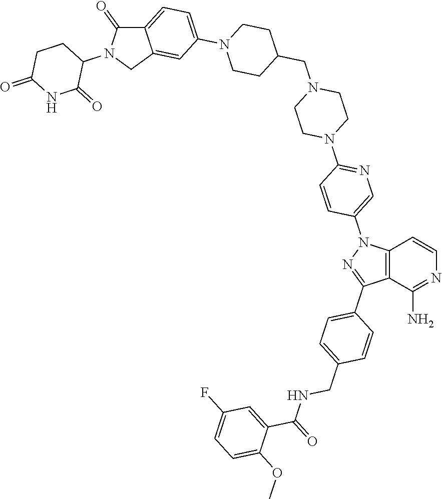 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ccnc(N)c23)cc1] |
| II-26 | 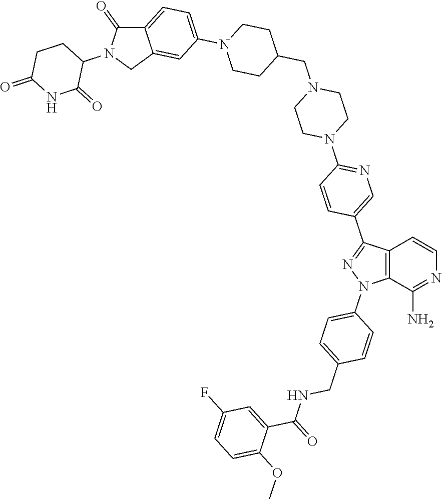 [SMILES: COc1ccc(F)cc1C(=O)NCc1ccc(-n2nc(-c3ccc(N4CCN(CC5CCN(c6ccc7c(c6)CN(C6CCC(=O)NC6=O)C7=O)CC5)CC4)nc3)c3ccnc(N)c32)cc1] |
| II-27 | 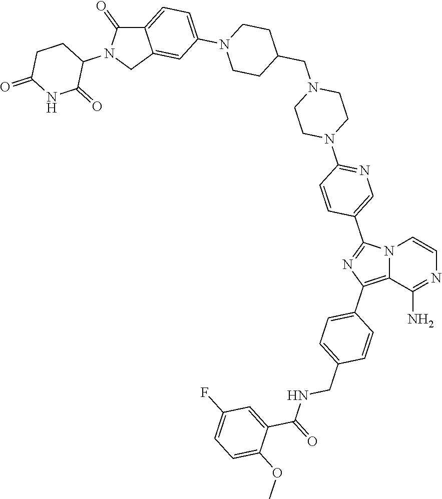 [SMILES: COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)nc3)n3ccnc(N)c23)cc1] |
| II-28 | 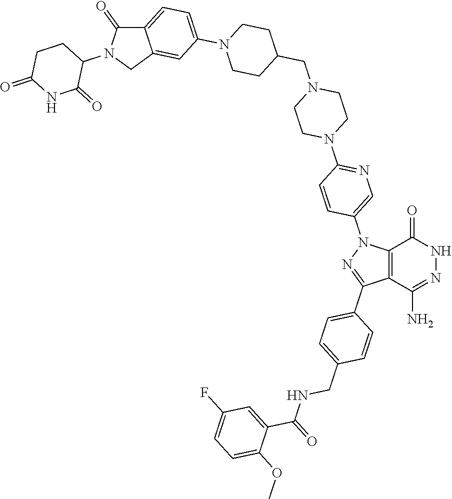 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3c(=O)[nH]nc(N)c23)cc1] |
| II-29 | 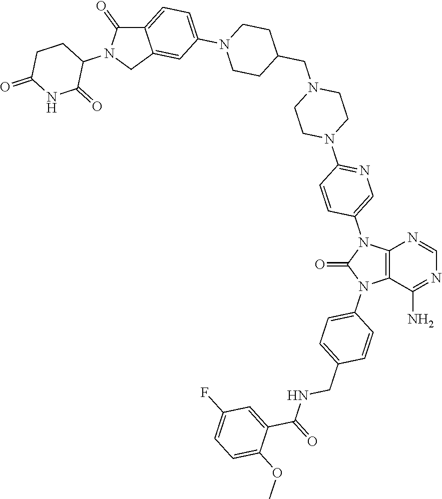 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)nc3)c3ncnc(N)c32)cc1] |
| II-30 | 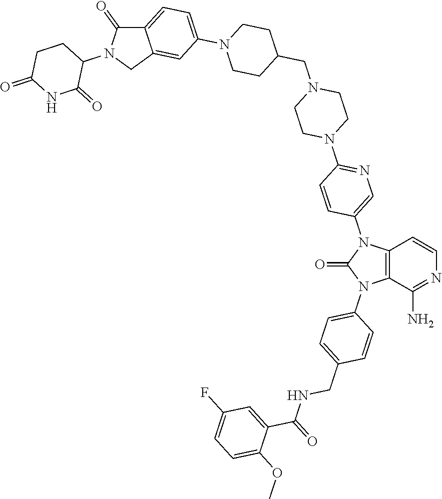 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)nc3)c3ccnc(N)c32)cc1] |
| II-31 | 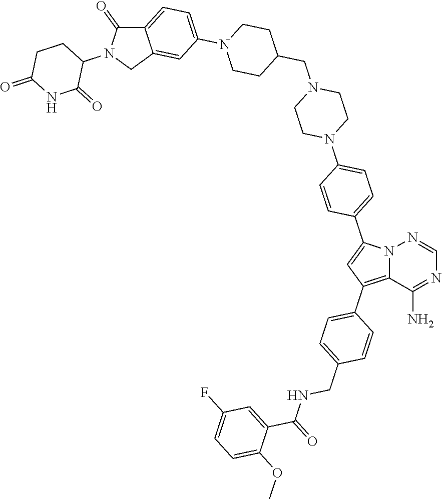 [SMILES: COc1ccc(F)cc1C(=O)NCc1ccc(-c2cc(-c3ccc(N4CCN(CC5CCN(c6ccc7c(c6)CN(C6CCC(=O)NC6=O)C7=O)CC5)CC4)cc3)n3ncnc(N)c23)cc1] |
| II-32 | 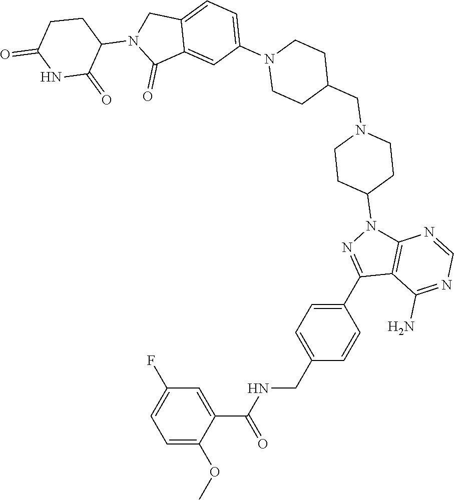 [SMILES: COc1ccc(F)cc1C(=O)NCc1ccc(-c2nn(C3CCN(CC4CCN(c5ccc6c(c5)C(=O)N(C5CCC(=O)NC5=O)C6)CC4)CC3)c3ncnc(N)c23)cc1] |
| II-33 | 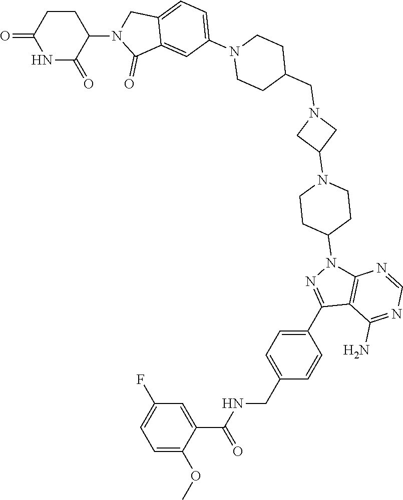 [SMILES: COc1ccc(F)cc1C(=O)NCc1ccc(-c2nn(C3CCN(C4CN(CC5CCN(c6ccc7c(c6)C(=O)N(C6CCC(=O)NC6=O)C7)CC5)C4)CC3)c3ncnc(N)c23)cc1] |
| II-34 | 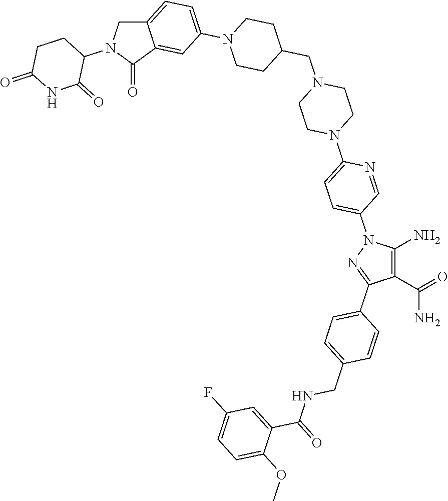 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)nc3)c(N)c2C(N)=O)cc1] |
| II-35 | 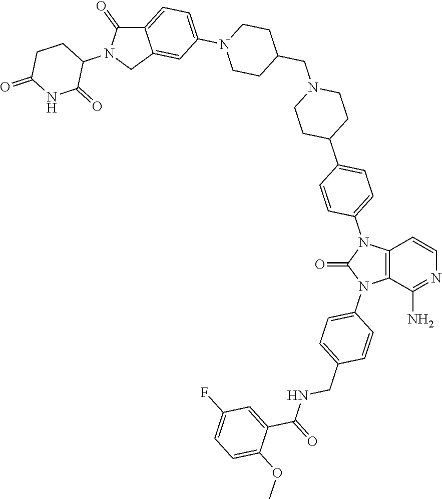 [SMILES: COc1ccc(F)cc1C(=O)NCc1ccc(-n2c(=O)n(-c3ccc(C4CCN(CC5CCN(c6ccc7c(c6)CN(C6CCC(=O)NC6=O)C7=O)CC5)CC4)cc3)c3ccnc(N)c32)cc1] |
| II-36 | 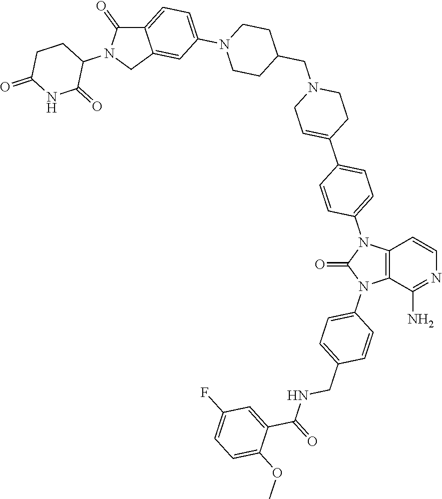 [SMILES: COc1ccc(F)cc1C(=O)NCc1ccc(-n2c(=O)n(-c3ccc(C4=CCN(CC5CCN(c6ccc7c(c6)CN(C6CCC(=O)NC6=O)C7=O)CC5)CC4)cc3)c3ccnc(N)c32)cc1] |
| II-37 | 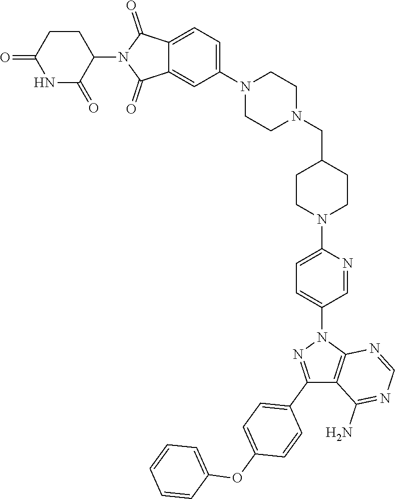 [SMILES: Nc1ncnc2c1c(-c1ccc(Oc3ccccc3)cc1)nn2-c1ccc(N2CCC(CN3CCN(c4ccc5c(c4)C(=O)N(C4CCC(=O)NC4=O)C5=O)CC3)CC2)nc1] |
| II-38 | 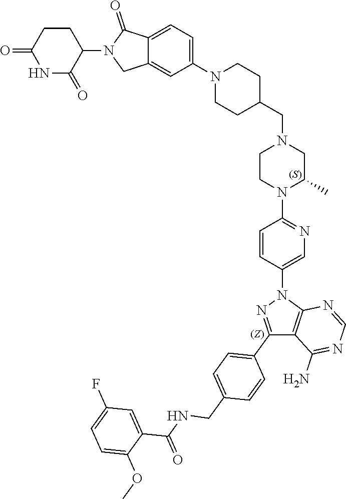 |
| II-39 | 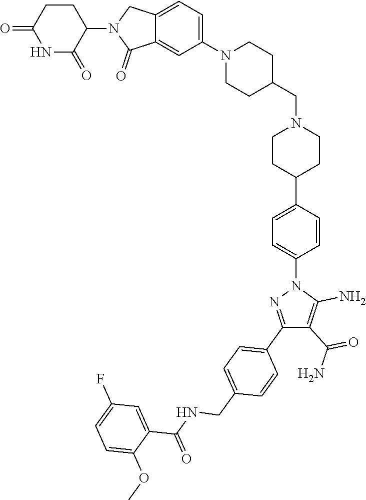 [SMILES: COc1ccc(F)cc1C(=O)NCc1ccc(-c2nn(-c3ccc(C4CCN(CC5CCN(c6ccc7c(c6)C(=O)N(C6CCC(=O)NC6=O)C7)CC5)CC4)cc3)c(N)c2C(N)=O)cc1] |

TABLE 3

| Se | |
|---|---|
| rial- | |
| No. | Structure |
| III-1 | 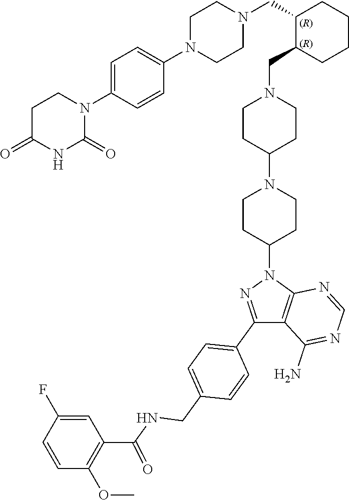 |
| III-2 | 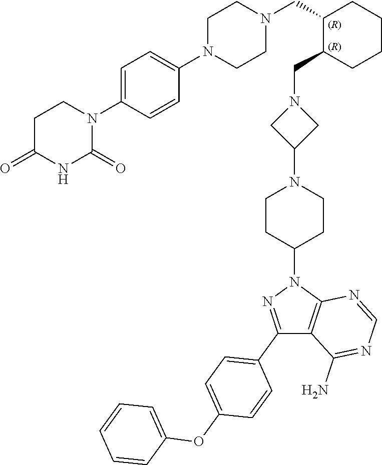 |
| III-3 | 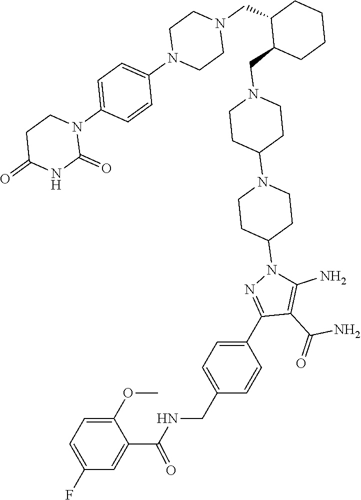 [SMILES: COc1ccc(F)cc1C(=O)NCc1ccc(-c2nn(C3CCN(C4CCN(C[C@@H]5CCCC[C@H]5CN5CCN(c6ccc(N7CCC(=O)NC7=O)cc6)CC5)CC4)CC3)c(N)c2C(N)=O)cc1] |
| III-4 | 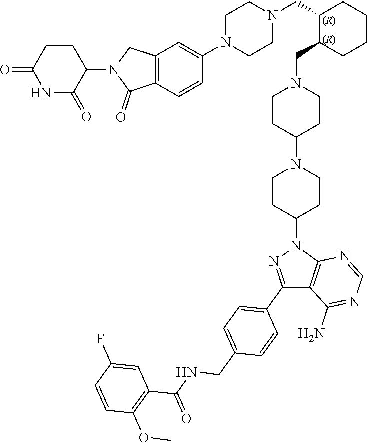 |
| III-5 | 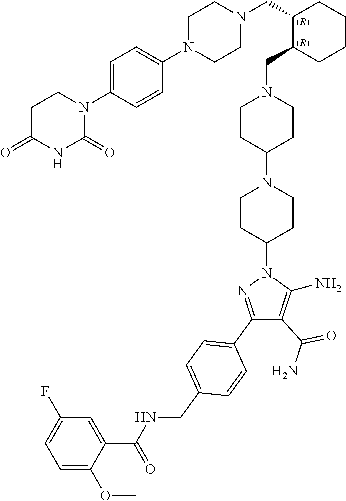 |
| III-6 | 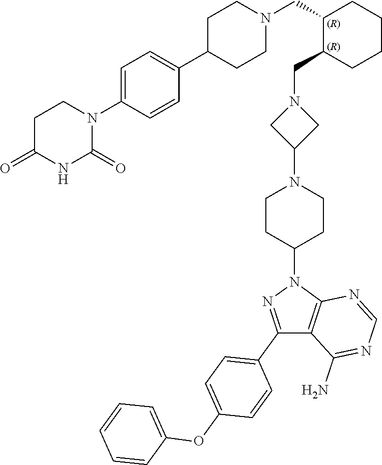 |
| III-7 | 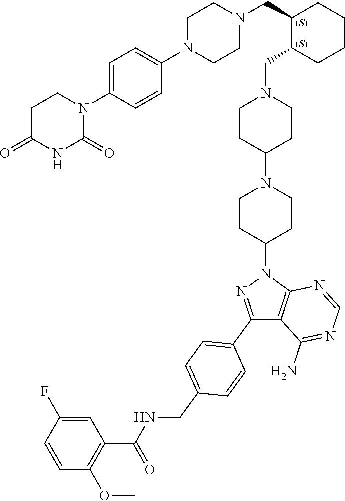 |
| III-8 | 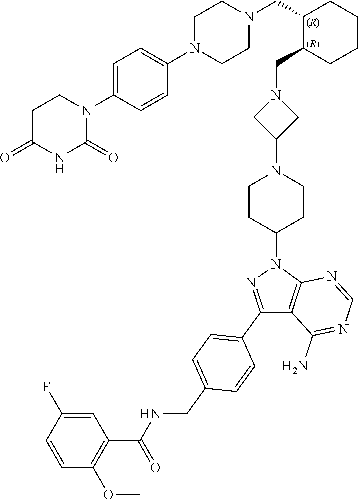 |
| III-9 | 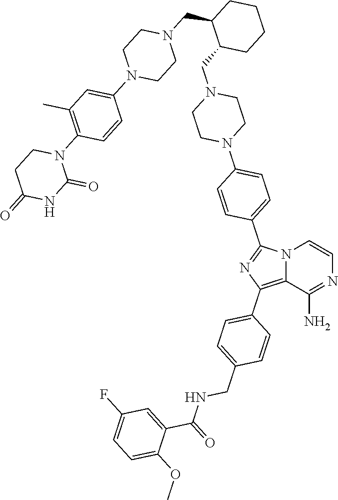 [SMILES: COc1ccc(F)cc1C(=O)NCc1ccc(-c2nc(-c3ccc(N4CCN(C[C@H]5CCCC[C@@H]5CN5CCN(c6ccc(N7CCC(=O)NC7=O)c(C)c6)CC5)CC4)cc3)n3ccnc(N)c23)cc1] |
| III- 10 | 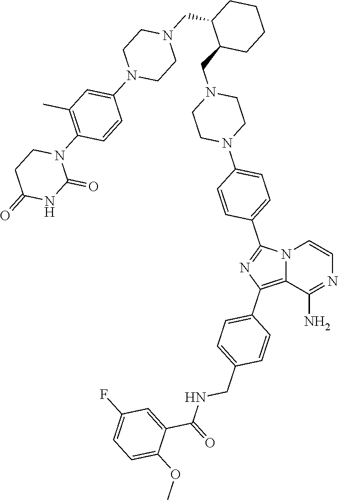 [SMILES: COc1ccc(F)cc1C(=O)NCc1ccc(-c2nc(-c3ccc(N4CCN(C[C@@H]5CCCC[C@H]5CN5CCN(c6ccc(N7CCC(=O)NC7=O)c(C)c6)CC5)CC4)cc3)n3ccnc(N)c23)cc1] |
| III- 11 | 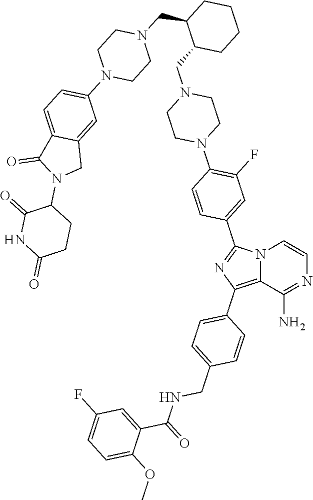 [SMILES: COc1ccc(F)cc1C(=O)NCc1ccc(-c2nc(-c3ccc(N4CCN(C[C@H]5CCCC[C@@H]5CN5CCN(c6ccc7c(c6)CN(C6CCC(=O)NC6=O)C7=O)CC5)CC4)c(F)c3)n3ccnc(N)c23)cc1] |
| III- 12 | 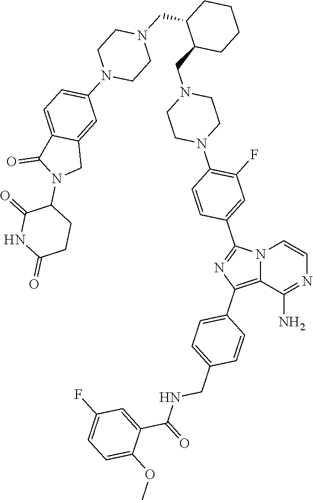 [SMILES: COc1ccc(F)cc1C(=O)NCc1ccc(-c2nc(-c3ccc(N4CCN(C[C@@H]5CCCC[C@H]5CN5CCN(c6ccc7c(c6)CN(C6CCC(=O)NC6=O)C7=O)CC5)CC4)c(F)c3)n3ccnc(N)c23)cc1] |
| III- 13 | 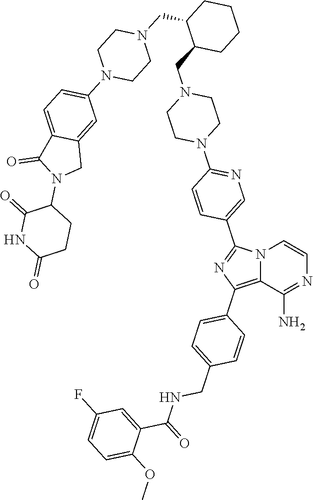 [SMILES: COc1ccc(F)cc1C(=O)NCc1ccc(-c2nc(-c3ccc(N4CCN(C[C@@H]5CCCC[C@H]5CN5CCN(c6ccc7c(c6)CN(C6CCC(=O)NC6=O)C7=O)CC5)CC4)nc3)n3ccnc(N)c23)cc1] |
| III- 14 | 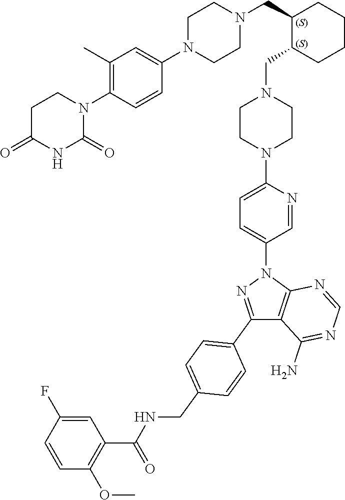 |
| III- 15 | 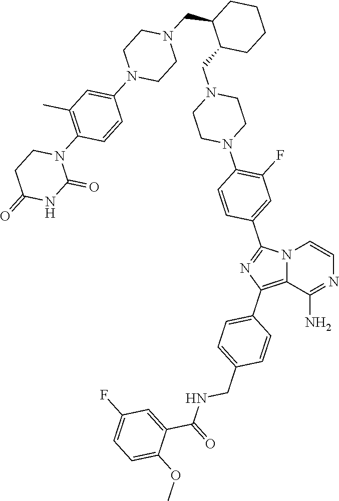 [SMILES: COc1ccc(F)cc1C(=O)NCc1ccc(-c2nc(-c3ccc(N4CCN(C[C@H]5CCCC[C@@H]5CN5CCN(c6ccc(N7CCC(=O)NC7=O)c(C)c6)CC5)CC4)c(F)c3)n3ccnc(N)c23)cc1] |
| III- 16 | 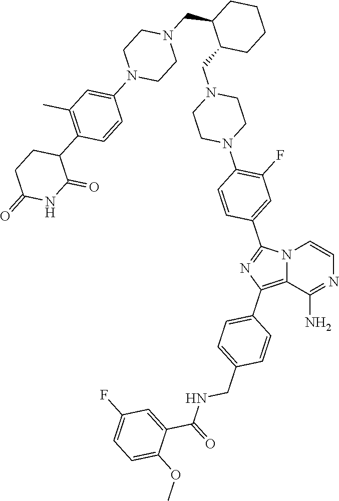 [SMILES: COc1ccc(F)cc1C(=O)NCc1ccc(-c2nc(-c3ccc(N4CCN(C[C@H]5CCCC[C@@H]5CN5CCN(c6ccc(C7CCC(=O)NC7=O)c(C)c6)CC5)CC4)c(F)c3)n3ccnc(N)c23)cc1] |
| III- 17 | 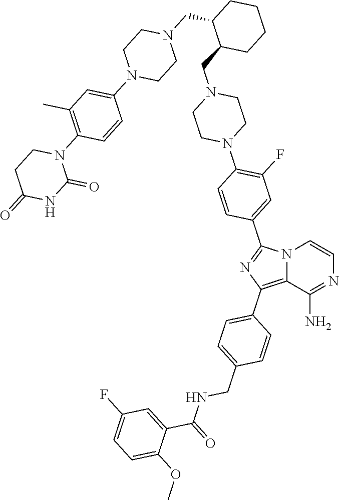 [SMILES: COc1ccc(F)cc1C(=O)NCc1ccc(-c2nc(-c3ccc(N4CCN(C[C@@H]5CCCC[C@H]5CN5CCN(c6ccc(N7CCC(=O)NC7=O)c(C)c6)CC5)CC4)c(F)c3)n3ccnc(N)c23)cc1] |
| III- 18 | 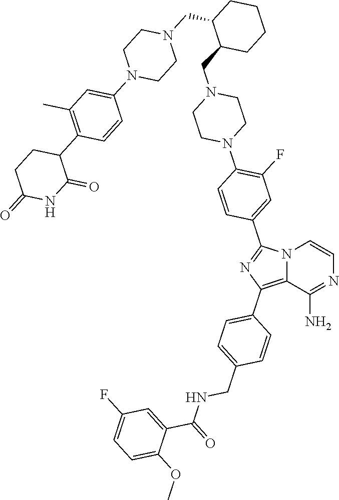 [SMILES: COc1ccc(F)cc1C(=O)NCc1ccc(-c2nc(-c3ccc(N4CCN(C[C@@H]5CCCC[C@H]5CN5CCN(c6ccc(C7CCC(=O)NC7=O)c(C)c6)CC5)CC4)c(F)c3)n3ccnc(N)c23)cc1] |
| III- 19 | 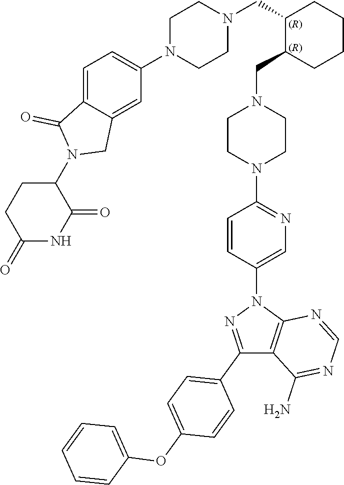 |
| III- 20 | 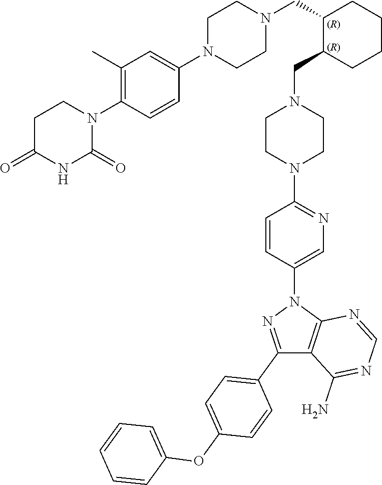 |

The present invention also provides a pharmaceutical composition, comprising the compound of any embodiment, or the isomer or pharmaceutically acceptable salt thereof, and pharmaceutically acceptable carriers.

The present invention also provides the use of the compound of any of the above embodiments, or the isomer or pharmaceutically acceptable salt thereof and the above pharmaceutical composition in the preparation of a drug for treating diseases related to the inhibition or degradation of BTK.

According to an embodiment of the present invention, the disease is selected from autoimmune diseases, inflammatory diseases, or cancers.

According to an embodiment of the present invention, the disease is selected from B cell malignancy, B cell lymphoma, diffuse large B cell lymphoma, chronic lymphocytic leukemia, small lymphocytic lymphoma, marginal zone lymphoma, non-Hodgkin's lymphoma, mantle cell lymphoma, follicular lymphoma, hairy cell leukemia, B-cell non-Hodgkin's lymphoma, Waldenstrom's macroglobulinemia, multiple myeloma, bone cancer, bone metastase, arthritis, multiple sclerosis, osteoporosis, irritable bowel syndrome, inflammatory bowel disease, Crohn's disease, lupus, Sjögren's syndrome, chronic graft-versus-host disease and a disorder associated with renal transplantation.

### DETAILED DESCRIPTION

The “halogen” described in the present invention refers to fluorine, chlorine, bromine, iodine, etc., and preferably fluorine and chlorine.

The “halogenated” described in the present invention means that any hydrogen atom in a substituent may be substituted with one or more halogen atoms which may be the same or different. “Halogen” is as defined above.

The heteroatom described in the present invention refers to O, N or S, and preferably N.

The “C₁₋₆ alkyl” described in the present invention refers to a straight or branched alkyl derived from a hydrocarbon moiety containing 1-6 carbon atoms by removing a hydrogen atom, such as methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, and 1-methyl-2-methylpropyl. The “C₁₋₆ alkyl” is preferably C₁₋₄ alkyl or C₁₋₃ alkyl.

The “C₂₋₈ alkenyl” described in the present invention refers to a straight or branched or cyclic olefin group derived from an olefin moiety of 2-8 carbon atoms containing a carbon-carbon double bond by removing a hydrogen atom, such as ethenyl, 1-propenyl, 2-propenyl, 1-butenyl, 2-butenyl, 1,3-butadienyl, 1-pentenyl, 2-pentenyl, 3-pentenyl, 1,3-pentadienyl, 1,4-pentadienyl, 1-hexenyl, and 1,4-hexadienyl.

The “C₂₋₈ alkynyl” described in the present invention refers to a straight or branched alkyne group derived from an alkyne moiety of 2-8 carbon atoms containing a carbon-carbon triple bond by removing a hydrogen atom, such as ethynyl, propynyl, 2-butynyl, 2-pentynyl, 3-pentynyl, 4-methyl-2-pentynyl, 2-hexynyl, and 3-hexynyl, etc. The “C₂₋₈ alkynyl” is preferably C₂₋₄ alkynyl or C₂₋₃ alkynyl.

The “C₁₋₆ alkoxy” described in the present invention refers to a group in which the “C₁₋₆ alkyl” defined above is attached to the parent molecule through an oxygen atom, i.e., a “C₁₋₆ alkyl-O—” group, such as methoxy, ethoxy, n-propoxy, isopropoxy, n-butoxy, tert-butoxy, n-pentoxy, neopentoxy and n-hexoxy, etc. The “C₁₋₆ alkoxy” is preferably C₁₋₄ alkoxy or C₁₋₃ alkoxy.

The “C₁₋₆ alkylamino”, “(C₁₋₆ alkyl)amino”, “C₁₋₆ alkylcarbonylamino”, “C₁₋₆ alkylsulfonylamino”, “C₁₋₆ alkylaminocarbonyl, “(C₁₋₆ alkyl)₂ amino-carbonyl”, “C₁₋₆ alkoxy-carbonyl”, “C₁₋₆ alkylsulfonyl”, “C₁₋₆ alkylthio, “C₁₋₆ alkylcarbonyl”, “aminocarbonyl”, and “hydroxy C₁₋₆ alkyl” described in the present invention refer to C₁₋₆ alkyl-NH—, (C₁₋₆ alkyl) (C₁₋₆ alkyl)N—, C₁₋₆ alkyl-C(O)—NH—, C₁₋₆ alkyl-S(O)₂—NH₂—, C₁₋₆ alkyl-NH—C(O)—, (C₁₋₆ alkyl) (C₁₋₆ alkyl)N—C(O)—, C₁₋₆ alkyl-O—C(O)—, C₁₋₆ alkyl-S(O)₂—, C₁₋₆ alkyl-S—, C₁₋₆ alkyl-C(O)—, NH₂—C(O)—, and OH—C₁₋₆ alkyl-, respectively; and the “C₁₋₆ alkyl” is as defined above, preferably “C₁₋₄ alkyl or C₁₋₃ alkyl”.

The fused ring described in the present invention refers to a condensed ring structure formed by two or more cyclic structures sharing two adjacent ring atoms (i.e., sharing one bond).

The monocycloalkyl described in the present invention is a cyclic hydrocarbon group containing 3-8 carbon atoms, including a 3-8 membered cycloalkyl group, a 4-7 membered cycloalkyl group, a 4-6 membered cycloalkyl group, and a 5-6 membered cycloalkyl group. Examples of the 3-8 membered cycloalkyl group include, but are not limited to, cyclopropyl, cyclobutyl, cyclopentyl, cyclohexyl, cycloheptyl, cyclooctyl, and the like.

The monoheterocyclyl refers to a saturated cyclic group in which at least one ring carbon atom is replaced by a heteroatom, preferably 1-3 heteroatoms, selected from O, S, and N. The monoheterocyclyl includes a 3-8 membered heterocyclyl group, a 3-6 membered heterocyclyl group, a 4-6 membered heterocyclyl group, a 4-7 membered heterocyclyl group, a 5-7 membered heterocyclyl group, a 5-6 membered heterocyclyl group, a 4-6 membered nitrogen-containing heterocyclyl group, a 5-6 membered oxygen-containing heterocyclyl group, a 3-8 membered nitrogen-containing heterocyclyl group, a 5-6 membered nitrogen-containing heterocyclyl group, a 5-6 membered saturated heterocyclyl group, and the like.

The heteroaryl described in the present invention refers to an aromatic cyclic group in which at least one ring carbon atom is replaced by a heteroatom selected from O, S, and N. The monoheteroaryl can be a 5-7 membered heteroaryl group or a 5-6 membered heteroaryl group, and examples thereof include, but are not limited to, furanyl, imidazolyl, isoxazolyl, thiazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thienyl, triazolyl and triazinyl.

The monocycloalkenyl described in the present invention refers to a partially saturated carbocyclic group, including a 3-8 membered cycloalkenyl group, a 4-7 membered cycloalkenyl group, and a 5-6 membered cycloalkenyl group.

The monocycloalkenyl described in the present invention refers to an unsaturated cyclic group in which at least one ring carbon atom is replaced by a heteroatom selected from O, S, and N, including a 3-8 membered heterocycloalkenyl group, a 4-7 membered heterocycloalkenyl group, and a 5-6 membered heterocycloalkenyl group.

The “heterocyclyl” described in the present invention refers to a 4-12 membered non-aromatic cyclic group in which at least one ring carbon atom is replaced by a heteroatom, preferably 1-3 heteroatoms, selected from O, S, and N, and in which carbon atoms, nitrogen atoms and sulfur atoms may be oxo-substituted.

The “4-12 membered heterocyclyl group” refers to a saturated monocyclic or bicyclic heterocyclyl system.

The bicyclic heterocyclyl system includes a condensed ring system, such as in fused or bridged form, and also include a spiro ring system.

The spiro heterocyclyl group may be a 6-12 membered spiro heterocyclyl group, a 7-11 membered spiro heterocyclyl group, or a 6-12 membered saturated spirocyclic group. The bridged heterocyclyl group may be a 6-7 membered bridged heterocyclyl group, a 6-8 membered bridged heterocyclyl group, a 6-12 membered bridged heterocyclyl group, a 7-11 membered bridged heterocyclyl group, or a 6-12 membered saturated bridged ring group. The fused heterocyclyl group may be a 6-12 membered fused heterocyclyl group, a 7-10 membered fused heterocyclyl group, or a 6-10 membered fused heterocyclyl group.

The fused heterocycloalkenyl group described in the present invention refers to an unsaturated fused ring structure in which at least one ring carbon atom is replaced by a heteroatom selected from O, S, and N, such as a 9-10 membered fused heterocycloalkenyl group.

The fused heteroaryl group described in the present invention refers to an aromatic fused ring structure in which at least one ring carbon atom is replaced by a heteroatom selected from O, S, and N, such as a 9-10 membered fused heteroaryl group.

The “isomer” described in the present invention refers to a stereoisomer and a tautomer.

The “stereoisomer” refers to an enantiomer that arise when a compound has an asymmetric atom or a cis-trans isomer that arise when a compound has a double bond or a ring structure. All enantiomers, diastereomers, racemates, cis-trans isomers, geometric isomers, epimers and mixtures thereof of the compound of formula (I) are included in the scope of the present invention.

The “tautomer” refers to a functional group isomer that arise due to the rapid movement of an atom in two positions in a molecule, which is a special functional group isomer. For example, tautomerism occurs in carbonyl compounds containing α-H, such as

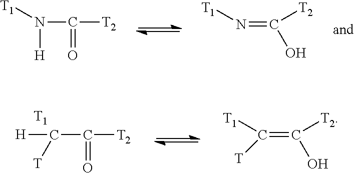

For example, other tautomerism phenomena occur by proton migration, such as phenol-keto tautomerism, nitroso-oxime tautomerism, and imine-enamine tautomerism.
T, T1, and T2 are independently any group that conforms to the bonding rules of the compound.

### BRIEF DESCRIPTION OF THE DRAWINGS

FIG. 1 depicts the degradation effects of compounds I-5 and I-24 on BTK in mino cells;

FIG. 2 depicts the degradation effects of compounds II-3 and II-2 on BTK in mino cells;

FIG. 3 depicts the degradation effects of compound I-19 on BTK in mino cells; and

FIG. 4 depicts the degradation effects of compound I-30 on BTK in mino cells.

### DETAILED DESCRIPTION OF THE INVENTION

The present invention is described below with reference to specific examples. Those skilled in the art will appreciate that these examples are only used for illustrating the present invention and are not intended to limit the scope of the present invention in any way.

The experimental methods in the following examples are all conventional methods unless otherwise specified. Unless specifically stated, medicinal raw materials and reagent materials used in the examples below are commercially available products.

### Example 1: Synthesis of N-(4-(4-amino-1-(1-(1-((1-(4-(2,4-dioxotetrahydropyrimidin-1(2H)-yl)phenyl) piperidin-4-yl)methyl) azetidin-3-yl) piperidin-4-yl)-1H-pyrazolo[3,4-d]pyrimidin-3-methyl)benzyl)-5-fluoro-2-methoxybenzamide (Compound I-1)

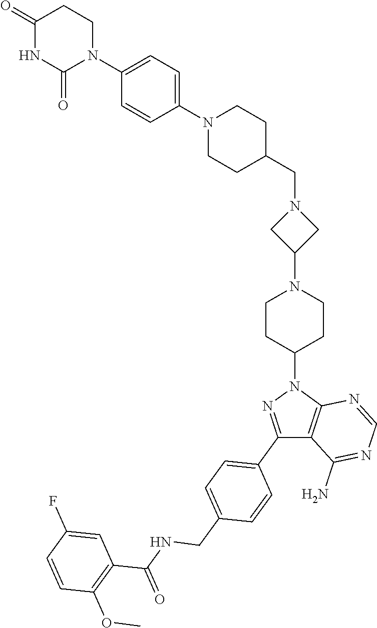 [SMILES: COc1ccc(F)cc1C(=O)NCc1ccc(-c2nn(C3CCN(C4CN(CC5CCN(c6ccc(N7CCC(=O)NC7=O)cc6)CC5)C4)CC3)c3ncnc(N)c23)cc1]

### Step 1:4-dimethoxymethyl-1-(4-nitrophenyl)piperidine

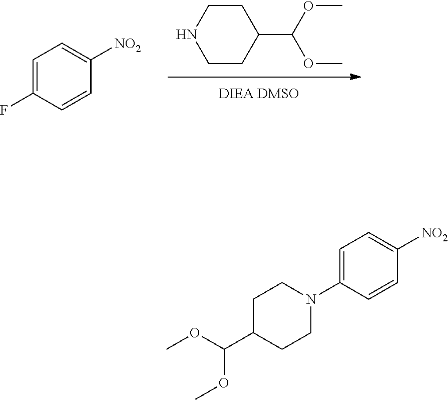 [SMILES: O=[N+]([O-])c1ccc(F)cc1 ; COC(OC)C1CCNCC1 ; COC(OC)C1CCN(c2ccc([N+](=O)[O-])cc2)CC1]

At room temperature, p-fluoronitrobenzene (8.06 g, 57.10 mmol), 4-(dimethoxymethyl)-piperidine (10.00 g, 62.80 mmol), DIEA (14.72 g, 114.20 mmol) and DMSO (100 mL) were added into a single-necked flask in sequence. Under nitrogen protection, the mixture was slowly heated to 90° C. and the reaction was allowed to proceed for 20 h before termination. The reaction solution was cooled, diluted with water, and extracted with EA. The organic phase was dried over anhydrous Na₂SO₄, filtered, concentrated and purified by column chromatography (EA/PE system) to afford 15.92 g of a yellow solid with a yield of 99%. MS (ESI) m/z: 281.1 [M+H]⁺.

### Step 2: Synthesis of 4-(4-dimethoxymethyl) piperidin-1-ylaniline

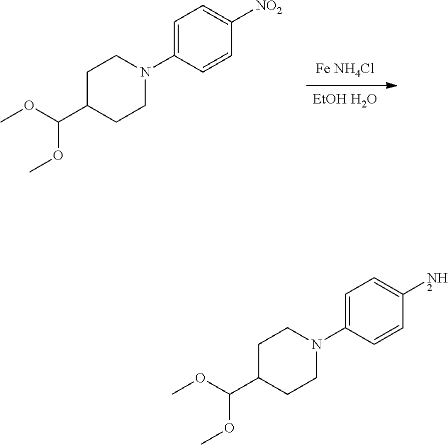 [SMILES: "EtOH H2O" ; COC(OC)C1CCN(c2ccc([N+](=O)[O-])cc2)CC1 ; "Fe NH4Cl" ; COC(OC)C1CCN(c2ccc(N)cc2)CC1]

At room temperature, 4-dimethoxymethyl-1-(4-nitrophenyl)piperidine (15.92 g, 24.00 mmol), NH₄Cl (18.18 g, 119.80 mmol), reduced iron powder (15.91 g, 119.80 mmol) and an aqueous ethanol solution (200 mL, ethanol:water=9:1) were added into a single-necked flask in sequence. The reaction was allowed to proceed at 80° C. for 18 h under nitrogen protection before termination. The reaction solution was concentrated, diluted with water and extracted with EA, and the filtrate was concentrated to afford 9.04 g of a crude product as a white solid with a yield of 63%. MS (ESI) m/z: 251.2 [M+H]⁺.

### Step 3: Synthesis of ethyl 3-(4-(4-(dimethoxymethyl) piperidin-1-yl)phenylamino) propanoate

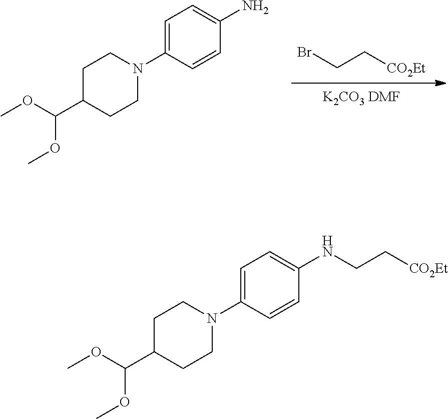

At room temperature, 4-(4-dimethoxymethyl) piperidin-1-ylaniline (9.04 g, 36.10 mmol), potassium carbonate (9.97 g, 72.20 mmol), ethyl 3-bromopropionate (9.81 g, 54.20 mmol) and DMF (150 mL) were added into a single-necked flask in sequence. The reaction was allowed to proceed at 80° C. for 16 h under nitrogen protection before termination. The reaction solution was diluted with water, and extracted with EA. The organic phase was dried over anhydrous Na₂SO₄, filtered, concentrated and purified by column chromatography (EA/PE system) to afford 7.23 g of a yellow solid. MS (ESI) m/z: 351.2 [M+H]⁺.

### Step 4: Synthesis of ethyl 3-(N-(4-(4-(dimethoxymethyl) piperidin-1-ylphenylcyanamido) propionate

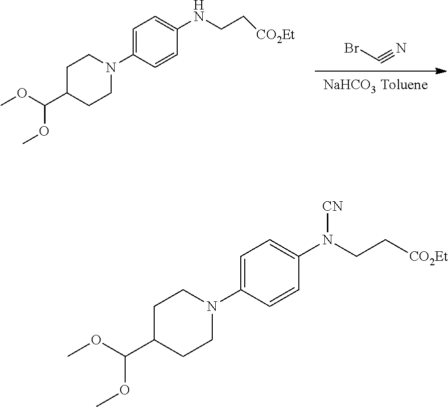

At room temperature, ethyl 3-(4-(4-(dimethoxymethyl) piperidin-1-yl)phenylamino) propanoate (7.23 g, 20.60 mmol), nitrile bromide (3.28 g, 31.00 mmol), sodium bicarbonate (5.20 g, 61.80 mmol) and toluene (150 mL) were added into a single-necked flask in sequence. The reaction was allowed to proceed at room temperature for 16 h under nitrogen protection before termination. The reaction solution was poured into a saturated sodium bicarbonate solution, and extracted with EA after no bubbles appeared. The organic phase was dried over anhydrous Na₂SO₄, filtered, concentrated and purified by column chromatography (EA/PE system) to afford 5.73 g of a yellow solid. MS (ESI) m/z: 376.2 [M+H]⁺.

### Step 5: Synthesis of ethyl 3-(1-(4-(4-(dimethoxymethyl) piperidin-1-ylphenyl)ureido)propionate

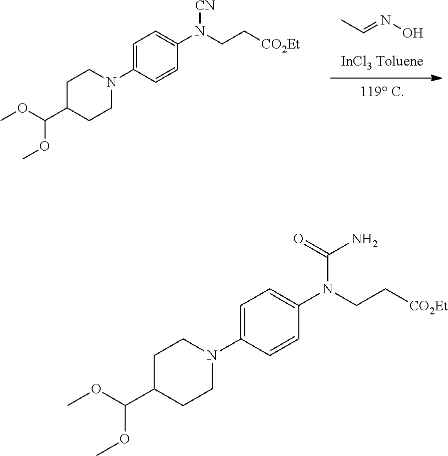

At room temperature, ethyl 3-(N-(4-(4-(dimethoxymethyl) piperidin-1-yl)phenyl) cyanamido) propionate (5.73 g, 15.10 mmol), acetaldehyde oxime (2.64 g, 67.40 mmol), InCl₃ (0.72 g, 4.50 mmol) and toluene (150 mL) were added into a single-necked flask in sequence. The reaction was allowed to proceed at 115° C. for 2 h under nitrogen protection before termination. The reaction solution was cooled to room temperature and concentrated to afford a red-brown viscous substance, which was washed with n-heptane and dried to afford 6.70 g of a red-brown solid. MS (ESI) m/z: 394.2 [M+H]⁺.

### Step 6:4-(4-dimethoxymethyl) piperidin-1-yl-phenyl)dihydropyrimidine-2,4-dione

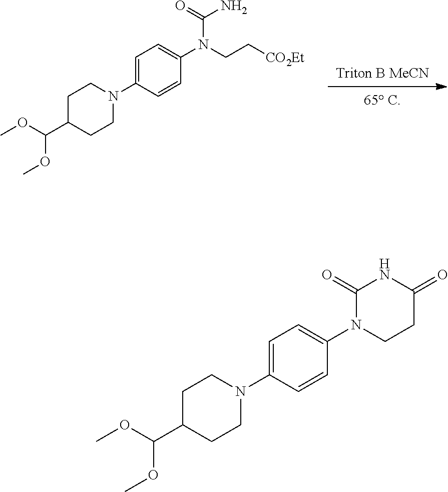 [SMILES: CCOC(=O)CCN(C(N)=O)c1ccc(N2CCC(C(OC)OC)CC2)cc1 ; "65° C." ; "Triton B MeCN" ; COC(OC)C1CCN(c2ccc(N3CCC(=O)NC3=O)cc2)CC1]

At room temperature, ethyl 3-(1-(4-(4-(dimethoxymethyl) piperidin-1-yl-phenyl) ureido) propanoate (6.70 g, 15.30 mmol), 40% Triton B in methanol (9.57 g, 22.90 mmol) and acetonitrile (100 mL) were added into a single-necked flask in sequence. The reaction was allowed to proceed at 65° C. for 1 h under nitrogen protection before termination. The reaction solution was concentrated and purified by column chromatography (MeOH/DCM system) to afford 2.32 g of a white solid. MS (ESI) m/z: 347.2 [M+H]⁺.

### Step 7: Synthesis of 4-(2,4-dioxatetrahydropyrimidine-1-phenyl)piperidine-4-carboxaldehyde

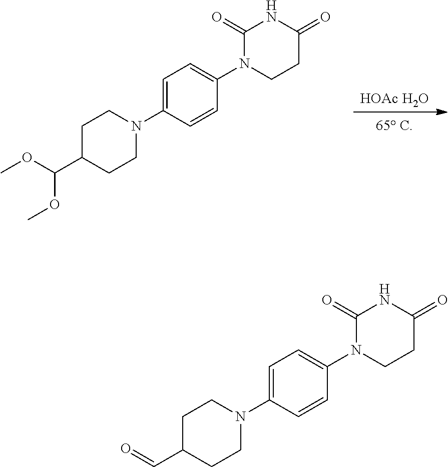 [SMILES: COC(OC)C1CCN(c2ccc(N3CCC(=O)NC3=O)cc2)CC1 ; "65° C." ; O=CC1CCN(c2ccc(N3CCC(=O)NC3=O)cc2)CC1 ; "HOAc H2O"]

At room temperature, 4-(4-dimethoxymethyl) piperidin-1-yl-phenyl)dihydropyrimidine-2,4-dione (2.23 g, 6.42 mmol), glacial acetic acid (20 mL) and water (20 mL) were added into a single-necked flask in sequence. The reaction was allowed to proceed at 65° C. for 4 h under nitrogen protection before termination. The reaction solution was concentrated and then purified by column chromatography (MeOH/DCM system) to afford 1.68 g of a white solid with a yield of 87%. MS (ESI) m/z: 302.4 [M+H]⁺.

### Step 8: Synthesis of (4-((5-fluoro-2-methoxybenzamido)methyl)phenyl)boronic acid

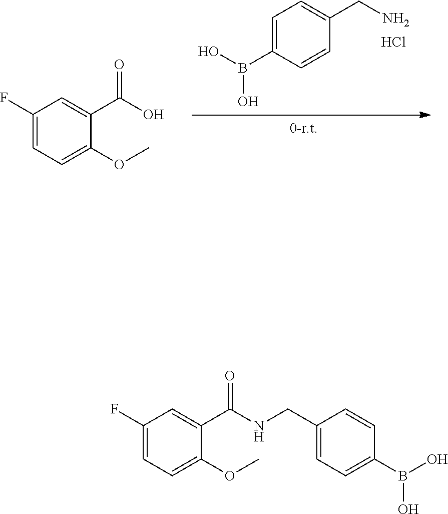 [SMILES: COc1ccc(F)cc1C(=O)O ; NCc1ccc(B(O)O)cc1 ; "0-r.t." ; Cl ; COc1ccc(F)cc1C(=O)NCc1ccc(B(O)O)cc1]

Under ice bath conditions, 5-fluoro-2-methoxybenzoic acid (2.00 g, 11.76 mmol), 4-aminomethylphenylborate hydrochloride (2.20 g, 11.76 mmol) and N-methylimidazole (2.41 g, 29.39 mmol) and DMF (40 mL) were added into a single-necked flask in sequence, TCFH (8.25 g, 29.39 mmol) was slowly added in portions. Under nitrogen protection, the mixture was warmed to room temperature and the reaction was allowed to proceed for 2 h before termination. The reaction solution was diluted with water, and extracted with BA. The organic phase was dried over anhydrous Na₂SO₄, filtered, and concentrated to afford 1.64 g of a crude product as a white solid with a yield of 46%. MS (ESI) m/z: 304.4 [M+H]⁺.

### Step 9: Synthesis of tert-butyl 4-(4-amino-3-bromo-1H-pyrazolo[3,4-d]pyrimidin-1-yl)piperidine-1-carboxylate

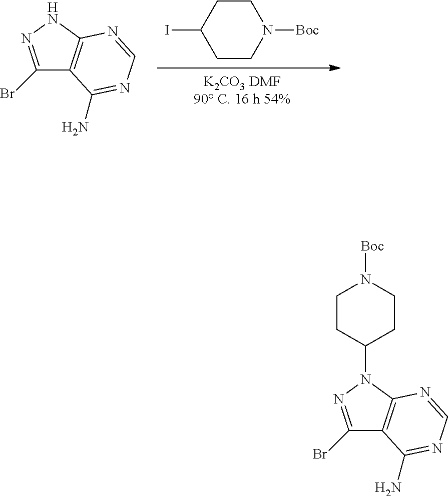

At room temperature, 3-bromo-1H-pyrazolo[3,4-d]pyrimidin-4-amine (3.00 g, 14.02 mmol), tert-butyl 4-iodopiperidine-1-carboxylate (6.54 g, 21.02 mmol), potassium carbonate (3.87 g, 28.03 mmol) and DMF (50 mL) were added into a single-necked flask in sequence, and the reaction was allowed to proceed at 90° C. for 16 h under nitrogen protection. The reaction solution was cooled to room temperature, added with water, and extracted twice with ethyl acetate. The combined organic phases were washed with water and saturated brine, dried over anhydrous sodium sulfate, filtered, concentrated and subjected to column chromatography (EA/PE system) to afford 3.00 g of a white solid with a yield of 54%.

### Step 10: Synthesis of tert-butyl 4-(4-amino-3-(4-((5-fluoro-2-methoxybenzamido)methyl)phenyl)-1H-pyrazolo[3,4-d]pyrimidin-1-yl)piperidine-1-carboxylate

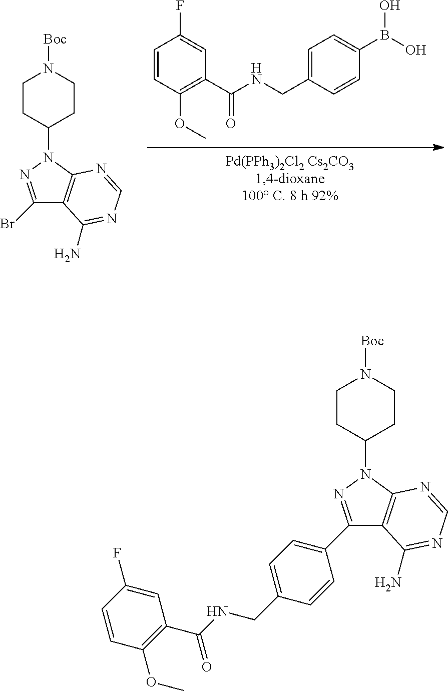 [SMILES: CC(C)(C)OC(=O)N1CCC(n2nc(Br)c3c(N)ncnc32)CC1 ; COc1ccc(F)cc1C(=O)NCc1ccc(B(O)O)cc1 ; "Pd(PPh3)2Cl2 Cs2CO31,4-dioxane100° C. 8 h 92%" ; COc1ccc(F)cc1C(=O)NCc1ccc(-c2nn(C3CCN(C(=O)OC(C)(C)C)CC3)c3ncnc(N)c23)cc1]

At room temperature, tert-butyl 4-(4-amino-3-bromo-1H-pyrazolo[3,4-d]pyrimidin-1-yl)piperidine-1-carboxylate (1.50 g, 3.78 mmol), (4-((5-fluoro-2-methoxybenzamido)methyl)phenyl) boric acid (1.72 g, 5.66 mmol), cesium carbonate (2.46 g, 7.55 mmol), Pd(PPh₃)₂Cl₂ (0.53 g, 0.75 mmol), 1,4-dioxane (30 mL), and H₂O (2 mL) were added into a single-necked flask in sequence, and the reaction was allowed to proceed at 100° C. for 6 h under nitrogen protection. The reaction solution was cooled to room temperature and filtered, and the filtrate was concentrated and subjected to column chromatography (MeOH/DCM system) to afford 2.00 g of a white solid with a yield of 92%.

### Step 11: Synthesis of N-(4-(4-amino-1-(piperidin-4-yl)-1H-pyrazolo[3,4-d]pyrimidin-3-yl)benzyl)-5-fluoro-2-methoxybenzamide

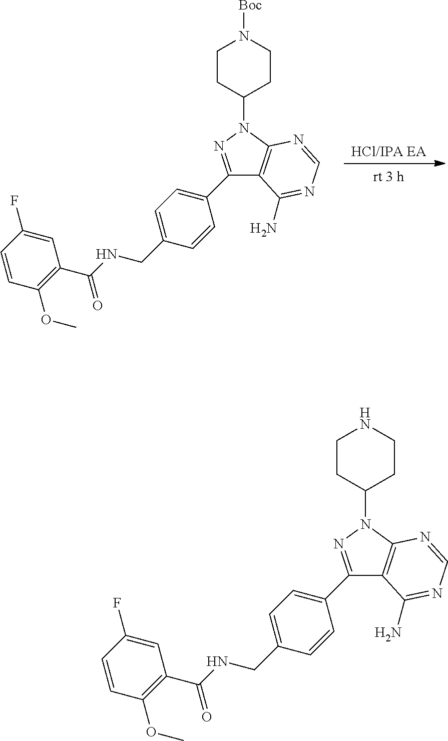 [SMILES: COc1ccc(F)cc1C(=O)NCc1ccc(-c2nn(C3CCN(C(=O)OC(C)(C)C)CC3)c3ncnc(N)c23)cc1 ; "HCl/IPA EA" ; "rt 3 h" ; COc1ccc(F)cc1C(=O)NCc1ccc(-c2nn(C3CCNCC3)c3ncnc(N)c23)cc1]

At room temperature, tert-butyl 4-(4-amino-3-(4-((5-fluoro-2-methoxybenzamide)methyl)phenyl)-1H-pyrazolo[3,4-d]pyrimidin-1-yl)piperidine-1-carboxylate (2.00 g, 3.47 mmol), isopropanol hydrochloride (10 mL), and EA (10 mL) were added into a single-necked flask in sequence and the reaction was allowed to proceed at room temperature for 3 h. The reaction solution was concentrated and treated with triethylamine to afford 2.00 g of a crude product as a white solid.

### Step 12: Synthesis of tert-butyl 3-(4-(4-amino-3-(4-((5-fluoro-2-methoxybenzamido)methyl)phenyl)-1H-pyrazolo[3,4-d]pyrimidin-1-yl) piperidin-1-methyl)azetidine-1-carboxylate

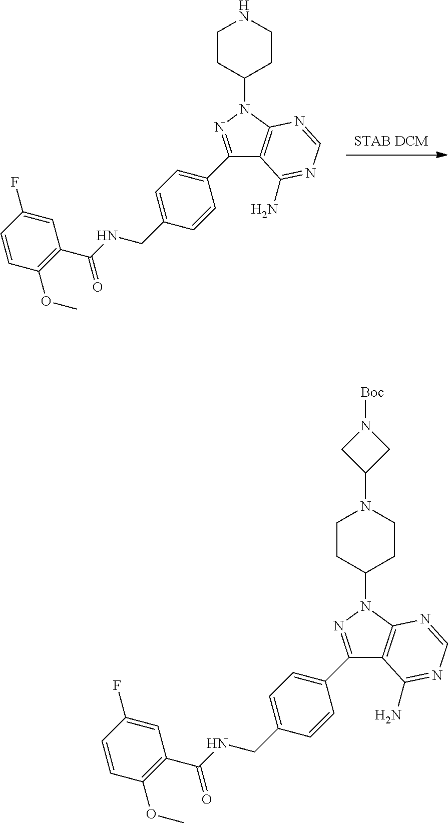

At room temperature, N-(4-(4-amino-1-(piperidin-4-yl)-1H-pyrazolo[3,4-d]pyrimidin-3-yl)benzyl)-5-fluoro-2-methoxybenzamide (0.50 g, 1.05 mmol), N-Boc-cyclobutanone (0.27 g, 1.57 mmol), sodium triacetoxyborohydride (0.33 g, 1.57 mmol) and DCM (30 mL) were added into a single-necked flask in sequence and the reaction was allowed to proceed at room temperature for 16 h. The reaction solution was concentrated and subjected to column chromatography (MeOH/DCM system) to afford 0.40 g of a yellow solid with a yield of 60%.

### Step 13: Synthesis of N-(4-(4-amino-1-(1-(azetidin-3-yl) piperidin-4-yl)-1H-pyrazolo[3,4-d]pyrimidine-3-methyl)benzyl)-5-fluoro-2-methoxybenzamide

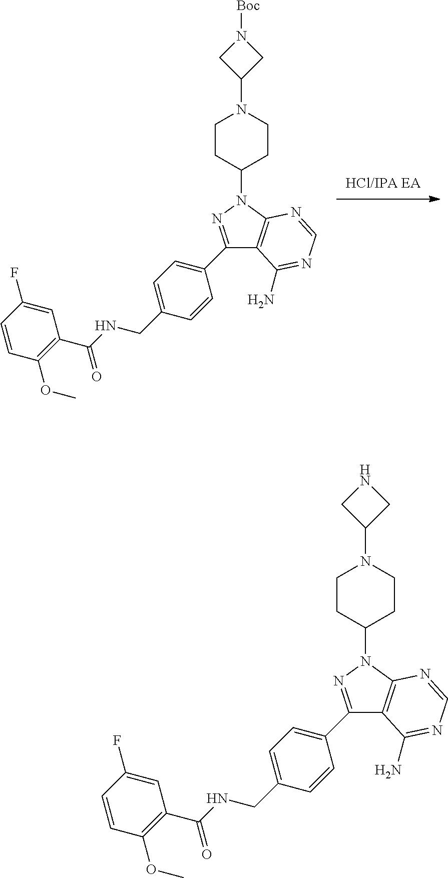 [SMILES: COc1ccc(F)cc1C(=O)NCc1ccc(-c2nn(C3CCN(C4CN(C(=O)OC(C)(C)C)C4)CC3)c3ncnc(N)c23)cc1 ; COc1ccc(F)cc1C(=O)NCc1ccc(-c2nn(C3CCN(C4CNC4)CC3)c3ncnc(N)c23)cc1]

At room temperature, tert-butyl 3-(4-(4-amino-3-(4-((5-fluoro-2-methoxybenzamido)methyl)phenyl)-1H-pyrazolo[3,4-d]pyrimidin-1-yl)piperidin-1-methyl) azetidine-1-carboxylate (0.40 g, 0.63 mmol), isopropanol hydrochloride (10 mL), and EA (10 mL) were added into a single-necked flask in sequence and the reaction was allowed to proceed at room temperature for 3 h. The reaction solution was concentrated and treated with triethylamine to afford 0.40 g of a crude product as a white solid.

### Step 14: Synthesis of N-(4-(4-amino-1-(1-(1-((1-(4-(2,4-dioxotetrahydropyrimidin-1(2H)-yl)phenyl) piperidin-4-yl)methyl) azetidin-3-yl) piperidin-4-yl)-1H-pyrazolo[3,4-d]pyrimidin-3-methyl)benzyl)-5-fluoro-2-methoxybenzamide

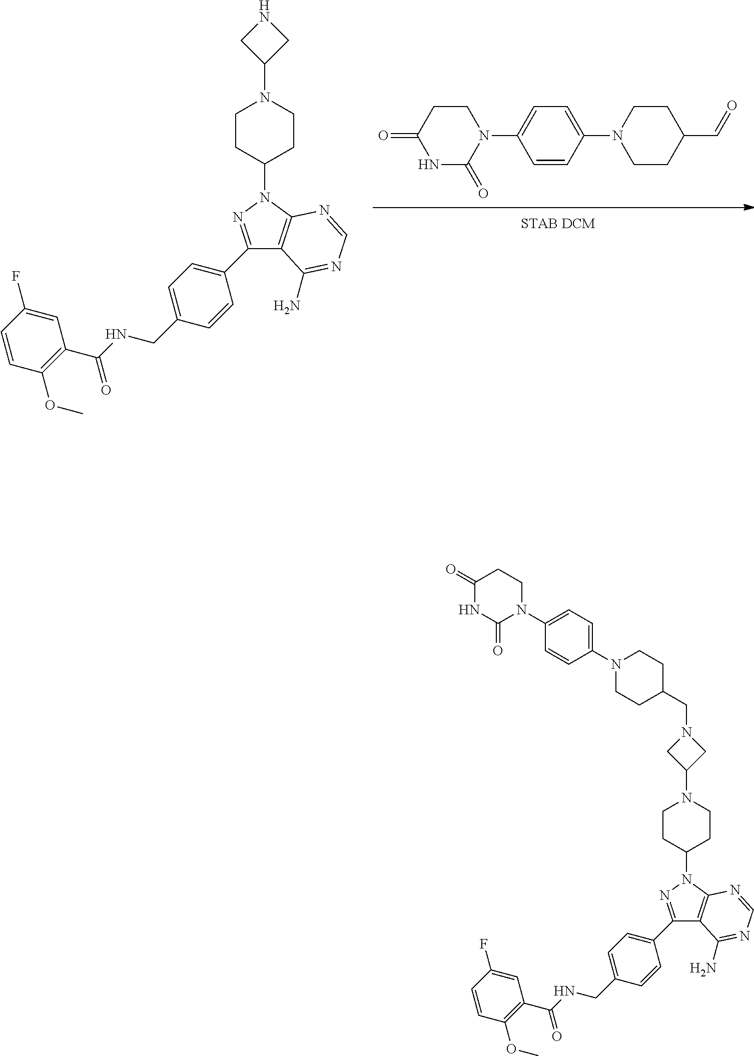 [SMILES: COc1ccc(F)cc1C(=O)NCc1ccc(-c2nn(C3CCN(C4CNC4)CC3)c3ncnc(N)c23)cc1 ; O=CC1CCN(c2ccc(N3CCC(=O)NC3=O)cc2)CC1 ; COc1ccc(F)cc1C(=O)NCc1ccc(-c2nn(C3CCN(C4CN(CC5CCN(c6ccc(N7CCC(=O)NC7=O)cc6)CC5)C4)CC3)c3ncnc(N)c23)cc1]

At room temperature, N-(4-(4-amino-1-(1-(azetidin-3-yl) piperidin-4-yl)-1H-pyrazolo[3,4-d]pyrimidin-3-methyl)benzyl)-5-fluoro-2-methoxybenzamide (0.10 g, 0.18 mmol), 1-(4-(2,4-dioxotetrahydropyrimidin-1(2H)-yl)phenyl)piperidine-4-carbaldehyde (0.07 g, 0.23 mmol), sodium triacetoxyborohydride (0.06 g, 0.27 mmol) and DCM (20 mL) were added into a single-necked flask in sequence and the reaction was allowed to proceed at room temperature for 1 h. The reaction solution was concentrated and subjected to column chromatography (MeOH/DCM system) to afford 0.09 g of a white solid with a yield of 60%, ¹H NMR (400 MHz, DMSO-d6) δ 10.26 (s, 1H), 8.89 (t, J=6.1 Hz, 1H), 8.24 (s, 1H), 7.65 (d, J=7.8 Hz, 2H), 7.57-7.48 (m, 3H), 7.40-7.31 (m, 1H), 7.20 (dd, J=9.2, 4.3 Hz, 1H), 7.13 (d, J=8.4 Hz, 2H), 6.92 (d, J=8.6 Hz, 2H), 4.73-4.63 (m, 1H), 4.59 (d, J=6.1 Hz, 2H), 3.91 (s, 3H), 3.73-3.62 (m, 5H), 3.02-2.91 (m, 3H), 2.85 (d, J=10.2 Hz, 2H), 2.71-2.57 (m, 4H), 2.47-2.38 (m, 2H), 2.27-2.12 (m, 2H), 2.00 (t, J=11.6 Hz, 2H), 1.94-1.86 (m, 4H), 1.76 (d, J=12.3 Hz, 2H), 1.55-1.42 (m, 1H), 1.28-1.10 (m, 3H). MS (ESI) m/z: 816.8 [M+H]⁺.

### Example 2: Synthesis of N-(4-(4-amino-1-(6-(4-((1-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-d]pyrimidin-3-yl)benzyl)-5-fluoro-2-methoxybenzamide (Compound I-2)

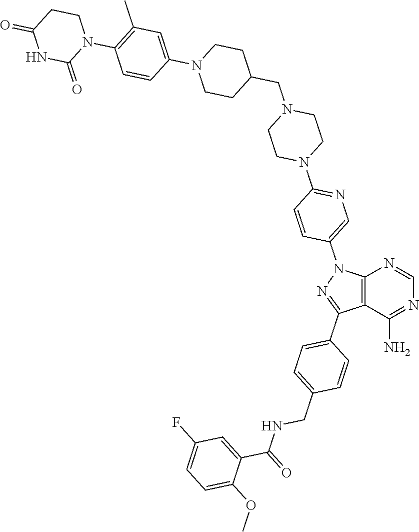 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1]

The synthesis method of N-(4-(4-amino-1-(6-(piperazin-1-yl)pyridin-3-yl)-1H-pyrazolo[3,4-d]pyrimidin-3-yl)benzyl)-5-fluoro-2-methoxybenzamide follows Example 1.

### Step 1: Synthesis of N-(4-(4-amino-1-(6-(4-((1-(4-(2,4-dioxotetrahydropyrimidin-1(2H)-yl)-3-methylphenyl)piperidin-4-yl))methyl)piperazin-1-yl)pyridin-3-yl)-1H-pyrazolo[3,4-d]pyrimidin-3-yl)benzyl)-5-fluoro-2-methoxybenzamide

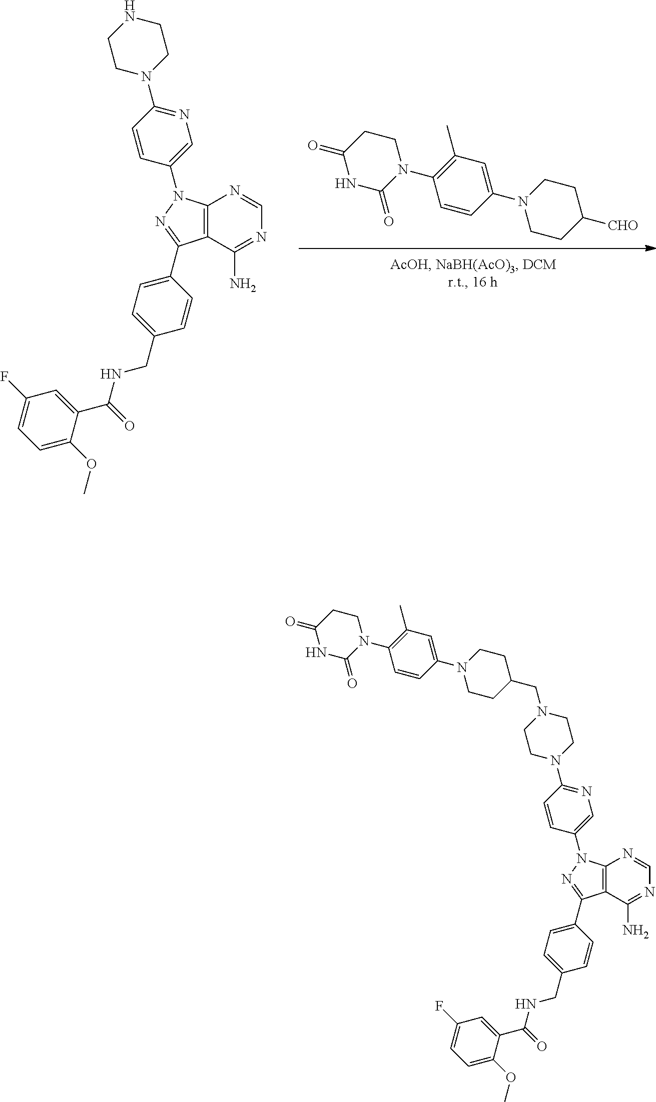 [SMILES: COc1ccc(F)cc1C(=O)NCc1ccc(-c2nn(-c3ccc(N4CCNCC4)nc3)c3ncnc(N)c23)cc1 ; Cc1cc(N2CCC(C=O)CC2)ccc1N1CCC(=O)NC1=O ; COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1]

N-(4-(4-amino-1-(6-(piperazin-1-yl)pyridin-3-yl)-1H-pyrazolo[3,4-d]pyrimidin-3-yl)benzyl)-5-fluoro-2-methoxybenzamide (0.05 g, 0.09 mmol), 1-(4-(2,4-dioxotetrahydropyrimidin-1(2H)-yl)-3-methylphenyl)piperidine-4-carbaldehyde (0.035 g, 0.11 mmol), dichloromethane (15 mL), acetic acid (0.05 g, 0.90 mmol), and sodium triacetoxyborohydride (0.03 g, 0.14 mmol) were added into a single-necked flask in sequence, and the mixture was stirred at room temperature for 16 h. The reaction solution was neutralized with a saturated aqueous sodium bicarbonate solution, the layers were separated, and the organic phase was concentrated, and subjected to column chromatography (MeOH/DCM system) to afford 0.055 g of a product with a yield of 72%.

¹H NMR (400 MHz, DMSO-d₆) δ 10.25 (s, 1H), 8.93-8.87 (m, 1H), 8.77 (s, 1H), 8.33 (s, 1H), 8.17 (d, J=9.0 Hz, 1H), 7.73 (d, J=7.8 Hz, 2H), 7.55 (d, J=8.2 Hz, 3H), 7.39-7.33 (m, 1H), 7.20 (dd, J=9.1, 4.1 Hz, 1H), 7.04 (d, J=8.6 Hz, 1H), 7.01 (d, J=9.3 Hz, 1H), 6.82 (s, 1H), 6.78 (d, J=8.6 Hz, 1H), 4.61 (d, J=5.9 Hz, 2H), 3.92 (s, 3H), 3.73-3.63 (m, 3H), 3.62-3.52 (m, 4H), 3.51-3.37 (m, 3H), 2.74-2.61 (m, 4H), 2.49-2.46 (m, 2H), 2.27-2.18 (m, 2H), 2.12 (s, 3H), 1.86-1.78 (m, 2H), 1.77-1.67 (m, 1H), 1.25-1.19 (m, 2H). MS (ESI) m/z: 853.8 [M+H]⁺.

### Example 3: Synthesis of N-(4-(4-amino-1-(6-(4-((1-(4-(2,4-dioxotetrahydropyrimidin-1(2H)-yl)phenyl) piperidin-4-yl)methyl)piperazin-1-yl)pyridin-3-yl)-1H-pyrazolo[3,4-d]pyrimidin-3-yl)benzyl)-5-fluoro-2-methoxybenzamide (Compound I-3)

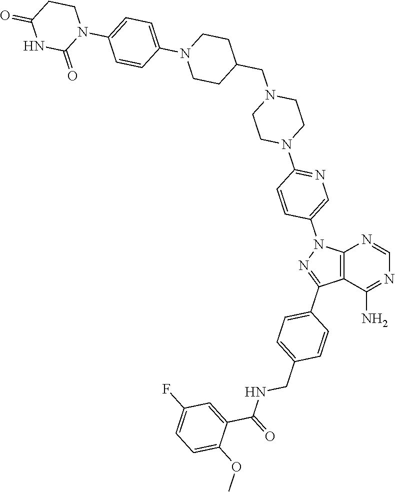 [SMILES: COc1ccc(F)cc1C(=O)NCc1ccc(-c2nn(-c3ccc(N4CCN(CC5CCN(c6ccc(N7CCC(=O)NC7=O)cc6)CC5)CC4)nc3)c3ncnc(N)c23)cc1]

The synthesis method of N-(4-(4-amino-1-(6-(piperazin-1-yl)pyridin-3-yl)-1H-pyrazolo[3,4-d]pyrimidin-3-yl)benzyl)-5-fluoro-2-methoxybenzamide follows Example 1.

### Step 1: Synthesis of N-(4-(4-amino-1-(6-(4-((1-(4-(2,4-dioxotetrahydropyrimidin-1(2H)-yl)phenyl) piperidin-4-yl)methyl)piperazin-1-yl)pyridin-3-yl)-1H-pyrazolo[3,4-d]pyrimidin-3-yl)benzyl)-5-fluoro-2-methoxybenzamide

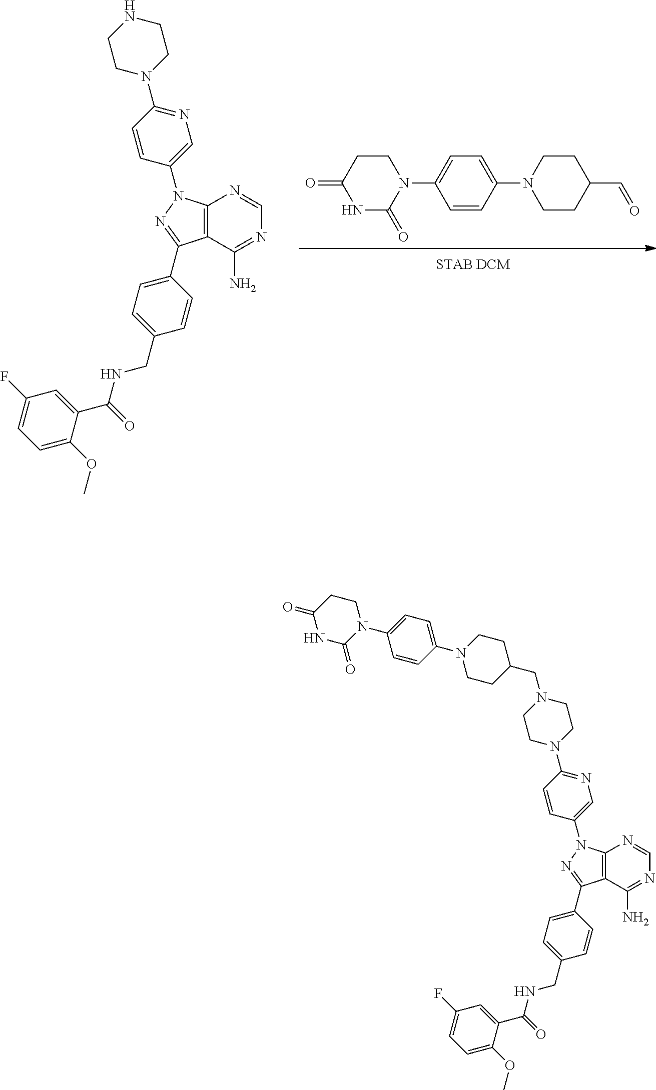

At room temperature, N-(4-(4-amino-1-(6-(piperazin-1-yl)pyridin-3-yl)-1H-pyrazolo[3,4-d]pyrimidin-3-yl)benzyl)-5-fluoro-2-methoxybenzamide (0.08 g, 0.14 mmol), 1-(4-(2,4-dioxotetrahydropyrimidin-1(2H)-yl)phenyl)piperidine-4-carbaldehyde (0.05 g, 0.17 mmol), sodium triacetoxyborohydride (0.05 g, 0.21 mmol) and DCM (20 mL) were added into a single-necked flask in sequence and the reaction was allowed to proceed at room temperature for 1 h. The reaction solution was concentrated and subjected to column chromatography (MeOH/DCM system) to afford 0.06 g of a yellow solid with a yield of 47%. ¹H NMR (400 MHz, DMSO-d6) δ 8.90 (t, J=6.1 Hz, 1H), 8.77 (d, J=2.6 Hz, 1H), 8.33 (s, 1H), 8.17 (dd, J=9.1, 2.7 Hz, 1H), 7.74 (d, J=7.8 Hz, 2H), 7.58-7.49 (m, 3H), 7.40-7.32 (m, 1H), 7.20 (dd, J=9.1, 4.3 Hz, 1H), 7.14 (d, J=8.5 Hz, 2H), 7.02 (d, J=9.3 Hz, 1H), 6.93 (d, J=8.6 Hz, 2H), 4.61 (d, J=6.1 Hz, 2H), 3.92 (s, 3H), 3.72-3.64 (m, 4H), 3.62-3.49 (m, 4H), 2.75-2.60 (m, 4H), 2.51-2.43 (m, 4H), 2.23 (d, J=7.1 Hz, 2H), 1.86-1.68 (m, 3H), 1.30-1.14 (m, 2H). MS (ESI) m/z: 840.8 [M+H]⁺.

### Example 4: Synthesis of N-(4-(4-amino-1-(6-(4-((1-(2-(2,6-dioxopiperidin-3-yl)-1-oxoisoindolin-5-yl) piperidin-4-yl)methyl)piperazin-1-yl)pyridin-3-yl)-1H-pyrazolo[3,4-d]pyrimidin-3-yl)phenyl)-5-fluoro-2-methoxybenzamide (Compound II-1)

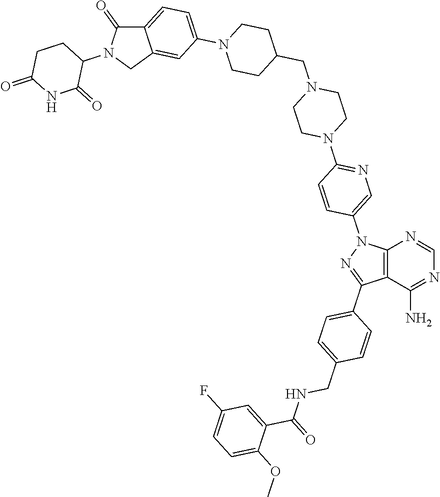 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1]

The synthesis method of N-(4-(4-amino-1-(6-(piperazin-1-yl)pyridin-3-yl)-1H-pyrazolo[3,4-d]pyrimidin-3-yl)benzyl)-5-fluoro-2-methoxybenzamide follows Example 1.

### Step 1: Synthesis of N-(4-(4-amino-1-(6-(4-((1-(2-(2,6-dioxopiperidin-3-yl)-1-oxoisoindolin-5-yl) piperidin-4-yl)methyl)piperazin-1-yl)pyridin-3-yl)-1H-pyrazolo[3,4-d]pyrimidin-3-yl)phenyl)-5-fluoro-2-methoxybenzamide

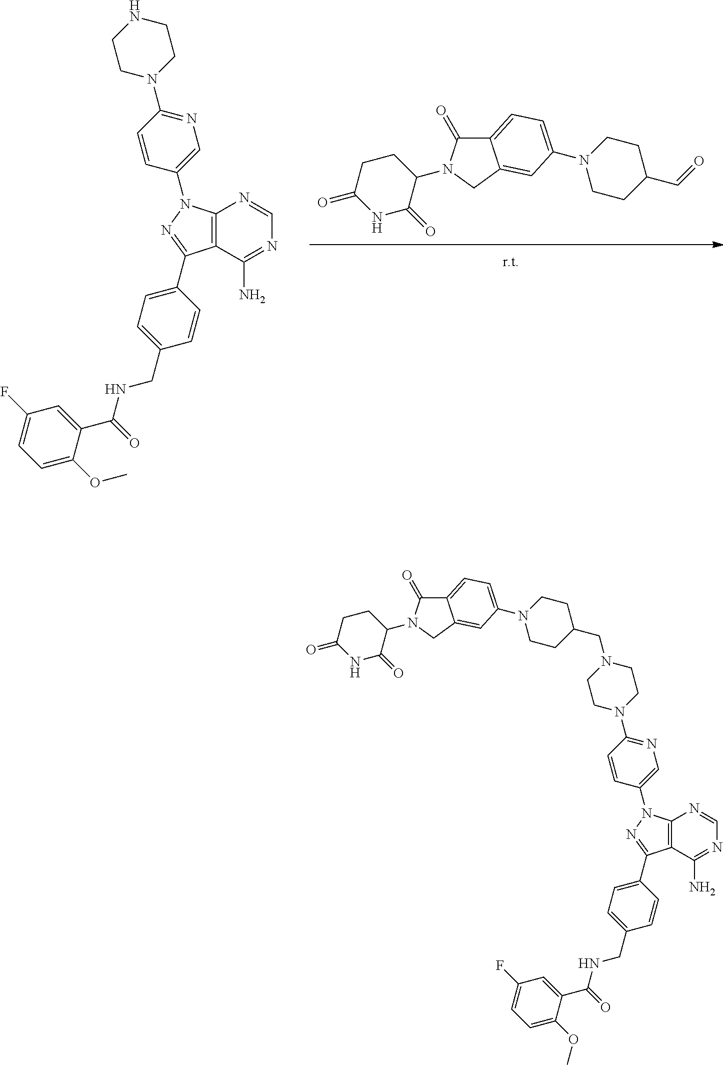 [SMILES: COc1ccc(F)cc1C(=O)NCc1ccc(-c2nn(-c3ccc(N4CCNCC4)nc3)c3ncnc(N)c23)cc1 ; O=CC1CCN(c2ccc3c(c2)CN(C2CCC(=O)NC2=O)C3=O)CC1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1]

At room temperature, N-(4-(4-amino-1-(6-(piperazin-1-yl)pyridin-3-yl)-1H-pyrazolo[3,4-d]pyrimidin-3-yl)phenyl)-5-fluoro-2-methoxybenzamide (0.08 g, 0.14 mmol), 1-(2-(2,6-dioxopiperidin-3-yl)-1-oxoisoindolin-5-yl)piperidine-4-carbaldehyde (0.06 g, 0.17 mmol), DCM (10 mL) and NaBH (OAc)₃ (0.05 g, 0.22 mmol) were added into a single-necked flask in sequence. The reaction was allowed to proceed at room temperature for 16 h under nitrogen protection before termination. The reaction solution was concentrated and purified by column chromatography (MeOH/DCM system) to afford 0.085 g of a white solid. The total yield of the two steps was 66%, 1H NMR (400 MHz, DMSO-d6) δ 8.76 (s, 1H), 8.33 (d, J=4.0 Hz, 1H), 8.16 (dd, J=9.2, 2.9 Hz, 1H), 7.78-7.70 (m, 2H), 7.60-7.47 (m, 4H), 7.37-7.32 (m, 1H), 7.21-7.17 (m, 1H), 7.10-6.97 (m, 3H), 5.04 (dd, J=12.9, 3.7 Hz, 1H), 4.37-4.27 (m, 1H), 4.25-4.13 (m, 1H), 3.91-3.82 (m, 5H), 3.60-3.52 (m, 4H), 2.93-2.80 (m, 3H), 2.68-2.53 (m, 2H), 2.48-2.29 (m, 5H), 2.23-2.17 (m, 2H), 1.97-1.81 (m, 5H), 1.25-1.17 (m, 2H). MS (ESI) m/z: 893.9 [M+H]⁺.

### Example 5: Synthesis of N-(4-(4-amino-1-(6-(4-((1-(2-(2,6-dioxopiperidin-3-yl)-1-oxoisoindolin-5-yl) piperidin-4-yl)methyl)piperazin-1-yl)pyridin-3-yl)-1H-pyrazolo[3,4-d]pyrimidin-3-yl)-2-methylbenzyl)-5-fluoro-2-methoxybenzamide (Compound II-2)

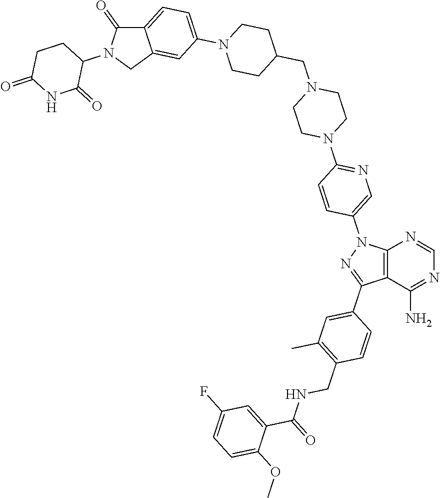 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1C]

The synthesis method of N-(4-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide follows Example 1.

### Step 1: Synthesis of N-(4-bromo-2-methylbenzyl)-5-fluoro-2-methoxybenzamide

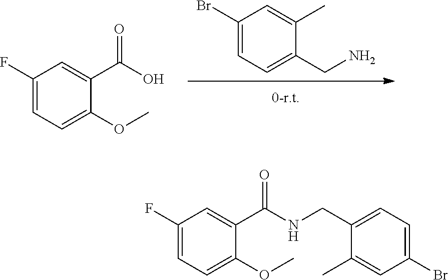

At 0° C., 5-fluoro-2-methoxybenzoic acid (1.00 g, 5.88 mmol), (4-bromo-2-methylphenyl) methanamine (1.18 g, 5.88 mmol), N-methylimidazole (1.21 g, 14.69 mmol) and DMF (20 mL) were added into a single-necked flask in sequence, TCFH (4.12 g, 14.69 mmol) was slowly added in portions. Under nitrogen protection, the mixture was warmed to room temperature and the reaction was allowed to proceed for 18 h before termination. The reaction was quenched with water and the reaction solution was extracted with EA. The organic phase was dried over anhydrous Na₂SO₄, filtered, concentrated and purified by column chromatography (EA/PE system) to afford 2.11 g of a crude product as a yellow solid. MS (ESI) m/z: 352.3 [M+H]⁺.

### Step 2: Synthesis of 5-fluoro-2-methoxy-N-(2-methyl-4-(4,4,5-trimethyl-1,3,2-dioxaborolan-2-yl)benzyl)benzamide

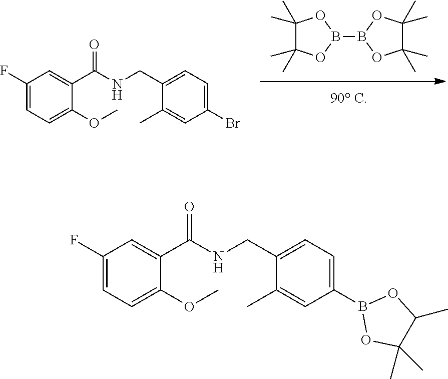

At room temperature, N-(4-bromo-2-methylbenzyl)-5-fluoro-2-methoxybenzamide (2.11 g, 5.99 mmol), bis(pinacolato)diboron (2.28 g, 8.99 mmol), Pd(dppf)Cl₂ (2.28 g, 8.99 mmol), KOAc (1.18 g, 11.98 mmol) and Dioxane (40 mL) were added into a single-necked flask in sequence, and the reaction was allowed to proceed at 90° C. for 8 h under nitrogen protection before termination. The reaction solution was filtered and rinsed with EA. The organic phase was filtered, concentrated and purified by column chromatography (EA/PE system) to afford 2.03 g of a light yellow solid. The yield of the two steps was 85%. MS (ESI) m/z: 400.6 [M+H]⁺.

### Step 3: Synthesis of tert-butyl 4-(5-(4-amino-3-(4-((5-fluoro-2-methoxybenzamido)methyl)-3-methylphenyl)-1H-pyrazolo[3,4-d]pyrimidin-1-yl)pyridin-2-yl) piperazine-1-carboxylate

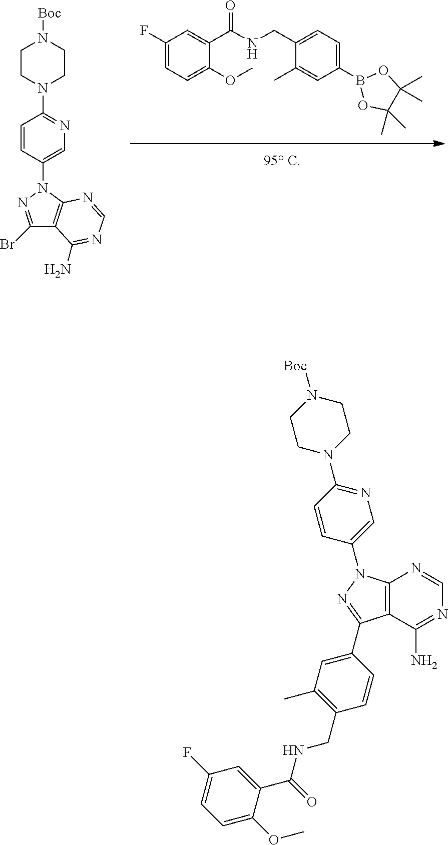 [SMILES: CC(C)(C)OC(=O)N1CCN(c2ccc(-n3nc(Br)c4c(N)ncnc43)cn2)CC1 ; COc1ccc(F)cc1C(=O)NCc1ccc(B2OC(C)(C)C(C)(C)O2)cc1C ; COc1ccc(F)cc1C(=O)NCc1ccc(-c2nn(-c3ccc(N4CCN(C(=O)OC(C)(C)C)CC4)nc3)c3ncnc(N)c23)cc1C]

At room temperature, tert-butyl 4-(5-(4-amino-3-bromo-1H-pyrazolo[3,4-d]pyrimidin-1-yl)pyridin-2-yl) piperazine-1-carboxylate (0.15 g, 0.32 mmol), 5-fluoro-2-methoxy-N-(2-methyl-4-(4,4,5-trimethyl-1,3,2-dioxaborolan-2-yl)benzyl)benzamide (0.32 g, 0.79 mmol), Cs₂CO₃ (0.31 g, 0.95 mmol), Pd(PPh₃)₂Cl₂ (0.07 g, 0.09 mmol) and Dioxane/H₂O (20 mL) were added into a single-necked flask in sequence. Under nitrogen protection, the mixture was slowly heated to 95° C. and the reaction was allowed to proceed for 16 h before termination. The reaction solution was cooled, filtered, and rinsed with EA, and the filtrate was dried over anhydrous Na₂SO₄, filtered, concentrated and purified by column chromatography (EA/PE system) to afford 0.14 g of a green solid with a yield of 66%. MS (ESI) m/z: 668.7 [M+H]⁺.

### Step 4: Synthesis of N-(4-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide

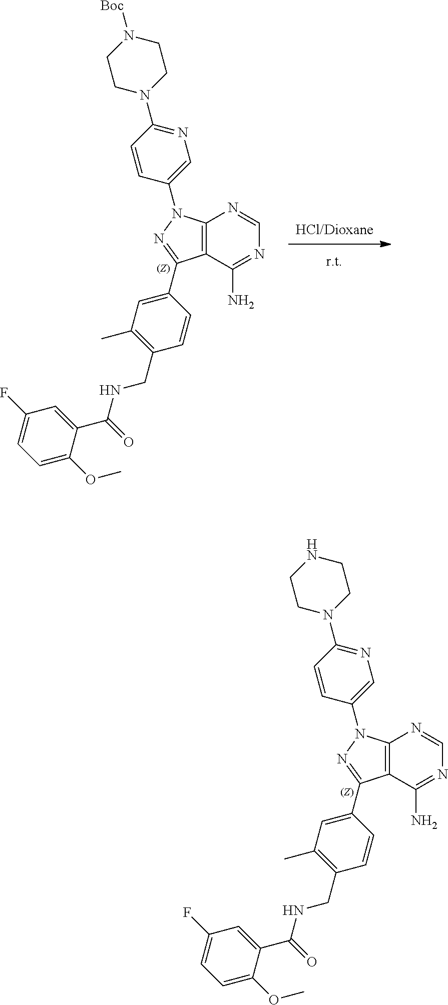 [SMILES: COc1ccc(F)cc1C(=O)NCc1ccc(-c2nn(-c3ccc(N4CCN(C(=O)OC(C)(C)C)CC4)nc3)c3ncnc(N)c23)cc1C ; COc1ccc(F)cc1C(=O)NCc1ccc(-c2nn(-c3ccc(N4CCNCC4)nc3)c3ncnc(N)c23)cc1C]

At room temperature, tert-butyl 4-(5-(4-amino-3-(4-((5-fluoro-2-methoxybenzamido)methyl)-3-methylphenyl)-1H-pyrazolo[3,4-d]pyrimidin-1-yl)pyridin-2-yl) piperazine-1-carboxylate (0.14 g, 0.21 mmol), DCM (10 mL), and HCl/Dioxane (10 mL) were added into a single-necked flask in sequence. The reaction was allowed to proceed at room temperature for 2 h under nitrogen protection before termination. The reaction was quenched, the layers were separated, and the aqueous layer was extracted with DCM. The organic phases were combined, dried over anhydrous Na₂SO₄, filtered, and concentrated to afford 0.12 g of a crude product as a white solid. MS (ESI) m/z: 568.6 [M+H]⁺.

### Step 5: Synthesis of N-(4-(4-amino-1-(6-(4-((1-(2-(2,6-dioxopiperidin-3-yl)-1-oxoisoindolin-5-yl) piperidin-4-yl)methyl)piperazin-1-yl)pyridin-3-yl)-1H-pyrazolo[3,4-d]pyrimidin-3-yl)-2-methylbenzyl)-5-fluoro-2-methoxybenzamide

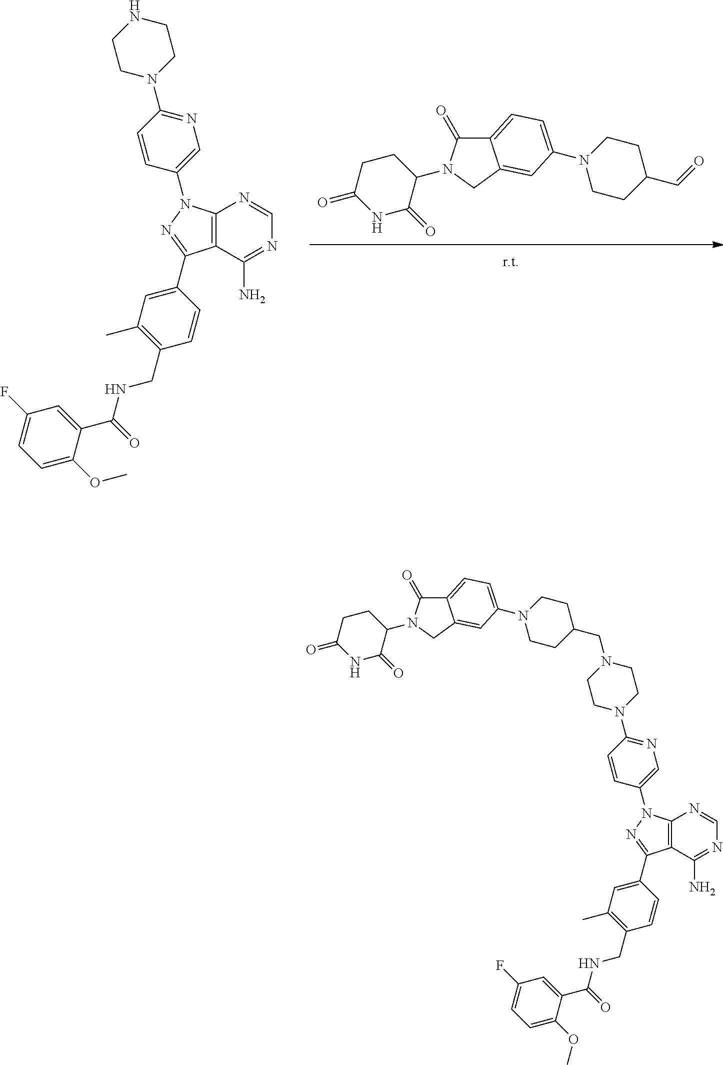 [SMILES: COc1ccc(F)cc1C(=O)NCc1ccc(-c2nn(-c3ccc(N4CCNCC4)nc3)c3ncnc(N)c23)cc1C ; O=CC1CCN(c2ccc3c(c2)CN(C2CCC(=O)NC2=O)C3=O)CC1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1C]

At room temperature, N-(4-(4-amino-1-(6-(piperazin-1-yl)pyridin-3-yl)-1H-pyrazolo[3,4-d]pyrimidin-3-yl]-2-methylbenzyl)-5-fluoro-2-methoxybenzamide (0.08 g, 0.14 mmol), 1-(2-(2,6-dioxopiperidin-3-yl)-1-oxoisoindolin-5-yl)piperidine-4-carbaldehyde (0.06 g, 0.17 mmol), DCM (10 mL) and NaBH (OAc)₃ (0.05 g, 0.22 mmol) were added into a single-necked flask in sequence. The reaction was allowed to proceed at room temperature for 16 h under nitrogen protection before termination. The reaction solution was concentrated and purified by column chromatography (MeOH/DCM system) to afford 0.08 g of a white solid. The total yield of the two steps was 63%. ¹H NMR (400 MHz, DMSO-d₆) δ 8.76 (s, 1H), 8.32 (d, J=4.0 Hz, 1H), 8.16 (d, J=8.8 Hz, 1H), 7.59-7.46 (m, 5H), 7.37-7.31 (m, 1H), 7.23-7.17 (m, 1H), 7.09-6.98 (m, 3H), 5.08-5.00 (m, 1H), 4.36-4.28 (m, 1H), 4.24-4.16 (m, 1H), 3.92-3.84 (m, 5H), 3.60-3.50 (m, 4H), 2.96-2.78 (m, 3H), 2.69-2.52 (m, 2H), 2.48-2.45 (m, 3H), 2.43 (s, 3H), 2.41-2.29 (m, 2H), 2.24-2.17 (m, 2H), 2.01-1.89 (m, 2H), 1.86-1.76 (m, 3H), 1.24-1.17 (m, 2H). MS (ESI) m/z: 907.9 [M+H]⁺.

### Example 6: Synthesis of N-(4-4-amino-1-(6-(4-(1-(2,6-dioxopiperidin-3-yl)-1,3-dioxoisoindolin-5-ylpiperidin-4-yl)methylpiperazin-1-ylpyridin-3-yl)-1H-pyrazolo[3,4-d]pyrimidin-3-yl)benzyl)-5-fluoro-2-methoxybenzamide (Compound II-3)

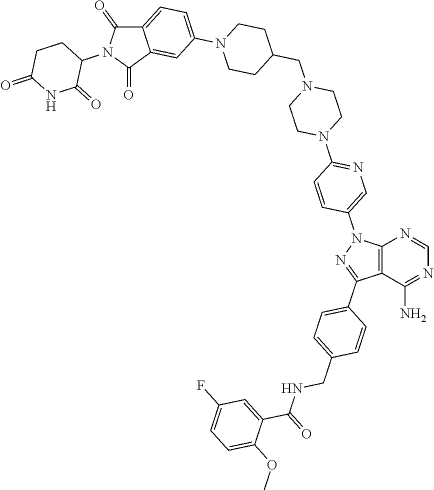 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1]

The synthesis method of 4-(4-amino-1-(6-piperazin-1-yl)pyridin-3-yl)-3-pyrazolopyrazolin-3-ylbenzyl)-5-fluoro-2-methoxybenzamide follows Example 1.

### Step 1: Synthesis of benzyl 4-(dimethoxymethyl)piperidine-1-carboxylate

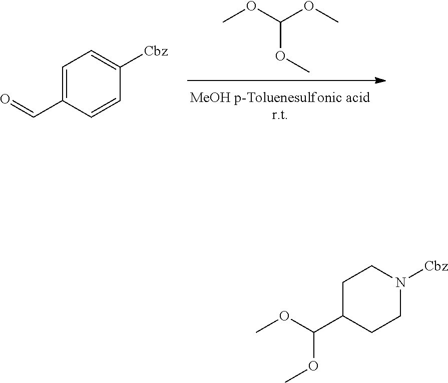

At room temperature, benzyl 4-formylbenzoate (10.00 g, 40.4 mmol), trimethyl orthoformate (21.45 g, 202.2 mmol), 4-methylbenzenesulfonic acid hydrate (1.53 g, 8.09 mmol) and MeOH (30 mL) were added into a single-necked flask in sequence. The reaction was allowed to proceed at room temperature for 24 h under nitrogen protection before termination. The reaction solution was cooled, then poured into a saturated sodium bicarbonate solution and extracted with EA. The organic phase was dried over anhydrous Na₂SO₄, filtered, concentrated and purified by column chromatography (EA/PE system) to afford 10.17 g of a colorless transparent liquid with a yield of 86.8%. MS (ESI) m/z: 294.16 [M+H]⁺.

### Step 2: Synthesis of 4-(dimethoxymethyl)piperidine

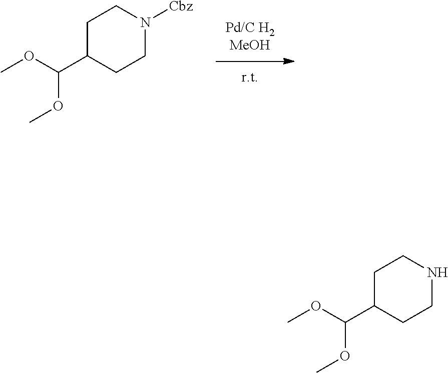 [SMILES: COC(OC)C1CCN(C(=O)OCc2ccccc2)CC1 ; COC(OC)C1CCNCC1]

At room temperature, benzyl 4-(dimethoxymethyl)piperidine-1-carboxylate (10.17 g, 34.7 mmol), Pd/C (10%, 1.5 g) and MeOH (50 mL) were added into a single-necked flask in sequence. The reaction was allowed to proceed at room temperature for 20 h under hydrogen protection before termination. The reaction solution was filtered to remove Pd/C, Pd/C was recovered, and the filtrate was collected and concentrated to afford 5.80 g of a light yellow liquid.

### Step 3: Synthesis of 5-(4-dimethoxymethyl) piperidin-1-yl-2-(2,6-dioxopiperidin-3-yl) isoindoline-1,3-dione

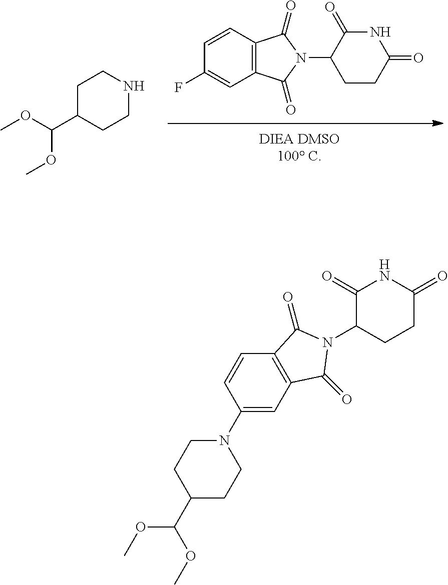 [SMILES: COC(OC)C1CCNCC1 ; O=C1CCC(N2C(=O)c3ccc(F)cc3C2=O)C(=O)N1 ; COC(OC)C1CCN(c2ccc3c(c2)C(=O)N(C2CCC(=O)NC2=O)C3=O)CC1]

At room temperature, 4-(dimethoxymethyl)piperidine (2.00 g, 12.6 mmol), 2-(2,6-dioxopiperidin-3-yl)-5-fluoroisoindoline-1,3-dione (2.89 g, 15.1 mmol), DIEA (2.71 g, 25.1 mmol) and DMSO (30 mL) were added into a single-necked flask in sequence. The reaction was allowed to proceed at 100° C. for 3 h under nitrogen protection before termination. The reaction solution was cooled to room temperature, then diluted with water, and filtered, and the filter cake was collected. The filter cake was dissolved in EA and then dried over anhydrous Na₂SO₄ and concentrated to afford 3.63 g of yellow solid with a yield of 83.5%. MS (ESI) m/z: 416.17 [M+H]⁺.

### Step 3: Synthesis of 1-(2-(2,6-dioxopiperidin-3-yl)-1,3-dioxoisoindolin-5-yl)piperidine-4-carboxaldehyde

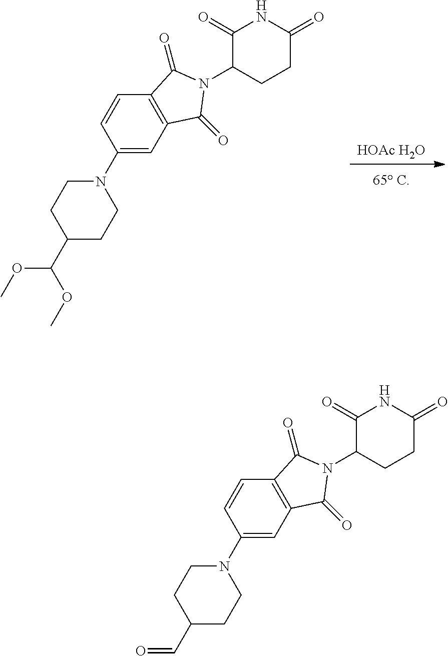 [SMILES: COC(OC)C1CCN(c2ccc3c(c2)C(=O)N(C2CCC(=O)NC2=O)C3=O)CC1 ; O=CC1CCN(c2ccc3c(c2)C(=O)N(C2CCC(=O)NC2=O)C3=O)CC1 ; "HOAc H2O"]

At room temperature, 5-(4-dimethoxymethyl) piperidin-1-yl-2-(2,6-dioxopiperidin-3-yl) isoindoline-1,3-dione (1.0 g, 2.4 mmol), glacial acetic acid (20 mL) and water (20 mL) were added into a single-necked flask in sequence. The reaction was allowed to proceed at 65° C. for 15 h under nitrogen protection before termination. The reaction solution was cooled, then concentrated, and purified by column chromatography (EA/PE system) to afford 0.79 g of a yellow solid with a yield of 89.0%. MS (ESI) m/z: 370.14 [M+H]⁺.

### Step 4: Synthesis of N-(4-4-amino-1-(6-(4-(1-(2,6-dioxopiperidin-3-yl)-1,3-dioxoisoindolin-5-ylpiperidin-4-yl)methylpiperazin-1-ylpyridin-3-yl)-1H-pyrazolo[3,4-d]pyrimidin-3-yl)benzyl)-5-fluoro-2-methoxybenzamide

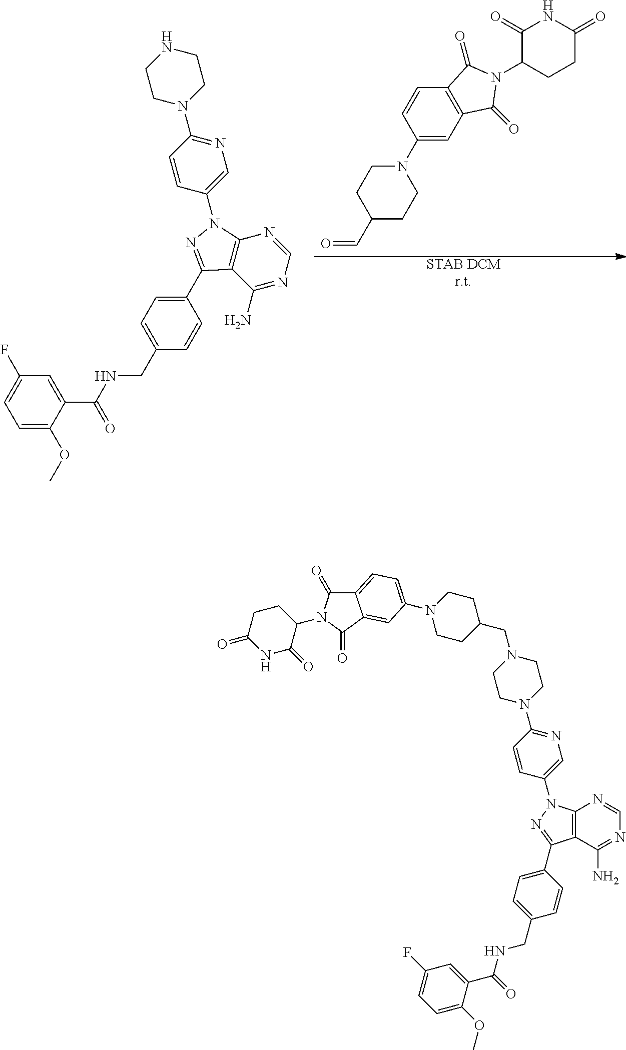

At room temperature, 1-(2-(2,6-dioxopiperidin-3-yl)-1,3-dioxoisoindolin-5-yl)piperidine-4-carboxaldehyde (27 mg, 0.07 mmol), 4-(4-amino-1-(6-piperazin-1-yl)pyridin-3-yl)-3-pyrazolopyrazolin-3-ylbenzyl)-5-fluoro-2-methoxybenzamide (50 mg, 0.09 mmol), STAB (23 mg, 0.11 mmol) and DCM (10 mL) were added into a single-necked flask in sequence. The reaction was allowed to proceed at room temperature for 3 h under nitrogen protection before termination. The reaction solution was cooled, concentrated and purified by column chromatography (MeOH/DCM system) to afford 0.040 g of a yellow solid with a yield of 54.2%, ¹H NMR (400 MHz, DMSO-d₆) δ 8.90 (t, J=5.8 Hz, 1H), 8.77 (d, J=2.9 Hz, 1H), 8.33 (s, 1H), 8.17 (d, J=9.0 Hz, 1H), 7.74 (d, J=7.6 Hz, 2H), 7.66 (d, J=8.4 Hz, 1H), 7.55 (d, J=7.9 Hz, 2H), 7.34 (d, J=13.1 Hz, 2H), 7.31-7.10 (m, 2H), 7.02 (d, J=9.3 Hz, 1H), 5.08 (dd, J=13.2, 5.1 Hz, 1H), 4.06 (d, J=13.0 Hz, 2H), 3.92 (s, 3H), 3.56 (m, 4H), 2.94 (dt, J=40.0, 13.7 Hz, 3H), 2.59 (d, J=20.0 Hz, 2H), 2.21 (d, J=6.7 Hz, 2H), 2.06 (d, J=21.6 Hz, 2H), 1.86 (t, J=18.4 Hz, 3H), 1.47-1.02 (m, 3H). MS (ESI) m/z: 907.82 [M+H]⁺.

### Example 7: Synthesis of N-(4-(4-amino-1-(1′-(((1R,2R)-2-((4-(4-(2,4-dioxotetrahydropyrimidin-1(2H)-yl)phenyl) piperazine))-1-yl)methyl)cyclohexyl)methyl)-[1,4′-bipiperidinyl]-4-yl)-1H-pyrazolo[3,4-d]pyrimidin-3-yl)benzyl)-5-fluoro-2-methoxybenzamide (Compound III-1)

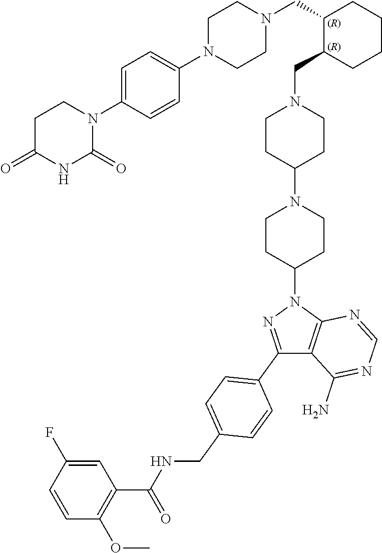

The synthesis method of N-(4-(4-amino-1-(piperidin-4-yl)-1H-pyrazolo[3,4-d]pyrimidin-3-yl)benzyl)-5-fluoro-2-methoxybenzamide follows Example 1.

### Step 1: tert-butyl 4-(4-(N-(3-ethoxy-3-oxopropyl) cyanamido)phenyl) piperazine-1-carboxylate

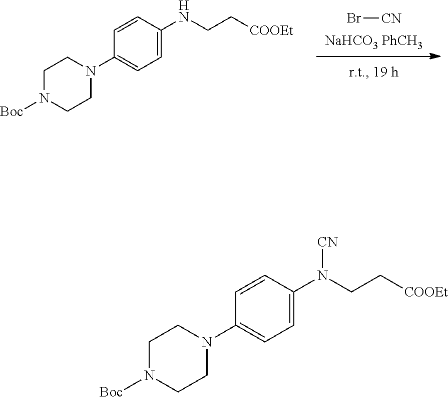

Tert-butyl 4-(4-((3-ethoxy-3-oxopropyl)amino)phenyl) piperazine-1-carboxylate (5.56 g, 14.73 mmol), sodium bicarbonate (3.71 g, 44.19 mmol), toluene (100 mL), and cyanogen bromide (3.12 g, 29.46 mmol) were added into a single-necked flask in sequence and the mixture was stirred at room temperature for 19 h. The reaction was quenched with water (50 mL). The layers were separated and the organic phase was concentrated to afford 6 g of a black oil, which was used directly in the next reaction without purification. MS (ESI) m/z: 403.4 [M+H]⁺.

### Step 2: tert-butyl 4-(4-(1-(3-ethoxy-3-oxopropyl) ureido)phenyl) piperazine-1-carboxylate

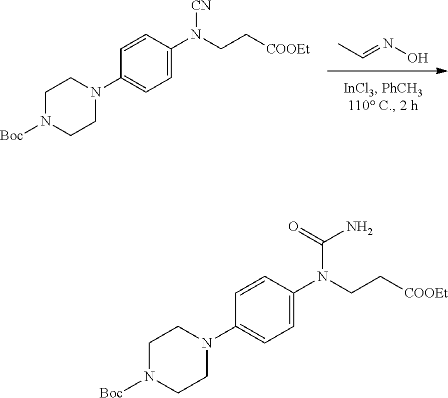

Crude tert-butyl 4-(4-(N-(3-ethoxy-3-oxopropyl) cyanamido)phenyl) piperazine-1-carboxylate (5.93 g, 14.73 mmol), toluene (150 mL), acetaldehyde oxime (2.60 g, 44.19 mmol) and indium chloride (0.98 g, 4.42 mmol) were added into a single-necked flask in sequence and the mixture was heated at 110° C. for 2 h. The reaction solution was concentrated, and subjected to column chromatography (MeOH/DCM system) to afford 4.04 g of a red waxy solid with a 2-step yield of 65%. MS (ESI) m/z: 421.3 [M+H]⁺.

### Step 3: tert-butyl 4-(4-(2,4-dioxotetrahydropyrimidin-1(2H)-yl)phenyl) piperazine-1-carboxylate

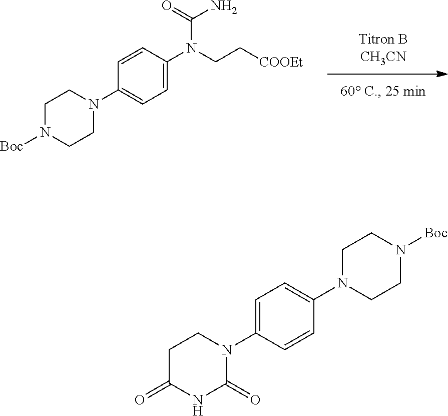 [SMILES: CCOC(=O)CCN(C(N)=O)c1ccc(N2CCN(C(=O)OC(C)(C)C)CC2)cc1 ; CC(C)(C)OC(=O)N1CCN(c2ccc(N3CCC(=O)NC3=O)cc2)CC1]

Tert-butyl 4-(4-(1-(3-ethoxy-3-oxopropyl) ureido)phenyl) piperazine-1-carboxylate (4.04 g, 9.60 mmol) and acetonitrile (100 mL) were added into a single-necked flask in sequence. The mixture were heated to 60° C., and then 40% benzyltrimethylammonium hydroxide in methanol (4.42 g, 10.56 mmol) was added, and the mixture was heated at 60° C. for 20 min. The reaction solution was concentrated and subjected to column chromatography (MeOH/DCM system) to afford 3.03 g of a yellow waxy solid with a yield of 84%. MS (ESI) m/z: 375.3 [M+H]⁺.

### Step 4:1-(4-(piperazin-1-yl)phenyl)dihydropyrimidine-2,4(1H,3H)-dione

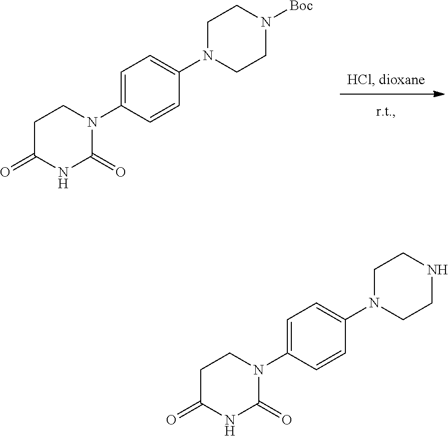 [SMILES: CC(C)(C)OC(=O)N1CCN(c2ccc(N3CCC(=O)NC3=O)cc2)CC1 ; O=C1CCN(c2ccc(N3CCNCC3)cc2)C(=O)N1]

Tert-butyl 4-(4-(2,4-dioxotetrahydropyrimidin-1(2H)-yl)phenyl) piperazine-1-carboxylate (3.03 g, 8.09 mmol), dioxane (30 mL), and hydrochloride in dioxane (50 mL) were added into a single-necked flask in sequence and the mixture was stirred at room temperature for 2 h. The reaction solution was concentrated to afford 2.74 g of a yellow solid with a yield of 98%. MS (ESI) m/z: 275.3 [M+H]⁺.

### Step 5: tert-butyl 4-(4-amino-3-(4-((5-fluoro-2-methoxybenzamido)methyl)phenyl)-1H-pyrazolo[3,4-d]pyrimidin-1-yl)-[1,4′-bipiperidine]-1′-carboxylate

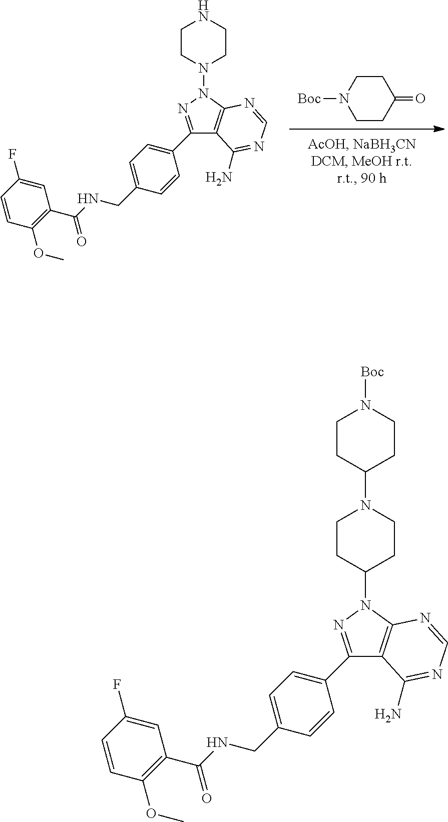 [SMILES: COc1ccc(F)cc1C(=O)NCc1ccc(-c2nn(N3CCNCC3)c3ncnc(N)c23)cc1 ; CC(C)(C)OC(=O)N1CCC(=O)CC1 ; COc1ccc(F)cc1C(=O)NCc1ccc(-c2nn(C3CCN(C4CCN(C(=O)OC(C)(C)C)CC4)CC3)c3ncnc(N)c23)cc1]

N-(4-(4-amino-1-(piperidin-4-yl)-1H-pyrazolo[3,4-d]pyrimidin-3-yl)benzyl)-5-fluoro-2-methoxybenzamide (1.80 g, 3.78 mmol), N-Boc piperidone (3.01 g, 15.12 mmol), dichloromethane (100 mL), methanol (10 mL), acetic acid (1.13 g, 18.9 mmol) and sodium cyanoborohydride (0.90 g, 15 mmol) were added into a single-necked flask in sequence, and the mixture was stirred at room temperature for 90 h. The reaction solution was neutralized with a saturated aqueous sodium bicarbonate solution, and the layers were separated. The aqueous phase was extracted with a DCM/MeOH mixed solvent, and the organic phases were combined, concentrated, and subjected to column chromatography (MeOH/DCM system) to afford 1.50 g of a yellow solid with a yield of 60%. MS (ESI) m/z: 659.6 [M+H]⁺.

### Step 6: N-(4-(1-([1,4′-bipiperidinyl]-4-yl)-4-amino-1H-pyrazolo[3,4-d]pyrimidin-3-yl)benzyl)-5-fluoro-2-methoxybenzamide

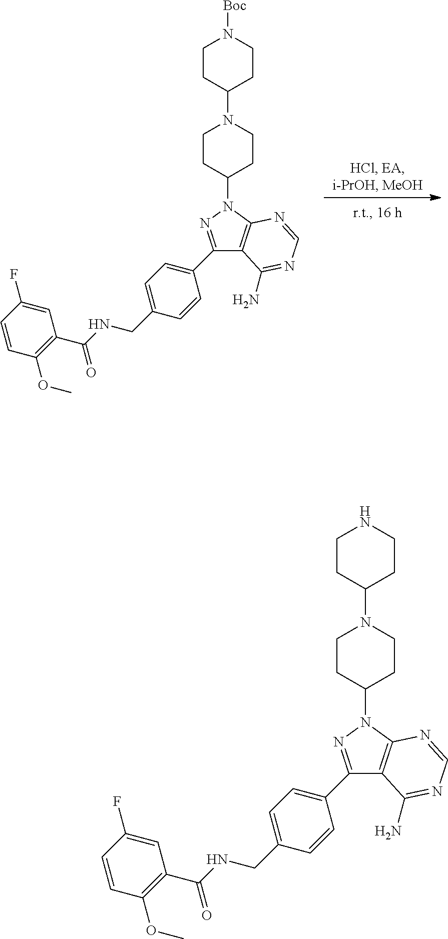 [SMILES: COc1ccc(F)cc1C(=O)NCc1ccc(-c2nn(C3CCN(C4CCN(C(=O)OC(C)(C)C)CC4)CC3)c3ncnc(N)c23)cc1 ; COc1ccc(F)cc1C(=O)NCc1ccc(-c2nn(C3CCN(C4CCNCC4)CC3)c3ncnc(N)c23)cc1]

Tert-butyl 4-(4-amino-3-(4-((5-fluoro-2-methoxybenzamido)methyl)phenyl)-1H-pyrazolo[3,4-d]pyrimidin-1-yl)-[1,4′-bipiperidine]-1′-carboxylate (1.5 g, 2.28 mmol), ethyl acetate (50 mL), methanol (10 mL), and hydrochloric acid in isopropanol (10 mL) were added into a single-necked flask in sequence and the mixture was stirred at room temperature for 16 h. The reaction solution was concentrated, neutralized with a saturated aqueous sodium bicarbonate solution, and evaporated to dryness. The residue was dissolved with a DCM/MeOH mixed solvent, insoluble matter was removed by filtration, and the filtrate was concentrated to afford 1.23 g of a brown solid with a yield of 96%. MS (ESI) m/z: 559.5 [M+H]⁺.

### Step 7: N-(4-(4-amino-1-(1′-(((1R,2R)-2-formylcyclohexyl)methyl)-[1,4′-bipiperidinyl]-4-yl)-1H-pyrazolin[3,4-d]pyrimidin-3-yl)benzyl)-5-fluoro-2-methoxybenzamide

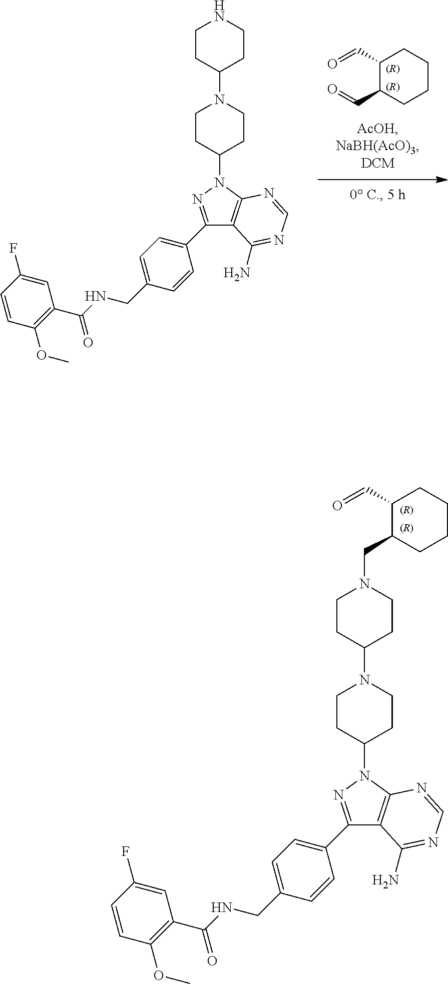

N-(4-(1-([1,4′-bipiperidinyl]-4-yl)-4-amino-1H-pyrazolo[3,4-d]pyrimidin-3-yl)benzyl)-5-fluoro-2-methoxybenzamide (0.38 g, 0.68 mmol), (1R,2R)-cyclohexane-1,2-dicarbaldehyde (0.76 g, 5.44 mmol), dichloromethane (30 mL), acetic acid (0.20 g, 3.4 mmol) and sodium triacetoxyborohydride (0.29 g, 1.36 mmol) were added into a single-necked flask in sequence under ice bath and the mixture was stirred in ice bath for 4 h. The reaction solution was neutralized with a saturated aqueous sodium bicarbonate solution, and the layers were separated. The aqueous phase was extracted with dichloromethane, and the organic phases were combined, concentrated, and subjected to column chromatography (MeOH/DCM system) to afford 0.31 g of a yellow solid with a yield of 67%. MS (ESI) m/z: 683.8 [M+H]⁺.

### Step 8: Synthesis of N-(4-(4-amino-1-(1′-(((1R,2R)-2-((4-(4-(2,4-dioxotetrahydropyrimidin-1(2H)-yl)phenyl) piperazine))-1-yl)methyl)cyclohexyl)methyl)-[1,4′-bipiperidinyl]-4-yl)-1H-pyrazolo[3,4-d]pyrimidin-3-yl)benzyl)-5-fluoro-2-methoxybenzamide

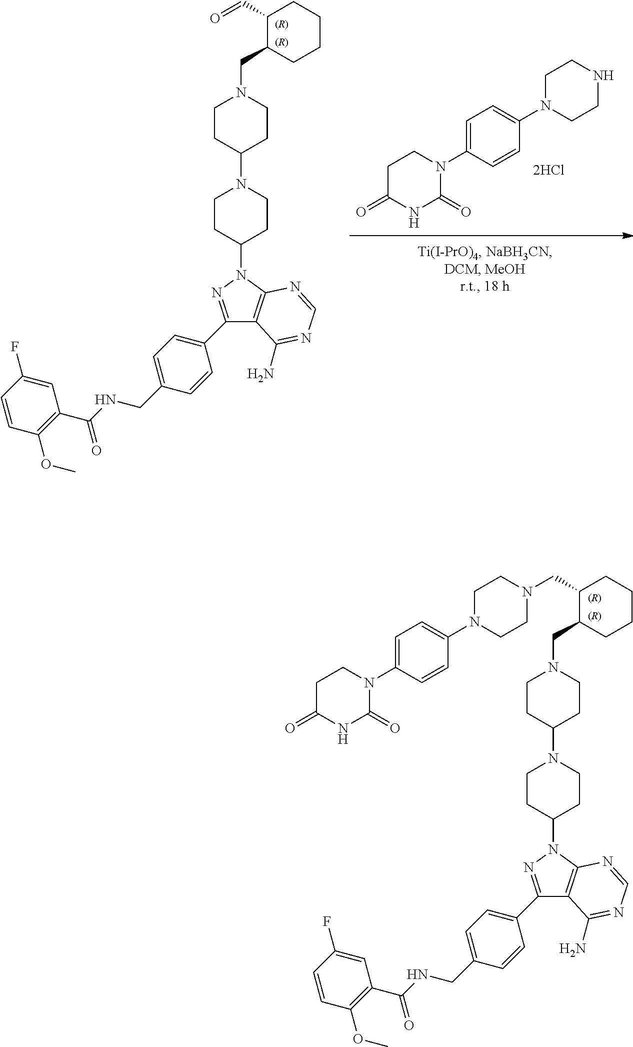

N-(4-(4-amino-1-(1′-(((1R,2R)-2-formylcyclohexyl)methyl)-[1,4′-bipiperidinyl]-4-yl)-1H-pyrazolin[3,4-d]pyrimidin-3-yl)benzyl)-5-fluoro-2-methoxybenzamide (0.07 g, 0.1 mmol), 1-(4-(piperazin-1-yl)phenyl)dihydropyrimidine-2,4(1H,3H)-dione hydrochloride (0.055 g, 0.15 mmol), dichloromethane (15 mL) and tetraisopropyl titanate (1.5 mL) were added into a single-necked flask in sequence and the mixture was stirred at room temperature for 15 h. Methanol (1.5 mL) and sodium cyanoborohydride (0.012 g, 0.2 mmol) were added, and the mixture was stirred at room temperature for another 6 h. Water (4 mL), dichloromethane (25 mL), and methanol (2.5 mL) were added, and the mixture was stirred for 15 min and filtered through celite. The filtrate was collected, and the filtered solid was slurried with a DCM/MeOH mixed solvent. The slurry was then filtered, and the filtrate was collected. The filtrates were combined, concentrated, and subjected to column chromatography (MeOH/DCM system) to afford 0.02 g of a white solid with a yield of 21%, 1H NMR (400 MHz, DMSO-d₆) δ 10.26 (s, 1H), 8.89 (t, J=6.1 Hz, 1H), 8.24 (s, 1H), 7.64 (d, J=8.0 Hz, 2H), 7.56-7.48 (m, 3H), 7.38-7.32 (m, 1H), 7.25-7.13 (m, 4H), 7.06-6.88 (m, 3H), 4.79-4.67 (m, 1H), 4.59 (d, J=6.0 Hz, 2H), 4.21-4.08 (m, 1H), 3.91 (s, 3H), 3.74-3.65 (m, 3H), 3.29-3.17 (m, 8H), 2.72-2.55 (m, 8H), 2.32-2.13 (m, 4H), 2.03-1.69 (m, 9H), 1.66-1.46 (m, 5H), 1.21-1.15 (m, 2H), 1.06-0.96 (m, 2H), 0.87-0.75 (m, 2H). MS (ESI) m/z: 941.9 [M+H]⁺.

### Example 9: Synthesis of 1-(4-(4-(((1R,2R)-2-((3-(4-(4-amino-3-(4-phenoxyphenyl)-1H-pyrazolo[3,4-d]pyrimidin-1-yl) piperidin-1-yl) azetidin-1-yl)methyl)cyclohexyl)methyl)piperazin-1-yl)phenyl)dihydropyrimidine-2,4(1H,3H)-dione (Compound III-2)

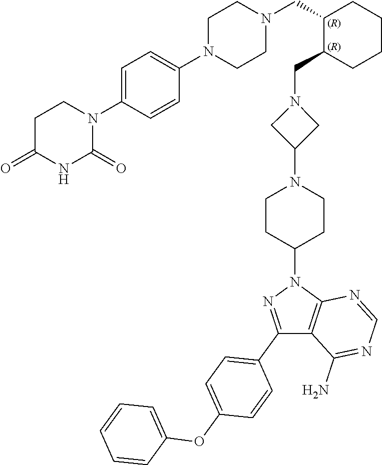

The synthesis method of (1R,2R)-2-((3-(4-(4-amino-3-(4-phenoxyphenyl)-1H-pyrazolo[3,4-d]pyrimidin-1-yl) piperidin-1-yl) azetidin-1-yl)methyl)cyclohexane-1-carbaldehyde follows Example 1.

### Step 1: Synthesis of 1-(4-(4-(((1R,2R)-2-((3-(4-(4-amino-3-(4-phenoxyphenyl)-1H-pyrazolo[3,4-d]pyrimidin-1-yl) piperidin-1-yl) azetidin-1-yl)methyl)cyclohexyl)methyl)piperazin-1-yl)phenyl)dihydropyrimidine-2,4(1H,3H)-dione

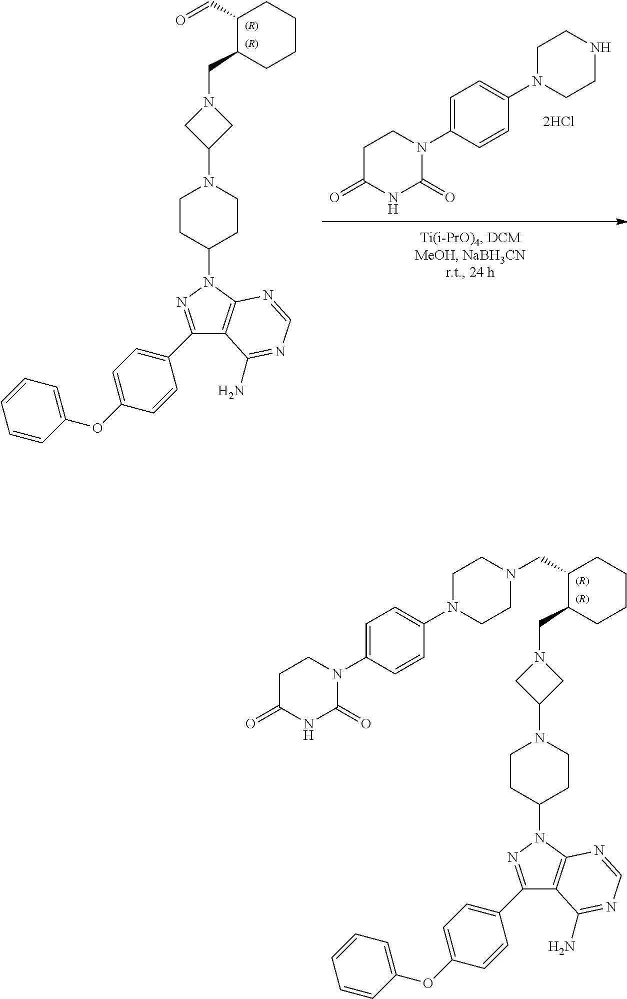

(1R,2R)-2-((3-(4-(4-amino-3-(4-phenoxyphenyl)-1H-pyrazolo[3,4-d]pyrimidin-1-yl) piperidin-1-yl) azetidin-1-yl)methyl)cyclohexane-1-carbaldehyde (0.14 g, 0.25 mmol), 1-(4-(piperazin-1-yl)phenyl)dihydropyrimidine-2,4(1H,3H)-dione hydrochloride (0.14 g, 0.38 mmol), dichloromethane (25 mL), and tetraisopropyl titanate (2.5 mL) were added into a single-necked flask in sequence and the mixture was stirred at room temperature for 18 h. Sodium cyanoborohydride (0.03 g, 0.5 mmol) and methanol (3 mL) were added to the reaction solution, and the mixture was stirred at room temperature for another 6 h. Water (5 mL), dichloromethane (25 mL), and methanol (2 mL) were added, and the mixture was filtered through celite. The filtrate was collected, and the filter cake was slurried with a DCM/MeOH mixed solvent. The slurry was then filtered, and the filtrate was collected. The filtrates were combined and concentrated. The concentrated filtrate was subjected to column chromatography (MeOH/DCM system) to afford 0.012 g of a white solid ¹H NMR (400 MHz, DMSO-d6) δ 10.24 (s, 1H), 8.25 (s, 1H), 7.73 (d, J=8.4 Hz, 2H), 7.41 (t, J=7.9 Hz, 2H), 7.21-7.11 (m, 4H), 7.09-7.03 (m, 3H), 6.98-6.83 (m, 2H), 4.81-4.73 (m, 1H), 4.01-3.81 (m, 4H), 3.66-3.51 (m, 6H), 3.19-3.16 (m, 1H), 3.15-3.11 (m, 1H), 3.09-2.94 (m, 4H), 2.88-2.66 (m, 5H), 2.64-2.59 (m, 2H), 2.38-2.23 (m, 3H), 2.05-1.95 (m, 3H), 1.91-1.84 (m, 1H), 1.76-1.57 (m, 5H), 1.23-1.16 (m, 2H), 1.07-0.93 (m, 2H). MS (ESI) m/z: 824.9 [M+H]⁺.

### Example 10: Synthesis of N-(4-(4-amino-1-(6-(4-((4-(2-(2,6-dioxopiperidin-3-yl)-3-oxoisoindolin-5-yl)piperazin-1-yl)methyl)piperidin-1-yl)pyridin-3-yl)-1H-pyrazolo[3,4-d]pyrimidin-3-yl)benzyl)-5-fluoro-2-methoxybenzamide (Compound II-4)

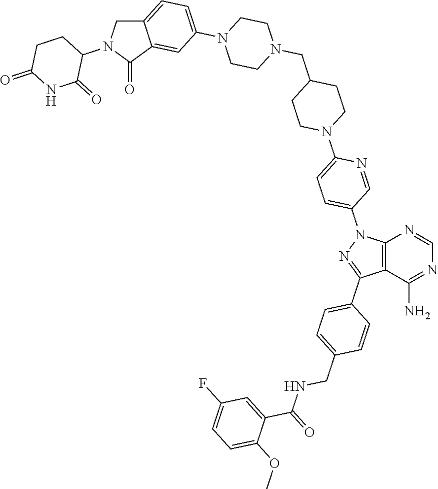 [SMILES: COc1ccc(F)cc1C(=O)NCc1ccc(-c2nn(-c3ccc(N4CCC(CN5CCN(c6ccc7c(c6)C(=O)N(C6CCC(=O)NC6=O)C7)CC5)CC4)nc3)c3ncnc(N)c23)cc1]

The synthesis method of N-(4-(4-amino-1-(6-(4-formylpiperidin-1-yl)pyridin-3-yl)-1H-pyrazolo[3,4-d]pyrimidin-3-yl)benzyl)-5-fluoro-2-methoxybenzamide follows Example 1.

### Step 1: Synthesis of 3-bromo-1-((2-(trimethylsilyl) ethoxy)methyl)-1H-pyrazolo[3,4-d]pyrimidine-4-amine

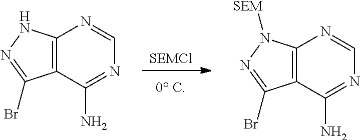 [SMILES: Nc1ncnc2[nH]nc(Br)c12 ; "SEMCl" ; C[Si](C)(C)CCOCn1nc(Br)c2c(N)ncnc21]

At 0° C., 3-bromo-1H-pyrazolo[3,4-d]pyrimidine-4-amine (5.00 g, 23.36 mmol) and DMF (50 mL) were added into a single-necked flask in sequence, NaH (1.40 g, 35.04 mmol) was slowly added, the reaction was allowed to proceed for half an hour after the addition, and SEMCI (4.67 g, 28.03 mmol) was slowly added dropwise. The reaction was allowed to proceed at 0° C. for 1.5 h under nitrogen protection before termination. The reaction was quenched with water, the reaction solution was diluted with EA, and the organic phase was dried over anhydrous Na₂SO₄, filtered, concentrated and purified by column chromatography (EA/PE system) to afford 4.24 g of a white solid with a yield of 53%. MS (ESI) m/z: 344.3 [M+H]⁺.

### Step 2: Synthesis of N-(4-(4-amino-1-((2-(trimethylsilyl) ethoxy)methyl)-1H-pyrazolo[3,4-d]pyrimidin-3-yl)benzyl)-5-fluoro-2-methoxybenzamide

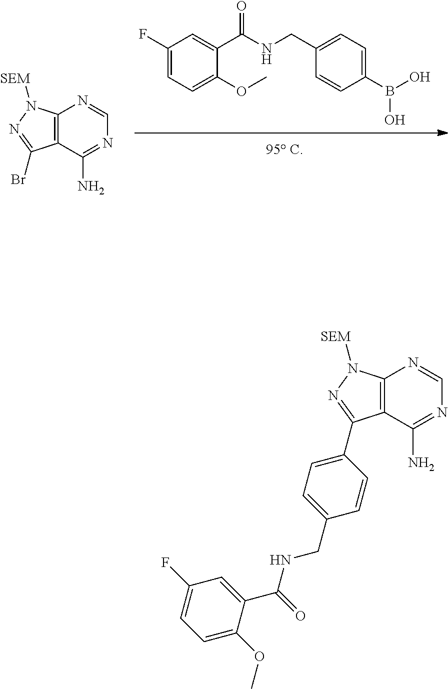 [SMILES: C[Si](C)(C)CCOCn1nc(Br)c2c(N)ncnc21 ; COc1ccc(F)cc1C(=O)NCc1ccc(B(O)O)cc1 ; COc1ccc(F)cc1C(=O)NCc1ccc(-c2nn(COCC[Si](C)(C)C)c3ncnc(N)c23)cc1]

At room temperature, 3-bromo-1-((2-(trimethylsilyl) ethoxy)methyl)-1H-pyrazolo[3,4-d]pyrimidin-4-amine (3.80 g, 11.00 mmol), (4-((5-fluoro-2-methoxybenzamido)methyl)phenyl)boronic acid (5.00 g, 16.50 mmol), Cs₂CO₃ (10.79 g, 33.11 mmol), Pd(PPh₃)₂Cl₂ (2.32 g, 3.31 mmol) and Dioxane/H₂O (80 mL) were added into a single-necked flask in sequence. Under nitrogen protection, the mixture was slowly heated to 95° C. and the reaction was allowed to proceed for 16 h before termination. The reaction solution was cooled, filtered, and rinsed with EA, and the filtrate was dried over anhydrous Na₂SO₄, filtered, concentrated and purified by column chromatography (EA/PE system) to afford 3.35 g of a red solid with a yield of 58%. MS (ESI) m/z: 523.6 [M+H]⁺.

### Step 3: Synthesis of N-(4-(4-amino-1H-pyrazolo[3,4-d]pyrimidin-3-yl)benzyl)-5-fluoro-2-methoxybenzamide

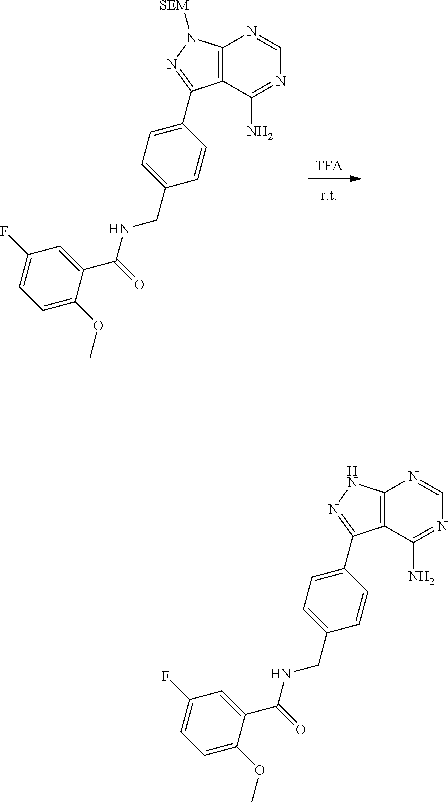 [SMILES: COc1ccc(F)cc1C(=O)NCc1ccc(-c2nn(COCC[Si](C)(C)C)c3ncnc(N)c23)cc1 ; "TFA" ; COc1ccc(F)cc1C(=O)NCc1ccc(-c2n[nH]c3ncnc(N)c23)cc1]

At room temperature, N-(4-(4-amino-1-((2-(trimethylsilyl) ethoxy)methyl)-1H-pyrazolo[3,4-d]pyrimidin-3-yl)benzyl)-5-fluoro-2-methoxybenzamide (3.35 g, 6.41 mmol), DCM (20 mL), and TFA (20 mL) were added into a single-necked flask in sequence. The reaction was allowed to proceed at room temperature for 16 h under nitrogen protection before termination. The reaction solution was concentrated and diluted with MeOH and the pH was adjusted to alkaline with ammonia. The reaction was stirred for 1.5 h, slurried with water for half an hour, filtered, and rinsed with water, and the filter cake was collected and concentrated to afford 2.40 g of a gray solid with a yield of 95%. MS (ESI) m/z: 393.5 [M+H]⁺.

### Step 4: Synthesis of (E)-N-(4-(4-(((dimethylamino)methylene)amino)-1H-pyrazolo[3,4-d]pyrimidin-3-yl)benzyl)-5-fluoro-2-methoxybenzamide

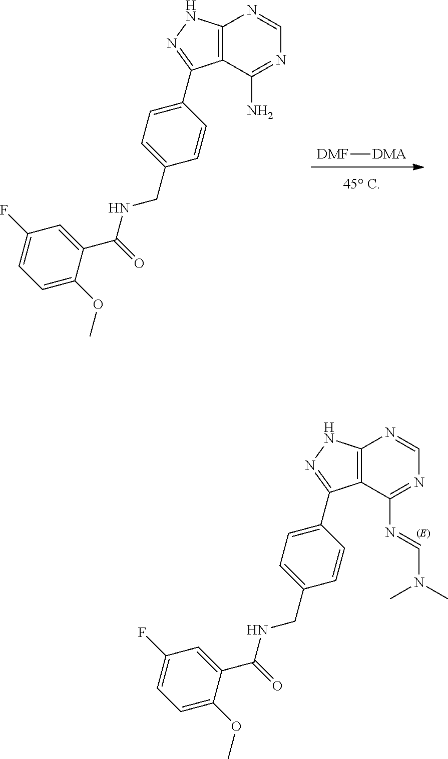

At room temperature, N-(4-(4-amino-1H-pyrazolo[3,4-d]pyrimidin-3-yl)benzyl)-5-fluoro-2-methoxybenzamide (2.40 g, 6.12 mmol) and DMF-DMA (30 mL) were added into a single-necked flask in sequence. Under nitrogen protection, the mixture was heated to 45° C. and the reaction was allowed to proceed for 2 h before termination. The reaction solution was concentrated and purified by column chromatography (MeOH/DCM system) to afford 2.12 g of a white solid with a yield of 77%. MS (ESI) m/z: 448.5 [M+H]⁺.

### Step 5: Synthesis of N-(4-(4-amino-1-(6-(4-(dimethoxymethyl) piperidin-1-yl) piperidin-3-yl)-1H-pyrazolo[3,4-d]pyrimidin-3-yl)benzyl)-5-fluoro-2-methoxybenzamide

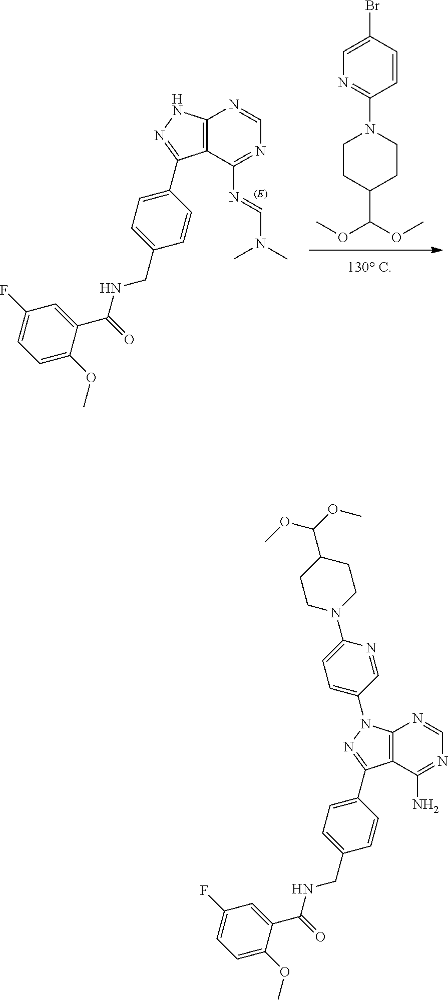

At room temperature, (E)-N-(4-(4-(((dimethylamino)methylene)amino)-1H-pyrazolo[3,4-d]pyrimidin-3-yl)benzyl)-5-fluoro-2-methoxybenzamide (0.30 g, 0.67 mmol), 5-bromo-2-(4-(dimethoxymethyl) piperidin-1-yl)pyridine (0.42 g, 1.34 mmol), trans-N¹,N²-dimethylcyclohexane-1,2-diamine (0.05 g, 0.34 mmol), Cs₂CO₃ (0.66 g, 2.01 mmol), CuI (0.06 g, 0.34 mmol) and DMSO (20 mL) were added into a single-necked flask in sequence. Under nitrogen protection, the mixture was heated to 130° C. and the reaction was allowed to proceed for 16 h before termination. The reaction solution was diluted with water, and extracted with EA. The organic phase was dried over anhydrous Na₂SO₄, filtered, concentrated and purified by column chromatography (EA/PE system) to afford 0.20 g of a crude product as a yellow oil. MS (ESI) m/z: 627.7 [M+H]⁺.

### Step 6: Synthesis of N-(4-(4-amino-1-(6-(4-formylpiperidin-1-yl)pyridin-3-yl)-1H-pyrazolo[3,4-d]pyrimidin-3-yl)benzyl)-5-fluoro-2-methoxybenzamide

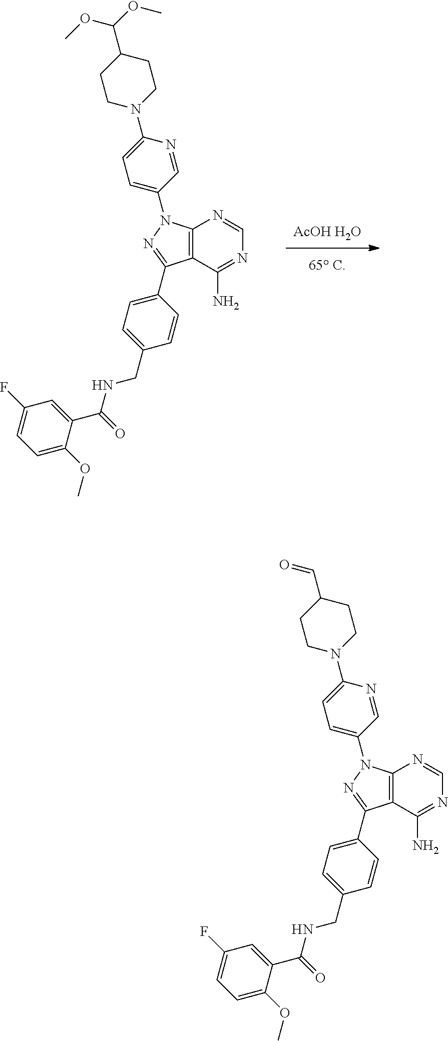 [SMILES: "AcOH H2O" ; COc1ccc(F)cc1C(=O)NCc1ccc(-c2nn(-c3ccc(N4CCC(C(OC)OC)CC4)nc3)c3ncnc(N)c23)cc1 ; COc1ccc(F)cc1C(=O)NCc1ccc(-c2nn(-c3ccc(N4CCC(C=O)CC4)nc3)c3ncnc(N)c23)cc1]

At room temperature, N-(4-(4-amino-1-(6-(4-(dimethoxymethyl) piperidin-1-yl) piperidin-3-yl)-1H-pyrazolo[3,4-d]pyrimidin-3-yl)benzyl)-5-fluoro-2-methoxybenzamide (0.20 g, 0.32 mmol), AcOH (10 mL) and H₂O (10 mL) were added into a single-necked flask in sequence. Under nitrogen protection, the mixture was heated to 65° C. and the reaction was allowed to proceed for 16 h before termination. The reaction solution was concentrated to dryness to afford 0.15 g of a crude product as a yellow oil. MS (ESI) m/z: 581.6 [M+H]⁺.

### Step 7: Synthesis of N-(4-(4-amino-1-(6-(4-((4-(2-(2,6-dioxopiperidin-3-yl)-3-oxoisoindolin-5-yl) piperazin-1-yl)methyl)piperidin-1-yl)pyridin-3-yl)-1H-pyrazolo[3,4-d]pyrimidin-3-yl)benzyl)-5-fluoro-2-methoxybenzamide

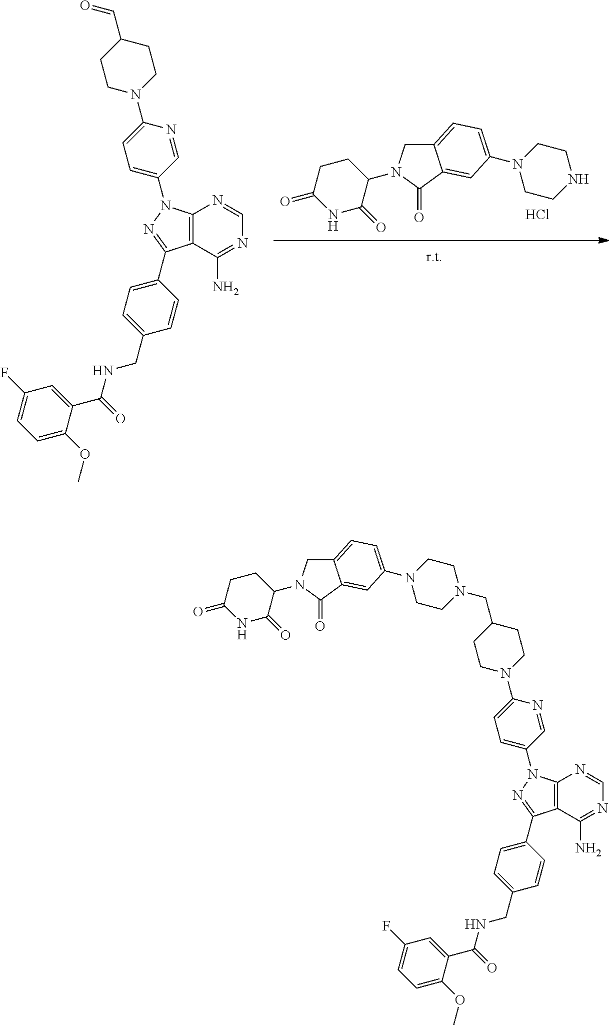 [SMILES: COc1ccc(F)cc1C(=O)NCc1ccc(-c2nn(-c3ccc(N4CCC(C=O)CC4)nc3)c3ncnc(N)c23)cc1 ; O=C1CCC(N2Cc3ccc(N4CCNCC4)cc3C2=O)C(=O)N1 ; Cl ; COc1ccc(F)cc1C(=O)NCc1ccc(-c2nn(-c3ccc(N4CCC(CN5CCN(c6ccc7c(c6)C(=O)N(C6CCC(=O)NC6=O)C7)CC5)CC4)nc3)c3ncnc(N)c23)cc1]

At room temperature, N-(4-(4-amino-1-(6-(4-formylpiperidin-1-yl)pyridin-3-yl)-1H-pyrazolo[3,4-d]pyrimidin-3-yl)benzyl)-5-fluoro-2-methoxybenzamide (0.15 g, 0.26 mmol), 3-(1-oxo-6-(piperazin-1-yl) isoindolin-2-yl)piperidine-2,6-dione hydrochloride (0.11 g, 0.31 mmol), DCM (20 mL) and NaBH (OAc)₃ (0.11 g, 0.52 mmol) were added into a single-necked flask in sequence. The reaction was allowed to proceed at room temperature for 16 h under nitrogen protection before termination. The reaction was quenched, and the reaction solution was concentrated and purified by column chromatography (MeOH/DCM system) to afford 0.010 g of a yellow solid. The total yield of the three steps was 5%, ¹H NMR (400 MHz, DMSO-d₆) δ 8.73 (d, J=2.7 Hz, 1H), 8.33 (s, 1H), 8.12 (dd, J=9.1, 2.8 Hz, 1H), 7.73 (d, J=7.8 Hz, 2H), 7.60-7.49 (m, 4H), 7.38-7.33 (m, 1H), 7.20 (dd, J=9.1, 4.3 Hz, 1H), 7.12-6.97 (m, 3H), 5.06 (dd, J=13.3, 5.1 Hz, 1H), 4.61 (d, J=6.1 Hz, 2H), 4.40-4.32 (m, 3H), 4.25-4.18 (m, 1H), 3.91 (s, 3H), 3.54-3.49 (m, 1H), 3.32-3.23 (m, 5H), 2.95-2.85 (m, 3H), 2.68-2.54 (m, 2H), 2.45-2.33 (m, 2H), 2.26-2.17 (m, 2H), 2.00-1.93 (m, 1H), 1.90-1.76 (m, 3H), 1.27-1.07 (m, 4H). MS (ESI) m/z: 893.8 [M+H]⁺.

### Example 10: Synthesis of N-(4-(4-amino-1-(6-(4-((1-(2-(2,6-dioxopiperidin-3-yl)-1-oxoisoindolin-5-yl) piperidin-4-yl)methyl)piperazin-1-yl)pyridin-3-yl)-1H-pyrazolo[3,4-d]pyrimidin-3-yl)-2-fluorobenzyl)-5-fluoro-2-methoxybenzamide

### (Compound II-5)

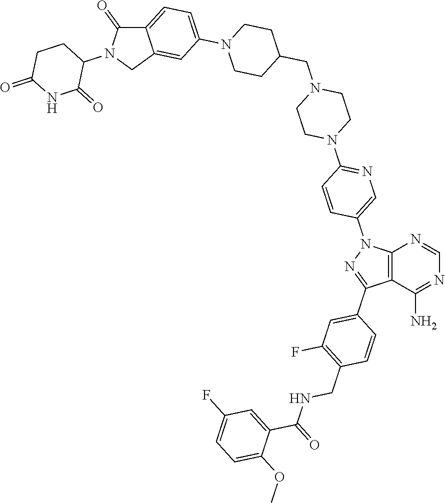 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1F]

The synthesis method of N-(4-(4-amino-1-(6-(piperazin-1-yl)pyridin-3-yl)-1H-pyrazolo[3,4-d]pyrimidin-3-yl)-2-fluorobenzyl)-5-fluoro-2-methoxybenzamide follows Example 1.

### Step 1: Synthesis of N-(4-bromo-2-fluorobenzyl)-5-fluoro-2-methoxybenzamide

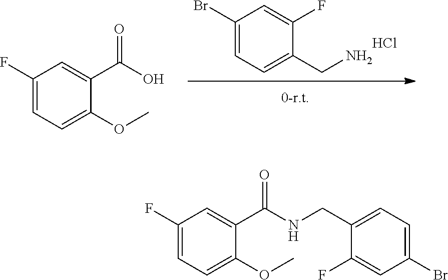 [SMILES: COc1ccc(F)cc1C(=O)O ; NCc1ccc(Br)cc1F ; Cl ; COc1ccc(F)cc1C(=O)NCc1ccc(Br)cc1F]

At 0° C., 5-fluoro-2-methoxybenzoic acid (2.00 g, 11.76 mmol), 4-bromo-2-fluorobenzylamine hydrochloride (2.83 g, 11.76 mmol), N-methylimidazole (3.38 g, 41.14 mmol) and DMF (30 mL) were added into a single-necked flask in sequence, TCFH (8.25 g, 29.39 mmol) was slowly added in portions. Under nitrogen protection, the mixture was warmed to room temperature and the reaction was allowed to proceed for 16 h before termination. The reaction was quenched with water and the reaction solution was extracted with EA. The organic phase was dried over anhydrous Na₂SO₄, filtered, concentrated and purified by column chromatography (EA/PE system) to afford 4.12 g of a white solid. MS (ESI) m/z: 356.3 [M+H]⁺.

### Step 2: Synthesis of 5-fluoro-N-(2-fluoro-4-(4,4,5-trimethyl-1,3,2-dioxaborolan-2-yl)benzyl)-2-methoxybenzamide

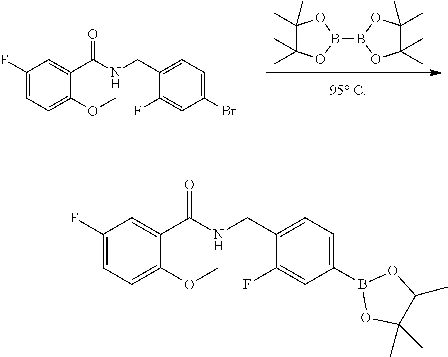

At room temperature, N-(4-bromo-2-fluorobenzyl)-5-fluoro-2-methoxybenzamide (4.12 g, 11.57 mmol), bis(pinacolato)diboron (4.41 g, 17.35 mmol), Pd(dppf)Cl₂ (0.84 g, 1.16 mmol), KOAc (2.27 g, 23.14 mmol) and Dioxane (60 mL) were added into a single-necked flask in sequence, and the reaction was allowed to proceed at 95° C. for 8 h under nitrogen protection before termination. The reaction solution was filtered and rinsed with EA. The organic phase was filtered, concentrated and purified by column chromatography (EA/PE system) to afford 5.70 g of a light yellow oil. MS (ESI) m/z: 404.6 [M+H]⁺.

### Step 3: Synthesis of tert-butyl 4-(5-(4-amino-3-(3-fluoro-4-((5-fluoro-2-methoxybenzamide)methyl)phenyl)-1H-pyrazolo[3,4-d]pyrimidin-1-yl)pyridin-2-yl) piperazine-1-carboxylate

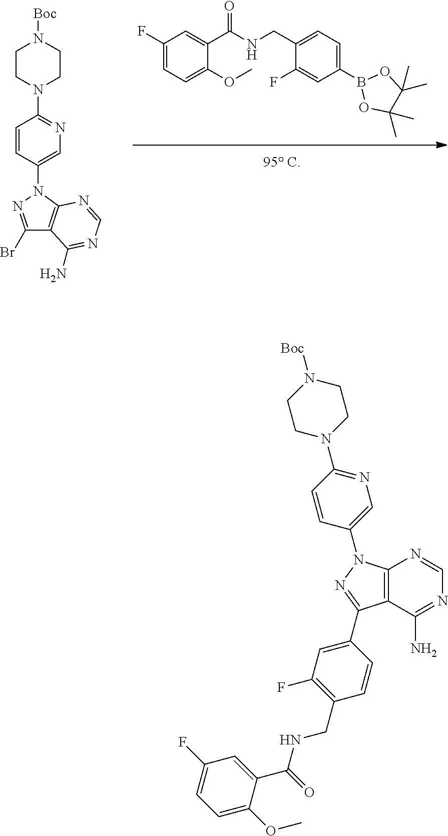 [SMILES: CC(C)(C)OC(=O)N1CCN(c2ccc(-n3nc(Br)c4c(N)ncnc43)cn2)CC1 ; COc1ccc(F)cc1C(=O)NCc1ccc(B2OC(C)(C)C(C)(C)O2)cc1F ; COc1ccc(F)cc1C(=O)NCc1ccc(-c2nn(-c3ccc(N4CCN(C(=O)OC(C)(C)C)CC4)nc3)c3ncnc(N)c23)cc1F]

At room temperature, tert-butyl 4-(5-(4-amino-3-bromo-1H-pyrazolo[3,4-d]pyrimidin-1-yl)pyridin-2-yl) piperazine-1-carboxylate (0.16 g, 0.34 mmol), 5-fluoro-N-(2-fluoro-4-(4,4,5-trimethyl-1,3,2-dioxaborolan-2-yl)benzyl)-2-methoxybenzamide (0.34 g, 0.84 mmol), Cs₂CO₃ (0.33 g, 1.01 mmol), Pd(PPh₃)₂Cl₂ (0.07 g, 0.10 mmol) and Dioxane/H₂O (20 mL) were added into a single-necked flask in sequence. Under nitrogen protection, the mixture was slowly heated to 95° C. and the reaction was allowed to proceed for 12 h before termination. The reaction solution was cooled, filtered, and rinsed with EA, and the filtrate was dried over anhydrous Na₂SO₄, filtered, concentrated and purified by column chromatography (EA/PE system) to afford 0.23 g of a yellow solid. MS (ESI) m/z: 672.7 [M+H]⁺.

### Step 4: Synthesis of N-(4-(4-amino-1-(6-(piperazin-1-yl)pyridin-3-yl)-1H-pyrazolo[3,4-d]pyrimidin-3-yl)-2-fluorobenzyl)-5-fluoro-2-methoxybenzamide

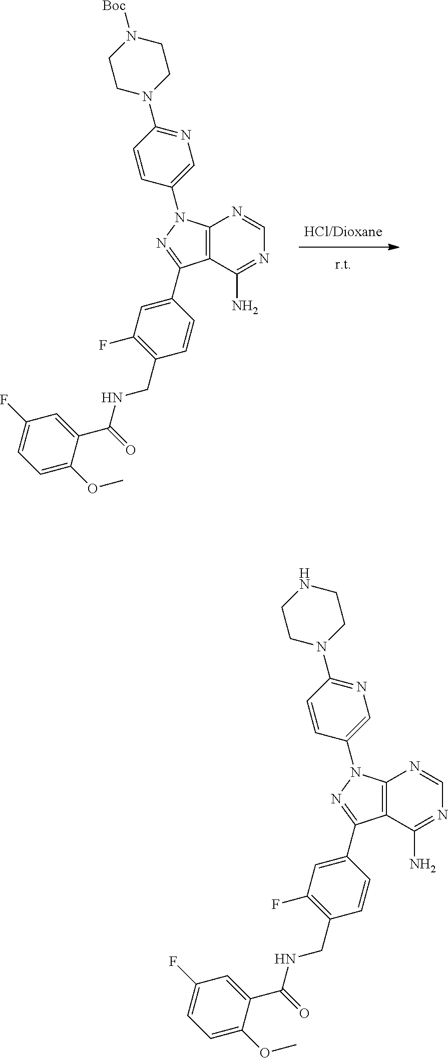 [SMILES: COc1ccc(F)cc1C(=O)NCc1ccc(-c2nn(-c3ccc(N4CCN(C(=O)OC(C)(C)C)CC4)nc3)c3ncnc(N)c23)cc1F ; COc1ccc(F)cc1C(=O)NCc1ccc(-c2nn(-c3ccc(N4CCNCC4)nc3)c3ncnc(N)c23)cc1F]

At room temperature, tert-butyl 4-(5-(4-amino-3-(3-(3-fluoro-4-((5-fluoro-2-methoxybenzamido)methyl)phenyl)-1H-pyrazolo[3,4-d]pyrimidin-1-yl)pyridin-2-yl) piperazine-1-carboxylate (0.23 g, 0.34 mmol), DCM (20 mL), and HCl/Dioxane (20 mL) were added into a single-necked flask in sequence. The reaction was allowed to proceed at room temperature for 2 h under nitrogen protection before termination. The reaction was quenched, the layers were separated, and the aqueous layer was extracted with DCM. The organic phases were combined, dried over anhydrous Na₂SO₄, filtered, and concentrated to afford 0.20 g of a crude product as a yellow solid. MS (ESI) m/z: 572.6 [M+H]⁺.

### Step 5: Synthesis of N-(4-(4-amino-1-(6-(4-((1-(2-(2,6-dioxopiperidin-3-yl)-1-oxoisoindolin-5-yl) piperidin-4-yl)methyl)piperazin-1-yl)pyridin-3-yl)-1H-pyrazolo[3,4-d]pyrimidin-3-yl)-2-fluorobenzyl)-5-fluoro-2-methoxybenzamide

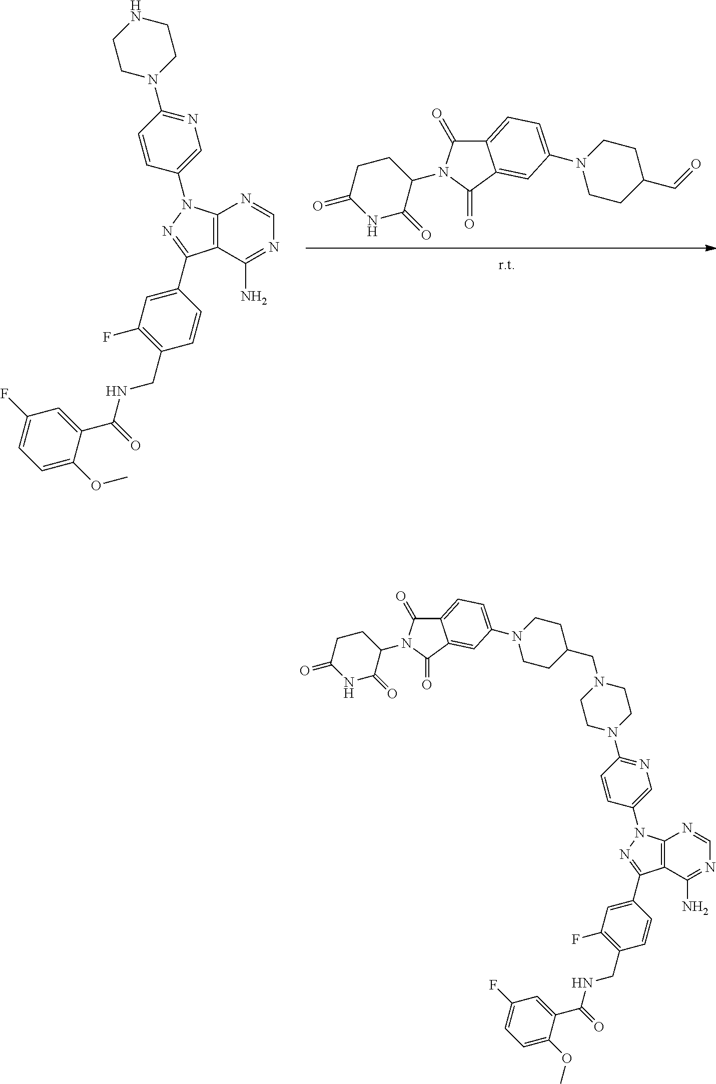 [SMILES: COc1ccc(F)cc1C(=O)NCc1ccc(-c2nn(-c3ccc(N4CCNCC4)nc3)c3ncnc(N)c23)cc1F ; O=CC1CCN(c2ccc3c(c2)C(=O)N(C2CCC(=O)NC2=O)C3=O)CC1 ; COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1F]

At room temperature, N-(4-(4-amino-1-(6-(piperazin-1-yl)pyridin-3-yl)-1H-pyrazolo[3,4-d]pyrimidin-3-yl)-2-fluorobenzyl)-5-fluoro-2-methoxybenzamide (0.20 g, 0.35 mmol), 1-(2-(2,6-dioxopiperidin-3-yl)-1-oxoisoindolin-5-yl)piperidine-4-carbaldehyde (0.12 g, 0.35 mmol), DCM (20 mL) and NaBH (OAc)₃ (0.11 g, 0.52 mmol) were added into a single-necked flask in sequence. The reaction was allowed to proceed at room temperature for 18 h under nitrogen protection before termination. The reaction solution was concentrated and purified by column chromatography (MeOH/DCM system) to afford 0.16 g of a yellow solid. The total yield of the four steps was 50%. ¹H NMR (400 MHz, DMSO-d₆) δ 8.76 (d, J=2.6 Hz, 1H), 8.33 (s, 1H), 8.16 (dd, J=9.0, 2.7 Hz, 1H), 7.59-7.48 (m, 5H), 7.38-7.33 (m, 1H), 7.20 (dd, J=9.1, 4.2 Hz, 1H), 7.08-6.99 (m, 3H), 5.05 (dd, J=13.3, 5.1 Hz, 1H), 4.63 (d, J=6.0 Hz, 2H), 4.35-4.28 (m, 1H), 4.23-4.16 (m, 1H), 3.96-3.84 (m, 5H), 3.61-3.52 (m, 4H), 2.95-2.80 (m, 3H), 2.67-2.56 (m, 2H), 2.48-2.27 (m, 5H), 2.26-2.15 (m, 2H), 1.99-1.91 (m, 2H), 1.85-1.78 (m, 3H), 1.25-1.16 (m, 2H). MS (ESI) m/z: 911.7 [M+H]⁺.

### Example 11: Synthesis of N-(4-(4-amino-1-(6-(4-((1-(2-(2,6-dioxopiperidin-3-yl)-1,3-dioxoisoindol-5-yl) piperidin-4-yl)methyl)piperazin-1-yl)pyridin-3-yl)-1H-pyrazolo[3,4-d]pyrimidin-3-yl)-2-methylbenzyl)-5-fluoro-2-methoxybenzamide (Compound II-6)

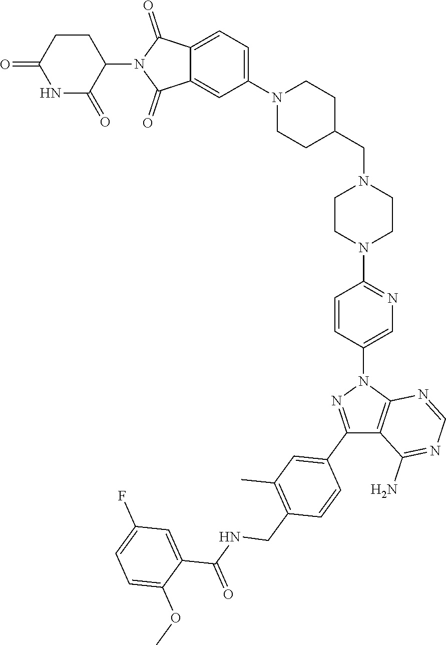 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1C]

The synthesis method of N-(4-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide follows Example 1.

### Step 1: Synthesis of N-(4-(4-amino-1-(6-(4-((1-(2-(2,6-dioxopiperidin-3-yl)-1,3-dioxoisoindol-5-yl) piperidin-4-yl)methyl)piperazin-1-yl)pyridin-3-yl)-1H-pyrazolo[3,4-d]pyrimidin-3-yl)-2-methylbenzyl)-5-fluoro-2-methoxybenzamide

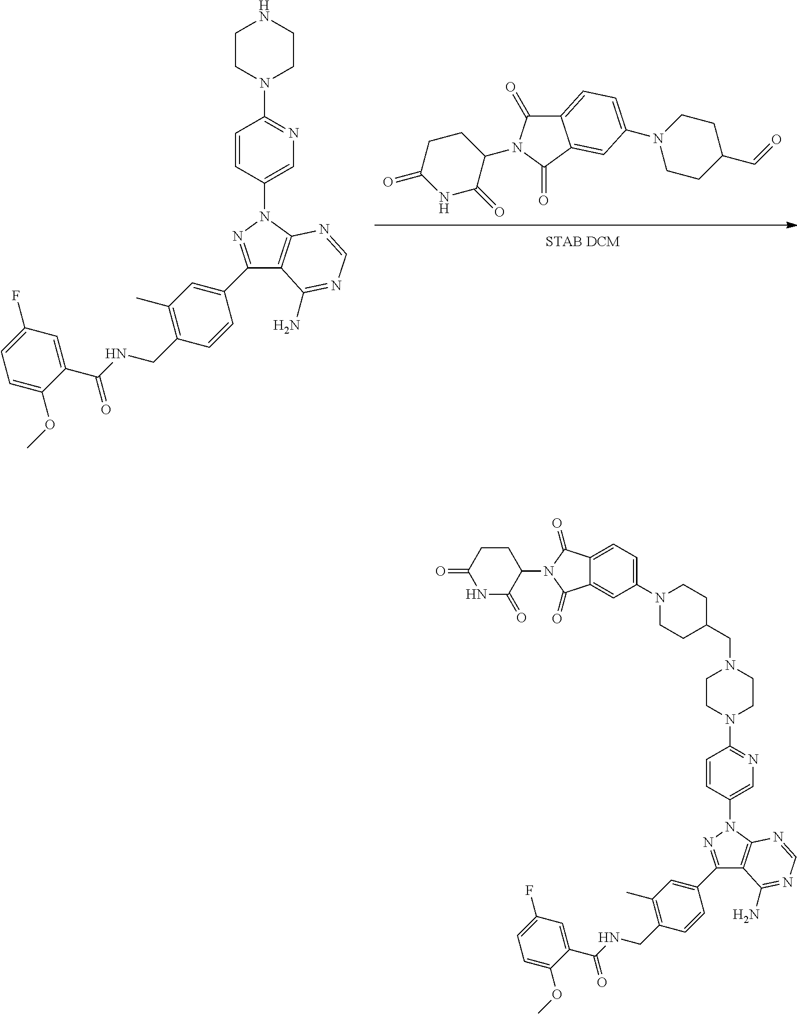 [SMILES: COc1ccc(F)cc1C(=O)NCc1ccc(-c2nn(-c3ccc(N4CCNCC4)nc3)c3ncnc(N)c23)cc1C ; O=CC1CCN(c2ccc3c(c2)C(=O)N(C2CCC(=O)NC2=O)C3=O)CC1 ; COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1C]

At room temperature, N-(4-(4-amino-1-(6-(piperazin-1-yl)pyridin-3-yl)-1H-pyrazolo[3,4-d]pyrimidin-3-yl]-2-methylbenzyl)-5-fluoro-2-methoxybenzamide (0.10 g, 0.18 mmol), 1-(2-(2,6-dioxopiperidin-3-yl)-1,3-dioxoisoindol-5-yl)piperidine-4-carbaldehyde (0.08 g, 0.21 mmol), sodium triacetoxyborohydride (0.06 g, 0.28 mmol) and DCM (20 mL) were added into a single-necked flask in sequence and the reaction was allowed to proceed at room temperature for 3 h. The reaction solution was concentrated and subjected to column chromatography (MeOH/DCM system) to afford 0.08 g of a yellow solid with a yield of 50%, ¹H NMR (400 MHz, DMSO-d₆) δ 8.84-8.72 (m, 2H), 8.33 (s, 1H), 8.16 (dd, J=9.0, 2.7 Hz, 1H), 7.66 (d, J=8.5 Hz, 1H), 7.60-7.44 (m, 4H), 7.39-7.29 (m, 2H), 7.26-7.15 (m, 2H), 7.01 (d, J=9.2 Hz, 1H), 5.07 (dd, J=12.9, 5.4 Hz, 1H), 4.57 (d, J=5.9 Hz, 2H), 4.06 (d, J=12.9 Hz, 2H), 3.91 (s, 3H), 3.61-3.48 (m, 3H), 3.42-3.36 (m, 2H), 3.04-2.82 (m, 3H), 2.65-2.54 (m, 1H), 2.54-2.43 (m, 9H), 2.20 (d, J=6.9 Hz, 2H), 2.01 (dd, J=10.7, 5.2 Hz, 1H), 1.92-1.74 (m, 3H), 1.24-1.07 (m, 2H). MS (ESI) m/z: 921.9 [M+H]⁺.

### Example 12: Synthesis of N-(4-(4-amino-1-(6-(1-((1-(2-(2,6-dioxopiperidin-3-yl)-1,3-dioxoisoindolin-5-yl) piperidin-4-yl)methyl)piperidin-4-yl)pyridin-3-yl)-1H-pyrazolo[3,4-d]pyrimidin-3-yl)benzyl)-5-fluoro-2-methoxybenzamide (Compound II-7)

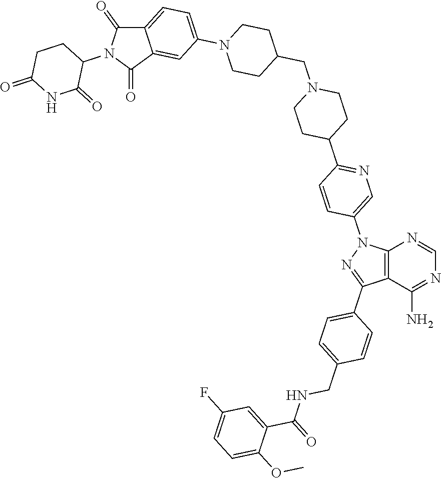 [SMILES: COc1ccc(F)cc1C(=O)NCc1ccc(-c2nn(-c3ccc(C4CCN(CC5CCN(c6ccc7c(c6)C(=O)N(C6CCC(=O)NC6=O)C7=O)CC5)CC4)nc3)c3ncnc(N)c23)cc1]

The synthesis method of N-(4-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide follows Example 1.

### Step 1: Synthesis of tert-butyl 4-(5-(4-amino-3-(4-((5-fluoro-2-methoxybenzamide)methyl)phenyl)-1H-pyrazolo[3,4-d]pyrimidin-1-yl)pyridin-2-yl)piperidine-1-carboxylate

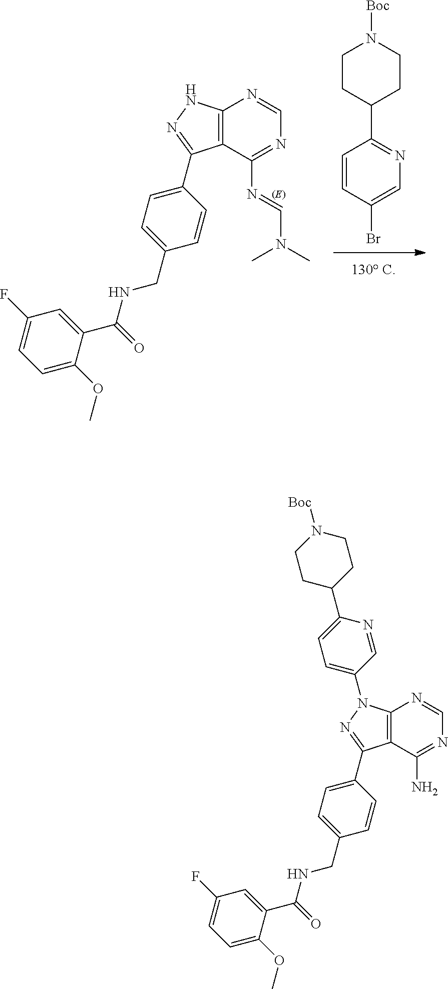 [SMILES: COc1ccc(F)cc1C(=O)NCc1ccc(-c2n[nH]c3ncnc(/N=C/N(C)C)c23)cc1 ; CC(C)(C)OC(=O)N1CCC(c2ccc(Br)cn2)CC1 ; COc1ccc(F)cc1C(=O)NCc1ccc(-c2nn(-c3ccc(C4CCN(C(=O)OC(C)(C)C)CC4)nc3)c3ncnc(N)c23)cc1]

At room temperature, (E)-N-(4-(4-(((dimethylamino)methylene)amino)-1H-pyrazolo[3,4-d]pyrimidin-3-yl)benzyl)-5-fluoro-2-methoxybenzamide (0.50 g, 1.12 mmol), tert-butyl 4-(5-bromopyridin-2-yl) piperidin-1-carboxylate (0.76 g, 2.23 mmol), trans-N¹,N²-dimethylcyclohexane-1,2-diamine (0.08 g, 0.56 mmol), Cs₂CO₃ (1.09 g, 3.35 mmol), CuI (0.11 g, 0.56 mmol) and DMSO (30 mL) were added into a single-necked flask in sequence. Under nitrogen protection, the mixture was heated to 130° C. and the reaction was allowed to proceed for 16 h before termination. The reaction solution was diluted with water and extracted with EA, and the organic phase was dried over anhydrous Na₂SO₄, filtered, concentrated and purified by column chromatography (EA/PE system) to afford 0.40 g of a white solid with a yield of 55%. MS (ESI) m/z: 653.7 [M+H]⁺.

### Step 2: Synthesis of N-(4-(4-amino-1-(6-(piperidin-4-yl)pyridin-3-yl)-1H-pyrazolo[3,4-d]pyrimidin-3-yl)benzyl)-5-fluoro-2-methoxybenzamide

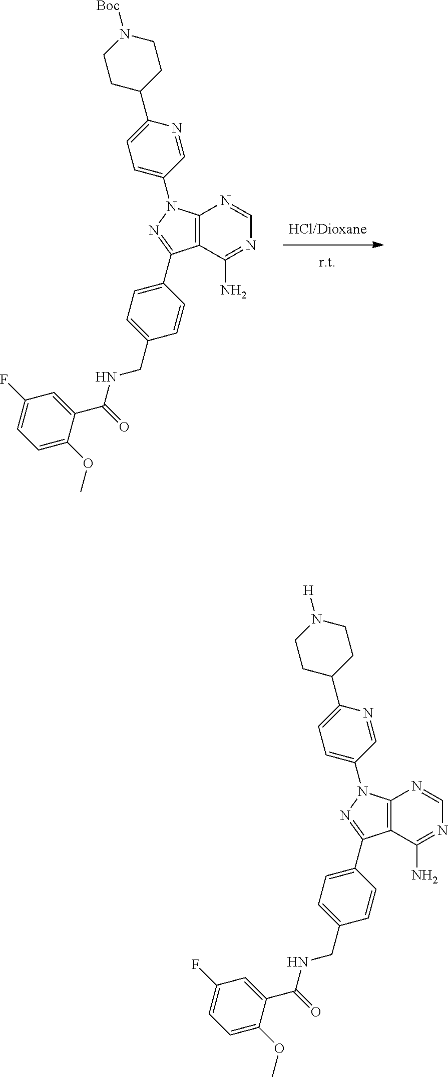 [SMILES: COc1ccc(F)cc1C(=O)NCc1ccc(-c2nn(-c3ccc(C4CCN(C(=O)OC(C)(C)C)CC4)nc3)c3ncnc(N)c23)cc1 ; COc1ccc(F)cc1C(=O)NCc1ccc(-c2nn(-c3ccc(C4CCNCC4)nc3)c3ncnc(N)c23)cc1]

At room temperature, tert-butyl 4-(5-(4-amino-3-(4-((5-fluoro-2-methoxybenzamido)methyl)phenyl)-1H-pyrazolo[3,4-d]pyrimidin-1-yl)pyridin-2-yl)piperidine-1-carboxylate (0.40 g, 0.61 mmol), DCM (20 mL), and HCl/Dioxane (20 mL) were added into a single-necked flask in sequence. The reaction was allowed to proceed at room temperature for 2 h under nitrogen protection before termination. The reaction solution was concentrated, treated with a saturated aqueous Na₂CO₃ solution, and extracted with DCM, and the organic phase was dried over anhydrous Na₂SO₄, filtered, and concentrated to afford 0.29 g of a crude product as a white solid with a yield of 86%. MS (ESI) m/z: 553.6 [M+H]⁺.

### Step 3: Synthesis of N-(4-(4-amino-1-(6-(1-((1-(2-(2,6-dioxopiperidin-3-yl)-1,3-dioxoisoindolin-5-yl)piperidin-4-yl)methyl)piperidin-4-yl)pyridin-3-yl)-1H-pyrazolo[3,4-d]pyrimidin-3-yl)benzyl)-5-fluoro-2-methoxybenzamide

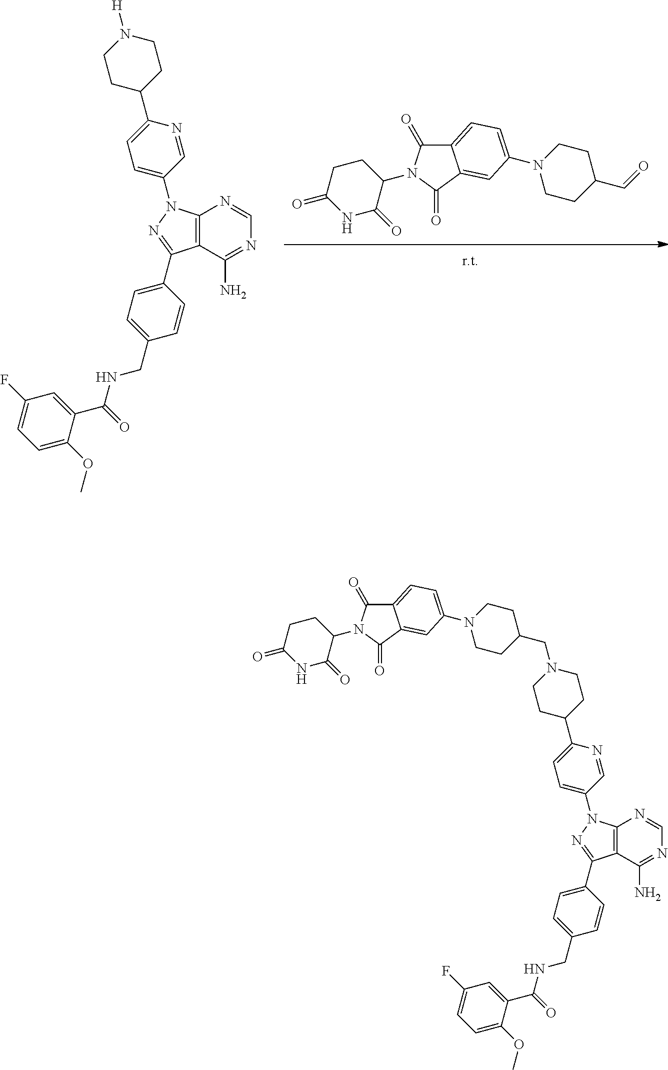 [SMILES: COc1ccc(F)cc1C(=O)NCc1ccc(-c2nn(-c3ccc(C4CCNCC4)nc3)c3ncnc(N)c23)cc1 ; O=CC1CCN(c2ccc3c(c2)C(=O)N(C2CCC(=O)NC2=O)C3=O)CC1 ; COc1ccc(F)cc1C(=O)NCc1ccc(-c2nn(-c3ccc(C4CCN(CC5CCN(c6ccc7c(c6)C(=O)N(C6CCC(=O)NC6=O)C7=O)CC5)CC4)nc3)c3ncnc(N)c23)cc1]

At room temperature, N-(4-(4-amino-1-(6-(piperidin-4-yl)pyridin-3-yl)-1H-pyrazolo[3,4-d]pyrimidin-3-yl)benzyl)-5-fluoro-2-methoxybenzamide (0.06 g, 0.11 mmol), 1-(2-(2,6-dioxopiperidin-3-yl)-1,3-dioxoisoindolin-5-yl)piperidine-4-carbaldehyde (0.04 g, 0.12 mmol), DCM (20 mL) and NaBH (OAc)₃ (0.05 g, 0.22 mmol) were added into a single-necked flask in sequence. The reaction was allowed to proceed at room temperature for 16 h under nitrogen protection before termination. The reaction was quenched, and the reaction solution was concentrated and purified by column chromatography (MeOH/DCM system) to afford 0.060 g of a yellow solid with a yield of 61%, ¹H NMR (400 MHz, DMSO-d₆) δ 9.27 (d, J=2.5 Hz, 1H), 8.46 (dd, J=8.5, 2.6 Hz, 1H), 8.38 (s, 1H), 7.75 (d, J=7.9 Hz, 2H), 7.65 (d, J=8.5 Hz, 1H), 7.59-7.47 (m, 4H), 7.38-7.30 (m, 2H), 7.24-7.18 (m, 2H), 5.06 (dd, J=12.9, 5.4 Hz, 1H), 4.61 (d, J=6.1 Hz, 2H), 4.07-4.00 (m, 2H), 3.91 (s, 3H), 3.18-3.14 (m, 1H), 3.04-2.83 (m, 6H), 2.76-2.69 (m, 1H), 2.60-2.53 (m, 2H), 2.30-1.94 (m, 6H), 1.87-1.78 (m, 6H), 1.22-1.12 (m, 2H). MS (ESI) m/z: 906.96 [M+H]⁺.

### Example 13: Synthesis of N-(4-(4-amino-1-(6-(1-((1-(4-(2,4-dioxotetrahydropyrimidin-1(2H)-yl)-3-methylphenyl)piperidin-4-yl)methyl)piperidin-4-yl)pyridin-3-yl)-1H-pyrazolo[3,4-d]pyrimidin-3-yl)benzyl)-5-fluoro-2-methoxybenzamide (Compound II-8)

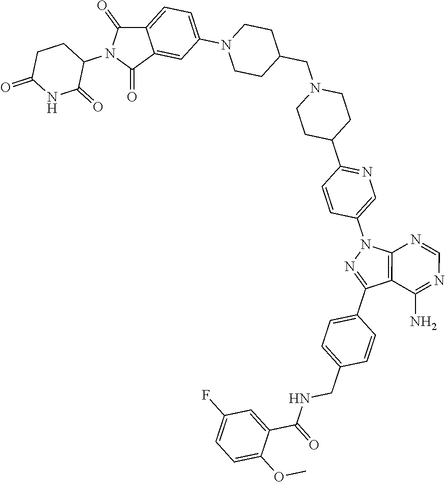 [SMILES: COc1ccc(F)cc1C(=O)NCc1ccc(-c2nn(-c3ccc(C4CCN(CC5CCN(c6ccc7c(c6)C(=O)N(C6CCC(=O)NC6=O)C7=O)CC5)CC4)nc3)c3ncnc(N)c23)cc1]

The synthesis method of N-(4-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide follows Example 1.

### Step 1: Synthesis of N-(4-(4-amino-1-(6-(1-((1-(4-(2,4-dioxotetrahydropyrimidine-1(2H)-yl)-3-methylphenyl)piperidin-4-yl)methyl)piperidin-4-yl)pyridin-3-yl)-1H-pyrazolo[3,4-d]pyrimidin-3-yl)benzyl)-5-fluoro-2-methoxybenzamide

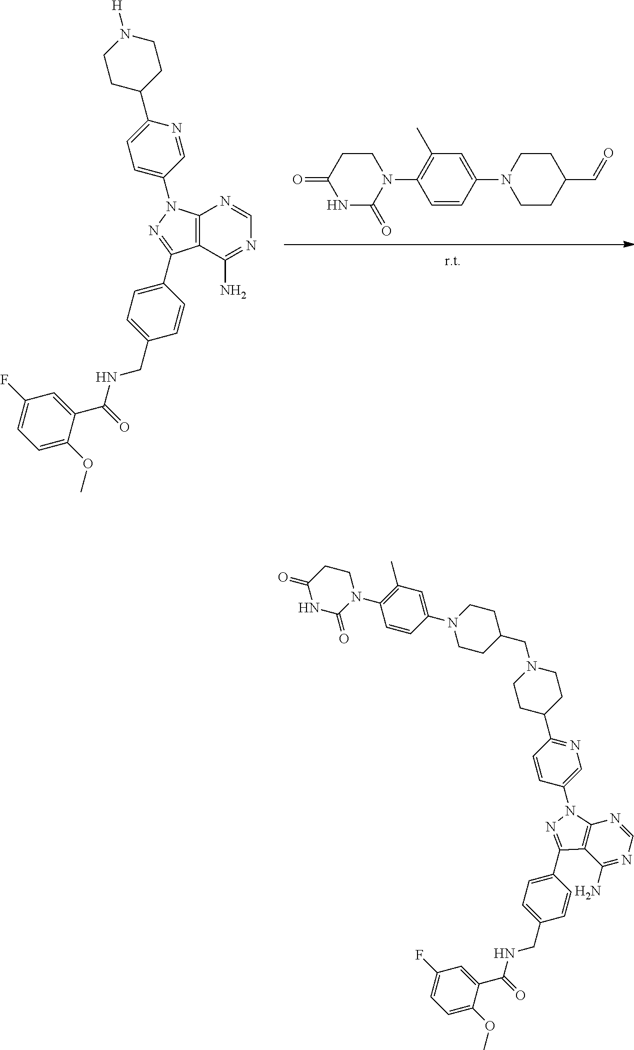 [SMILES: COc1ccc(F)cc1C(=O)NCc1ccc(-c2nn(-c3ccc(C4CCNCC4)nc3)c3ncnc(N)c23)cc1 ; Cc1cc(N2CCC(C=O)CC2)ccc1N1CCC(=O)NC1=O ; COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c3ncnc(N)c23)cc1]

At room temperature, N-(4-(4-amino-1-(6-(piperidin-4-yl)pyridin-3-yl)-1H-pyrazolo[3,4-d]pyrimidin-3-yl)benzyl)-5-fluoro-2-methoxybenzamide (0.06 g, 0.11 mmol), 1-(4-(2,4-dioxotetrahydropyrimidine-1(2H)-yl)-3-methylphenyl)piperidine-4-carbaldehyde (0.04 g, 0.12 mmol), DCM (20 mL) and NaBH (OAc)₃ (0.05 g, 0.22 mmol) were added into a single-necked flask in sequence. The reaction was allowed to proceed at room temperature for 16 h under nitrogen protection before termination. The reaction was quenched, and the reaction solution was concentrated and purified by column chromatography (MeOH/DCM system) to afford 0.035 g of a yellow solid with a yield of 38%, ¹H NMR (400 MHz, DMSO-d₆) δ 9.28 (d, J=2.6 Hz, 1H), 8.47 (dd, J=8.5, 2.6 Hz, 1H), 8.38 (s, 1H), 7.75 (d, J=8.1 Hz, 2H), 7.57-7.49 (m, 4H), 7.37-7.32 (m, 1H), 7.20 (dd, J=9.1, 4.3 Hz, 1H), 7.04 (d, J=8.6 Hz, 1H), 6.82-6.75 (m, 2H), 4.61 (d, J=6.1 Hz, 2H), 3.91 (s, 3H), 3.71-3.63 (m, 3H), 3.50-3.43 (m, 2H), 3.17 (s, 1H), 3.06-2.95 (m, 2H), 2.76-2.62 (m, 5H), 2.33-2.20 (m, 2H), 2.11-2.00 (m, 5H), 1.87-1.72 (m, 7H), 1.24-1.18 (m, 2H). MS (ESI) m/z: 852.7 [M+H]⁺.

### Example 14: Synthesis of N-(4-(4-amino-1-(6-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-d]pyrimidin-3-yl)-2-methylbenzyl)-5-fluoro-2-methoxybenzamide (Compound I-4)

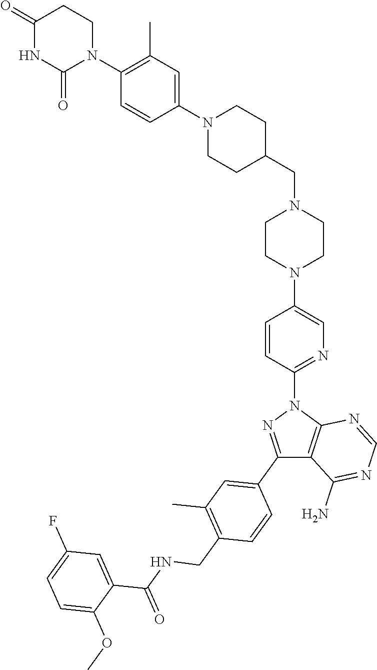 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cn3)c3ncnc(N)c23)cc1C]

The synthesis method of N-(4-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide follows Example 1.

### Step 1: Synthesis of N-(4-bromo-2-methylbenzyl)-5-fluoro-2-methoxybenzamide

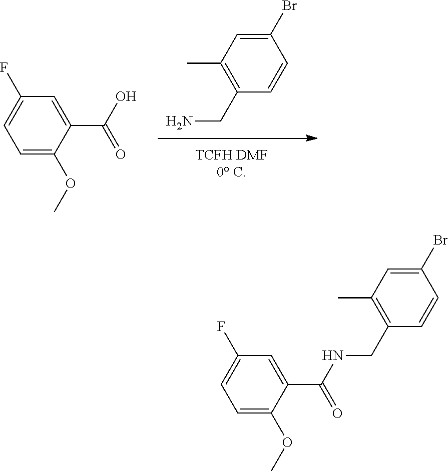

Under ice-water bath conditions, 4-bromo-2-methylphenylmethylamine (5.00 g, 24.90 mmol), 5-fluoro-2-methoxybenzoic acid (4.23 g, 24.90 mmol), tetramethylchloroformamidinium hexafluorophosphate (14.00 g, 50.00 mmol), N-methylimidazole (8.21 g, 100.00 mmol) and DMF (50 mL) were added into a single-necked flask in sequence. The reaction was allowed to proceed at room temperature for 15 h under nitrogen protection before termination. The reaction solution was diluted with water and extracted with EA, and the organic phase was dried over anhydrous Na₂SO₄, filtered, concentrated and purified by column chromatography (EA/PE system) to afford 9.48 g of a white solid with a yield of 99%. MS (ESI) m/z: 352.03 [M+H]⁺.

### Step 2: Synthesis of 5-fluoro-2-methoxy-N-(2-methyl-4-(4,4,5,5-tetramethyl-1,3,2-dioxaborolan-2-yl)benzyl)benzamide

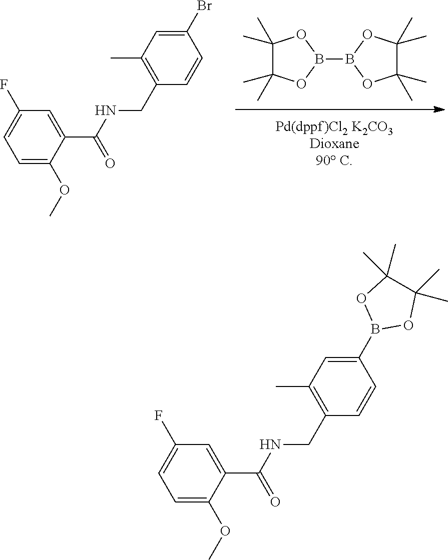

At room temperature, N-(4-bromo-2-methylbenzyl)-5-fluoro-2-methoxybenzamide (4.0 g, 11.4 mmol), bis(pinacolato)diboron (4.34 g, 17.10 mmol), potassium acetate (2.23 g, 22.80 mmol), 1,1′-bis(diphenylphosphino) ferrocenepalladium (II) dichloride (0.80 g, 1.10 mmol) and 1,4-dioxane (30 mL) were added into a single-necked flask in sequence. The reaction was allowed to proceed at 90° C. for 6 h under nitrogen protection before termination. The reaction solution was cooled to room temperature and filtered, and the filtrate was concentrated and purified by column chromatography (EA/PE system) to afford 1.60 g of a light yellow oily liquid with a yield of 29%.

### Step 3: Synthesis of 3-bromo-1-(2-(trimethylsilyl) ethoxy)methyl-1H-pyrazolo[3,4-d]pyrimidin-4-amine

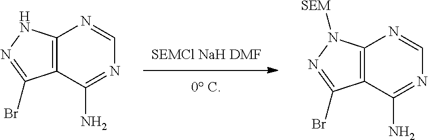 [SMILES: Nc1ncnc2[nH]nc(Br)c12 ; C[Si](C)(C)CCOCn1nc(Br)c2c(N)ncnc21]

Under ice-water bath conditions, 3-bromo-1H-pyrazolo[3,4-d]pyrimidin-4-amine (2.00 g, 12.60 mmol), sodium hydride (60%, 0.17 g, 7.01 mmol) and DMF (20 mL) were added into a single-necked flask in sequence. After stirring for 0.5 h, 2-(trimethylsilyl) ethoxymethyl chloride (0.94 g, 5.61 mmol) was added. The reaction was allowed to proceed at room temperature for 0.5 h under nitrogen protection before termination. A saturated ammonium chloride solution was added, and the mixture was extracted with EA. The organic phase was dried over anhydrous Na₂SO₄ and filtered, and the filtrate was concentrated and purified by column chromatography (BA/PE system) to afford 1.20 g of a white solid with a yield of 75%. MS (ESI) m/z: 345.29 [M+H]⁺.

### Step 4: Synthesis of N-(4-amino-1-(2-(trimethylsilyl) ethoxy)methyl)-1H-pyrazolo[3,4-d]pyrimidin-3-yl)-2-methylbenzyl)-5-fluoro-2-methoxybenzamide

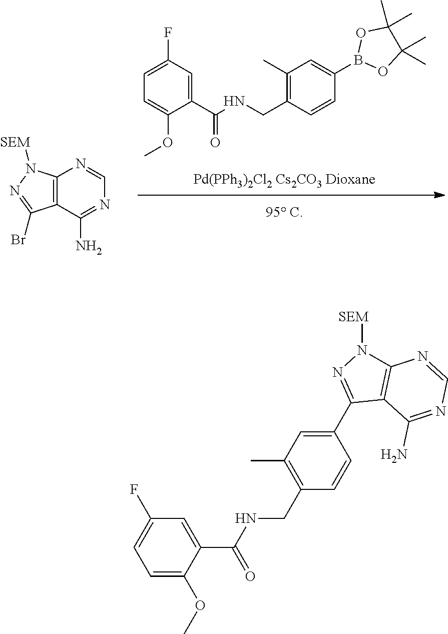 [SMILES: C[Si](C)(C)CCOCn1nc(Br)c2c(N)ncnc21 ; COc1ccc(F)cc1C(=O)NCc1ccc(B2OC(C)(C)C(C)(C)O2)cc1C ; "Pd(PPh3)2Cl2 Cs2CO3 Dioxane" ; COc1ccc(F)cc1C(=O)NCc1ccc(-c2nn(COCC[Si](C)(C)C)c3ncnc(N)c23)cc1C]

At room temperature, 3-bromo-1-(2-(trimethylsilyl) ethoxy)methyl-1H-pyrazolo[3,4-d]pyrimidin-4-amine (1.20 g, 3.40 mmol), 5-fluoro-2-methoxy-N-(2-methyl-4-(4,4,5,5-tetramethyl-1,3,2-dioxaborolan-2-yl)benzyl)benzamide (2.08 g, 5.20 mmol), cesium carbonate (3.41 g, 10.50 mmol), bis(triphenylphosphine) palladium dichloride (0.48 g, 0.68 mmol), Dioxane (20 mL) and water (1 mL) were added into a single-necked flask in sequence. The reaction was allowed to proceed at 95° C. for 20 h under nitrogen protection before termination. The reaction solution was cooled to room temperature and filtered, and the filtrate was dried over anhydrous Na₂SO₄, concentrated and purified by column chromatography (MeOH/DCM system) to afford 2.28 g of a black oil. MS (ESI) m/z: 537.24 [M+H]⁺.

### Step 5: Synthesis N-(4-aminopyrazolo[3,4-d]pyrimidin-3-yl)-2-methylbenzyl)-5-fluoro-2-methoxybenzamide

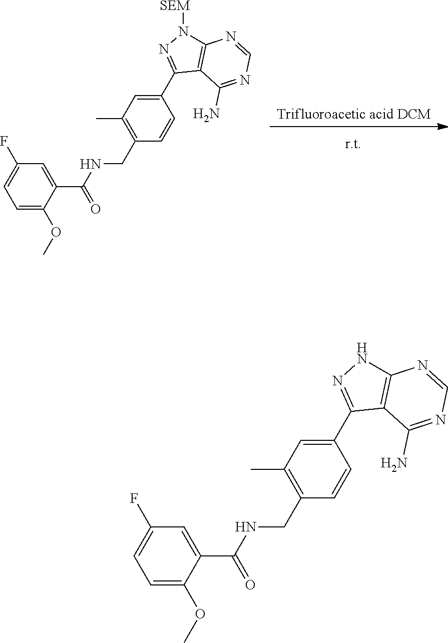 [SMILES: COc1ccc(F)cc1C(=O)NCc1ccc(-c2nn(COCC[Si](C)(C)C)c3ncnc(N)c23)cc1C ; "Trifluoroacetic acid DCM" ; COc1ccc(F)cc1C(=O)NCc1ccc(-c2n[nH]c3ncnc(N)c23)cc1C]

Under ice-water bath conditions, N-(4-amino-1-(2-(trimethylsilyl) ethoxy)methyl)-1H-pyrazolo[3,4-d]pyrimidin-3-yl)-2-methylbenzyl)-5-fluoro-2-methoxybenzamide (2.28 g, 40.01 mmol), trifluoroacetic acid (10 mL) and DCM (10 mL) were added into a single-necked flask in sequence. The reaction was allowed to proceed at room temperature for 16 h under nitrogen protection before termination. The reaction solution was concentrated and then dissolved with methanol, and the pH was adjusted to alkaline with ammonia water. The alkaline solution was diluted with water. The filter cake was collected by filtration and dried to afford 0.72 g of a crude product as a grey solid. MS (ESI) m/z: 407.16 [M+H]⁺.

### Step 6: Synthesis of tert-butyl 4-(5-(4-amino-3-(4-((5-fluoro-2-methoxybenzamido)methyl)-3-methylphenyl)-1H-pyrazolo[3,4-d]pyrimidin-1-yl)pyridin-2-yl) piperazine-1-carboxylate

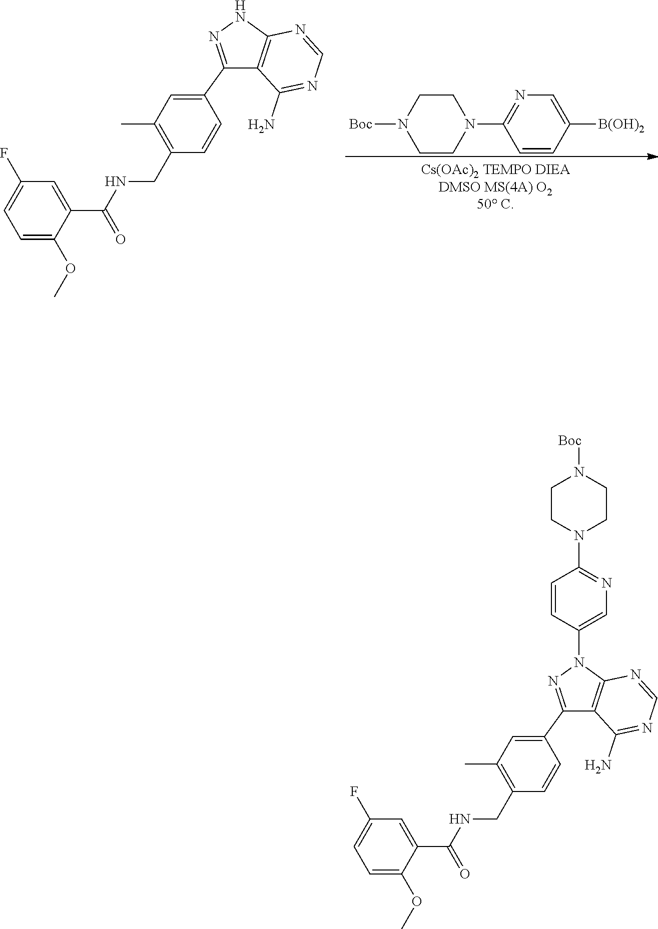 [SMILES: COc1ccc(F)cc1C(=O)NCc1ccc(-c2n[nH]c3ncnc(N)c23)cc1C ; CC(C)(C)OC(=O)N1CCN(c2ccc(B(O)O)cn2)CC1 ; COc1ccc(F)cc1C(=O)NCc1ccc(-c2nn(-c3ccc(N4CCN(C(=O)OC(C)(C)C)CC4)nc3)c3ncnc(N)c23)cc1C]

At room temperature, N-(4-aminopyrazolo[3,4-d]pyrimidin-3-yl)-2-methylbenzyl)-5-fluoro-2-methoxybenzamide (0.72 g, 1.72 mmol), (6-(4-(tert-butoxycarbonyl) piperazin-1-yl)pyridine-3-boronic acid (0.60 g, 1.95 mmol), copper acetate (0.35 g, 1.72 mmol), TEMPO (0.28 g, 1.95 mmol), DIEA (0.92 g, 7.09 mmol), MS (4A) (0.20 g) and DMSO (30 mL) were added into a single-necked flask in sequence. The reaction was allowed to proceed at 50° C. for 20 h under oxygen atmosphere before termination. The reaction solution was cooled to room temperature, filtered, diluted with water, and extracted with EA, and the organic phases were combined, dried over anhydrous Na₂SO₄, concentrated, and purified by column chromatography (PE/EA system) to afford 0.40 g of a black oily liquid with a yield of 34%. MS (ESI) m/z: 668.30 [M+H]⁺.

### Step 7: Synthesis of N-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide

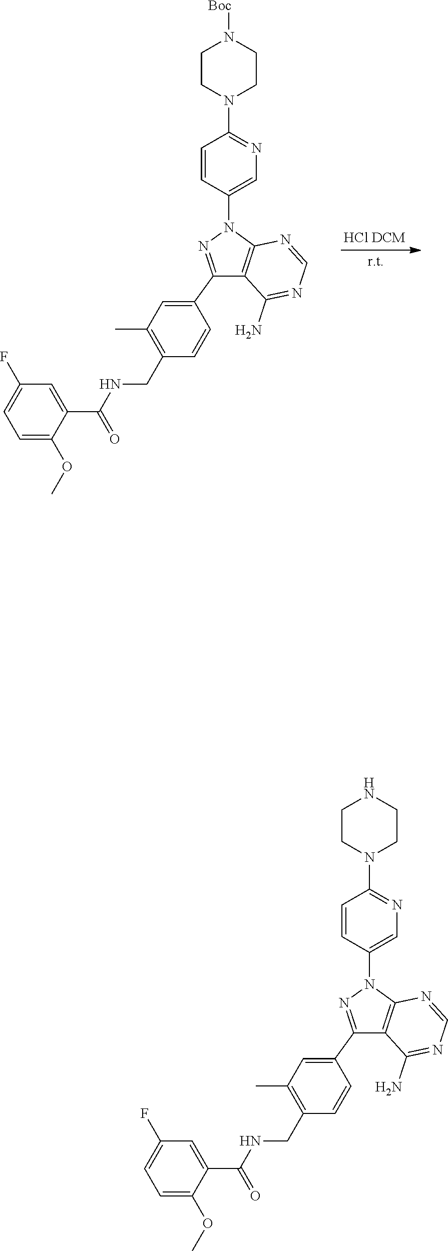 [SMILES: COc1ccc(F)cc1C(=O)NCc1ccc(-c2nn(-c3ccc(N4CCN(C(=O)OC(C)(C)C)CC4)nc3)c3ncnc(N)c23)cc1C ; COc1ccc(F)cc1C(=O)NCc1ccc(-c2nn(-c3ccc(N4CCNCC4)nc3)c3ncnc(N)c23)cc1C]

At room temperature, tert-butyl 4-(5-(4-amino-3-(4-((5-fluoro-2-methoxybenzamido)methyl)-3-methylphenyl)-1H-pyrazolo[3,4-d]pyrimidin-1-yl)pyridin-2-yl) piperazine-1-carboxylate (0.40 g, 0.70 mmol), hydrochloride in dioxane (20 mL) and DCM (20 mL) were added into a single-necked flask in sequence. The reaction was allowed to proceed at room temperature for 2 h under oxygen atmosphere before termination. The reaction solution was concentrated, diluted with saturated Na₂CO₃ and extracted with a MeOH/DCM mixed solvent, and the organic phases were combined, dried over anhydrous Na₂SO₄ and concentrated to afford 0.17 g of a brown solid. MS (ESI) m/z: 568.30 [M+H]⁺.

### Step 8: Synthesis of N-(4-(4-amino-1-(6-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-d]pyrimidin-3-yl)-2-methylbenzyl)-5-fluoro-2-methoxybenzamide

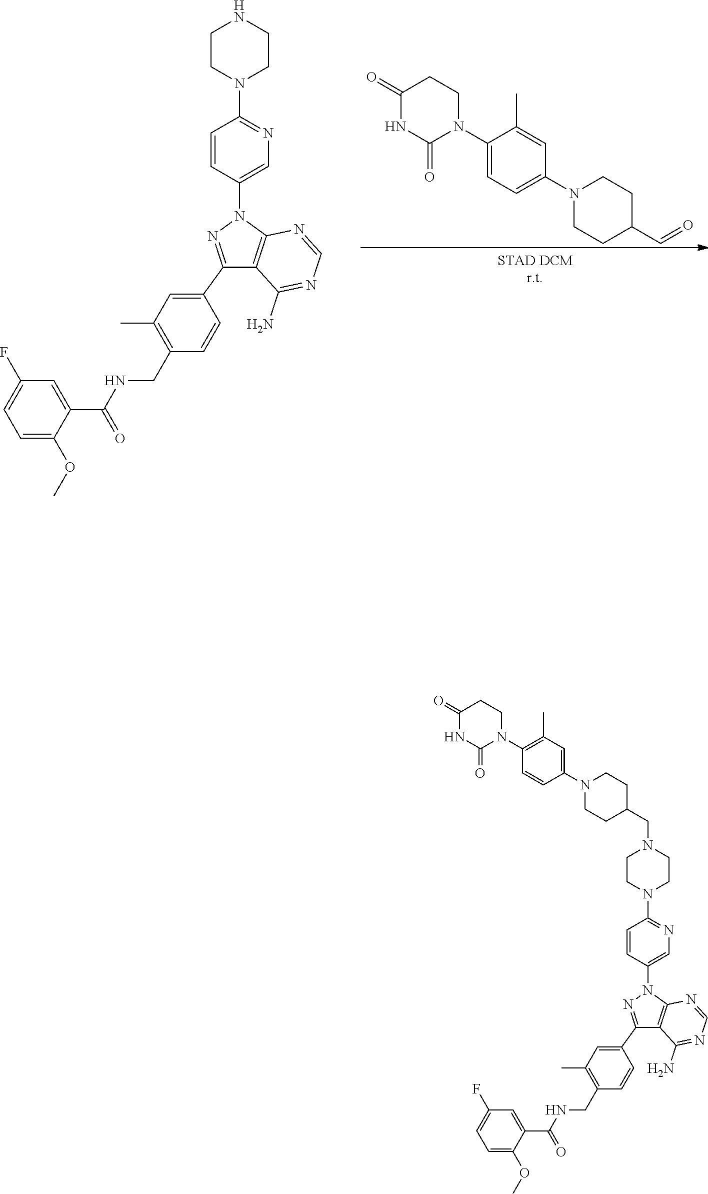 [SMILES: COc1ccc(F)cc1C(=O)NCc1ccc(-c2nn(-c3ccc(N4CCNCC4)nc3)c3ncnc(N)c23)cc1C ; Cc1cc(N2CCC(C=O)CC2)ccc1N1CCC(=O)NC1=O ; COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1C]

At room temperature, 1-(4-(2,4-dioxotetrahydropyrimidin-1(2H)-yl)-3-methylphenyl)piperidine-4-carbaldehyde (0.04 g, 0.08 mmol), N-(4-amino-1-(6-(piperazin-1-yl)pyridin-3-yl)-1H-pyrazolo[3,4-d]pyrimidin-3-yl)-2-methylbenzyl)-5-fluoro-2-methoxybenzamide (0.04 g, 0.06 mmol), STAB (0.02 g, 0.09 mmol) and DCM (10 mL g) were added into a single-necked flask in sequence. The reaction was allowed to proceed at room temperature for 2 h under nitrogen protection before termination. The reaction solution was cooled to room temperature, concentrated and purified by column chromatography (MeOH/DCM system) to afford 0.03 g of a white solid with a yield of 58%, 1H NMR (400 MHz, DMSO-d6) δ 8.85-8.71 (m, 2H), 8.33 (s, 1H), 8.17 (d, J=9.1 Hz, 1H), 7.62-7.41 (m, 4H), 7.41-7.30 (m, 1H), 7.24-7.14 (m, 1H), 7.03 (t, J=10.1 Hz, 2H), 6.84-6.74 (m, 2H), 4.57 (d, J=5.9 Hz, 2H), 3.91 (s, 3H), 3.70 (d, J=12.2 Hz, 3H), 3.62-3.51 (m, 4H), 3.51-3.36 (m, 5H), 2.80-2.57 (m, 4H), 2.44 (s, 3H), 2.23 (d, J=6.7 Hz, 2H), 2.12 (s, 3H), 1.91-1.70 (m, 4H), 1.31-1.14 (m, 3H). MS (ESI) m/z: 867.71 [M+H]⁺.

### Example 15: Synthesis of N-(4-(4-amino-1-(4-(1-((1-(4-(2,4-dioxotetrahydropyrimidin-1(2H)-yl)-3-methylphenyl)piperidin-4-yl)methyl)piperidinyl)phenyl)-1H-pyrazolo[3,4-d]pyrimidin-3-yl)benzyl)-5-fluoro-2-methoxybenzamide (Compound I-5)

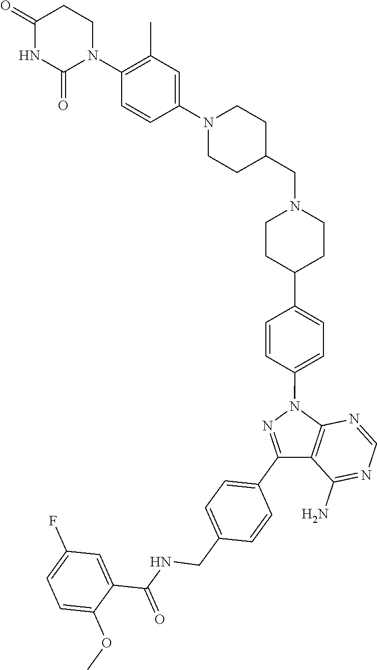 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1]

The synthesis method of N-(4-(4-amino-1-(4-(-4-formylpiperidin-1-yl)phenyl)-1H-pyrazolo[3,4-d]pyrimidin-3-yl)benzyl)-5-fluoro-2-methoxybenzamide follows Example 1.

### Step 1: Synthesis of tert-butyl 4-(4-(4-amino-3-(4-((5-fluoro-2-methoxybenzamido)methyl)phenyl)-1H-pyrazolo[3,4-d]pyrimidin-1-yl)phenyl)piperidine-1-carboxylate

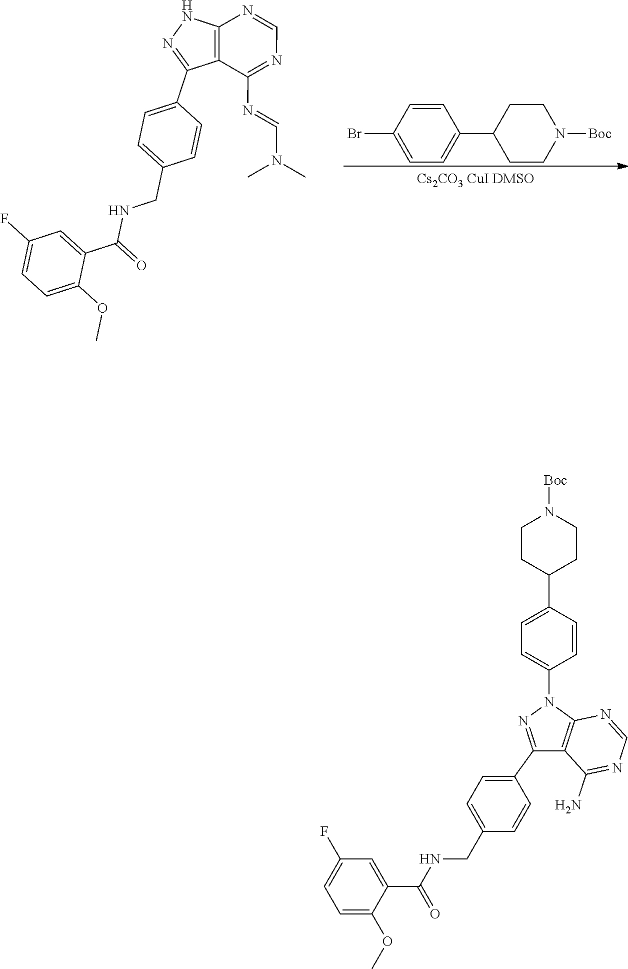

At room temperature, N-(4-(4-(((dimethylamino)methylene)amino)-1H-pyrazolo[3,4-d]pyrimidin-3-yl)benzyl)-5-fluoro-2-methoxybenzamide (0.40 g, 0.90 mmol), tert-butyl 4-(4-bromophenyl)piperidine-1-carboxylate (0.61 g, 1.79 mmol), cesium carbonate (0.87 g, 2.68 mmol), trans-1,4-diaminocyclohexane (0.06 g, 0.45 mmol), CuI (0.09 g, 0.45 mmol), and DMSO (20 mL) were added into a single-necked flask in sequence, and the reaction was allowed to proceed at 130° C. for 16 h under nitrogen protection. The reaction solution was cooled to room temperature, added with water, and extracted twice with EA. The organic phases were combined, washed with water and saturated brine, dried over anhydrous sodium sulfate and filtered, and the filtrate was concentrated and subjected to column chromatography (EA/PE system) to afford 0.20 g of a yellow solid with a yield of 34%.

### Step 2: Synthesis of N-(4-(4-amino-1-(4-(piperidin-4-yl)phenyl)-1H-pyrazolo[3,4-d]pyrimidin-3-yl)benzyl)-5-fluoro-2-methoxybenzamide

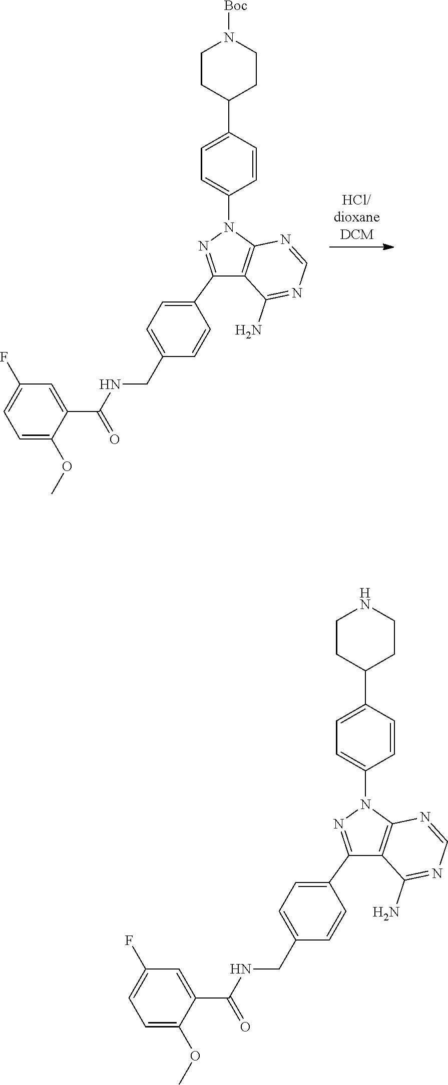 [SMILES: COc1ccc(F)cc1C(=O)NCc1ccc(-c2nn(-c3ccc(C4CCN(C(=O)OC(C)(C)C)CC4)cc3)c3ncnc(N)c23)cc1 ; COc1ccc(F)cc1C(=O)NCc1ccc(-c2nn(-c3ccc(C4CCNCC4)cc3)c3ncnc(N)c23)cc1]

At room temperature, tert-butyl 4-(4-(4-amino-3-(4-((5-fluoro-2-methoxybenzamido)methyl)phenyl)-1H-pyrazolo[3,4-d]pyrimidin-1-yl)phenyl)piperidine-1-carboxylate (0.20 g, 0.31 mmol), hydrochloride in dioxane (5 mL), and DCM (5 mL) were added into a single-necked flask in sequence and the reaction was allowed to proceed at room temperature for 3 h. The reaction solution was treated with triethylamine and concentrated to afford 0.16 g of a crude product as a yellow solid.

### Step 3: Synthesis of N-(4-(4-amino-1-(4-(1-((1-(4-(2,4-dioxotetrahydropyrimidin-1(2H)-yl)-3-methylphenyl)piperidin-4-yl)methyl)piperidinyl)phenyl)-1H-pyrazolo[3,4-d]pyrimidin-3-yl)benzyl)-5-fluoro-2-methoxybenzamide

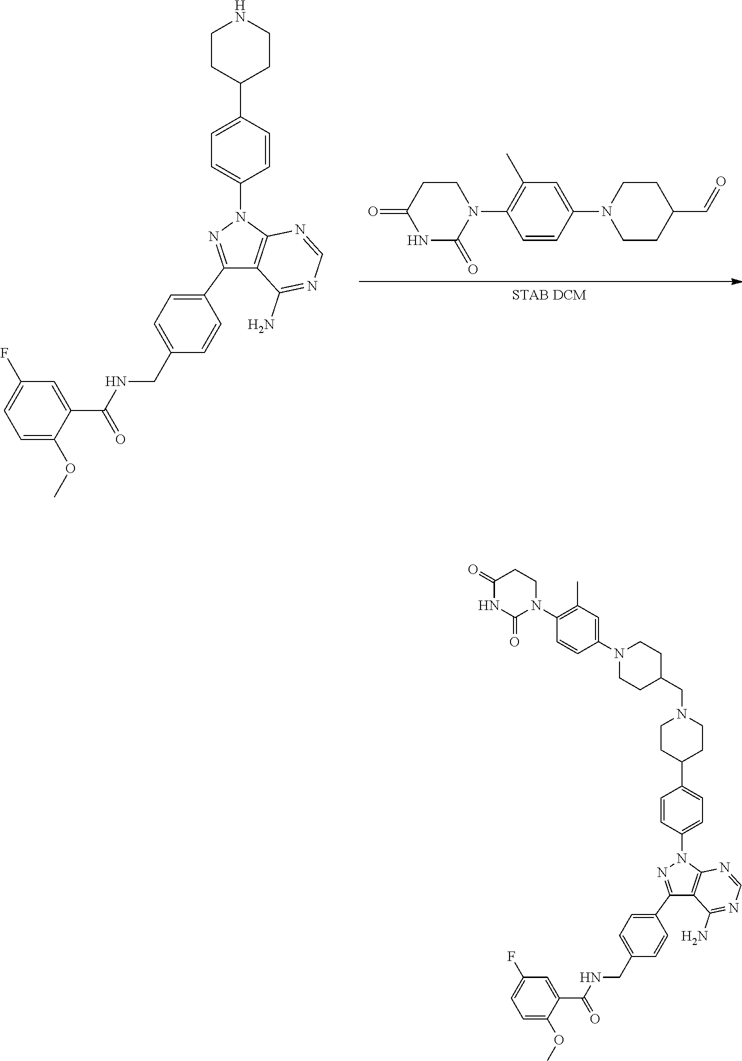 [SMILES: COc1ccc(F)cc1C(=O)NCc1ccc(-c2nn(-c3ccc(C4CCNCC4)cc3)c3ncnc(N)c23)cc1 ; Cc1cc(N2CCC(C=O)CC2)ccc1N1CCC(=O)NC1=O ; COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1]

At room temperature, N-(4-(4-amino-1-(4-(-4-formylpiperidin-1-yl)phenyl)-1H-pyrazolo[3,4-d]pyrimidin-3-yl)benzyl)-5-fluoro-2-methoxybenzamide (0.08 g, 0.15 mmol), 1-(4-(2,4-dioxotetrahydropyrimidin-1(2H)-yl)-3-methylphenyl)piperidine-4-carbaldehyde (0.06 g, 0.17 mmol), sodium triacetoxyborohydride (0.05 g, 0.22 mmol) and DCM (20 mL) were added into a single-necked flask in sequence, and the reaction was allowed to proceed at room temperature for 3 h. The reaction solution was concentrated and subjected to column chromatography (MeOH/DCM system) to afford 0.03 g of a yellow solid with a yield of 22%. ¹H NMR (400 MHz, DMSO-d₆) δ 8.93 (t, J=6.2 Hz, 1H), 8.37 (s, 1H), 8.13 (d, J=8.2 Hz, 2H), 7.74 (d, J=7.8 Hz, 2H), 7.58-7.50 (m, 3H), 7.45 (d, J=8.2 Hz, 2H), 7.39-7.31 (m, 1H), 7.20 (dd, J=9.1, 4.2 Hz, 1H), 7.05 (d, J=8.6 Hz, 1H), 6.85-6.73 (m, 2H), 4.61 (d, J=6.2 Hz, 2H), 3.91 (d, J=3.4 Hz, 2H), 3.74-3.61 (m, 4H), 3.10-2.98 (m, 10H), 2.77-2.64 (m, 6H), 2.12 (s, 3H), 1.90-1.80 (m, 5H). MS (ESI) m/z: 851.9 [M+H]⁺.

### Example 16: Synthesis of N-(4-(4-amino-1-(4-(1-((1-(4-(2,4-dioxotetrahydropyrimidin-1(2H)-yl)phenyl) piperidin-4-yl)methyl)piperidinyl)phenyl)-1H-pyrazolo[3,4-d]pyrimidin-3-yl)benzyl)-5-fluoro-2-methoxybenzamide (Compound I-6)

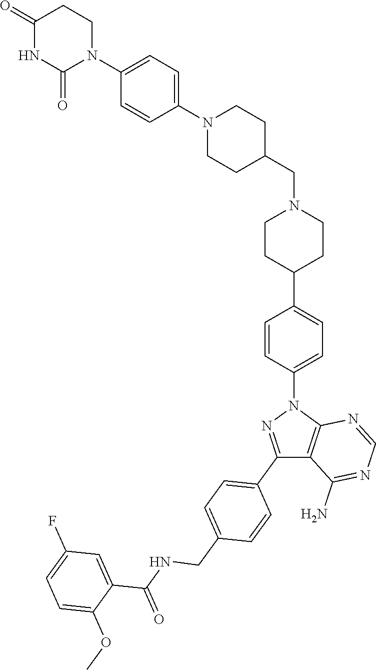 [SMILES: COc1ccc(F)cc1C(=O)NCc1ccc(-c2nn(-c3ccc(C4CCN(CC5CCN(c6ccc(N7CCC(=O)NC7=O)cc6)CC5)CC4)cc3)c3ncnc(N)c23)cc1]

The synthesis method of N-(4-(4-amino-1-(4-(-4-formylpiperidin-1-yl)phenyl)-1H-pyrazolo[3,4-d]pyrimidin-3-yl)benzyl)-5-fluoro-2-methoxybenzamide follows Example 1.

### Step 1: Synthesis of N-(4-(4-amino-1-(4-(1-((1-(4-(2,4-dioxotetrahydropyrimidin-1(2H)-yl)phenyl) piperidin-4-yl)methyl)piperidinyl)phenyl)-1H-pyrazolo[3,4-d]pyrimidin-3-yl)benzyl)-S-fluoro-2-methoxybenzamide

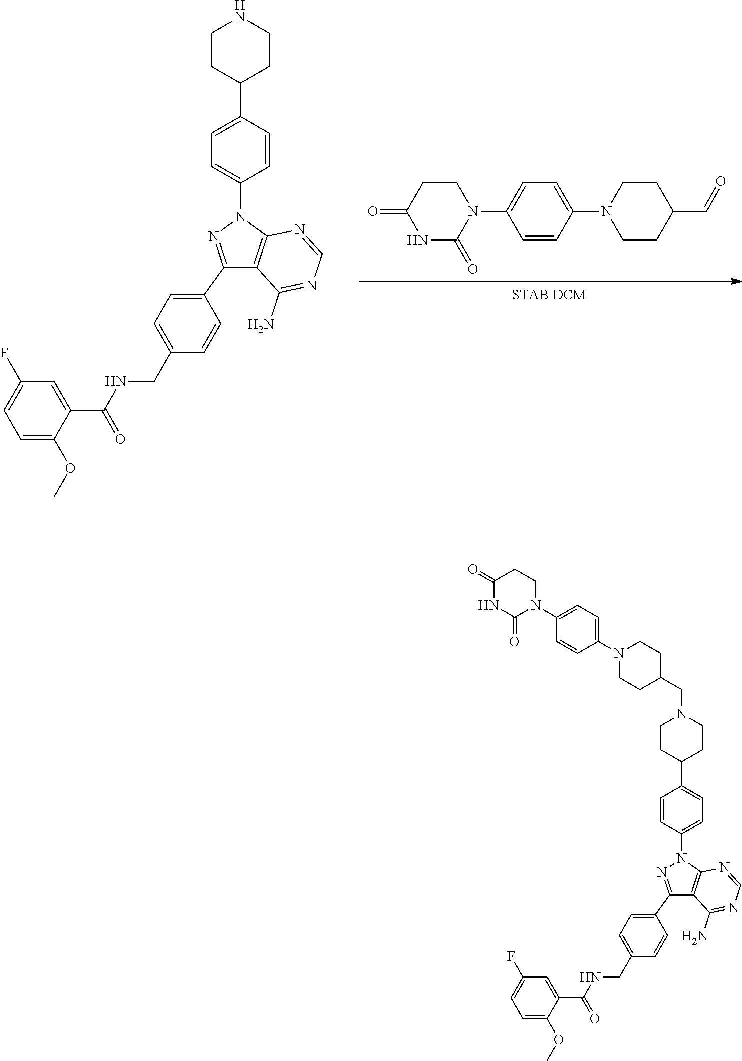 [SMILES: COc1ccc(F)cc1C(=O)NCc1ccc(-c2nn(-c3ccc(C4CCNCC4)cc3)c3ncnc(N)c23)cc1 ; O=CC1CCN(c2ccc(N3CCC(=O)NC3=O)cc2)CC1 ; COc1ccc(F)cc1C(=O)NCc1ccc(-c2nn(-c3ccc(C4CCN(CC5CCN(c6ccc(N7CCC(=O)NC7=O)cc6)CC5)CC4)cc3)c3ncnc(N)c23)cc1]

At room temperature, N-(4-(4-amino-1-(4-(-4-formylpiperidin-1-yl)phenyl)-1H-pyrazolo[3,4-d]pyrimidin-3-yl)benzyl)-5-fluoro-2-methoxybenzamide (0.08 g, 0.15 mmol), 1-(4-(2,4-dioxotetrahydropyrimidin-1(2H)-yl)phenyl)piperidine-4-carbaldehyde (0.05 g, 0.17 mmol), sodium triacetoxyborohydride (0.05 g, 0.22 mmol) and DCM (20 mL) were added into a single-necked flask in sequence, and the reaction was allowed to proceed at room temperature for 3 h. The reaction solution was concentrated and subjected to column chromatography (MeOH/DCM system) to afford 0.02 g of a white solid with a yield of 16%, ¹H NMR (400 MHz, DMSO-d₆) δ 8.92 (t, J=6.1 Hz, 1H), 8.37 (s, 1H), 8.12 (d, J=8.2 Hz, 2H), 7.75 (d, J=7.9 Hz, 2H), 7.59-7.50 (m, 3H), 7.45 (d, J=8.2 Hz, 2H), 7.40-7.31 (m, 1H), 7.21 (dd, J=9.1, 4.2 Hz, 1H), 7.14 (d, J=8.7 Hz, 2H), 6.93 (d, J=8.7 Hz, 2H), 4.62 (d, J=6.2 Hz, 2H), 3.91 (d, J=3.4 Hz, 2H), 3.74-3.59 (m, 4H), 3.42-3.37 (m, 1H), 3.06-2.88 (m, 2H), 2.75-2.57 (m, 5H), 2.29-2.11 (m, 2H), 2.07-1.91 (m, 2H), 1.89-1.60 (m, 8H), 1.31-1.07 (m, 3H). MS (ESI) m/z: 837.8 [M+H]⁺.

### Example 17: Synthesis of N-(4-(4-amino-1-(6-(1-((1-(4-(2,4-dioxotetrahydropyrimidin-1(2H)-yl)-3-methylphenyl)piperidin-4-yl)methyl)piperidin-4-yl)pyridin-3-yl)-1H-pyrazolo[3,4-d]pyrimidin-3-yl)benzyl)-5-fluoro-2-methoxybenzamide (Compound I-7)

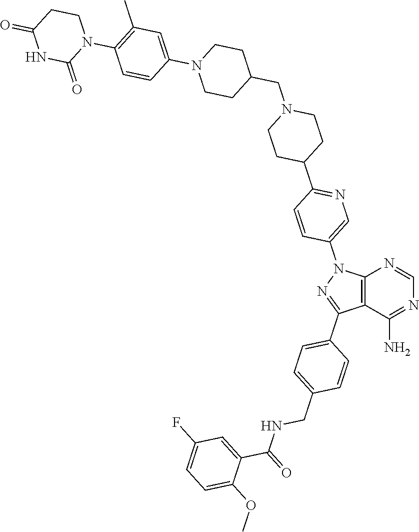 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c3ncnc(N)c23)cc1]

The synthesis method of N-(4-(4-amino-1-(6-(piperidin-4-yl)pyridin-3-yl)-1H-pyrazolo[3,4-d]pyrimidin-3-yl)benzyl)-5-fluoro-2-methoxybenzamide follows Example 1.

### Step 1: Synthesis of N-(4-(4-amino-1-(6-(1-((1-(4-(2,4-dioxotetrahydropyrimidin-1(2H)-yl)-3-methylphenyl)piperidin-4-yl)methyl)piperidin-4-yl)pyridin-3-yl)-1H-pyrazolo[3,4-d]pyrimidin-3-yl)benzyl)-5-fluoro-2-methoxybenzamide

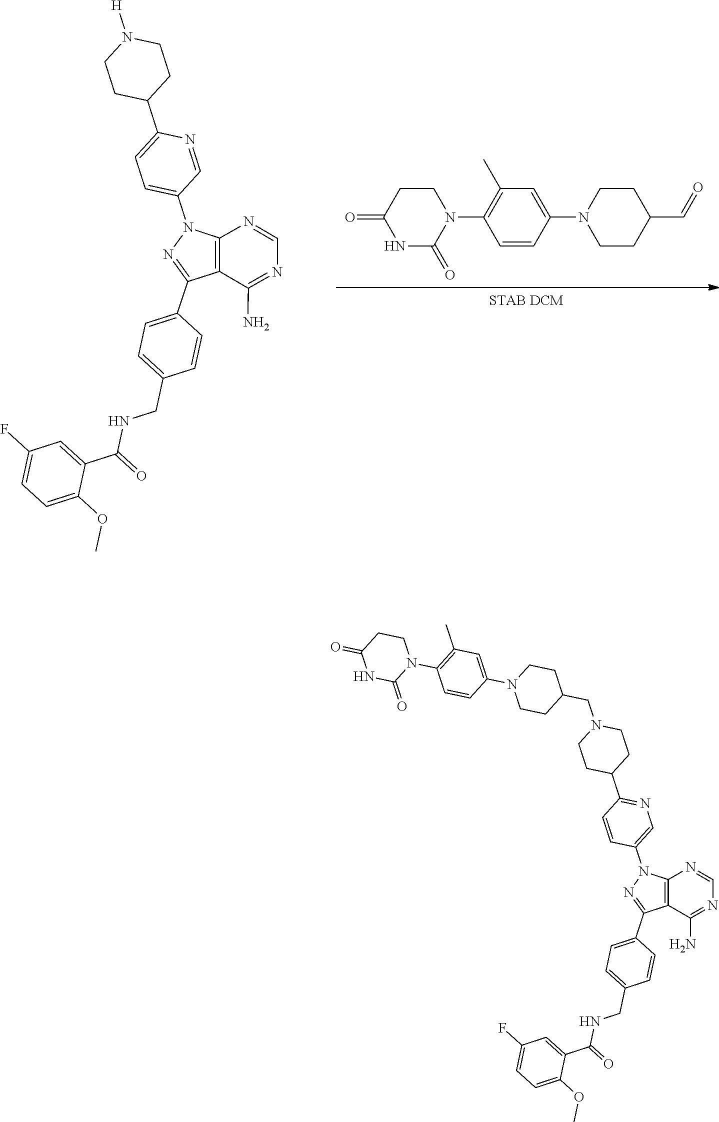 [SMILES: COc1ccc(F)cc1C(=O)NCc1ccc(-c2nn(-c3ccc(C4CCNCC4)nc3)c3ncnc(N)c23)cc1 ; Cc1cc(N2CCC(C=O)CC2)ccc1N1CCC(=O)NC1=O ; COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c3ncnc(N)c23)cc1]

At room temperature, N-(4-(4-amino-1-(6-(piperidin-4-yl)pyridin-3-yl)-1H-pyrazolo[3,4-d]pyrimidin-3-yl)benzyl)-5-fluoro-2-methoxybenzamide (0.06 g, 0.11 mmol), 1-(4-(2,4-dioxotetrahydropyrimidine-1(2H)-yl)-3-methylphenyl)piperidine-4-carbaldehyde (0.04 g, 0.12 mmol), DCM (20 mL) and NaBH (OAc)₃ (0.05 g, 0.22 mmol) were added into a single-necked flask in sequence. The reaction was allowed to proceed at room temperature for 16 h under nitrogen protection before termination. The reaction was quenched, and the reaction solution was concentrated and purified by column chromatography (MeOH/DCM system) to afford 0.035 g of a yellow solid with a yield of 38%, ¹H NMR (400 MHz, DMSO-d₆) δ 9.28 (d, J=2.6 Hz, 1H), 8.47 (dd, J=8.5, 2.6 Hz, 1H), 8.38 (s, 1H), 7.75 (d, J=8.1 Hz, 2H), 7.57-7.49 (m, 4H), 7.37-7.32 (m, 1H), 7.20 (dd, J=9.1, 4.3 Hz, 1H), 7.04 (d, J=8.6 Hz, 1H), 6.82-6.75 (m, 2H), 4.61 (d, J=6.1 Hz, 2H), 3.91 (s, 3H), 3.71-3.63 (m, 3H), 3.50-3.43 (m, 2H), 3.17 (s, 1H), 3.06-2.95 (m, 2H), 2.76-2.62 (m, 5H), 2.33-2.20 (m, 2H), 2.11-2.00 (m, 5H), 1.87-1.72 (m, 7H), 1.24-1.18 (m, 2H). MS (ESI) m/z: 852.7 [M+H]⁺.

### Example 18: Synthesis of N-(4-(4-amino-1-(6-(1-(4-(2,6-dioxopiperidin-3-yl)-3-methylphenyl)piperidin-4-yl)pyridin-3-yl)-1H-pyrazolo[3,4-d]pyrimidin-3-yl)benzyl)-5-fluoro-2-methoxybenzamide (Compound I-39)

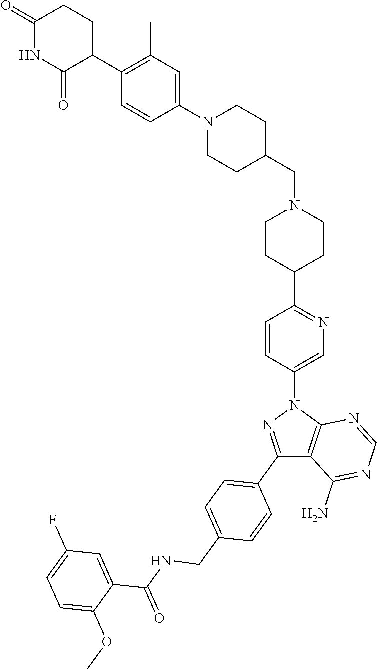 [SMILES: COc1ccc(F)cc1C(=O)NCc1ccc(-c2nn(-c3ccc(C4CCN(CC5CCN(c6ccc(C7CCC(=O)NC7=O)c(C)c6)CC5)CC4)nc3)c3ncnc(N)c23)cc1]

The synthesis method of N-(4-amino-1-(6-(piperidin-4-yl)pyridin-3-yl)-1H-pyrazolo[3,4-d]pyrimidin-3-yl)benzyl)-5-fluoro-2-methoxybenzamide follows Example 1.

### Step 1: Synthesis of 1-(4-bromo-3-methylphenyl)-4-dimethoxymethylpiperidine

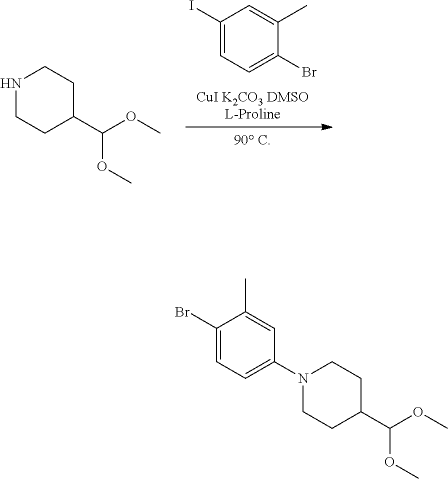

At room temperature, 4-(dimethoxymethyl)piperidine (1.13 g, 7.07 mmol), 1-bromo-4-iodo-2-methylbenzene (2.07 g, 7.07 mmol), cuprous iodide (0.27 g, 1.77 mmol), anhydrous potassium carbonate (7.96 g, 14.14 mmol), L-proline (0.33 g, 2.83 mmol) and DMSO (50 mL) were added into a single-necked flask in sequence. The reaction was allowed to proceed at 90° C. for 18 h under nitrogen protection before termination. The reaction solution was cooled to room temperature, diluted with water, and extracted with EA, and the organic phase was dried over anhydrous Na₂SO₄, filtered, concentrated and purified by column chromatography (EA/PE system) to afford 1.07 g of a light yellow solid with a yield of 46%.

### Step 2: Synthesis of 2,6-bis(benzyloxy)-3-(4-(4-(dimethoxymethyl) piperidin-1-yl)phenyl)-5-methylpyridine

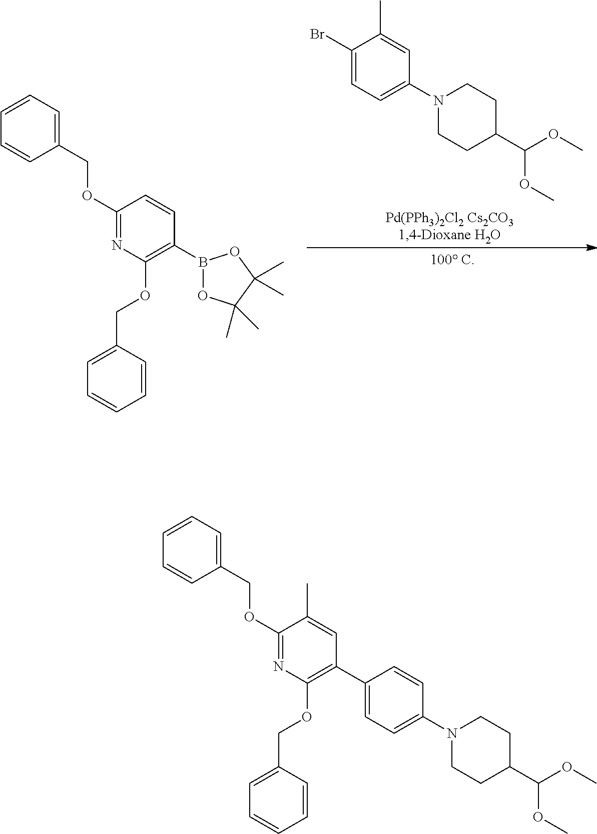 [SMILES: CC1(C)OB(c2ccc(OCc3ccccc3)nc2OCc2ccccc2)OC1(C)C ; COC(OC)C1CCN(c2ccc(Br)c(C)c2)CC1 ; COC(OC)C1CCN(c2ccc(-c3cc(C)c(OCc4ccccc4)nc3OCc3ccccc3)cc2)CC1]

At room temperature, 1-(4-bromo-3-methylphenyl)-4-dimethoxymethylpiperidine (1.07 g, 3.27 mmol), 2,6-bis(benzyloxy)-3-(4,4,5,5-tetramethyl-1,3,2-dioxaborolan-2-yl)pyridine (2.0 g, 4.79 mmol), bis(triphenylphosphine) palladium dichloride (0.46 g, 0.65 mmol), cesium carbonate (3.20 g, 9.81 mmol), 1,4-dioxane (40 mL) and water (2 mL) were added into a single-necked flask in sequence. The reaction was allowed to proceed at 100° C. for 16 h under nitrogen protection before termination. The reaction solution was cooled to room temperature, dried with anhydrous Na₂SO₄ and filtered, and the filtrate was concentrated and purified by column chromatography (EA/PE system) to afford 0.81 g of a white solid with a yield of 46%.

### Step 3: Synthesis of 3-(4-(4-(dimethoxymethyl) piperidin-1-yl)-2-methylphenyl)piperidine-2,6-dione

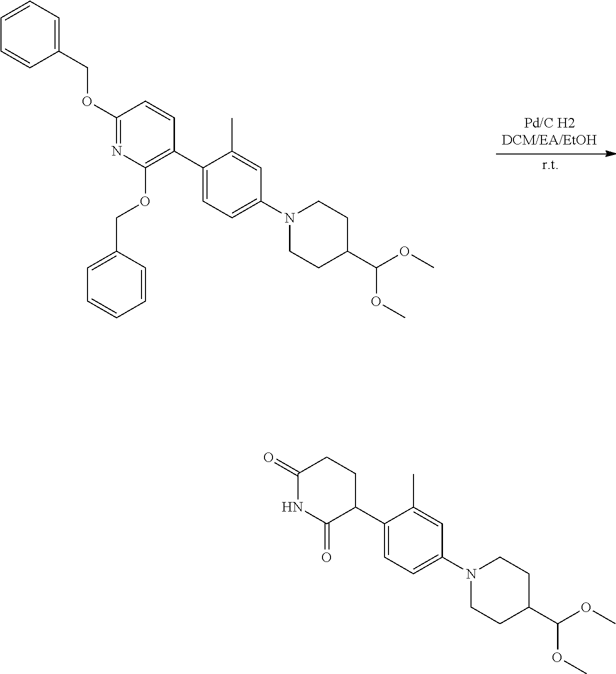 [SMILES: COC(OC)C1CCN(c2ccc(-c3ccc(OCc4ccccc4)nc3OCc3ccccc3)c(C)c2)CC1 ; COC(OC)C1CCN(c2ccc(C3CCC(=O)NC3=O)c(C)c2)CC1]

At room temperature, 2,6-bis(benzyloxy)-3-(4-(4-(dimethoxymethyl) piperidin-1-yl)phenyl)-5-methylpyridine (0.81 g, 1.50 mmol), palladium on carbon (10%, 0.49 g) and a EtOH/EA/DCM mixed solvent (44 mL) were added into a single-necked flask in sequence. The reaction was allowed to proceed at room temperature for 6 h under hydrogen atmosphere before termination. The reaction solution was filtered and the filtrate was concentrated to afford 0.13 g of a crude product as a white solid.

### Step 4: Synthesis of 1-(4-(2,6-dioxopiperidin-3-yl)-3-methylphenyl)piperidine-4-carboxaldehyde

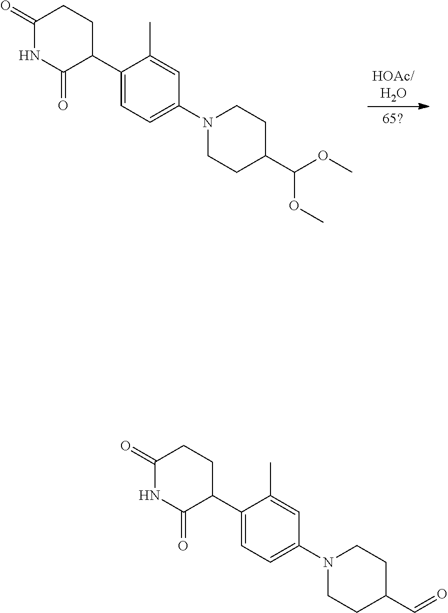 [SMILES: COC(OC)C1CCN(c2ccc(C3CCC(=O)NC3=O)c(C)c2)CC1 ; Cc1cc(N2CCC(C=O)CC2)ccc1C1CCC(=O)NC1=O]

At room temperature, 3-(4-(4-(dimethoxymethyl) piperidin-1-yl)-2-methylphenyl)piperidine-2,6-dione (0.13 g, 0.41 mmol), glacial acetic acid (10 mL) and water (10 mL) were added into a single-necked flask in sequence. The reaction was allowed to proceed at 65° C. for 16 h under nitrogen protection before termination. The reaction solution was cooled to room temperature and concentrated to afford 0.10 g of a crude product as a purple solid.

### Step 5: Synthesis of N-(4-(4-amino-1-(6-(1-(4-(2,6-dioxopiperidin-3-yl)-3-methylphenyl)piperidin-4-yl)pyridin-3-yl)-1H-pyrazolo[3,4-d]pyrimidin-3-yl)benzyl)-5-fluoro-2-methoxybenzamide

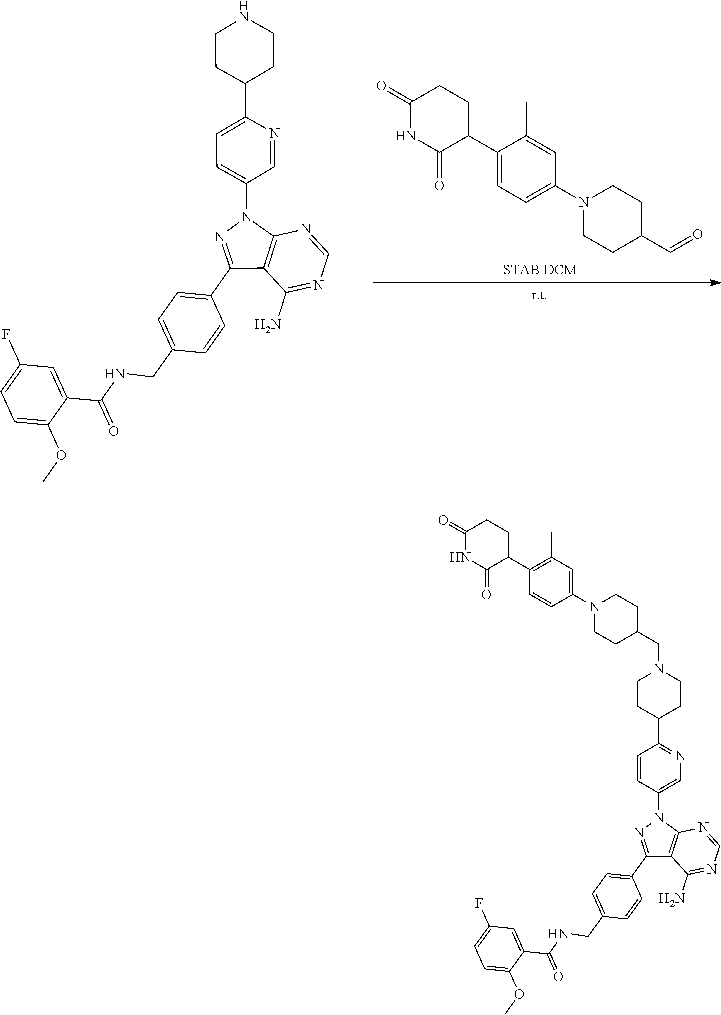 [SMILES: COc1ccc(F)cc1C(=O)NCc1ccc(-c2nn(-c3ccc(C4CCNCC4)nc3)c3ncnc(N)c23)cc1 ; Cc1cc(N2CCC(C=O)CC2)ccc1C1CCC(=O)NC1=O ; COc1ccc(F)cc1C(=O)NCc1ccc(-c2nn(-c3ccc(C4CCN(CC5CCN(c6ccc(C7CCC(=O)NC7=O)c(C)c6)CC5)CC4)nc3)c3ncnc(N)c23)cc1]

At room temperature, 1-(4-(2,6-dioxopiperidin-3-yl)-3-methylphenyl)piperidine-4-carbaldehyde (0.03 g, 0.09 mmol), N-(4-amino-1-(6-(piperidin-4-yl)pyridin-3-yl)-1H-pyrazolo[3,4-d]pyrimidin-3-yl)benzyl)-5-fluoro-2-methoxybenzamide (0.04 g, 0.07 mmol), STAB (0.02 g, 0.11 mmol) and DCM (10 mL) were added into a single-necked flask in sequence. The reaction was allowed to proceed at room temperature for 2 h under nitrogen protection before termination. The reaction solution was concentrated and purified by column chromatography (MeOH/DCM system) to afford 0.02 g of a white solid with a yield of 26%, ¹H NMR (400 MHz, DMSO-d₆) δ 9.29 (s, 1H), 8.92 (t, J=6.1 Hz, 1H), 8.44 (m, 2H), 7.76 (d, J=7.7 Hz, 2H), 7.61-7.45 (m, 3H), 7.36 (m, 1H), 7.21 (m, 1H), 6.91 (m, 1H), 6.81-6.58 (m, 2H), 4.55 (d, J=55.9 Hz, 3H), 3.92 (s, 3H), 3.67 (m, 2H), 3.02 (m, 2H), 2.63 (m, 6H), 2.15 (m, 8H), 1.90-1.52 (m, 7H), 1.23 (m, 3H). MS (ESI) m/z: 851.7 [M+H]⁺.

### Example 19: Synthesis of N-(4-(4-amino-1-(6-(1-(4-(2,6-dioxopiperidin-3-yl)phenyl) piperidin-4-yl)pyridin-3-yl)pyridin-3-yl)-1H-pyrazolo[3,4-d]pyrimidin-3-yl)benzyl)-5-fluoro-2-methoxybenzamide (Compound I-24)

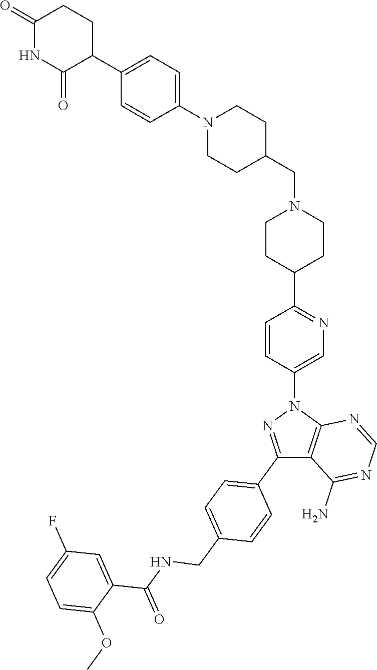 [SMILES: COc1ccc(F)cc1C(=O)NCc1ccc(-c2nn(-c3ccc(C4CCN(CC5CCN(c6ccc(C7CCC(=O)NC7=O)cc6)CC5)CC4)nc3)c3ncnc(N)c23)cc1]

The synthesis method of N-(4-amino-1-(6-(piperidin-4-yl)pyridin-3-yl)-1H-pyrazolo[3,4-d]pyrimidin-3-yl)benzyl)-5-fluoro-2-methoxybenzamide follows Example 1.

### Step 1: Synthesis of 1-(4-bromophenyl)-4-dimethoxymethylpiperidine

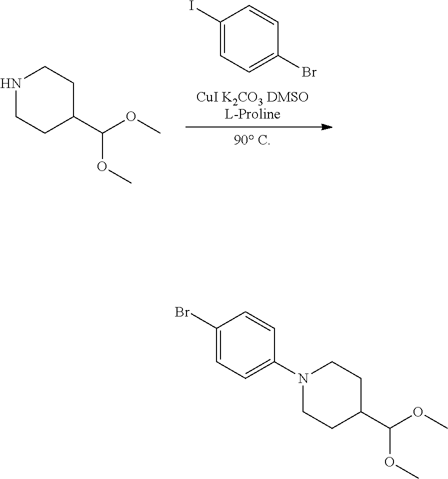 [SMILES: COC(OC)C1CCNCC1 ; Brc1ccc(I)cc1 ; COC(OC)C1CCN(c2ccc(Br)cc2)CC1]

At room temperature, 4-(dimethoxymethyl)piperidine (1.13 g, 7.07 mmol), 1-bromo-4-iodobenzene (2.0 g, 7.07 mmol), cuprous iodide (0.27 g, 1.77 mmol), anhydrous potassium carbonate (7.96 g, 14.14 mmol), L-proline (0.33 g, 2.83 mmol) and DMSO (50 mL) were added into a single-necked flask in sequence. The reaction was allowed to proceed at 90° C. for 18 h under nitrogen protection before termination. The reaction solution was cooled to room temperature, diluted with water, and extracted with EA, and the organic phase was dried over anhydrous Na₂SO₄, filtered, concentrated and purified by column chromatography (EA/PE system) to afford 1.49 g of a light yellow solid with a yield of 68%.

### Step 2: Synthesis of 2,6-bis(benzyloxy)-3-(4-(4-(dimethoxymethyl) piperidin-1-yl)phenyl)pyridine

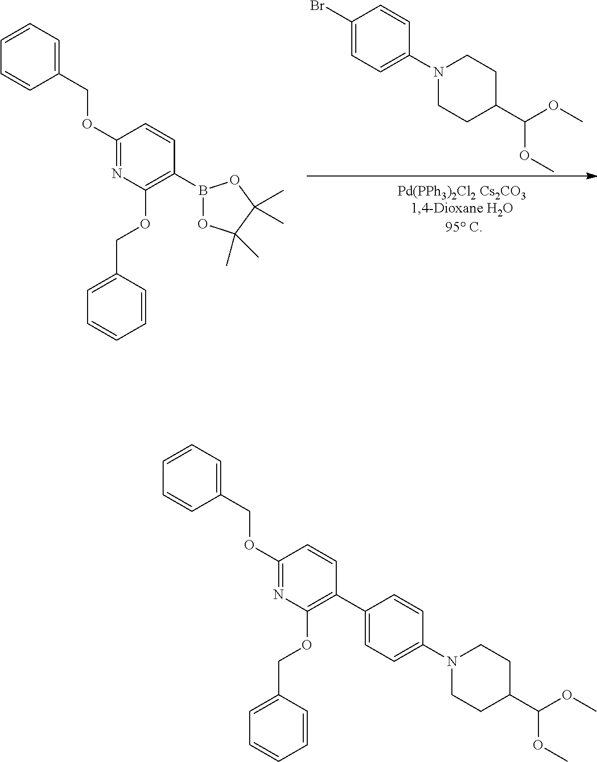 [SMILES: CC1(C)OB(c2ccc(OCc3ccccc3)nc2OCc2ccccc2)OC1(C)C ; COC(OC)C1CCN(c2ccc(Br)cc2)CC1 ; COC(OC)C1CCN(c2ccc(-c3ccc(OCc4ccccc4)nc3OCc3ccccc3)cc2)CC1]

At room temperature, 1-(4-bromophenyl)-4-dimethoxymethylpiperidine (1.49 g, 4.76 mmol), 2,6-bis(benzyloxy)-3-(4,4,5,5-tetramethyl-1,3,2-dioxaborolan-2-yl)pyridine (2.98 g, 7.14 mmol), bis(triphenylphosphine) palladium dichloride (0.67 g, 0.95 mmol), cesium carbonate (4.65 g, 14.28 mmol), 1,4-dioxane (30 mL) and water (1 mL) were added into a single-necked flask in sequence. The reaction was allowed to proceed at 95° C. for 16 h under nitrogen protection before termination. The reaction solution was cooled to room temperature, dried with anhydrous Na₂SO₄ and filtered, and the filtrate was concentrated and purified by column chromatography (EA/PE system) to afford 0.50 g of a white solid with a yield of 20%.

### Step 3: Synthesis of 3-(4-(4-(dimethoxymethyl) piperidin-1-yl)phenyl)piperidine-2,6-dione

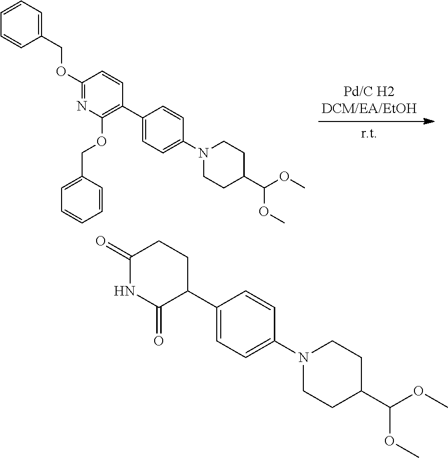

At room temperature, 2,6-bis(benzyloxy)-3-(4-(4-(dimethoxymethyl) piperidin-1-yl)phenyl)pyridine (0.50 g, 0.95 mmol), palladium on carbon (10%, 0.30 g) and a EtOH/EA/DCM mixed solvent (44 mL) were added into a single-necked flask in sequence. The reaction was allowed to proceed at room temperature for 6 h under hydrogen atmosphere before termination. The reaction solution was filtered and the filtrate was concentrated to afford 0.27 g of a crude product as a white solid.

### Step 4: Synthesis of 1-(4-(2,6-dioxopiperidin-3-yl)phenyl)piperidine-4-carboxaldehyde

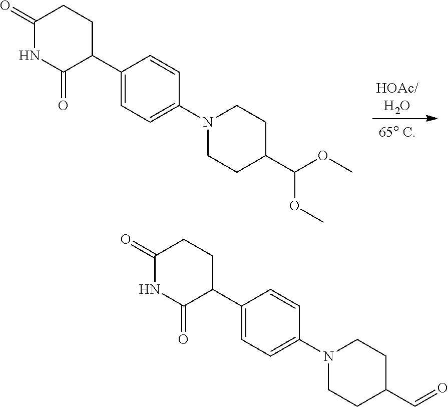 [SMILES: COC(OC)C1CCN(c2ccc(C3CCC(=O)NC3=O)cc2)CC1 ; O=CC1CCN(c2ccc(C3CCC(=O)NC3=O)cc2)CC1]

At room temperature, 3-(4-(4-(dimethoxymethyl) piperidin-1-yl)phenyl)piperidine-2,6-dione (0.27 g, 0.78 mmol), glacial acetic acid (10 mL) and water (10 mL) were added into a single-necked flask in sequence. The reaction was allowed to proceed at 65° C. for 16 h under nitrogen protection before termination. The reaction solution was cooled to room temperature, concentrated, and purified by column chromatography (MeOH/DCM system) to afford 0.22 g of a light yellow solid with a yield of 94%.

### Step 5: Synthesis of N-(4-(4-amino-1-(6-(1-(4-(2,6-dioxopiperidin-3-yl)phenyl) piperidin-4-yl)pyridin-3-yl)pyridin-3-yl)-1H-pyrazolo[3,4-d]pyrimidin-3-yl)benzyl)-5-fluoro-2-methoxybenzamide

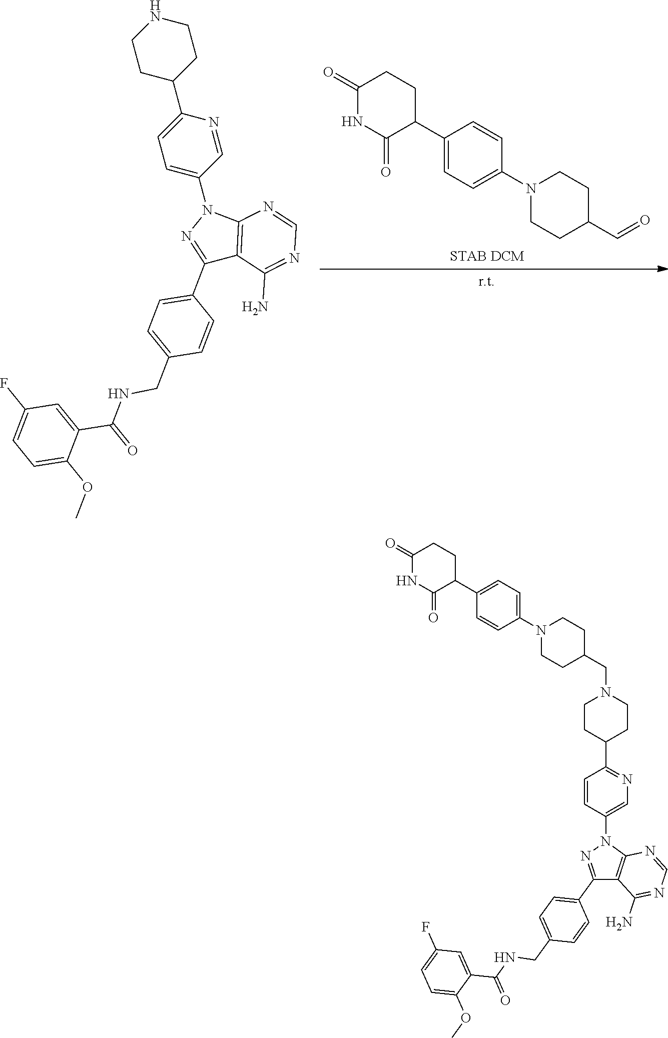 [SMILES: COc1ccc(F)cc1C(=O)NCc1ccc(-c2nn(-c3ccc(C4CCNCC4)nc3)c3ncnc(N)c23)cc1 ; O=CC1CCN(c2ccc(C3CCC(=O)NC3=O)cc2)CC1 ; COc1ccc(F)cc1C(=O)NCc1ccc(-c2nn(-c3ccc(C4CCN(CC5CCN(c6ccc(C7CCC(=O)NC7=O)cc6)CC5)CC4)nc3)c3ncnc(N)c23)cc1]

At room temperature, 1-(4-(2,6-dioxopiperidin-3-yl)phenyl)piperidine-4-carbaldehyde (0.03 g, 0.08 mmol), N-(4-amino-1-(6-(piperidin-4-yl)pyridin-3-yl)-1H-pyrazolo[3,4-d]pyrimidin-3-yl)benzyl)-5-fluoro-2-methoxybenzamide (0.04 g, 0.06 mmol), STAB (0.02 g, 0.10 mmol) and DCM (10 mL) were added into a single-necked flask in sequence. The reaction was allowed to proceed at room temperature for 2 h under nitrogen protection before termination. The reaction solution was concentrated and purified by column chromatography (MeOH/DCM system) to afford 0.04 g of a white solid with a yield of 66%, ¹H NMR (400 MHz, DMSO-d₆) δ 9.29 (s, 1H), 8.92 (t, J=6.1 Hz, 1H), 8.48 (d, J=7.9 Hz, 1H), 8.39 (s, 1H), 7.76 (d, J=7.7 Hz, 2H), 7.64-7.48 (m, 4H), 7.36 (m, 1H), 7.21 (m, 1H), 7.04 (d, J=8.2 Hz, 2H), 6.90 (d, J=8.3 Hz, 2H), 4.62 (d, J=6.2 Hz, 2H), 3.92 (s, 3H), 3.78-3.59 (m, 3H), 3.39 (m, 1H), 2.99 (m, 2H), 2.84-2.56 (m, 5H), 2.08 (m, 3H), 1.95-1.59 (m, 8H), 1.23 (m, 3H). MS (ESI) m/z: 837.7 [M+H]⁺.

### Example 20: Synthesis of N-(4-(8-amino-3-(4-((1-(4-(2,4-dioxotetrahydropyrimidin-1(2H)-yl)-3-methylphenyl)piperidin-4-yl)methyl)piperazin-1-yl)phenyl)imidazo[1,5-a]pyrazin-1-yl]benzyl)-5-fluoro-2-methoxybenzamide (Compound I-40)

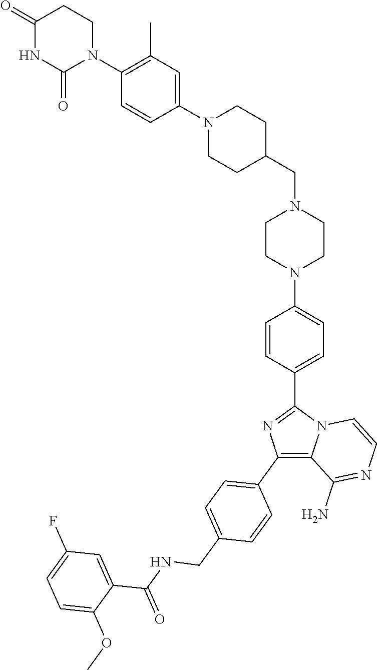 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1]

### Step 1 and Step 2: Synthesis of tert-butyl 4-(4-(8-amino-1-(4-((5-fluoro-2-methoxybenzamido)methyl)phenyl)imidazo[1,5-a]pyrazin-3-yl)phenyl) piperazine-1-carboxylate

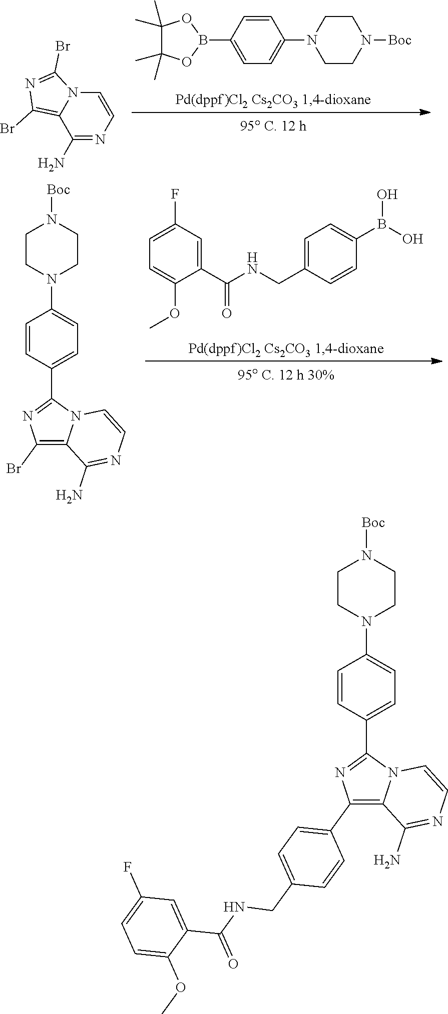

At room temperature, 1,3-dibromoimidazo[1,5-a]pyrazin-8-amine (0.30 g, 1.03 mmol), tert-butyl 4-(4-(4,4,5,5-tetramethyl-1,3,2-dioxaborolan-2-yl)phenyl) piperazine-1-carboxylate (0.40 g, 1.13 mmol), cesium carbonate (0.67 g, 2.06 mmol), Pd(dppf)Cl₂ (0.07 g, 0.10 mmol), 1,4-dioxane (10 mL), ethylene glycol monomethyl ether (5 mL), and H₂O (1 mL) were added into a single-necked flask in sequence. Under nitrogen protection, the mixture was heated to 95° C. and the reaction was allowed to proceed for 12 h. The reaction solution was cooled to room temperature, and (4-((5-fluoro-2-methoxybenzamido)methyl)phenyl)boronic acid (0.34 g, 1.13 mmol) and Pd(dppf)Cl₂ (0.07 g, 0.14 mmol) were added. Under nitrogen protection, the mixture was heated to 95° C. and the reaction was allowed to proceed for another 12 h. The reaction solution was cooled to room temperature and filtered, and the filtrate was concentrated and purified by column chromatography (MeOH/DCM system) to afford 0.20 g of a yellow solid with a yield of 30%.

### Step 3: Synthesis of N-(4-(8-amino-3-(4-(piperazin-1-yl)phenyl)imidazo[1,5-a]pyrazin-1-yl]benzyl)-5-fluoro-2-methoxybenzamide

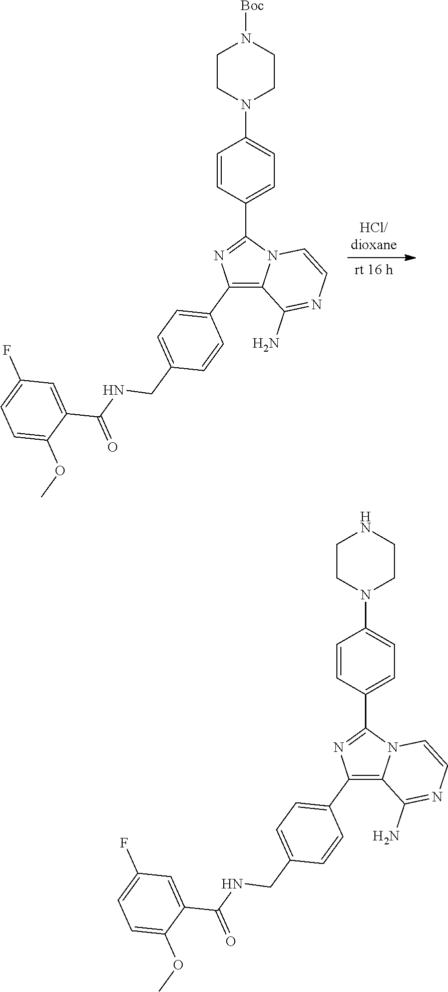 [SMILES: COc1ccc(F)cc1C(=O)NCc1ccc(-c2nc(-c3ccc(N4CCN(C(=O)OC(C)(C)C)CC4)cc3)n3ccnc(N)c23)cc1 ; COc1ccc(F)cc1C(=O)NCc1ccc(-c2nc(-c3ccc(N4CCNCC4)cc3)n3ccnc(N)c23)cc1]

At room temperature, tert-butyl 4-(4-(8-amino-1-(4-((5-fluoro-2-methoxybenzamido)methyl)phenyl)imidazo[1,5-a]pyrazin-3-yl)phenyl) piperazine-1-carboxylate (0.20 g, 0.31 mmol), HCl-dioxane (5 mL) and DCM (5 mL) were added into a single-necked flask in sequence and the reaction was allowed to proceed for 16 h. The reaction solution was concentrated, and the concentrated reaction solution was treated with aqueous ammonia and concentrated to afford 0.18 g of a crude product as a yellow solid.

### Step 4: Synthesis of N-(4-(8-amino-3-(4-((1-(4-(2,4-dioxotetrahydropyrimidin-1(2H)-yl)-3-methylphenyl)piperidin-4-yl)methyl)piperazin-1-yl)phenyl)imidazo[1,5-a]pyrazin-1-yl]benzyl)-5-fluoro-2-methoxybenzamide

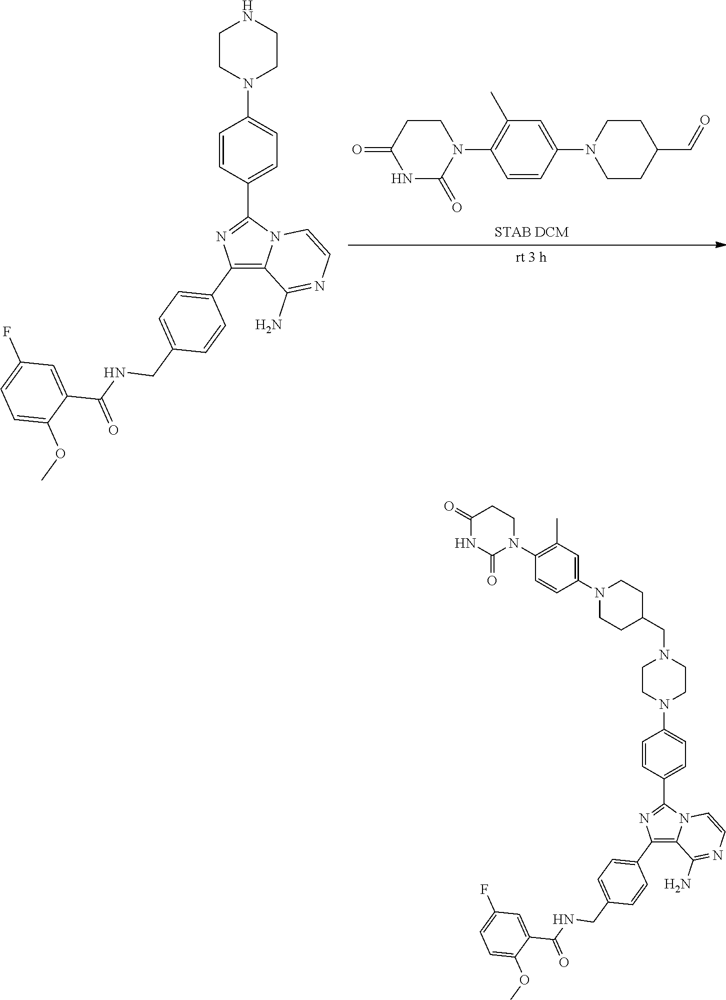 [SMILES: COc1ccc(F)cc1C(=O)NCc1ccc(-c2nc(-c3ccc(N4CCNCC4)cc3)n3ccnc(N)c23)cc1 ; Cc1cc(N2CCC(C=O)CC2)ccc1N1CCC(=O)NC1=O ; COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1]

At room temperature, N-(4-(8-amino-3-(4-(piperazin-1-yl)phenyl)imidazo[1,5-a]pyrazin-1-yl]benzyl)-5-fluoro-2-methoxybenzamide (0.10 g, 0.18 mmol), 1-(4-(2,4-dioxotetrahydropyrimidin-1(2H)-yl)-3-methylphenyl)piperidine-4-carbaldehyde (0.07 g, 0.22 mmol), sodium triacetoxyborohydride (0.06 g, 0.27 mmol) and DCM (20 mL) were added into a single-necked flask in sequence and the reaction was allowed to proceed at room temperature for 3 h. The reaction solution was concentrated and purified by column chromatography (MeOH/DCM system) to afford 0.08 g of a product with a yield of 52%. ¹H NMR (400 MHz, DMSO-d₆) δ 10.26 (s, 1H), 8.88 (t, J=6.1 Hz, 1H), 7.81-7.62 (m, 5H), 7.57-7.46 (m, 3H), 7.39-7.31 (m, 1H), 7.20 (dd, J=9.2, 4.2 Hz, 1H), 7.08 (dt, J=15.4, 8.9 Hz, 4H), 6.84-6.75 (m, 2H), 6.08 (s, 2H), 4.60 (d, J=6.1 Hz, 2H), 3.91 (s, 3H), 3.77-3.57 (m, 3H), 3.52-3.43 (m, 1H), 3.30-3.09 (m, 5H), 2.68 (dt, J=15.2, 7.9 Hz, 3H), 2.52 (s, 1H), 2.24 (d, J=7.0 Hz, 2H), 2.12 (s, 3H), 1.87-1.66 (m, 3H), 1.22 (d, J=12.0 Hz, 2H). MS (ESI) m/z: 851.8 [M+H]⁺.

### Example 21: Synthesis of N-(4-(8-amino-3-(6-(4-(1-(4-(2,4-dioxotetrahydropyrimidin-1(2H)-yl)-3-methylphenyl)piperidin-4-yl)methyl)piperazin-1-yl)pyridin-3-yl) imidazo[1,5-a]pyrazin-1-yl]benzyl)-5-fluoro-2-methoxybenzamide (Compound I-30)

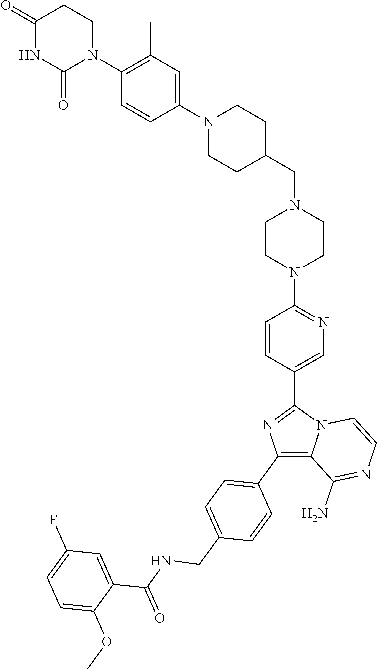 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)nc3)n3ccnc(N)c23)cc1]

### Step 1 and Step 2: Synthesis of tert-butyl 4-(5-(8-amino-1-(4-((5-fluoro-2-methoxybenzamido)methyl)phenyl)imidazo[1,5-a]pyrazin-3-yl)pyridin-2-yl) piperazine-1-carboxylate

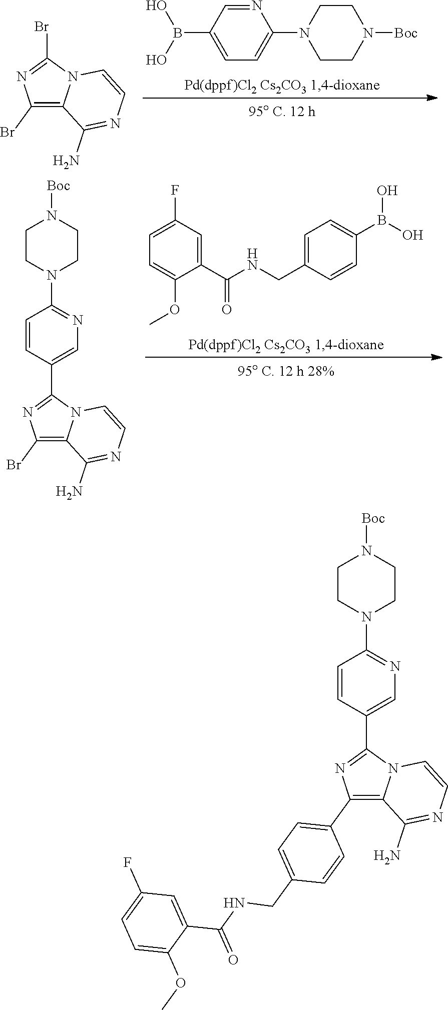

At room temperature, 1,3-dibromoimidazo[1,5-a]pyrazin-8-amine (0.25 g, 0.86 mmol), (6-(4-(tert-butoxycarbonyl) piperazin-1-yl)pyridin-3-yl) boronic acid (0.26 g, 0.86 mmol), cesium carbonate (0.56 g, 1.72 mmol), Pd(dppf)Cl₂ (0.06 g, 0.08 mmol), 1,4-dioxane (10 mL), ethylene glycol monomethyl ether (5 mL) and H₂O (1 mL) were added into a single-necked flask in sequence. Under nitrogen protection, the mixture was heated to 95° C. and the reaction was allowed to proceed for 12 h. The reaction solution was cooled to room temperature, and (4-((5-fluoro-2-methoxybenzamido)methyl)phenyl)boronic acid (0.31 g, 1.03 mmol) and Pd(dppf)Cl₂ (0.06 g, 0.08 mmol) were added. Under nitrogen protection, the mixture was heated to 95° C. and the reaction was allowed to proceed for another 12 h. The reaction solution was cooled to room temperature and filtered, and the filtrate was concentrated and purified by column chromatography (MeOH/DCM system) to afford 0.16 g of a yellow solid with a yield of 28%.

### Step 3: Synthesis of N-(4-(8-amino-3-(6-(piperazin-1-yl)pyridin-3-yl) imidazo[1,5-a]pyrazin-1-yl)benzyl)-5-fluoro-2-methoxybenzamide

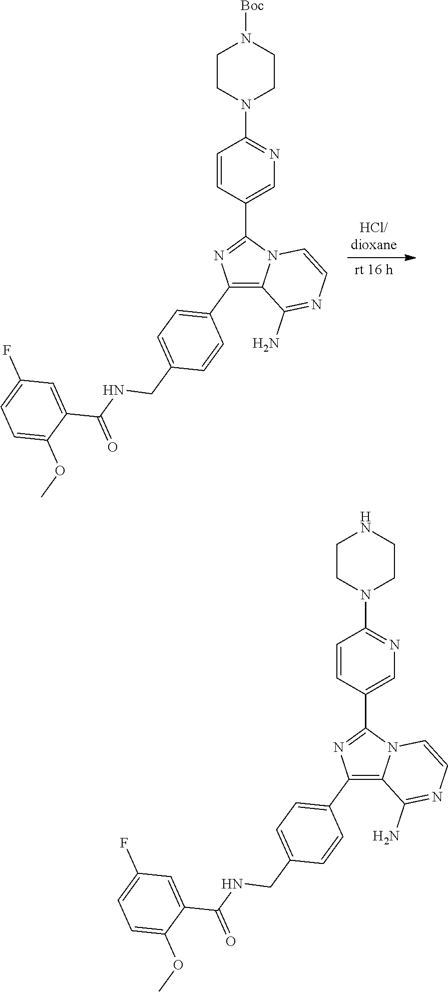 [SMILES: COc1ccc(F)cc1C(=O)NCc1ccc(-c2nc(-c3ccc(N4CCN(C(=O)OC(C)(C)C)CC4)nc3)n3ccnc(N)c23)cc1 ; COc1ccc(F)cc1C(=O)NCc1ccc(-c2nc(-c3ccc(N4CCNCC4)nc3)n3ccnc(N)c23)cc1]

At room temperature, tert-butyl 4-(5-(8-amino-1-(4-((5-fluoro-2-methoxybenzamido)methyl)phenyl)imidazo[1,5-a]pyrazin-3-yl)pyridin-2-yl) piperazine-1-carboxylate (0.16 g, 0.24 mmol), HCl-dioxane (5 mL) and DCM (5 mL) were added into a single-necked flask in sequence and the reaction was allowed to proceed for 16 h. The reaction solution was concentrated, and the concentrated reaction solution was treated with aqueous ammonia and concentrated to afford 0.10 g of a crude product as a yellow solid.

### Step 4: Synthesis of N-(4-(8-amino-3-(6-(4-(1-(4-(2,4-dioxotetrahydropyrimidin-1(2H)-yl)-3-methylphenyl)piperidin-4-yl)methyl)piperazin-1-yl)pyridin-3-yl) imidazo[1,5-a]pyrazin-1-yl]benzyl)-5-fluoro-2-methoxybenzamide

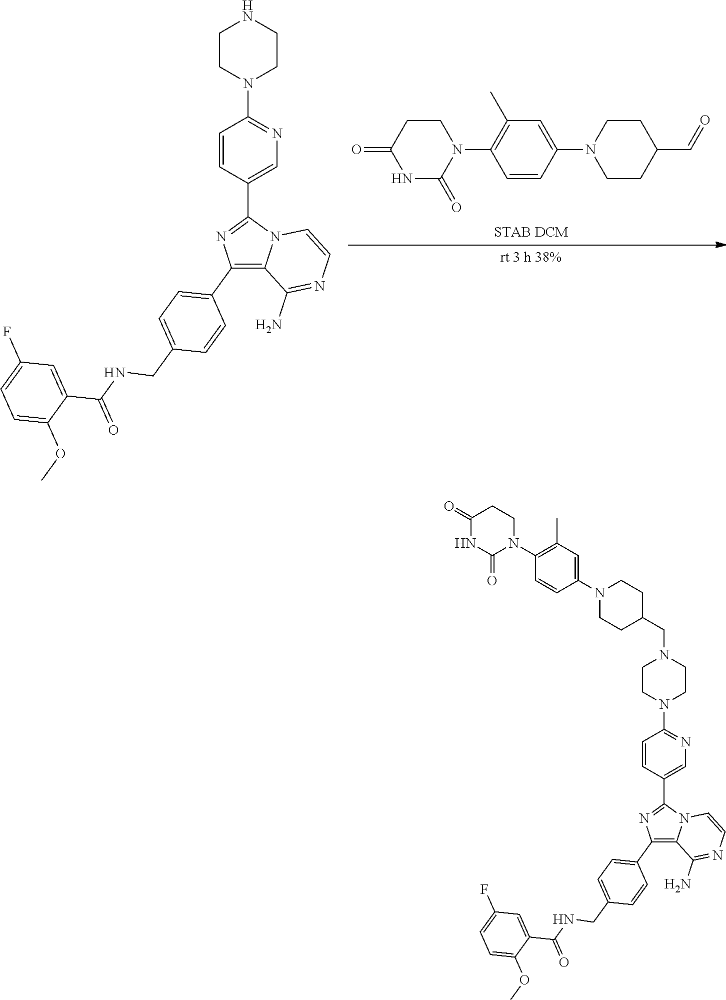 [SMILES: COc1ccc(F)cc1C(=O)NCc1ccc(-c2nc(-c3ccc(N4CCNCC4)nc3)n3ccnc(N)c23)cc1 ; Cc1cc(N2CCC(C=O)CC2)ccc1N1CCC(=O)NC1=O ; COc1ccc(F)cc1C(=O)NCc1ccc(-c2nc(-c3ccc(N4CCN(CC5CCN(c6ccc(N7CCC(=O)NC7=O)c(C)c6)CC5)CC4)nc3)n3ccnc(N)c23)cc1]

At room temperature, N-(4-(8-amino-3-(6-(piperazin-1-yl)pyridin-3-yl) imidazo[1,5-a]pyrazin-1-yl)benzyl)-5-fluoro-2-methoxybenzamide (0.10 g, 0.18 mmol), 1-(4-(2,4-dioxotetrahydropyrimidin-1(2H)-yl)-3-methylphenyl)piperidine-4-carbaldehyde (0.07 g, 0.22 mmol), sodium triacetoxyborohydride (0.06 g, 0.27 mmol) and DCM (20 mL) were added into a single-necked flask in sequence and the reaction was allowed to proceed at room temperature for 3 h. The reaction solution was concentrated and purified by column chromatography (MeOH/DCM system) to afford 0.06 g of a yellow solid with a yield of 39%, 1H NMR (400 MHz, DMSO-d₆) δ 10.25 (s, 1H), 8.88 (t, J=6.1 Hz, 1H), 8.57 (d, J=2.4 Hz, 1H), 7.96 (dd, J=8.9, 2.4 Hz, 1H), 7.67 (dd, J=17.7, 6.4 Hz, 3H), 7.58-7.46 (m, 3H), 7.40-7.30 (m, 1H), 7.20 (dd, J=9.1, 4.3 Hz, 1H), 7.10-6.95 (m, 3H), 6.84-6.75 (m, 2H), 6.09 (s, 2H), 4.60 (d, J=6.1 Hz, 2H), 3.91 (s, 3H), 3.75-3.57 (m, 6H), 3.52-3.42 (m, 1H), 2.87-2.56 (m, 4H), 2.50-2.45 (m, 3H), 2.23 (d, J=7.0 Hz, 2H), 2.12 (s, 3H), 1.88-1.66 (m, 4H), 1.31-1.13 (m, 2H). MS (ESI) m/z: 852.6 [M+H]⁺.

### Example 22: Synthesis of N-(4-(8-amino-3-(4-((1-(4-(2,6-dioxopyrimidin-3-yl)-3-methylphenyl)piperidin-4-yl)methyl)piperazin-1-yl)phenyl)imidazo[1,5-a]pyrazin-1-yl]benzyl)-5-fluoro-2-methoxybenzamide (Compound I-19)

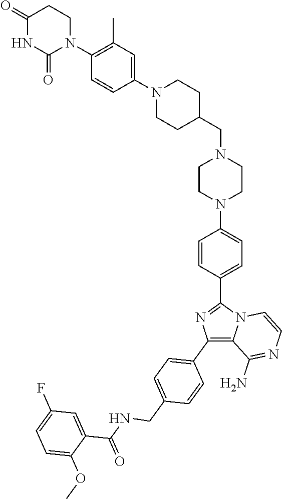 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1]

The synthesis method of N-(4-(8-amino-3-(4-(piperazin-1-yl)phenyl)imidazo[1,5-a]pyrazin-1-yl]benzyl)-5-fluoro-2-methoxybenzamide follows Example 20.

### Step 1: Synthesis of N-(4-(8-amino-3-(4-((1-(4-(2,6-dioxopyrimidin-3-yl)-3-methylphenyl)piperidin-4-yl)methyl)piperazin-1-yl)phenyl)imidazo[1,5-a]pyrazin-1-yl]benzyl)-5-fluoro-2-methoxybenzamide

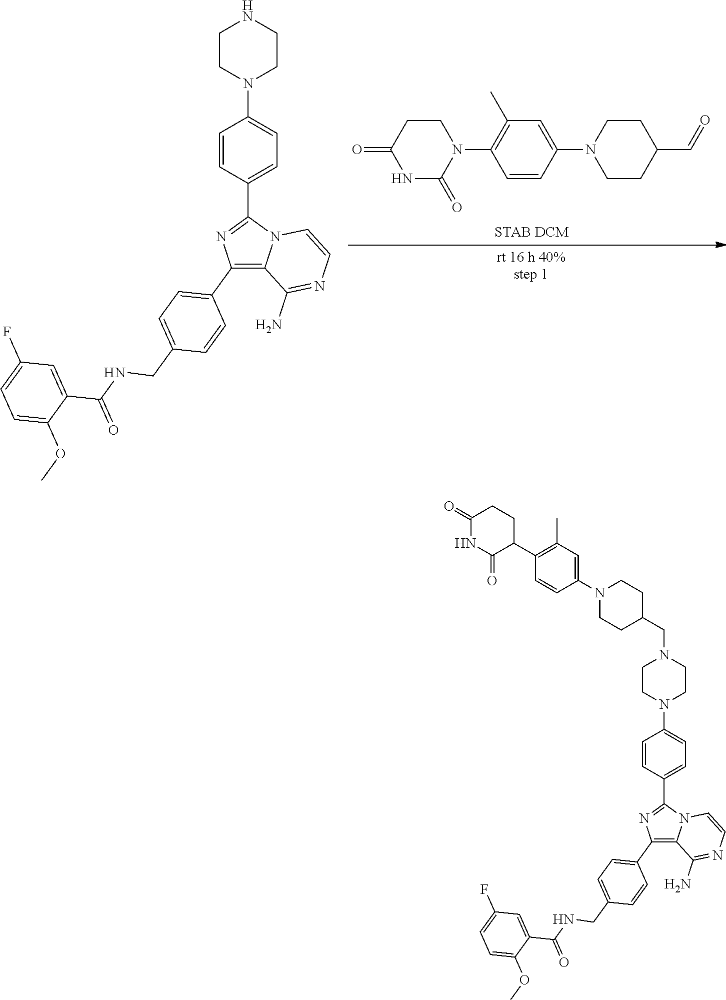 [SMILES: COc1ccc(F)cc1C(=O)NCc1ccc(-c2nc(-c3ccc(N4CCNCC4)cc3)n3ccnc(N)c23)cc1 ; Cc1cc(N2CCC(C=O)CC2)ccc1N1CCC(=O)NC1=O ; COc1ccc(F)cc1C(=O)NCc1ccc(-c2nc(-c3ccc(N4CCN(CC5CCN(c6ccc(C7CCC(=O)NC7=O)c(C)c6)CC5)CC4)cc3)n3ccnc(N)c23)cc1]

At room temperature, N-(4-(8-amino-3-(4-(piperazin-1-yl)phenyl)imidazo[1,5-a]pyrazin-1-yl]benzyl)-5-fluoro-2-methoxybenzamide (0.05 g, 0.09 mmol), 1-(4-(2,6-dioxopiperidin-3-yl)-3-methylphenyl)piperidine-4-carbaldehyde (0.03 g, 0.10 mmol), sodium triacetoxy borohydride (0.03 g, 0.13 mmol) and DCM (20 mL) were added into a single-necked flask in sequence and the reaction was allowed to proceed at room temperature for 16 h. The reaction solution was concentrated and purified by column chromatography (MeOH/DCM system) to afford 0.04 g of a yellow solid with a yield of 40%. MS (ESI) m/z: 850.6 [M+H]⁺.

### Example 23: Synthesis of (R)—N-(4-(4-amino-1-(6-(4-(1-(4-(2,4-dioxotetrahydropyrimidin-1(2H)-yl)-3-methylphenyl)piperidin-4-yl)methyl)-2-methylpiperazin-1-yl)pyridin-3-yl)-1H-pyrazolo[3,4-d]pyrimidin-3-yl)benzyl)-5-fluoro-2-methoxybenzamide (Compound I-41)

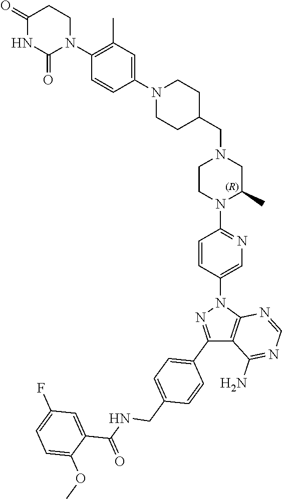

### Step 1: Synthesis of (R)-tert-butyl 4-(5-bromopyridin-2-yl)-3-methylpiperazine-1-carboxylate

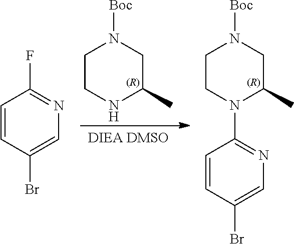

At room temperature, 5-bromo-2-fluoropyridine (5.00 g, 28.41 mmol), (R)-tert-butyl 3-methylpiperazine-1-carboxylate (6.26 g, 31.25 mmol), DIEA (7.34 g, 56.82 mmol) and DMSO (30 mL) were added into a single-necked flask in sequence. Under nitrogen protection, the mixture was heated to 120° C. and the reaction was allowed to proceed for 16 h before termination. The reaction solution was cooled to room temperature, diluted with water, and extracted with EA, and the organic phase was dried over anhydrous Na₂SO₄, filtered, concentrated and purified by column chromatography (EA/PE system) to afford 1.50 g of a yellow oil with a yield of 15%.

### Step 2: Synthesis of (R)-tert-butyl 4-(5-(4-amino-3-(4-((5-fluoro-2-methoxybenzamido)methyl)phenyl)-1H-pyrazolo[3,4-d]pyrimidin-1-yl)pyridin-2-yl)-3-methylpiperazine-1-carboxylate

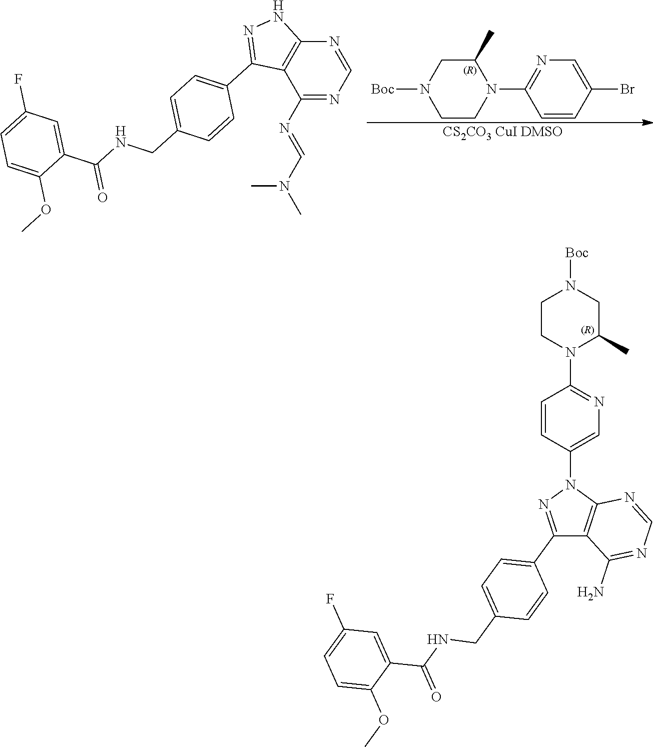

At room temperature, (R)-tort-butyl 4-(5-bromopyridin-2-yl)-3-methylpiperazine-1-carboxylate (0.60 g, 1.68 mmol), (E)-N-(4-(4-(((dimethylamino)methylene)amino)-1H-pyrazolo[3,4-d]pyrimidin-3-yl)benzyl)-5-fluoro-2-methoxybenzamide (0.50 g, 1.12 mmol), cesium carbonate (1.09 g, 3.35 mmol), trans-1,4-diaminocyclohexane (0.08 g, 0.56 mmol), CuI (0.01 g, 0.56 mmol), and DMSO (20 mL) were added into a single-necked flask in sequence. Under nitrogen protection, the mixture was heated to 135° C. and the reaction was allowed to proceed for 16 h before termination. The reaction solution was cooled to room temperature, diluted with water, and extracted with EA, and the organic phase was dried over anhydrous Na₂SO₄, filtered, concentrated and purified by column chromatography (EA/PE system) to afford 0.20 g of a yellow oil with a yield of 26%.

### Step 3: Synthesis of (R)—N-(4-(4-amino-1-(6-(2-methylpiperazin-1-yl)pyridin-3-yl)-1H-pyrazolo[3,4-d]pyrimidin-3-yl)benzyl)-5-fluoro-2-methoxybenzamide

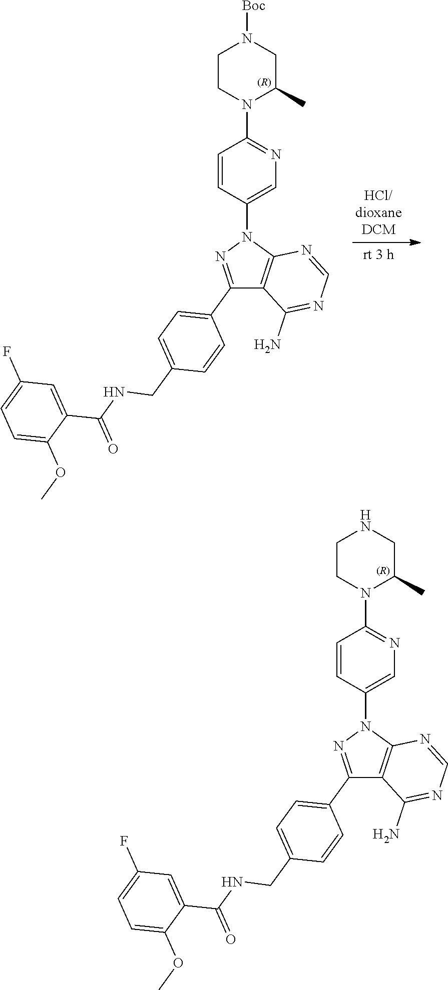

At room temperature, (R)-tert-butyl 4-(5-(4-amino-3-(4-((5-fluoro-2-methoxybenzamido)methyl)phenyl)-1H-pyrazolo[3,4-d]pyrimidin-1-yl)pyridin-2-yl)-3-methylpiperazine-1-carboxylate (0.20 g, 0.30 mmol), hydrochloride in dioxane (5 mL), and DCM (10 mL) were added into a single-necked flask in sequence and the reaction was allowed to proceed at room temperature for 3 h. The reaction solution was concentrated and treated with aqueous ammonia to afford 0.16 g of a crude product as a yellow solid.

### Step 4: Synthesis of (R)—N-(4-(4-amino-1-(6-(4-(1-(4-(2,4-dioxotetrahydropyrimidin-1(2H)-yl)-3-methylphenyl)piperidin-4-yl)methyl)-2-methylpiperazin-1-yl)pyridin-3-yl)-1H-pyrazolo[3,4-d]pyrimidin-3-yl)benzyl)-5-fluoro-2-methoxybenzamide

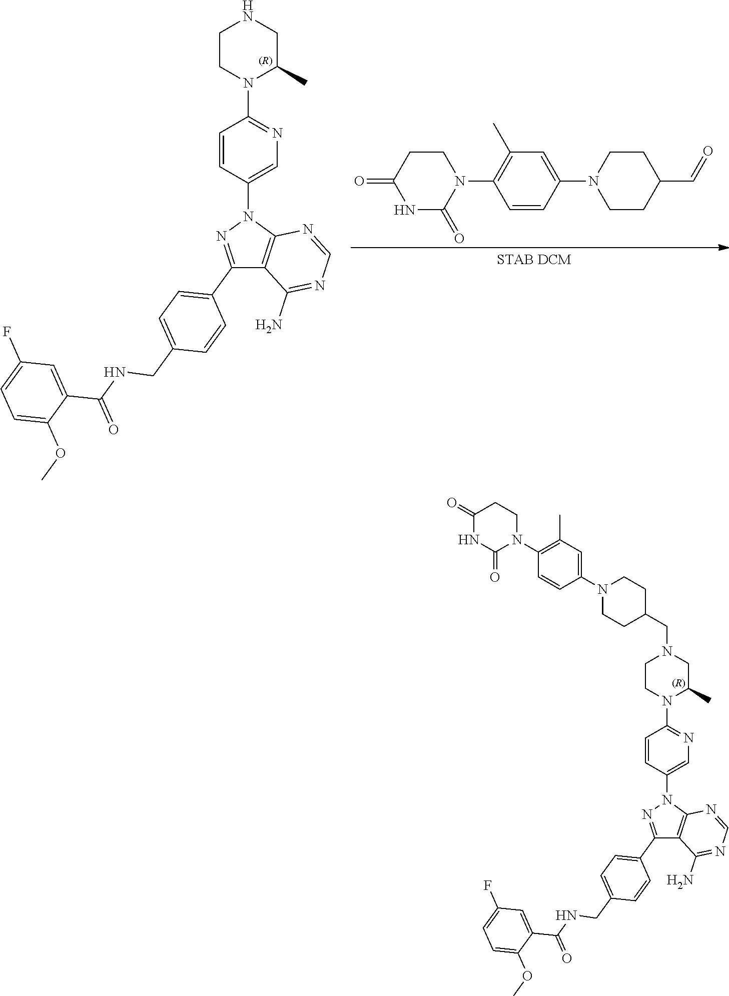

At room temperature, (R)—N-(4-(4-amino-1-(6-(2-methylpiperazin-1-yl)pyridin-3-yl)-1H-pyrazolo[3,4-d]pyrimidin-3-yl)benzyl)-5-fluoro-2-methoxybenzamide (0.08 g, 0.14 mmol), 1-(4-(2,4-dioxotetrahydropyrimidin-1(2H)-yl)-3-methylphenyl)piperidine-4-carbaldehyde (0.05 g, 0.17 mmol), sodium triacetoxyborohydride (0.04 g, 0.21 mmol) and DCM (20 mL) were added into a single-necked flask in sequence and the reaction was allowed to proceed at room temperature for 1 h. The reaction solution was concentrated and subjected to column chromatography (MeOH/DCM system) to afford 0.004 g of a white solid with a yield of 3%. MS (ESI) m/z: 867.8 [M+H]⁺.

### Example 24: Synthesis of N-(4-(8-amino-3-(4-(4-(1-(4-(2,6-dioxopiperidin-3-yl)phenyl) piperidin-4-yl)methyl)piperazin-1-yl)phenyl)imidazo[1,5-a]pyrazin-1-yl]benzyl)-5-fluoro-2-methoxybenzamide (Compound I-18)

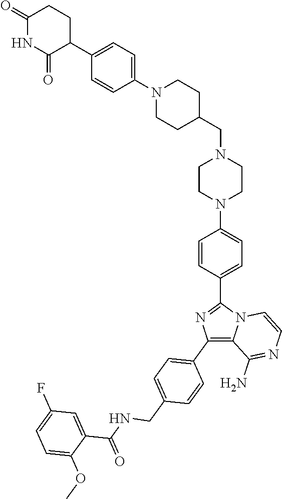 [SMILES: COc1ccc(F)cc1C(=O)NCc1ccc(-c2nc(-c3ccc(N4CCN(CC5CCN(c6ccc(C7CCC(=O)NC7=O)cc6)CC5)CC4)cc3)n3ccnc(N)c23)cc1]

The synthesis method of N-(4-(8-amino-3-(4-(piperazin-1-yl)phenyl)imidazo[1,5-a]pyrazin-1-yl]benzyl)-5-fluoro-2-methoxybenzamide follows Example 20.

### Step 1: Synthesis of N-(4-(8-amino-3-(4-(4-(1-(4-(2,6-dioxopiperidin-3-yl)phenyl) piperidin-4-yl)methyl)piperazin-1-yl)phenyl)imidazo[1,5-a]pyrazin-1-yl]benzyl)-5-fluoro-2-methoxybenzamide

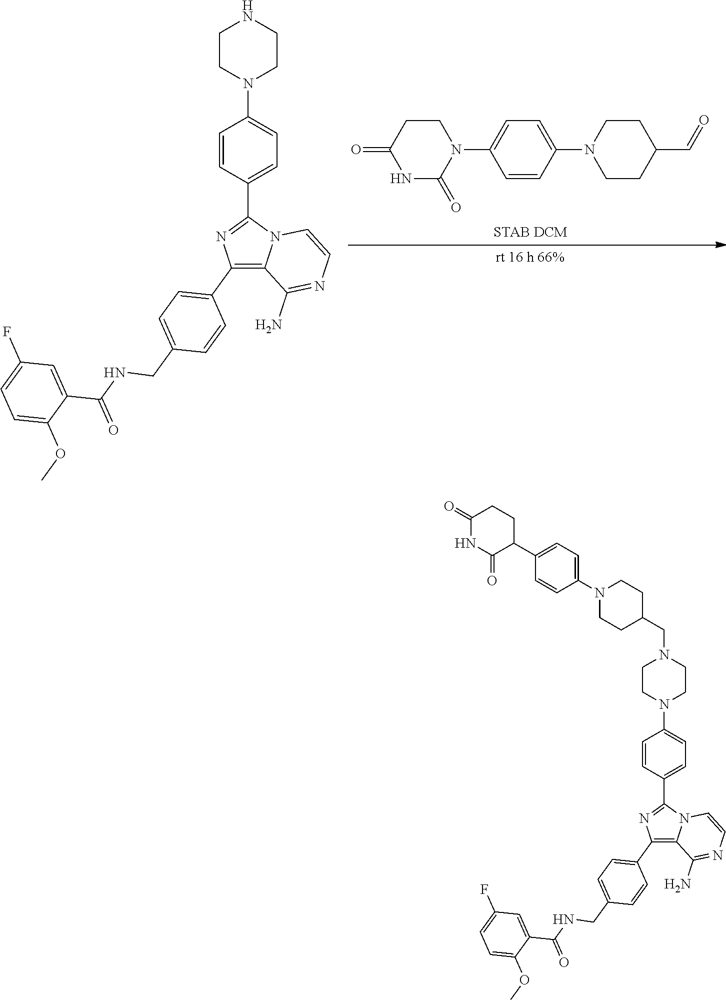 [SMILES: COc1ccc(F)cc1C(=O)NCc1ccc(-c2nc(-c3ccc(N4CCNCC4)cc3)n3ccnc(N)c23)cc1 ; O=CC1CCN(c2ccc(N3CCC(=O)NC3=O)cc2)CC1 ; COc1ccc(F)cc1C(=O)NCc1ccc(-c2nc(-c3ccc(N4CCN(CC5CCN(c6ccc(C7CCC(=O)NC7=O)cc6)CC5)CC4)cc3)n3ccnc(N)c23)cc1]

At room temperature, N-(4-(8-amino-3-(4-(piperazin-1-yl)phenyl)imidazo[1,5-a]pyrazin-1-yl]benzyl)-5-fluoro-2-methoxybenzamide (0.05 g, 0.09 mmol), 1-(4-(2,6-dioxopiperidin-3-yl)phenyl)piperidine-4-carbaldehyde (0.03 g, 0.10 mmol), sodium triacetoxyborohydride (0.03 g, 0.13 mmol) and DCM (20 mL) were added into a single-necked flask in sequence and the reaction was allowed to proceed at room temperature for 16 h. The reaction solution was concentrated and purified by column chromatography (MeOH/DCM system) to afford 0.05 g of a yellow solid with a yield of 66%, ¹H NMR (400 MHz, DMSO-d₆) δ 10.77 (s, 1H), 8.96-8.82 (m, 1H), 7.67 (dd, J=17.8, 7.3 Hz, 4H), 7.59-7.46 (m, 3H), 7.37-7.31 (m, 1H), 7.24-7.16 (m, 1H), 7.14-7.01 (m, 4H), 6.89 (d, J=8.4 Hz, 2H), 6.08 (s, 2H), 4.60 (d, J=6.1 Hz, 2H), 3.91 (s, 3H), 3.77-3.64 (m, 3H), 3.31-3.14 (m, 6H), 2.75-2.54 (m, 6H), 2.25 (s, 2H), 2.19-2.06 (m, 1H), 2.06-1.96 (m, 1H), 1.88-1.63 (m, 3H), 1.33-1.15 (m, 2H). MS (ESI) m/z: 836.6 [M+H]⁺.

### Example 25: Synthesis of N-(4-(4-amino-1-(4-(-4-(4-(2,4-dioxotetrahydropyrimidin-1(2H)-yl)-3-methylphenyl) piperazin-1-yl)methyl)piperidin-1-yl)phenyl)-1H-pyrazolo[3,4-d]pyrimidin-3-yl)benzyl)-5-fluoro-2-methoxybenzamide (Compound I-42)

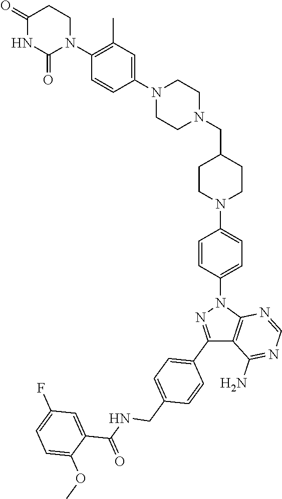 [SMILES: COc1ccc(F)cc1C(=O)NCc1ccc(-c2nn(-c3ccc(N4CCC(CN5CCN(c6ccc(N7CCC(=O)NC7=O)c(C)c6)CC5)CC4)cc3)c3ncnc(N)c23)cc1]

The synthesis method of N-(4-(4-amino-1-(4-(-4-formylpiperidin-1-yl)phenyl)-1H-pyrazolo[3,4-d]pyrimidin-3-yl)benzyl)-5-fluoro-2-methoxybenzamide follows Example 1.

### Step 1: Synthesis of N-(4-(4-amino-1-(4-(-4-(4-(2,4-dioxotetrahydropyrimidin-1(2H)-yl)-3-methylphenyl) piperazin-1-yl)methyl)piperidin-1-yl)phenyl)-1H-pyrazolo[3,4-d]pyrimidin-3-yl)benzyl)-5-fluoro-2-methoxybenzamide

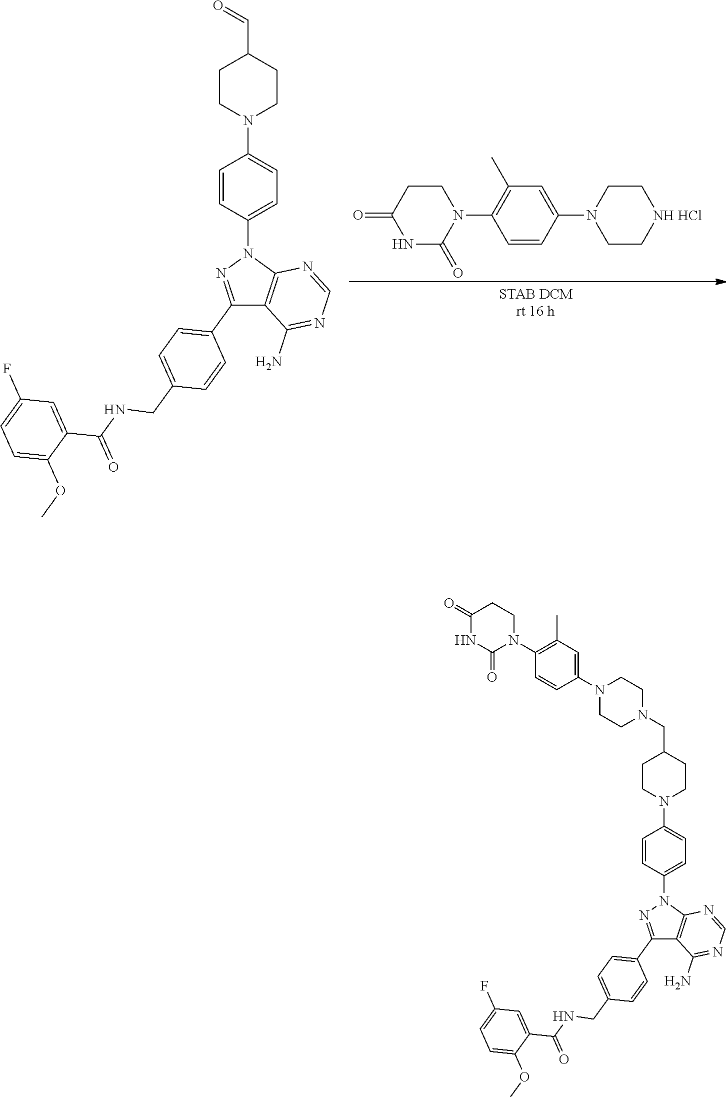

At room temperature, N-(4-(4-amino-1-(4-(-4-formylpiperidin-1-yl)phenyl)-1H-pyrazolo[3,4-d]pyrimidin-3-yl)benzyl)-5-fluoro-2-methoxybenzamide (0.10 g, 0.17 mmol), 1-(2-methyl-4-(piperazin-1-yl)phenyl)dihydropyrimidine-2,4(1H,3H)-dione hydrochloride (0.06 g, 0.21 mmol), sodium triacetoxyborohydride (0.06 g, 0.27 mmol) and DCM (20 mL) were added into a single-necked flask in sequence and the reaction was allowed to proceed at room temperature for 16 h. The reaction solution was concentrated and purified by column chromatography (MeOH/DCM system) to afford 0.002 g of a yellow solid with a yield of 13%. MS (ESI) m/z: 852.8 [M+H]⁺.

### Example 26: Synthesis of N-(4-(8-amino-3-(6-(4-(1-(4-(2,4-dioxotetrahydropyrimidin-1(2H)-yl)-3-methylphenyl)piperidin-4-yl)methyl)piperazin-1-yl)pyridin-3-yl) imidazo[1,5-a]pyrazin-1-yl]benzyl)-5-fluoro-2-methoxybenzamide (Compound I-43)

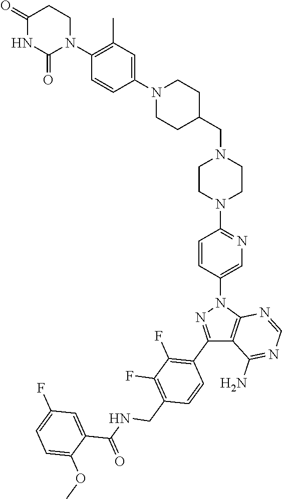 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)c(F)c1F]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

### Step 1: Synthesis of N-(4-(4-amino-1-(6-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-d]pyrimidin-3-yl)-2,3-difluorobenzyl)-5-fluoro-2-methoxybenzamide

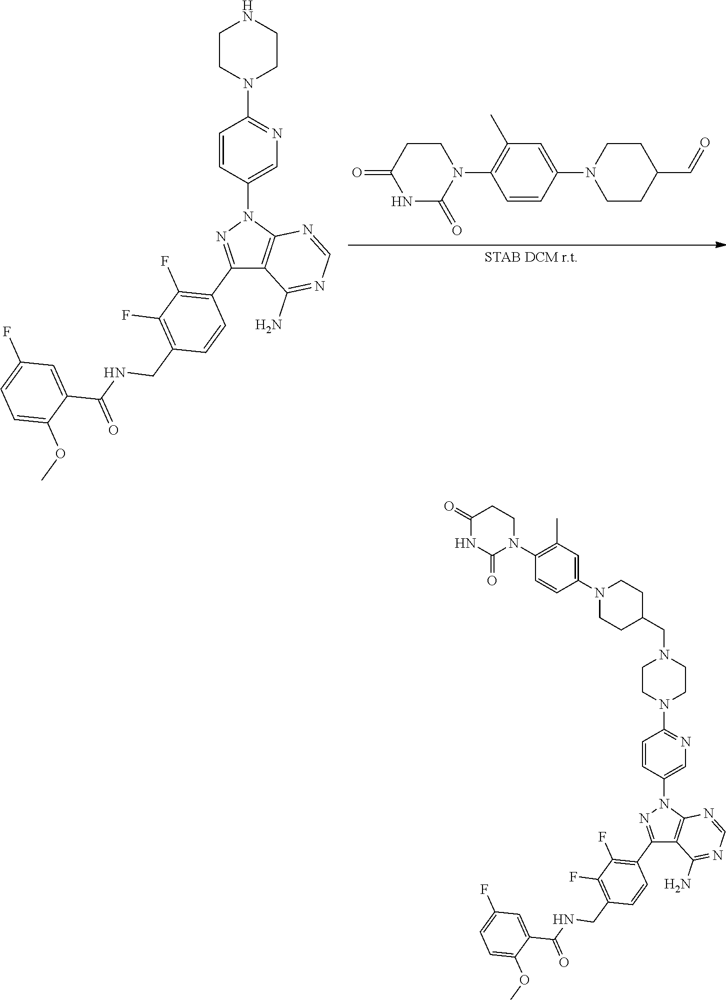 [SMILES: COc1ccc(F)cc1C(=O)NCc1ccc(-c2nn(-c3ccc(N4CCNCC4)nc3)c3ncnc(N)c23)c(F)c1F ; Cc1cc(N2CCC(C=O)CC2)ccc1N1CCC(=O)NC1=O ; COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)c(F)c1F]

At room temperature, 1-(4-(2,4-dioxotetrahydropyrimidin-1(2H)-yl)-3-methylphenyl)piperidine-4-carbaldehyde (0.05 g, 0.15 mmol), N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide (0.06 g, 0.10 mmol), STAB (0.03 g, 0.15 mmol) and dichloromethane (10 mL) were added into a single-necked flask in sequence. The reaction was allowed to proceed at room temperature for 2 h under nitrogen protection before termination. The reaction solution was concentrated and purified by column chromatography (MeOH/DCM system) to afford 0.05 g of a white solid with a yield of 55%. ¹H NMR (400 MHz, DMSO-d₆) δ 8.94 (t, J=6.0 Hz, 1H), 8.74 (d, J=2.7 Hz, 1H), 8.31 (s, 1H), 8.14 (m, 1H), 7.54 (m, 1H), 7.50-7.43 (m, 1H), 7.42-7.31 (m, 2H), 7.21 (m, 1H), 7.03 (m, 2H), 6.87-6.72 (m, 2H), 4.66 (d, J=6.0 Hz, 2H), 3.92 (s, 3H), 3.68 (m, 3H), 3.56 (m, 4H), 3.47 (m, 3H), 2.84-2.61 (m, 5H), 2.22 (m, 2H), 2.12 (s, 3H), 1.88-1.64 (m, 4H), 1.32-1.09 (m, 2H). MS (ESI) m/z: 889.7 [M+H]⁺.

### Example 27: Synthesis of N-(4-(4-amino-1-(4-(1-((1-(4-(2,4-dioxotetrahydropyrimidin-1(2H)-yl)-3-methylphenyl)piperidin-4-yl)methyl)piperidinyl)phenyl)-1H-pyrazolo[3,4-d]pyrimidin-3-yl)-2-fluorobenzyl)-5-fluoro-2-methoxybenzamide (Compound I-44)

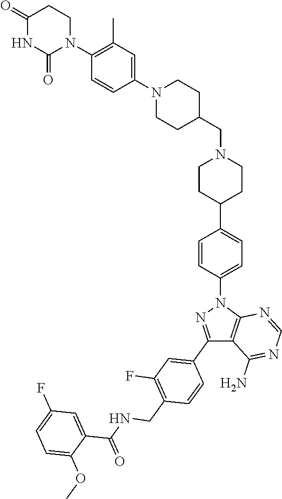 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1F]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

Step 1: Synthesis of N-(4-(4-amino-1-(4-(1-((1-(4-(2,4-dioxotetrahydropyrimidin-1(2H)-yl)-3-methylphenyl)piperidin-4-yl)methyl)piperidinyl)phenyl)-1H-pyrazolo[3,4-d]pyrimidin-3-yl)-2-fluorobenzyl)-5-fluoro-2-methoxybenzamide

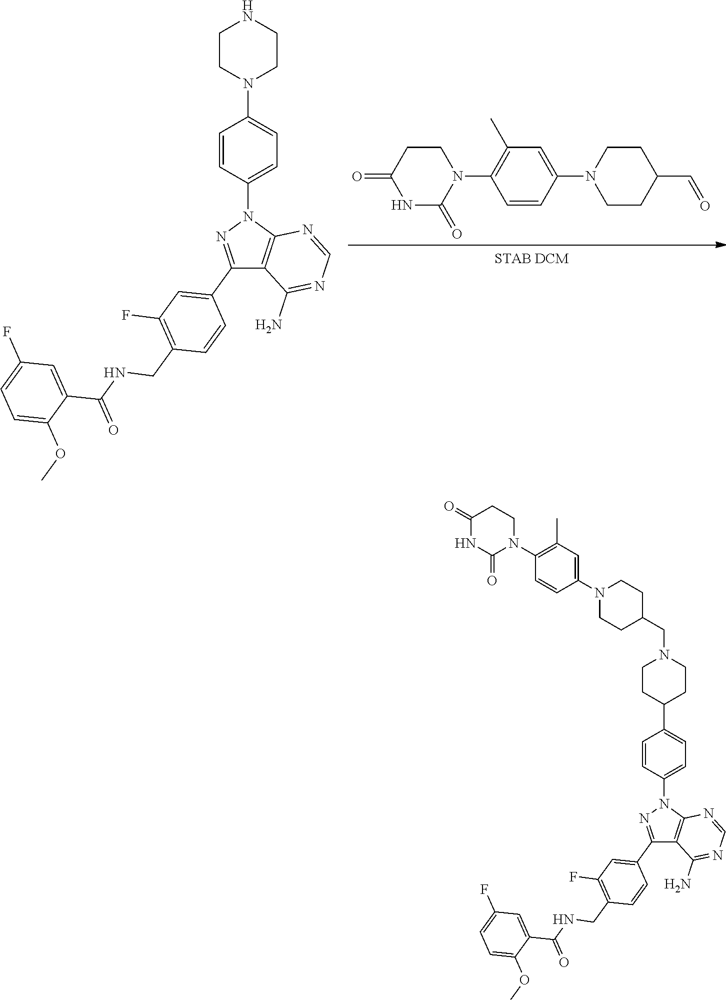 [SMILES: COc1ccc(F)cc1C(=O)NCc1ccc(-c2nn(-c3ccc(N4CCNCC4)cc3)c3ncnc(N)c23)cc1F ; Cc1cc(N2CCC(C=O)CC2)ccc1N1CCC(=O)NC1=O ; COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1F]

At room temperature, N-(4-(4-amino-1-(4-(piperidin-4-yl)phenyl)-1H-pyrazolo[3,4-d]pyrimidin-3-yl)-2-fluorobenzyl)-5-fluoro-2-methoxybenzamide (0.08 g, 0.14 mmol), 1-(4-(2,4-dioxotetrahydropyrimidin-1(2H)-yl)-3-methylphenyl)piperidine-4-carbaldehyde (0.05 g, 0.17 mmol), sodium triacetoxyborohydride (0.04 g, 0.21 mmol) and DCM (20 mL) were added into a single-necked flask in sequence and the reaction was allowed to proceed at room temperature for 1 h. The reaction solution was concentrated and subjected to column chromatography (MeOH/DCM system) to afford 0.04 g of a white solid with a yield of 30%, ¹H NMR (400 MHz, DMSO-d₆) δ 10.26 (s, 1H), 8.90 (t, J=6.1 Hz, 1H), 8.37 (s, 1H), 8.11 (d, J=8.3 Hz, 2H), 7.65-7.52 (m, 4H), 7.45 (d, J=8.4 Hz, 2H), 7.40-7.33 (m, 1H), 7.21 (dd, J=9.2, 4.3 Hz, 1H), 7.04 (d, J=8.5 Hz, 1H), 6.84-6.75 (m, 2H), 4.64 (d, J=6.1 Hz, 2H), 3.93 (s, 3H), 3.76-3.61 (m, 3H), 3.01 (d, J=10.4 Hz, 2H), 2.76-2.54 (m, 5H), 2.30-2.18 (m, 2H), 2.12 (s, 3H), 2.08-1.97 (m, 2H), 1.86-1.66 (m, 8H), 1.27-1.16 (m, 2H). MS (ESI) m/z: 869.8 [M+H]⁺.

### Example 28: Synthesis of N-(4-(4-amino-1-(4-(1-((1-(4-(2,4-dioxotetrahydropyrimidin-1(2H)-yl)-3-methylphenyl)piperidin-4-yl)methyl)piperidinyl)phenyl)-1H-pyrazolo[3,4-d]pyrimidin-3-yl)-2-fluorobenzyl)-5-fluoro-2-methoxybenzamide (Compound I-12)

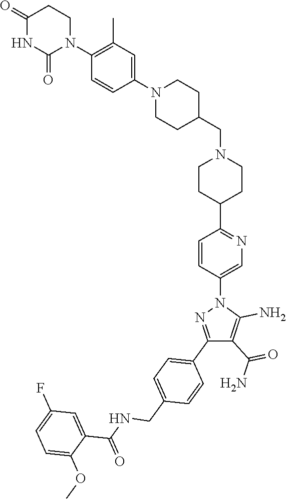 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c(N)c2C(N)=O)cc1]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

### Step 1: Synthesis of N-(4-(5-amino-4-cyano-1H-pyrazol-3-yl)benzyl)-5-fluoro-2-methoxybenzamide

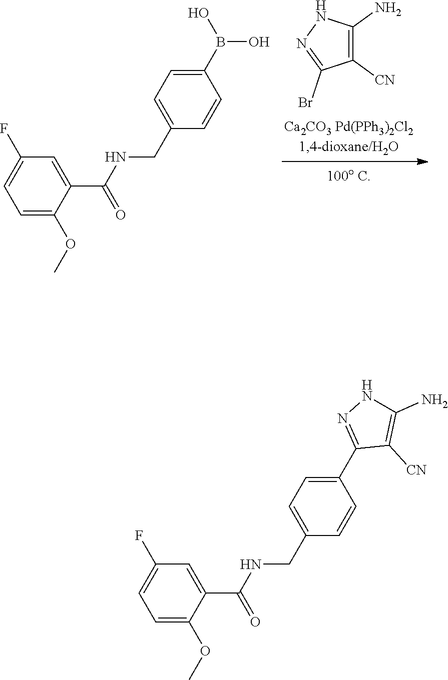 [SMILES: COc1ccc(F)cc1C(=O)NCc1ccc(B(O)O)cc1 ; N#Cc1c(Br)n[nH]c1N ; COc1ccc(F)cc1C(=O)NCc1ccc(-c2n[nH]c(N)c2C#N)cc1]

At room temperature, 5-amino-3-bromopyrazole-4-carbonitrile (1.0 g, 5.38 mmol), 4-((5-fluoro-2-methoxybenzamide)methyl)phenylboronic acid (2.44 g, 8.03 mmol), cesium carbonate (5.26 g, 16.14 mmol), bis(triphenylphosphine) palladium dichloride (0.75 g, 1.08 mmol), 1,4-dioxane (20 mL) and water (1 mL) were added into a single-necked flask in sequence. The reaction was allowed to proceed at 100° C. for 20 h under nitrogen protection before termination. The reaction solution was dried over anhydrous Na₂SO₄, filtered, concentrated and purified by column chromatography (MeOH/DCM system), to afford 0.50 g of a white solid with a yield of 25%.

### Step 2: Synthesis of tert-butyl 4-(5-amino-4-cyano-3-(4-(5-fluoro-2-methoxybenzamido)methyl)phenyl-1H-pyrazol-1-yl)pyridin-2-yl) piperazine-1-carboxylate

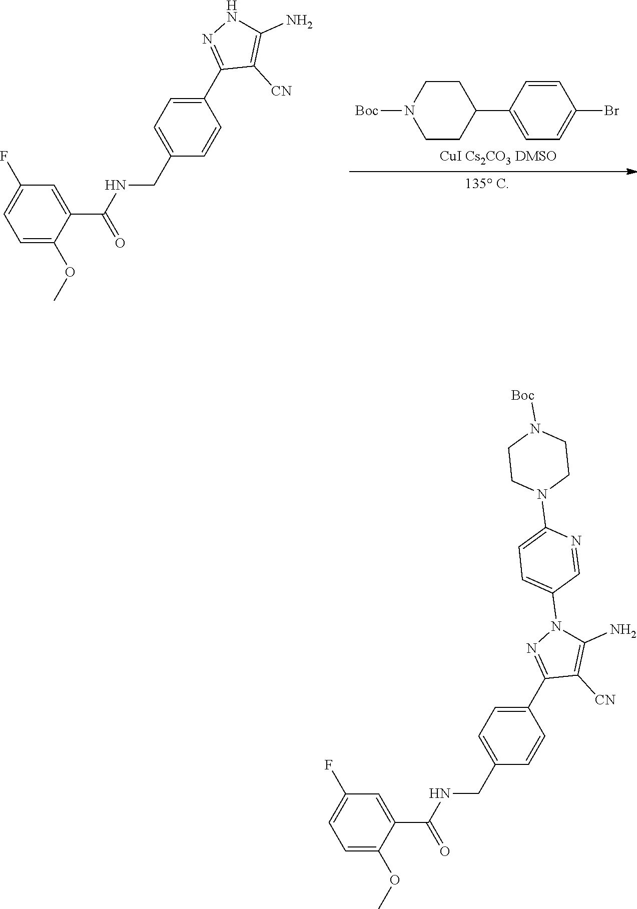 [SMILES: COc1ccc(F)cc1C(=O)NCc1ccc(-c2n[nH]c(N)c2C#N)cc1 ; CC(C)(C)OC(=O)N1CCC(c2ccc(Br)cc2)CC1 ; COc1ccc(F)cc1C(=O)NCc1ccc(-c2nn(-c3ccc(N4CCN(C(=O)OC(C)(C)C)CC4)nc3)c(N)c2C#N)cc1]

At room temperature, N-(4-(5-amino-4-cyano-1H-pyrazol-3-yl)benzyl)-5-fluoro-2-methoxybenzamide (0.25 g, 0.68 mmol), (4-(4-(tert-butoxycarbonyl) piperazin-1-yl)phenylboronic acid (0.35 g, 1.03 mmol), trans-N,N′-dimethyl-1,2-cyclohexanediamine (0.05 g, 0.34 mmol), cesium carbonate (0.67 g, 2.05 mmol), cuprous iodide (0.07 g, 0.34 mmol) and DMSO (20 mL) were added into a single-necked flask in sequence. The reaction was allowed to proceed at 135° C. for 6 h under nitrogen protection before termination. The reaction solution was diluted with water and extracted with EA, and the organic phase was dried over anhydrous Na₂SO₄, filtered, concentrated and purified by column chromatography (EA/PE system) to afford 0.05 g of a black oil with a yield of 12%.

### Step 3: Synthesis of 5-amino-3-(4-(5-fluoro-2-methoxybenzamidomethyl)phenyl)-1-(6-(piperazin-1-yl)pyridin-3-yl)-1H-pyrazole-4-carboxamide

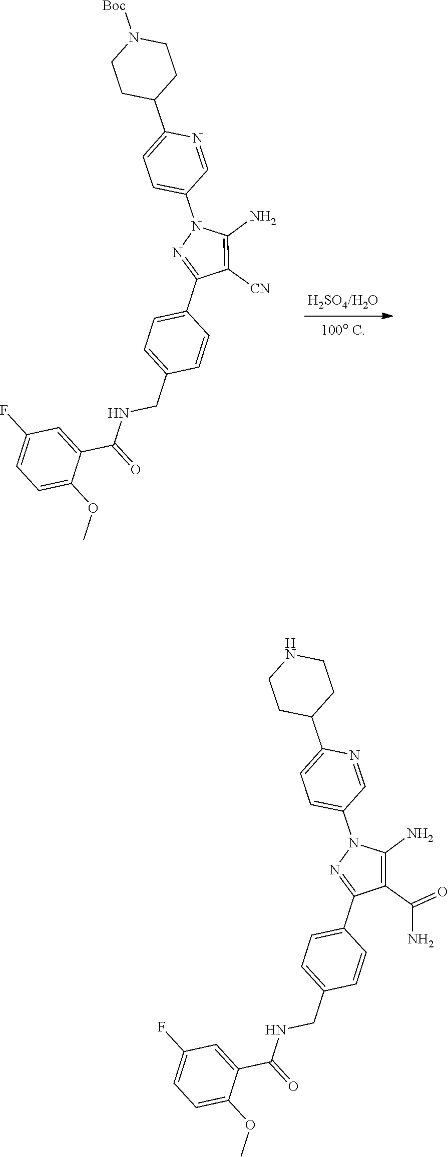 [SMILES: COc1ccc(F)cc1C(=O)NCc1ccc(-c2nn(-c3ccc(C4CCN(C(=O)OC(C)(C)C)CC4)nc3)c(N)c2C#N)cc1 ; COc1ccc(F)cc1C(=O)NCc1ccc(-c2nn(-c3ccc(C4CCNCC4)nc3)c(N)c2C(N)=O)cc1]

Under ice-water bath conditions, tert-butyl 4-(5-amino-4-cyano-3-(4-(5-fluoro-2-methoxybenzamido)methyl)phenyl-1H-pyrazol-1-yl)pyridin-2-yl) piperazine-1-carboxylate (0.05 g, 0.10 mmol) and a sulfuric acid solution (10 mL) were added into a single-necked flask in sequence. The reaction was allowed to proceed at 100° C. for 1 h under nitrogen protection before termination. The reaction solution was cooled to room temperature and diluted with ice water, and the pH was adjusted to alkaline with a saturated sodium carbonate solution. The alkaline solution was extracted with EA, and the organic phase was dried over anhydrous Na₂SO₄ and filtered, and the filtrate was concentrated to afford 0.03 g of a crude product as a brown solid.

### Step Synthesis of 5-amino-1-(6-(4-(2,4-dioxotetrahydropyrimidin-1(2H)-yl)-3-methylphenyl)piperidin-4-yl)methyl)piperazin-1-yl)pyridin-3-yl)-3-(4-((5-fluoro-2-methoxybenzamidomethyl)phenyl)-1H-pyrazole-4-carboxamide

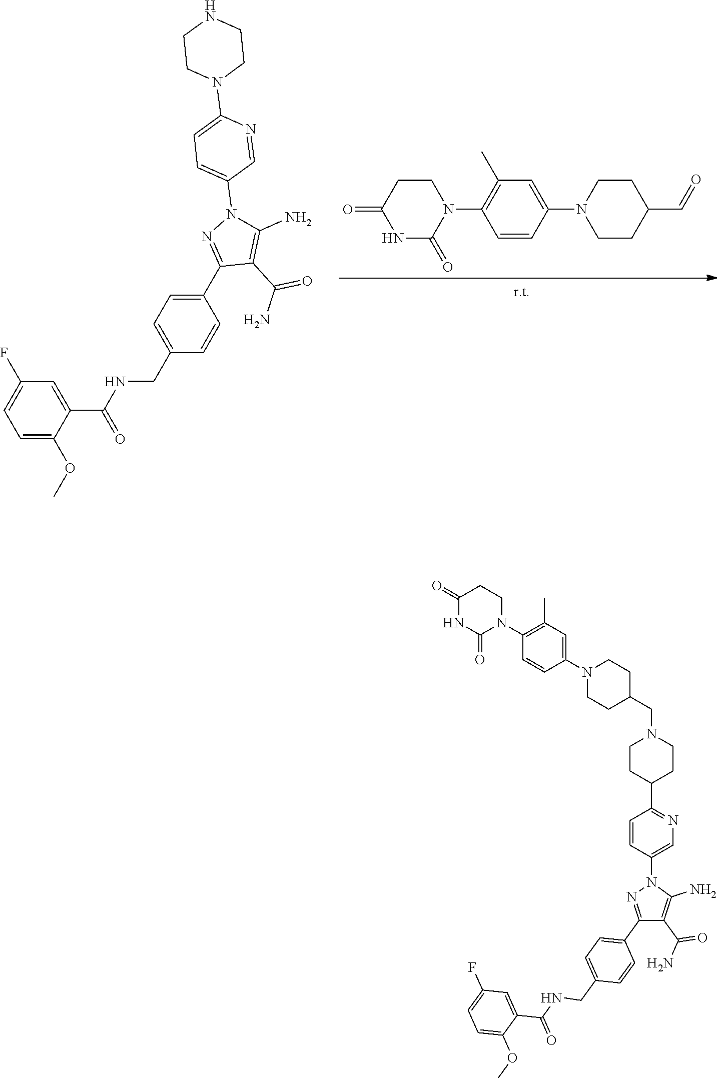 [SMILES: COc1ccc(F)cc1C(=O)NCc1ccc(-c2nn(-c3ccc(N4CCNCC4)nc3)c(N)c2C(N)=O)cc1 ; Cc1cc(N2CCC(C=O)CC2)ccc1N1CCC(=O)NC1=O ; COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c(N)c2C(N)=O)cc1]

At room temperature, 5-amino-3-(4-(5-fluoro-2-methoxybenzamidomethyl)phenyl)-1-(6-(piperazin-1-yl)pyridin-3-yl)-1H-pyrazole-4-carboxamide (0.03 g, 0.05 mmol), 1-(4-(2,4-dioxotetrahydropyrimidin-1(2H)-yl)-3-methylphenyl)piperidine-4-carboxaldehyde (0.02 g, 0.06 mmol), sodium triacetoxyborohydride (0.02 g, 0.07 mmol) and DCM (10 mL) were added into a single-necked flask in sequence. The reaction was allowed to proceed at room temperature for 2 h under nitrogen protection before termination. The reaction solution was purified on a thick preparative plate (MeOH/DCM system) to afford 0.02 g of a white solid with a yield of 55%, 1H NMR (400 MHz, DMSO-d₆) δ 8.87 (t, J=6.1 Hz, 1H), 8.76 (s, 1H), 7.95 (d, J=8.3 Hz, 1H), 7.63-7.42 (m, 6H), 7.35 (m, 1H), 7.19 (m, 1H), 7.05 (d, J=8.5 Hz, 1H), 6.86-6.73 (m, 2H), 6.59 (s, 2H), 4.57 (d, J=6.1 Hz, 2H), 3.90 (s, 3H), 3.68 (m, 3H), 3.47 (m, 2H), 3.01 (m, 2H), 2.70 (m, 5H), 2.12 (s, 3H), 1.95-1.76 (m, 6H), 1.22 (m, 5H). MS (ESI) m/z: 843.66 [M+H]⁺.

### Example 29: Synthesis of 5-amino-1-(4-(2,4-dioxotetrahydropyrimidin-1(2H)-yl)-3-methylphenyl)piperidin-4-yl)methyl)piperazin-1-yl)phenyl)-3-(4-((5-fluoro-2-methoxybenzamidomethyl)phenyl)-1H-pyrazole-4-carboxamide (Compound I-8)

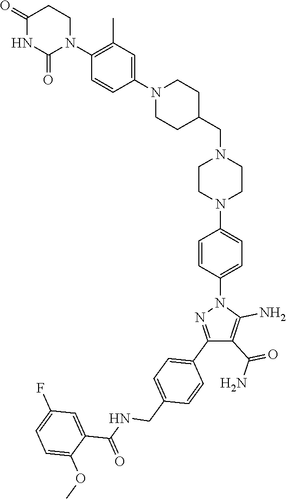 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c(N)c2C(N)=O)cc1]

The synthesis method of 5-amino-3-(4-(5-fluoro-2-methoxybenzamidomethyl)phenyl)-1-(4-(piperazin-1-yl)phenyl)-1H-pyrazole-4-carboxamide follows Example 28.

### Step 1: Synthesis of 5-amino-1-(4-(2,4-dioxotetrahydropyrimidin-1(2H)-yl)-3-methylphenyl)piperidin-4-yl)methyl)piperazin-1-yl)phenyl)-3-(4-((5-fluoro-2-methoxybenzamidomethyl)phenyl)-1H-pyrazole-4-carboxamide

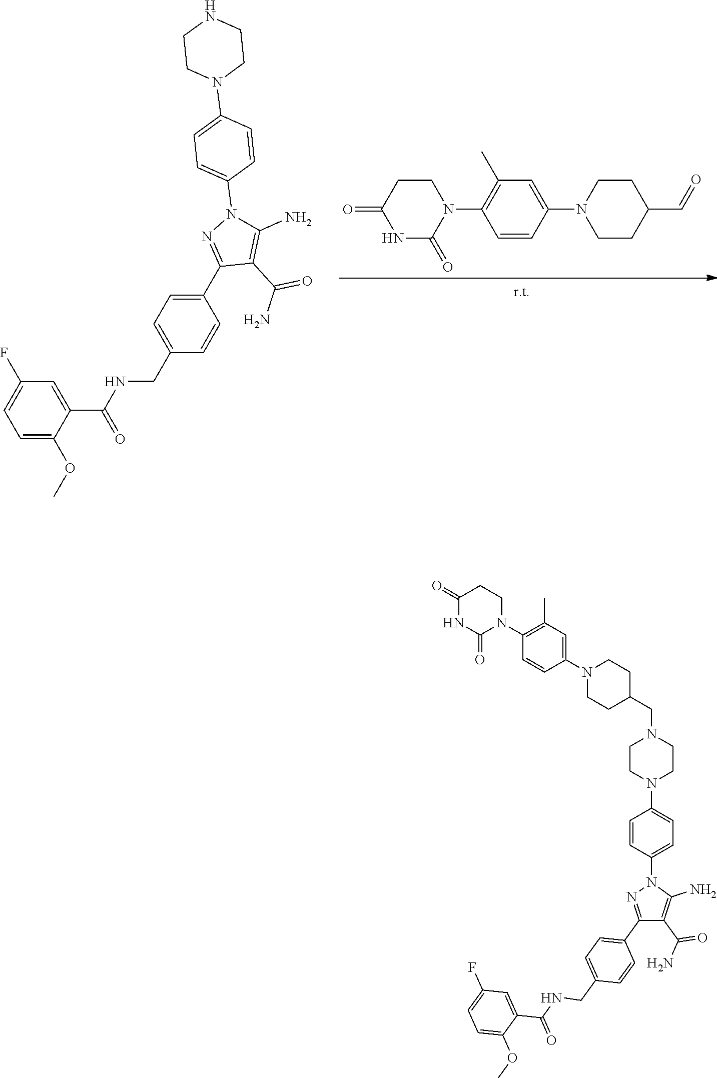 [SMILES: COc1ccc(F)cc1C(=O)NCc1ccc(-c2nn(-c3ccc(N4CCNCC4)cc3)c(N)c2C(N)=O)cc1 ; Cc1cc(N2CCC(C=O)CC2)ccc1N1CCC(=O)NC1=O ; COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c(N)c2C(N)=O)cc1]

At room temperature, 5-amino-3-(4-(5-fluoro-2-methoxybenzamidomethyl)phenyl)-1-(4-(piperazin-1-yl)phenyl)-1H-pyrazole-4-carboxamide (0.10 g, 0.18 mmol), 1-(4-(2,4-dioxotetrahydropyrimidin-1(2H)-yl)-3-methylphenyl)piperidine-4-carboxaldehyde (0.08 g, 0.24 mmol), sodium triacetoxyborohydride (0.06 g, 0.27 mmol) and DCM (10 mL) were added into a single-necked flask in sequence. The reaction was allowed to proceed at room temperature for 2 h under nitrogen protection before termination. The reaction solution was concentrated and purified by column chromatography (MeOH/DCM system) to afford 0.03 g of a white solid with a yield of 20%, 3H NMR (400 MHz, DMSO-d₆) δ 8.86 (s, 1H), 7.77-7.49 (m, 3H), 7.48-7.31 (m, 5H), 7.19 (m, 1H), 7.06 (m, 3H), 6.86-6.74 (m, 2H), 6.29 (s, 2H), 4.57 (d, J=6.1 Hz, 2H), 3.90 (s, 3H), 3.68 (m, 3H), 3.47 (m, 1H), 3.21 (m, 4H), 2.70 (m, 4H), 2.23 (m, 2H), 2.12 (s, 3H), 1.91-1.65 (m, 3H), 1.33-1.12 (m, 3H). MS (ESI) m/z: 843.50 [M+H]⁺.

### Example 30: Synthesis of 5-amino-1-(4-(1-(1-(4-(2,4-dioxotetrahydropyrimidin-1(2H)-yl)-3-methylphenyl)piperidin-4-yl)methyl)piperidin-4-yl)phenyl)-3-(4-(((5-fluoro-2-methoxybenzamido)methyl)phenyl)-1H-pyrazole-4-carboxamide (Compound I-11)

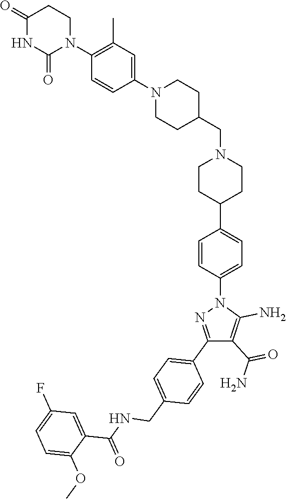 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c(N)c2C(N)=O)cc1]

The synthesis method of 5-amino-3-(4-((5-fluoro-2-methoxy benzamido)methyl)phenyl)-1-(4-(piperidin-4-yl)phenyl)-1H-pyrazole-4-carboxamide follows Example 28.

### Step 1: Synthesis of 5-amino-1-(4-(1-(1-(4-(2,4-dioxotetrahydropyrimidin-1(2H)-yl)-3-methylphenyl)piperidin-4-yl)methyl)piperidin-4-yl)phenyl)-3-(4-(((5-fluoro-2-methoxybenzamido)methyl)phenyl)-1H-pyrazole-4-carboxamide

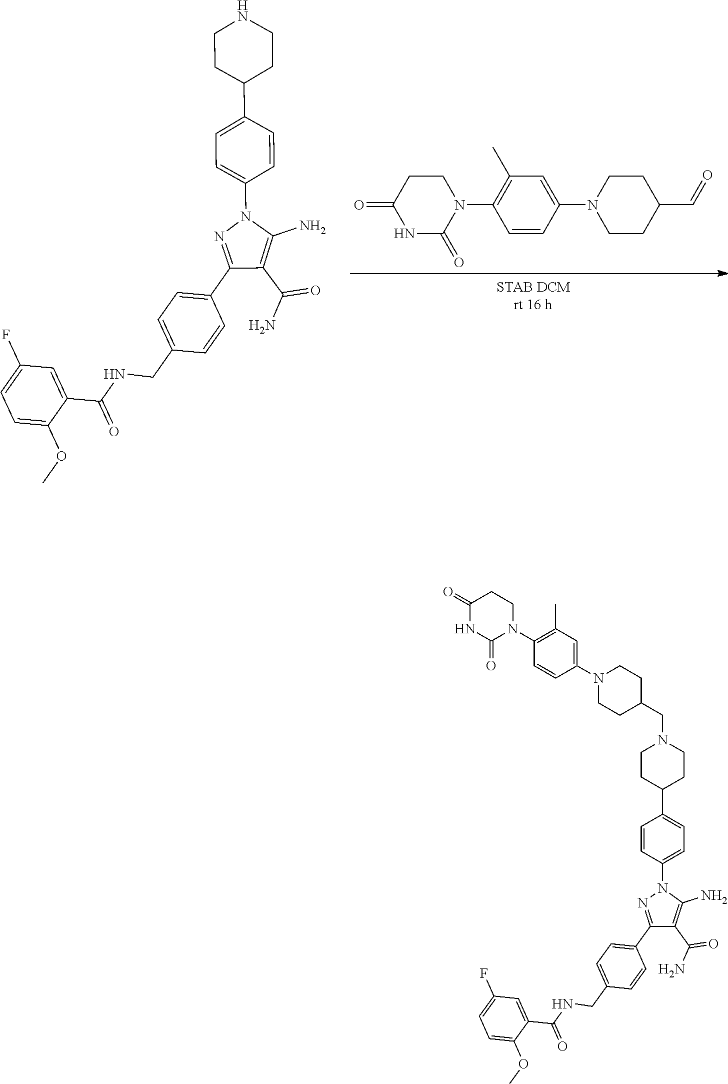 [SMILES: COc1ccc(F)cc1C(=O)NCc1ccc(-c2nn(-c3ccc(C4CCNCC4)cc3)c(N)c2C(N)=O)cc1 ; Cc1cc(N2CCC(C=O)CC2)ccc1N1CCC(=O)NC1=O ; COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c(N)c2C(N)=O)cc1]

At room temperature, S-amino-3-(4-((5-fluoro-2-methoxybenzamido)methyl)phenyl)-1-(4-(piperidin-4-yl)phenyl)-1H-pyrazole-4-carboxamide (0.10 g, 0.18 mmol), 1-(4-(2,4-dioxotetrahydropyrimidin-1(2H)-yl)-3-methylphenyl)piperidine-4-carboxaldehyde (0.07 g, 0.22 mmol), sodium triacetoxyborohydride (0.06 g, 0.28 mmol) and DCM (20 mL) were added into a single-necked flask in sequence and the reaction was allowed to proceed at room temperature for 16 h. The reaction solution was concentrated and subjected to column chromatography (MeOH/DCM system) to afford 0.07 g of a yellow solid with a yield of 45%, ¹H NMR (400 MHz, DMSO-d₆) δ 10.25 (s, 1H), 8.87 (t, J=6.2 Hz, 1H), 7.55 (d, J=8.0 Hz, 4H), 7.41 (dt, J=25.5, 8.7 Hz, 5H), 7.24-6.97 (m, 3H), 6.86-6.74 (m, 2H), 6.46 (s, 1H), 4.57 (d, J=6.1 Hz, 2H), 3.89 (d, J=9.2 Hz, 3H), 3.71 (d, J=12.8 Hz, 3H), 3.50-3.43 (m, 1H), 3.02 (s, 2H), 2.80-2.57 (m, 5H), 2.12 (s, 3H), 1.83 (d, J=12.7 Hz, 8H), 1.23 (d, J=11.2 Hz, 2H). MS (ESI) m/z: 842.7 [M+H]⁺.

### Example 31: Synthesis of N-(4-(8-amino-3-(4-((1-(4-(2,4-dioxotetrahydropyrimidin-1(2H)-yl)-3-methylphenyl)piperidin-4-yl)methyl)piperazin-1-yl)phenyl)imidazo[1,5-a]pyrazin-1-yl]-2-fluorobenzyl)-5-fluoro-2-methoxybenzamide (Compound I-45)

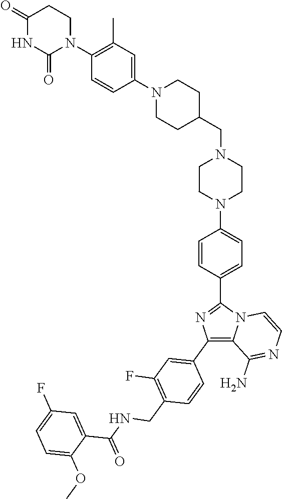 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1F]

The synthesis method of N-(4-(8-amino-3-(4-(piperazin-1-yl)phenyl)imidazo[1,5-a]pyrazin-1-yl]-2-fluorobenzyl)-5-fluoro-2-methoxybenzamide follows Example 20.

### Step 1: Synthesis of N-(4-(8-amino-3-(4-((1-(4-(2,4-dioxotetrahydropyrimidin-1(2H)-yl)-3-methylphenyl)piperidin-4-yl)methyl)piperazin-1-yl)phenyl)imidazo[1,5-a]pyrazin-1-yl]-2-fluorobenzyl)-5-fluoro-2-methoxybenzamide

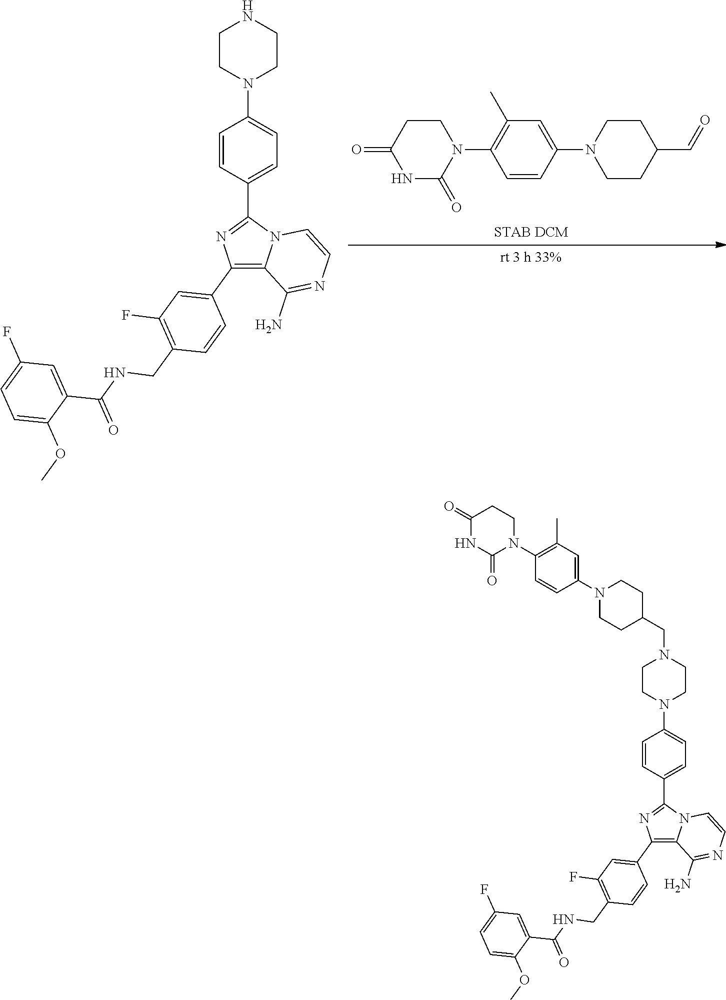 [SMILES: COc1ccc(F)cc1C(=O)NCc1ccc(-c2nc(-c3ccc(N4CCNCC4)cc3)n3ccnc(N)c23)cc1F ; Cc1cc(N2CCC(C=O)CC2)ccc1N1CCC(=O)NC1=O ; COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1F]

At room temperature, N-(4-(8-amino-3-(4-(piperazin-1-yl)phenyl)imidazo[1,5-a]pyrazin-1-yl]-2-fluorobenzyl)-5-fluoro-2-methoxybenzamide (0.10 g, 0.18 mmol), 1-(4-(2,4-dioxotetrahydropyrimidin-1(2H)-yl)-3-methylphenyl)piperidine-4-carbaldehyde (0.07 g, 0.21 mmol), sodium triacetoxyborohydride (0.06 g, 0.26 mmol) and DCM (20 mL) were added into a single-necked flask in sequence and the reaction was allowed to proceed at room temperature for 3 h. The reaction solution was concentrated and purified by column chromatography (MeOH/DCM system) to afford 0.05 g of a yellow solid with a yield of 33%, ¹H NMR (400 MHz, DMSO-d₆) δ 10.26 (s, 1H), 8.88 (t, J=6.1 Hz, 1H), 7.73-7.65 (m, 2H), 7.57-7.44 (m, 2H), 7.41-7.17 (m, 3H), 7.14-7.01 (m, 3H), 6.84-6.75 (m, 2H), 6.18 (s, 1H), 4.62 (d, J=6.0 Hz, 1H), 3.92 (s, 3H), 3.75-3.59 (m, 4H), 3.55-3.40 (m, 2H), 3.28-3.05 (m, 5H), 2.77-2.62 (m, 4H), 2.56-2.51 (m, 2H), 2.28-2.16 (m, 3H), 2.12 (s, 3H), 1.85-1.63 (m, 4H), 1.29-1.16 (m, 2H). MS (ESI) m/z: 869.7 [M+H]⁺.

### Example 32: Synthesis of N-(4-(4-amino-1-(4-(4-(2,4-dioxotetrahydropyrimidin-1(2H)-yl)-3-methylphenyl)piperidin-4-yl)methyl)piperazin-1-yl)phenyl)-1H-pyrazolo[3,4-d]pyrimidin-3-yl)benzyl)-5-fluoro-2-methoxybenzamide (Compound I-46)

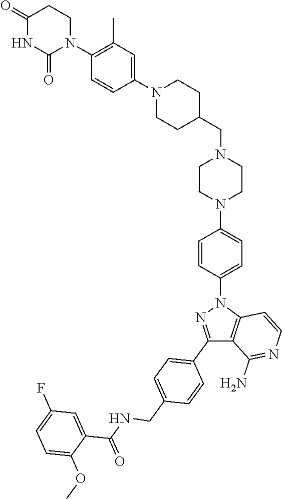 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ccnc(N)c23)cc1]

### Step 1: Synthesis of (N-(4-aminopyrazolo[4,3-c]pyridin-3-yl)benzyl)-5-fluoro-2-methoxybenzamide

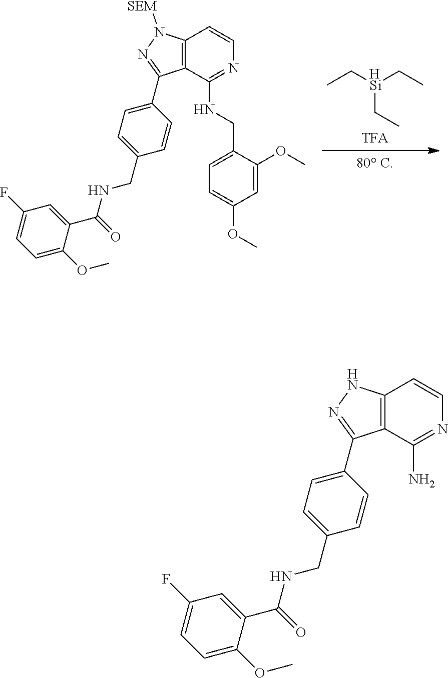

At room temperature, N-(4-(2,4-dimethoxybenzyl)amino)-1-(2-(trimethylsilyl) ethoxy)methyl-1H-pyrazolo[4,3-c]pyridin-3-yl)benzyl)-5-fluoro-2-methoxybenzamide (2.94 g, 4.38 mmol), trifluoroacetic acid (50 mL) and triethylsilane (5 mL) were added into a single-necked flask in sequence. The reaction was allowed to proceed at 80° C. for 1 h under nitrogen protection before termination. The reaction solution was cooled to room temperature, concentrated, and dissolved with a small amount of methanol, and the pH was adjusted to alkaline with ammonia water. The alkaline solution was diluted with water. The filter cake was collected by filtration and dried to afford 2.34 g of a crude product as a white solid.

### Step 2 Synthesis of tert-butyl 4-(4-(4-amino-3-(4-(5-fluoro-2-methoxybenzamido)methylphenyl)-1H-pyrazolo[4,3-c]pyridin-1-yl)phenyl) piperazine-1-carboxylate

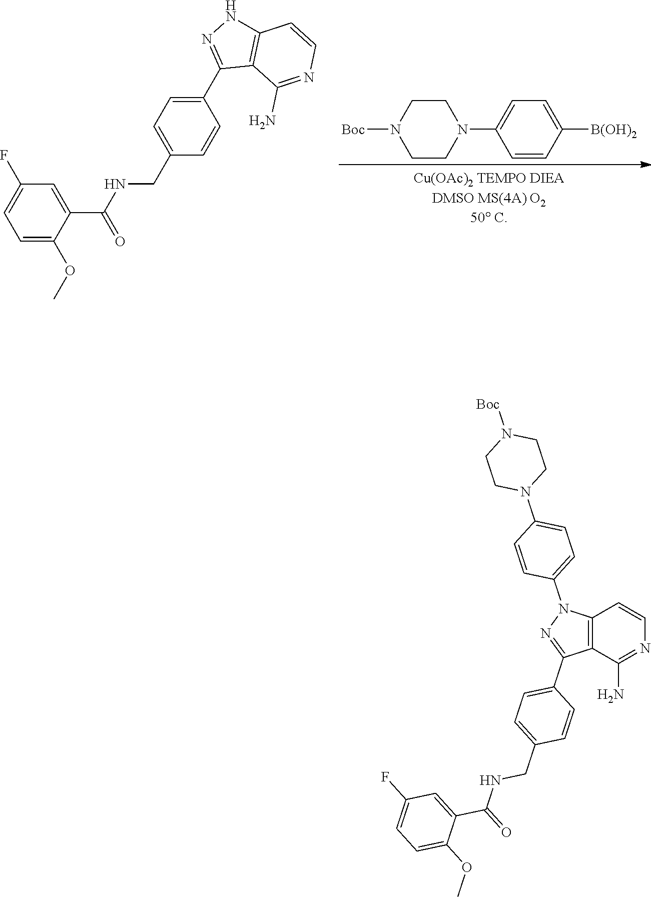 [SMILES: COc1ccc(F)cc1C(=O)NCc1ccc(-c2n[nH]c3ccnc(N)c23)cc1 ; CC(C)(C)OC(=O)N1CCN(c2ccc(B(O)O)cc2)CC1 ; COc1ccc(F)cc1C(=O)NCc1ccc(-c2nn(-c3ccc(N4CCN(C(=O)OC(C)(C)C)CC4)cc3)c3ccnc(N)c23)cc1]

At room temperature, (N-(4-aminopyrazolo[4,3-c]pyridin-3-yl)benzyl)-5-fluoro-2-methoxybenzamide (0.30 g, 0.77 mmol), (4-(4-(tert-butoxycarbonyl) piperazin-1-yl)phenylboronic acid (0.28 g, 0.92 mmol), copper acetate (0.15 g, 0.77 mmol), TEMPO (0.12 g, 0.77 mmol), DIEA (0.30 g, 2.30 mmol), MS (4A) (0.1 g) and DMSO (20 mL) were added into a single-necked flask in sequence. The reaction was allowed to proceed at 50° C. for 16 h under oxygen atmosphere before termination. The reaction solution was cooled to room temperature, diluted with water, and extracted with EA, and the organic phase was dried over anhydrous Na₂SO₄, filtered, concentrated and purified by column chromatography (MeOH/DCM system) to afford 0.07 g of a black solid with a yield of 4%.

### Step 3: Synthesis of N-(4-amino-1-(4-(4-piperazin-1-yl)phenyl)-1H-pyrazolo[3,4-c]pyrimidin-3-yl)benzyl)-5-fluoro-2-methoxybenzamide

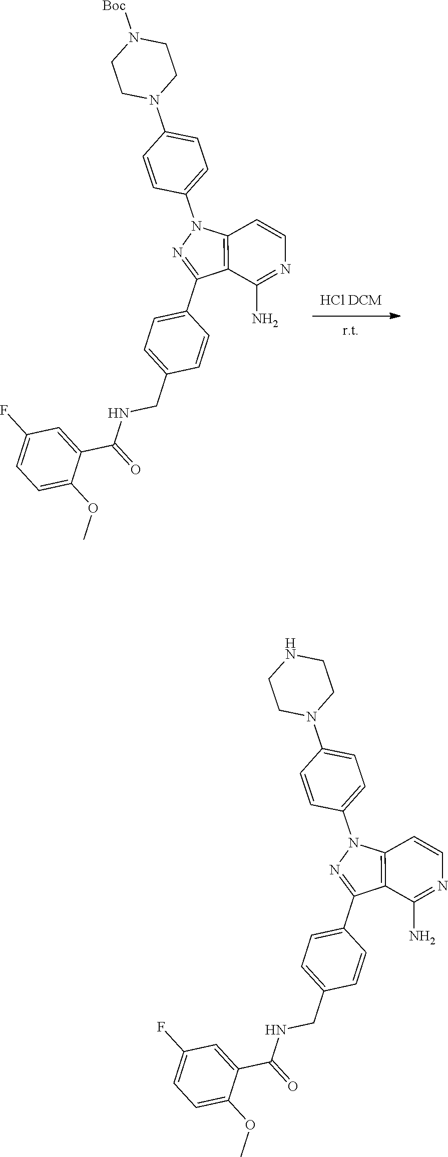 [SMILES: COc1ccc(F)cc1C(=O)NCc1ccc(-c2nn(-c3ccc(N4CCN(C(=O)OC(C)(C)C)CC4)cc3)c3ccnc(N)c23)cc1 ; COc1ccc(F)cc1C(=O)NCc1ccc(-c2nn(-c3ccc(N4CCNCC4)cc3)c3ccnc(N)c23)cc1]

At room temperature, tert-butyl 4-(4-(4-amino-3-(4-(5-fluoro-2-methoxybenzamido)methylphenyl)-1H-pyrazolo[4,3-c]pyridin-1-yl)phenyl) piperazine-1-carboxylate (0.07 g, 0.04 mmol), hydrogen chloride (in 1,4-dioxane, 5 mL) and dichloromethane (10 mL) were added into a single-necked flask in sequence. The reaction was allowed to proceed at room temperature for 1 h under nitrogen protection before termination. The reaction solution was concentrated and diluted with a small amount of methanol, and the pH was adjusted to alkaline with ammonia water. The alkaline solution was concentrated to afford 0.07 g of a crude product as a brown solid.

### Step 4: Synthesis of N-(4-(4-amino-1-(4-(4-(2,4-dioxotetrahydropyrimidin-1(2H)-yl)-3-methylphenyl)piperidin-4-yl)methyl)piperazin-1-yl)phenyl)-1H-pyrazolo[3,4-d]pyrimidin-3-yl)benzyl)-5-fluoro-2-methoxybenzamide

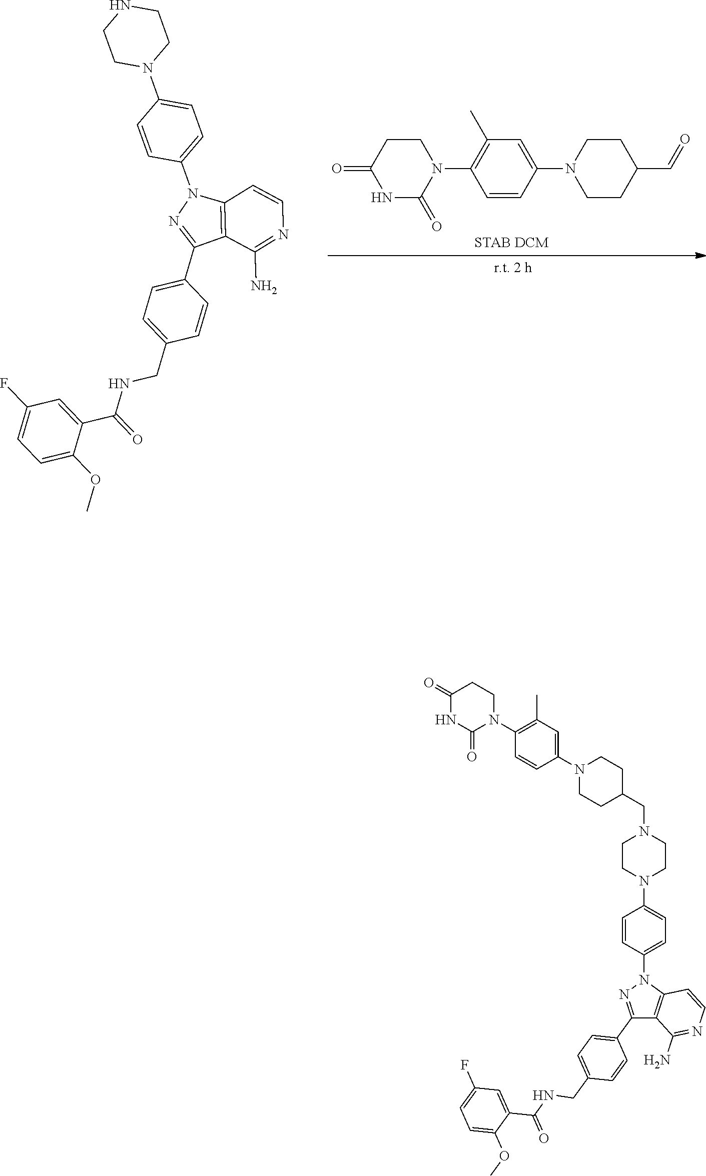 [SMILES: COc1ccc(F)cc1C(=O)NCc1ccc(-c2nn(-c3ccc(N4CCNCC4)cc3)c3ccnc(N)c23)cc1 ; Cc1cc(N2CCC(C=O)CC2)ccc1N1CCC(=O)NC1=O ; COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ccnc(N)c23)cc1]

At room temperature, 1-(4-(2,4-dioxotetrahydropyrimidin-1(2H)-yl)-3-methylphenyl)piperidine-4-carbaldehyde (0.02 g, 0.06 mmol), N-(4-amino-1-(4-(4-piperazin-1-yl)phenyl)-1H-pyrazolo[3,4-c]pyrimidin-3-yl)benzyl)-5-fluoro-2-methoxybenzamide (0.07 g, 0.04 mmol), sodium triacetoxyborohydride (0.01 g, 0.06 mmol) and dichloromethane (15 mL) were added into a single-necked flask in sequence. The reaction was allowed to proceed at room temperature for 2 h under nitrogen protection before termination. The reaction solution was concentrated and purified on a thick preparative plate to afford 0.003 g of a yellow solid with a yield of 3%. MS (ESI) m/z: 851.83 [M+H]⁺.

### Example 33: Synthesis of N-(4-(4-amino-1-(4-(4-(2,4-dioxotetrahydropyrimidin-1(2H)-yl)-3-methylphenyl)piperidin-4-yl)methyl)piperazin-1-yl)phenyl)-1H-pyrazolo[3,4-d]pyrimidin-3-yl)benzyl)-5-fluoro-2-methoxybenzamide (Compound I-9)

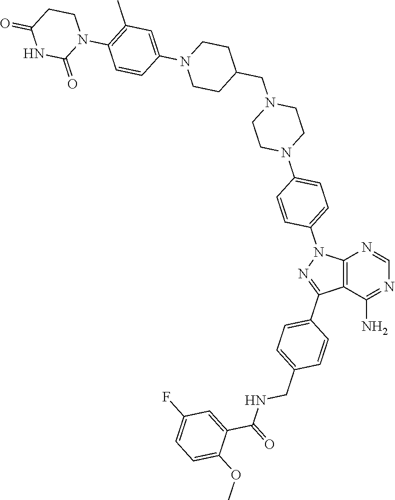 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ncnc(N)c23)cc1]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

### Step 1: Synthesis of tert-butyl 4-(4-(4-amino-3-(4-(5-fluoro-2-methoxybenzamido)methyl)phenyl)-1H-pyrazolo[3,4-d]pyrimidin-1-yl)phenyl) piperazine-1-carboxylate

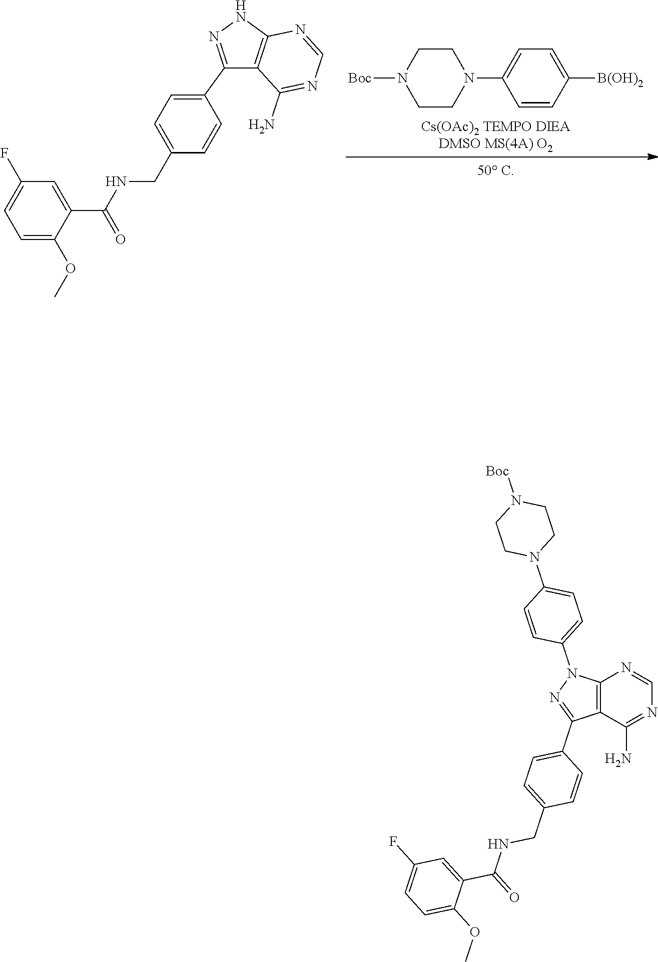 [SMILES: COc1ccc(F)cc1C(=O)NCc1ccc(-c2n[nH]c3ncnc(N)c23)cc1 ; CC(C)(C)OC(=O)N1CCN(c2ccc(B(O)O)cc2)CC1 ; COc1ccc(F)cc1C(=O)NCc1ccc(-c2nn(-c3ccc(N4CCN(C(=O)OC(C)(C)C)CC4)cc3)c3ncnc(N)c23)cc1]

At room temperature, N-(4-aminopyrazolo[3,4-d]pyrimidin-3-yl)benzyl)-5-fluoro-2-methoxybenzamide (0.34 g, 0.87 mmol), (4-(4-(tert-butoxycarbonyl) piperazin-1-yl)phenylboronic acid (0.32 g, 1.04 mmol), copper acetate (0.17 g, 0.87 mmol), TEMPO (0.14 g, 0.87 mmol), DIEA (0.34 g, 2.61 mmol), MS (4A) (0.1 g) and DMSO (20 mL) were added into a single-necked flask in sequence. The reaction was allowed to proceed at 50° C. for 18 h under oxygen atmosphere before termination. The reaction solution was cooled to room temperature, diluted with water, and extracted with EA, and the organic phase was dried over anhydrous Na₂SO₄, filtered, concentrated and purified by column chromatography (EA/PE system) to afford 0.11 g of a black solid with a yield of 21%.

### Step 2: Synthesis of N-(4-amino-1-(4-(4-piperazin-1-yl)phenyl)-1H-pyrazolo[3,4-d]pyrimidin-3-yl)benzyl)-5-fluoro-2-methoxybenzamide

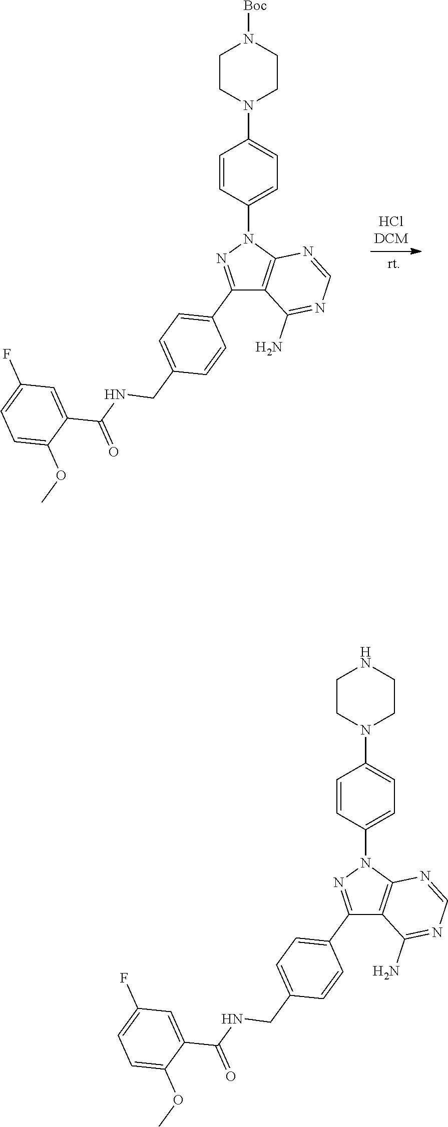 [SMILES: COc1ccc(F)cc1C(=O)NCc1ccc(-c2nn(-c3ccc(N4CCN(C(=O)OC(C)(C)C)CC4)cc3)c3ncnc(N)c23)cc1 ; COc1ccc(F)cc1C(=O)NCc1ccc(-c2nn(-c3ccc(N4CCNCC4)cc3)c3ncnc(N)c23)cc1]

At room temperature, tert-butyl 4-(4-(4-amino-3-(4-(5-fluoro-2-methoxybenzamido)methyl)phenyl)-1H-pyrazolo[3,4-d]pyrimidin-1-yl)phenyl) piperazine-1-carboxylate (0.11 g, 0.20 mmol), hydrogen chloride (in 1,4-dioxane, 8 mL) and dichloromethane (8 mL) were added into a single-necked flask in sequence. The reaction was allowed to proceed at room temperature for 1 h under nitrogen protection before termination. The reaction solution was concentrated and diluted with a small amount of methanol, and the pH was adjusted to alkaline with ammonia water. The alkaline solution was concentrated to afford 0.10 g of a crude product as a brown solid.

### Step 3: Synthesis of N-(4-(4-amino-1-(4-(4-(2,4-dioxotetrahydropyrimidin-1(2H)-yl)-3-methylphenyl)piperidin-4-yl)methyl)piperazin-1-yl)phenyl)-1H-pyrazolo[3,4-d]pyrimidin-3-yl)benzyl)-5-fluoro-2-methoxybenzamide

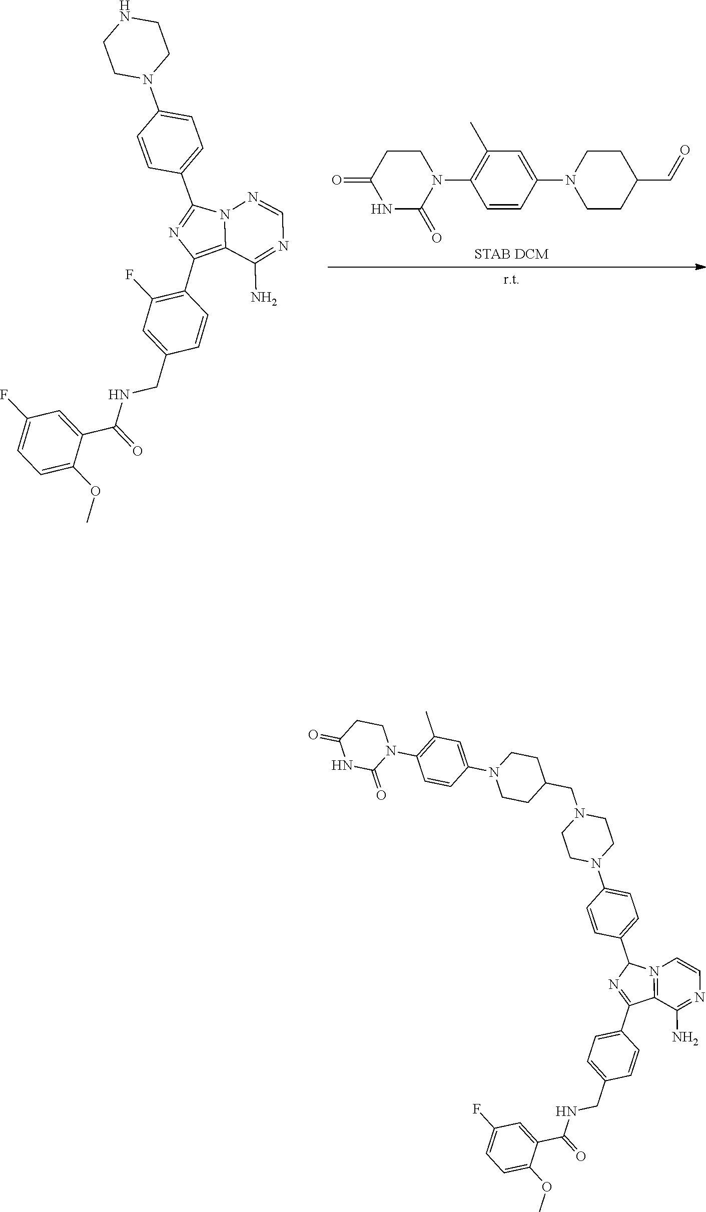

At room temperature, 1-(4-(2,4-dioxotetrahydropyrimidin-1(2H)-yl)-3-methylphenyl)piperidine-4-carbaldehyde (0.08 g, 0.26 mmol), N-(4-amino-1-(4-(4-piperazin-1-yl)phenyl)-1H-pyrazolo[3,4-d]pyrimidin-3-yl)benzyl)-5-fluoro-2-methoxybenzamide (0.10 g, 0.20 mmol), sodium triacetoxyborohydride (0.06 g, 0.30 mmol) and dichloromethane (15 mL) were added into a single-necked flask in sequence. The reaction was allowed to proceed at room temperature for 2 h under nitrogen protection before termination. The reaction solution was concentrated and purified by column chromatography (MeOH/DCM system) to afford 0.04 g of a yellow solid with a yield of 23%, ¹H NMR (400 MHz, DMSO-d₆) δ 8.91 (s, 1H), 8.34 (m, 1H), 7.96 (m, 2H), 7.64 (m, 5H), 7.35 (m, 1H), 7.24-6.97 (m, 4H), 6.79 (m, 2H), 4.61 (m, 2H), 3.92 (s, 3H), 3.68 (m, 6H), 3.22 (m, 6H), 2.68 (m, 5H), 2.24 (m, 1H), 2.12 (s, 3H), 1.86 (m, 3H), 1.23 (m, 2H). MS (ESI) m/z: 853.01 [M+H]⁺.

### Example 34: Synthesis of N-(4-(8-amino-3-(4-(4-(4-(2,4-dioxotetrahydropyrimidin-1(2H)-yl)-3-methylphenyl)piperidin-4-yl)methyl)piperazin-1-yl)phenyl)imidazo[1,5-a]pyrazin-1-yl)-3-fluorobenzyl)-5-fluoro-2-methoxybenzamide (Compound I-47)

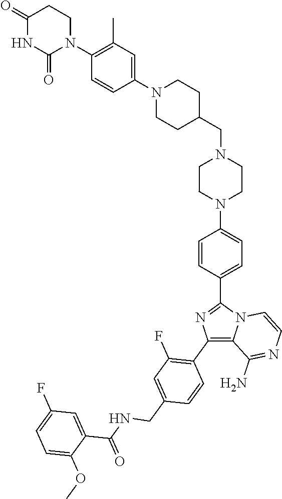 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)c(F)c1]

The synthesis method of N-(4-(8-amino-3-(4-(piperazin-1-yl)phenyl)imidazo[1,5-a]pyrazin-1-yl)-3-fluorobenzyl)-5-fluoro-2-methoxybenzamide follows Example 20.

### Step 1: Synthesis of N-(4-(8-amino-3-(4-(4-(4-(2,4-dioxotetrahydropyrimidin-1(2H)-yl)-3-methylphenyl)piperidin-4-yl)methyl)piperazin-1-yl)phenyl)imidazo[1,5-a]pyrazin-1-yl)-3-fluorobenzyl)-5-fluoro-2-methoxybenzamide

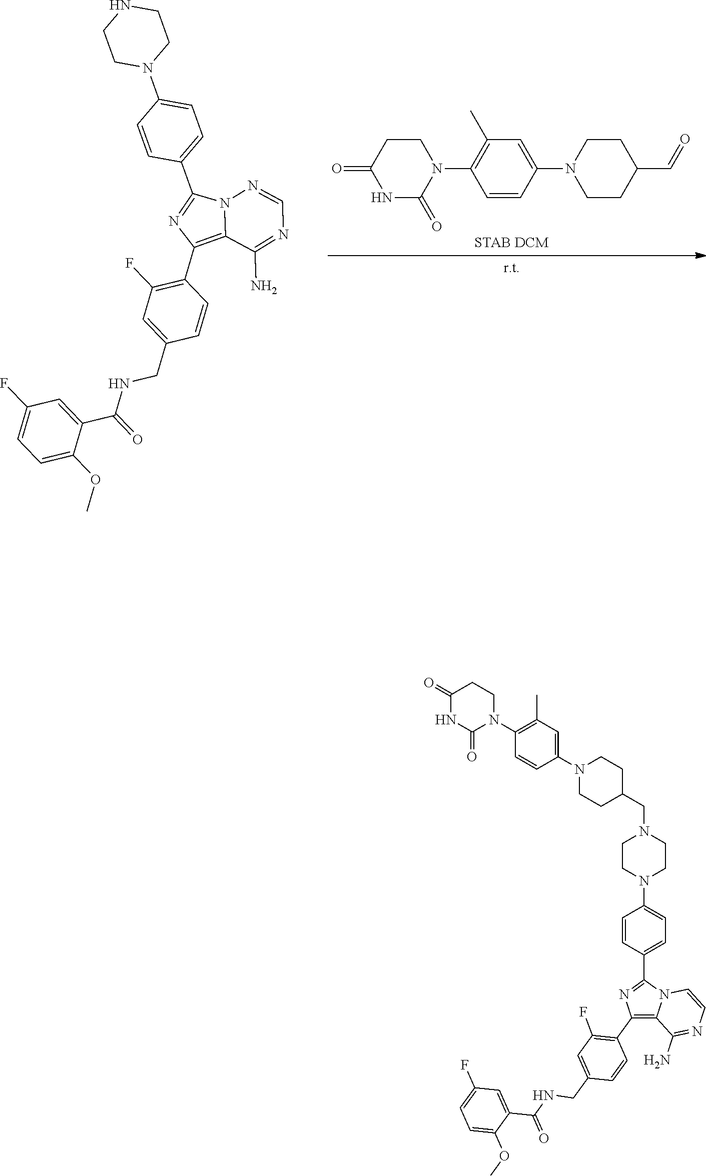 [SMILES: COc1ccc(F)cc1C(=O)NCc1ccc(-c2nc(-c3ccc(N4CCNCC4)cc3)n3ncnc(N)c23)c(F)c1 ; Cc1cc(N2CCC(C=O)CC2)ccc1N1CCC(=O)NC1=O ; COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)c(F)c1]

At room temperature, 1-(4-(2,4-dioxotetrahydropyrimidin-1(2H)-yl)-3-methylphenyl)piperidine-4-carbaldehyde (0.07 g, 0.23 mmol), N-(4-(8-amino-3-(4-(piperazin-1-yl)phenyl)imidazo[1,5-a]pyrazin-1-yl)-3-fluorobenzyl)-5-fluoro-2-methoxybenzamide (0.14 g, 0.18 mmol), sodium triacetoxyborohydride (0.06 g, 0.27 mmol) and dichloromethane (20 mL) were added into a single-necked flask in sequence. The reaction was allowed to proceed at room temperature for 2 h under nitrogen protection before termination. The reaction solution was concentrated and purified by column chromatography (MeOH/DCM=1/10) to afford 0.090 g of a white solid with a yield of 58%. MS (ESI) m/z: 869.77 [M+H]⁺.

### Example 35: Synthesis of N-(4-(4-amino-1-(4-(-4-(1-(4-(2,4-dioxotetrahydropyrimidin-1(2H)-yl)-3-methylphenyl)piperidin-4-yl)methyl)piperazin-1-yl)phenyl)-2-oxo-1,2-dihydro-3H-imidazo[4,5-c]pyridin-3-yl)benzyl)-5-fluoro-2-methoxybenzamide (Compound I-48)

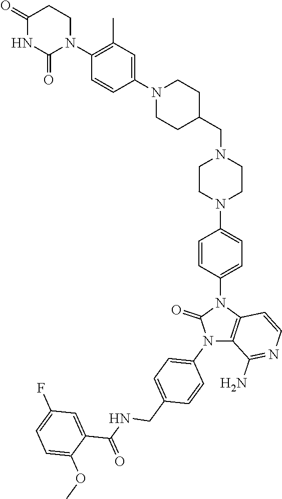 [SMILES: COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)cc3)c3ccnc(N)c32)cc1]

### Step 1: Synthesis of tert-butyl 4-(4-((2-chloro-3-nitropyridin-4-yl)amino)phenyl) piperazine-1-carboxylate

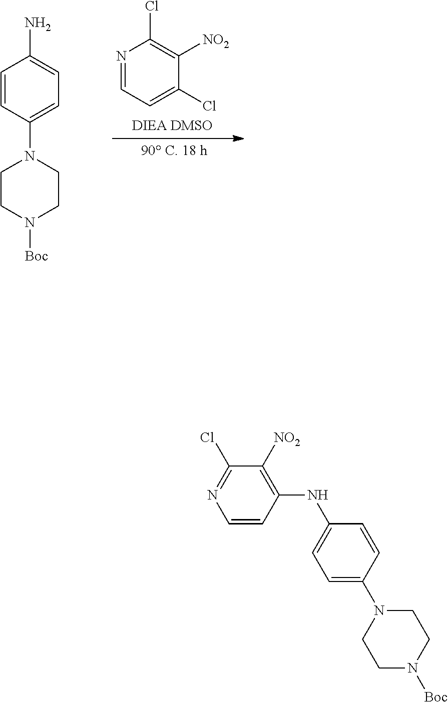 [SMILES: CC(C)(C)OC(=O)N1CCN(c2ccc(N)cc2)CC1 ; O=[N+]([O-])c1c(Cl)ccnc1Cl ; CC(C)(C)OC(=O)N1CCN(c2ccc(Nc3ccnc(Cl)c3[N+](=O)[O-])cc2)CC1]

At room temperature, tert-butyl 4-(4-aminophenyl) piperazine-1-carboxylate (5.00 g, 18.03 mmol), 2,4-dichloro-3-nitropyridine (4.18 g, 21.63 mmol), DIEA (4.67 g, 36.06 mmol) and DMSO (30 mL) were added into a single-necked flask in sequence. The mixture was heated to 95° C. and the reaction was allowed to proceed for 18 h. The reaction solution was cooled to room temperature and diluted with EA. The organic phase was washed with water and saturated brine, dried over anhydrous sodium sulfate, concentrated and subjected to column chromatography (EA/PE system) to afford 2.80 g of a yellow solid with a yield of 36%.

### Step 2: Synthesis of tert-butyl 4-(4-((2-(bis(4-methoxybenzyl)amino)-3-nitropyridin-4-yl)amino)phenyl) piperazine-1-carboxylate

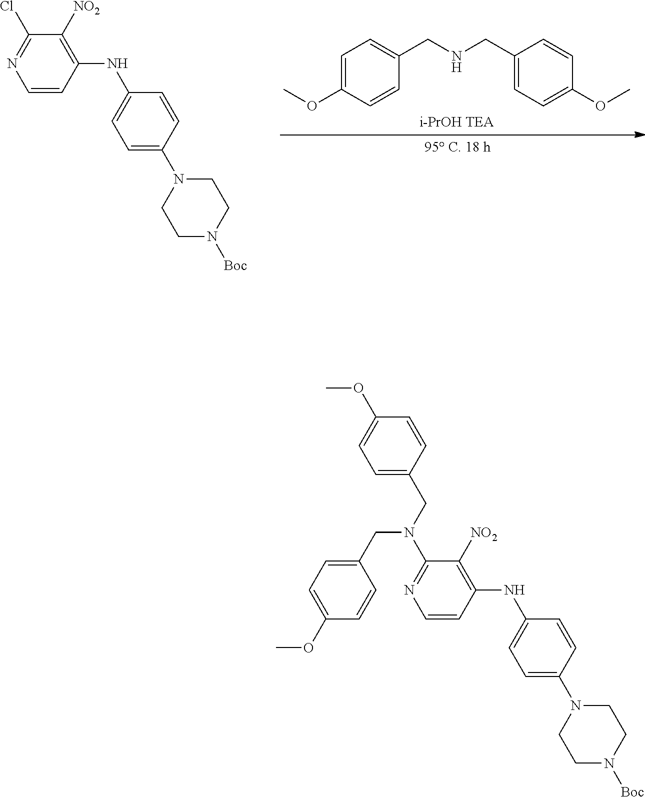

At room temperature, tert-butyl 4-(4-((2-chloro-3-nitropyridin-4-yl)amino)phenyl) piperazine-1-carboxylate (2.21 g, 5.09 mmol), bis(4-methoxybenzyl)amine (1.80 g, 6.62 mmol), triethylamine (1.03 g, 10.18 mmol) and isopropanol (30 mL) were added into a single-necked flask in sequence. The mixture was heated to 95° C. and the reaction was allowed to proceed for 18 h. The reaction solution was cooled to room temperature and diluted with EA. The organic phase was washed with water and saturated brine, dried over anhydrous sodium sulfate, concentrated and subjected to column chromatography (EA/PE system) to afford 3.00 g of a yellow solid with a yield of 90%. Two batches were fed in parallel, totaling 6.00 g.

### Step 3: Synthesis of tert-butyl 4-(4-((3-amino-2-(bis(4-methoxybenzyl)amino)pyridin-4-yl)amino)phenyl) piperazine-1-carboxylate

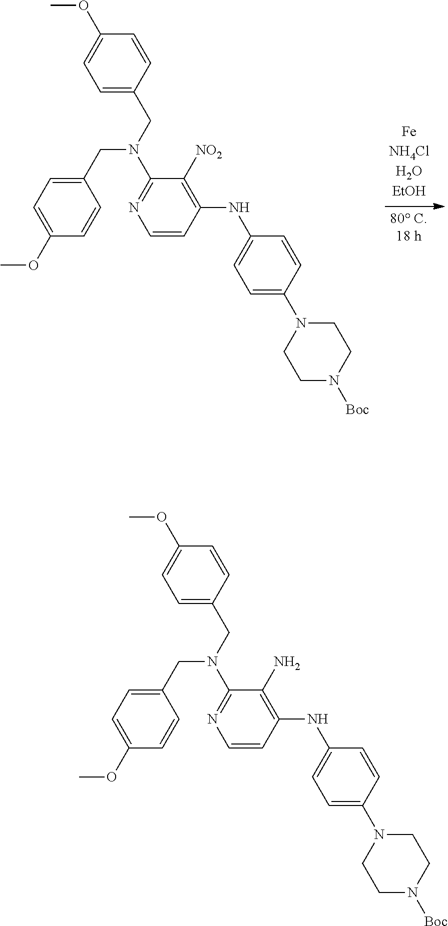 [SMILES: COc1ccc(CN(Cc2ccc(OC)cc2)c2nccc(Nc3ccc(N4CCN(C(=O)OC(C)(C)C)CC4)cc3)c2[N+](=O)[O-])cc1 ; COc1ccc(CN(Cc2ccc(OC)cc2)c2nccc(Nc3ccc(N4CCN(C(=O)OC(C)(C)C)CC4)cc3)c2N)cc1]

At room temperature, tert-butyl 4-(4-((2-(bis(4-methoxybenzyl)amino)-3-nitropyridin-4-yl)amino)phenyl) piperazine-1-carboxylate (3.90 g, 5.96 mmol), iron powder (1.66 g, 29.78 mmol), ammonium chloride (1.60 g, 29.78 mmol), ethanol (80 mL) and water (20 mL) were added into a single-necked flask in sequence. The mixture was heated to 80° C. and the reaction was allowed to proceed for 18 h. The reaction solution was cooled to room temperature and filtered. The filtrate was diluted with EA. The organic phase was washed with water and saturated brine, dried over anhydrous sodium sulfate, and concentrated to afford 3.25 g of a yellow solid.

### Step 4: Synthesis of tert-butyl 4-(4-(4-bis(4-methoxybenzyl)amino)-2-oxo-2,3-dihydro-1H-imidazo[4,5-c]pyridin-1-yl)phenyl) piperazine-1-carboxylate

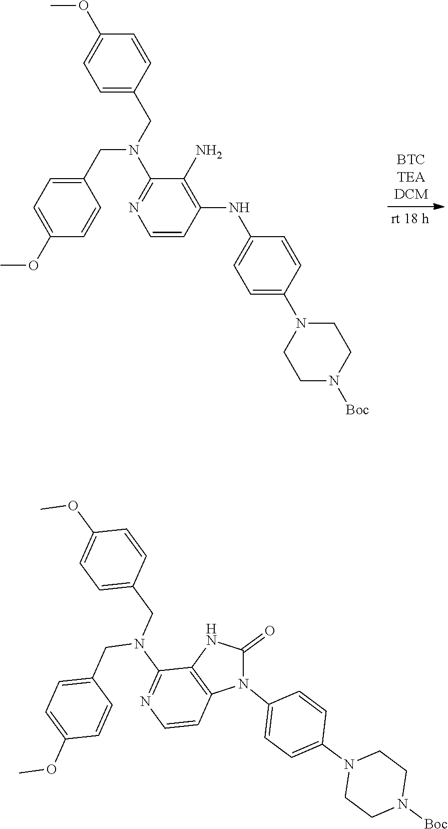 [SMILES: COc1ccc(CN(Cc2ccc(OC)cc2)c2nccc(Nc3ccc(N4CCN(C(=O)OC(C)(C)C)CC4)cc3)c2N)cc1 ; COc1ccc(CN(Cc2ccc(OC)cc2)c2nccc3c2[nH]c(=O)n3-c2ccc(N3CCN(C(=O)OC(C)(C)C)CC3)cc2)cc1]

Under ice-water bath conditions, tert-butyl 4-(4-(4-bis(4-methoxybenzyl)amino)-2-oxo-2,3-dihydro-1H-imidazo[4,5-c]pyridin-1-yl)phenyl) piperazine-1-carboxylate (3.70 g, 5.69 mmol), triethylamine (1.66 g, 29.78 mmol), triphosgene (0.66 g, 2.24 mmol) and DCM (50 mL) were added into a single-necked flask in sequence and the reaction was allowed to proceed at room temperature for 18 h. Two batches were combined, concentrated, and purified by column chromatography (EA/PE system) to afford 3.70 g of a yellow solid with a yield of 90%.

### Step 5: Synthesis of 4-amino-1-(4-(piperazin-1-yl)phenyl)-1,3-dihydro-2H-imidazo[4,5-c]pyridin-2-one

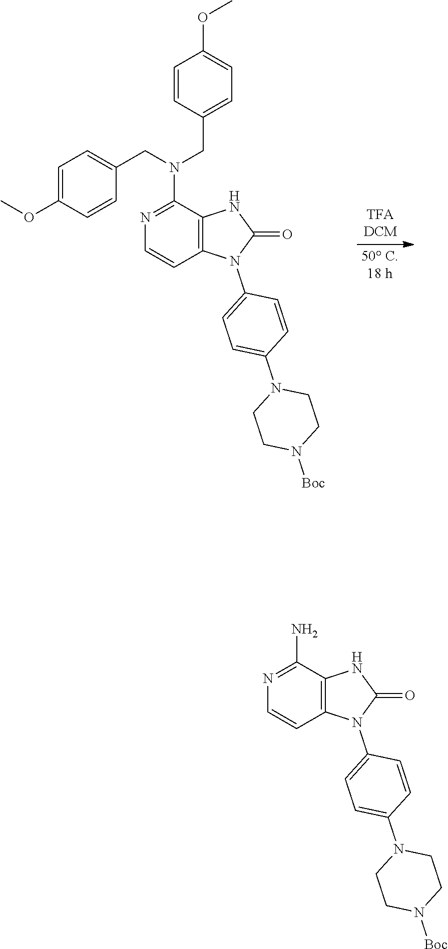 [SMILES: COc1ccc(CN(Cc2ccc(OC)cc2)c2nccc3c2[nH]c(=O)n3-c2ccc(N3CCN(C(=O)OC(C)(C)C)CC3)cc2)cc1 ; CC(C)(C)OC(=O)N1CCN(c2ccc(-n3c(=O)[nH]c4c(N)nccc43)cc2)CC1]

At room temperature, tert-butyl 4-(4-(4-bis(4-methoxybenzyl)amino)-2-oxo-2,3-dihydro-1H-imidazo[4,5-c]pyridin-1-yl)phenyl) piperazine-1-carboxylate (3.70 g, 5.69 mmol), TFA (15 mL) and DCM (15 mL) were added into a single-necked flask in sequence. The mixture was heated to 50° C. and the reaction was allowed to proceed for 18 h. The reaction solution was cooled to room temperature and concentrated to afford 3.0 g of an oil crude product, which was directly used in the next step.

### Step 6: Synthesis of tert-butyl 4-(4-(4-amino-2-oxo-2,3-dihydro-1H-imidazo[4,5-c]pyridin-1-yl)phenyl) piperazine-1-carboxylate

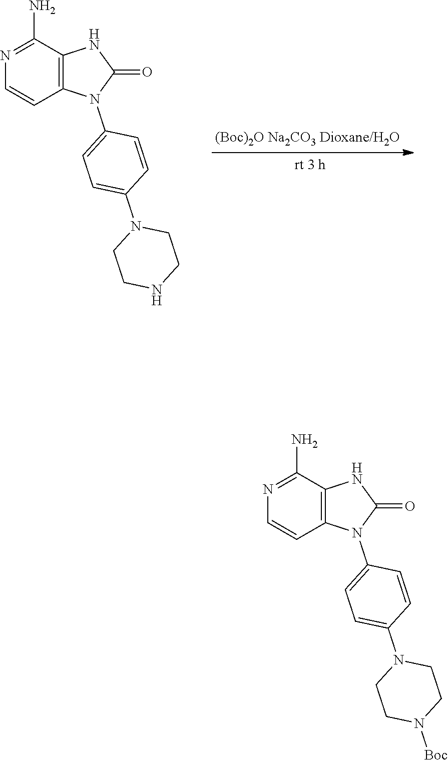 [SMILES: Nc1nccc2c1[nH]c(=O)n2-c1ccc(N2CCNCC2)cc1 ; CC(C)(C)OC(=O)N1CCN(c2ccc(-n3c(=O)[nH]c4c(N)nccc43)cc2)CC1]

At room temperature, 4-amino-1-(4-(piperazin-1-yl)phenyl)-1,3-dihydro-2H-imidazo[4,5-c]pyridin-2-one (1.77 g, 5.69 mmol), (Boc)₂O (1.24 g, 5.97 mmol), sodium carbonate (1.21 g, 11.38 mmol), 1,4-dioxane (30 mL) and H₂O (30 mL) were added into a single-necked flask in sequence and the reaction was allowed to proceed at room temperature for 3 b. The reaction solution was extracted for three times with DCM, and the organic phases were combined, dried, concentrated, and slurried with a (EA/PE) mixed solvent. The slurry was filtered. The filter cake was dried to afford 2.42 g of a crude product as a yellow solid.

### Step 7: Synthesis of (E)-tert-butyl 4-(4-(((dimethylamino)methylene)amino)-2-oxo-2,3-dihydro-1H-imidazo[4,5-c]pyridin-1-yl)phenyl) piperazine-1-carboxylate

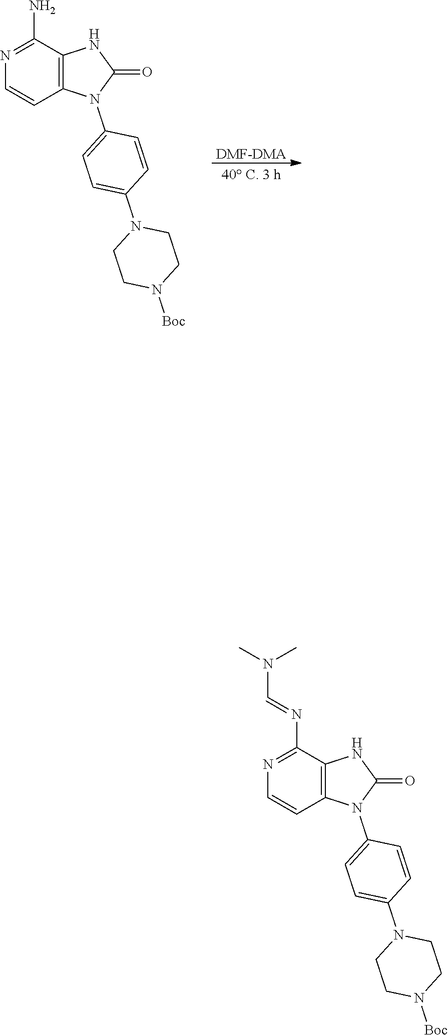

At room temperature, tert-butyl 4-(4-(4-amino-2-oxo-2,3-dihydro-1H-imidazo[4,5-c]pyridin-1-yl)phenyl) piperazine-1-carboxylate (2.42 g, 5.90 mmol) and DMF-DMA (30 mL) were added into a single-necked flask in sequence. The mixture was heated to 40° C. and the reaction was allowed to proceed for 4 h. The reaction solution was cooled to room temperature and concentrated. The residue was slurried using PE and filtered. The filter cake was dried to afford 1.52 g of a crude product as a yellow solid.

### Step 8: Synthesis of (E)-tert-butyl 4-(4-(4-(((dimethylamino)methylene)amino)-3-(4-(5-fluoro-2-methoxybenzamido)methyl)phenyl)-2-oxo-2,3-dihydro-1H-imidazo[4,5-c]pyridin-1-yl)phenyl) piperazine-1-carboxylate

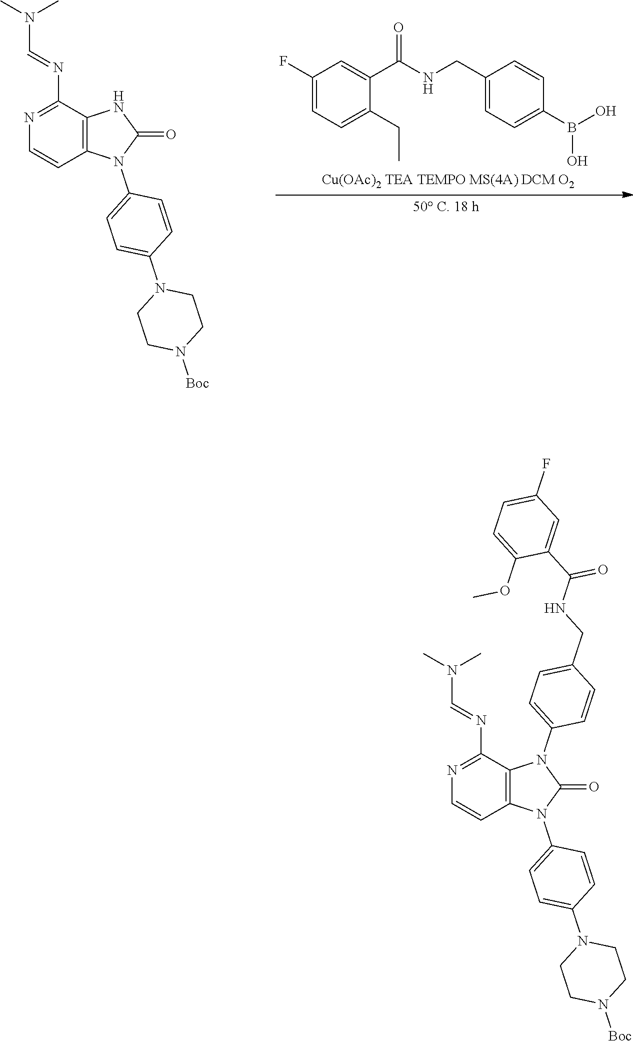 [SMILES: CN(C)/C=N/c1nccc2c1[nH]c(=O)n2-c1ccc(N2CCN(C(=O)OC(C)(C)C)CC2)cc1 ; CCc1ccc(F)cc1C(=O)NCc1ccc(B(O)O)cc1 ; COc1ccc(F)cc1C(=O)NCc1ccc(-n2c(=O)n(-c3ccc(N4CCN(C(=O)OC(C)(C)C)CC4)cc3)c3ccnc(/N=C/N(C)C)c32)cc1]

At room temperature, (E)-tert-butyl 4-(4-(((dimethylamino)methylene)amino)-2-oxo-2,3-dihydro-1H-imidazo[4,5-c]pyridin-1-yl)phenyl) piperazine-1-carboxylate (0.46 g, 1.00 mmol), (4-((5-fluoro-2-methoxybenzamido)methyl)phenyl)boronic acid (0.61 g, 2.00 mmol), copper acetate (0.10 g, 0.50 mmol), TEMPO (0.17 g, 1.10 mmol), DIEA (0.56 g, 4.00 mmol), DMSO (30 mL) and 4A molecular sieves (0.50 g) were added into a single-necked flask in sequence. The mixture was heated to 50° C. and the reaction was allowed to proceed for 18 h. The reaction solution was cooled to room temperature and filtered. The filtrate was diluted with EA. The organic phase was washed with water and saturated brine, dried over anhydrous sodium sulfate and filtered, and the filtrate was concentrated and subjected to column chromatography (MeOH/DCM) to afford 0.41 g of a yellow solid.

### Step 9: Synthesis of N-(4-(4-amino-2-oxo-1-(4-(piperazin-1-yl)phenyl)-1,2-dihydro-3H-imidazo[4,5-c]pyridin-3-yl)benzyl)-5-fluoro-2-methoxybenzamide

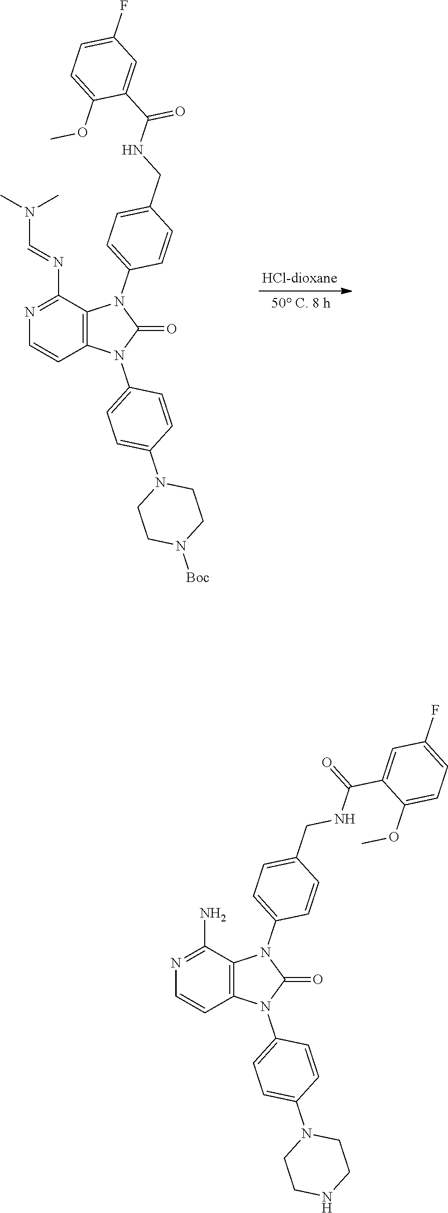 [SMILES: COc1ccc(F)cc1C(=O)NCc1ccc(-n2c(=O)n(-c3ccc(N4CCN(C(=O)OC(C)(C)C)CC4)cc3)c3ccnc(/N=C/N(C)C)c32)cc1 ; COc1ccc(F)cc1C(=O)NCc1ccc(-n2c(=O)n(-c3ccc(N4CCNCC4)cc3)c3ccnc(N)c32)cc1]

At room temperature, (E)-ter-butyl 4-(4-(4-(((dimethylamino)methylene)amino)-3-(4-(5-fluoro-2-methoxybenzamido)methyl)phenyl)-2-oxo-2,3-dihydro-1H-imidazo[4,5-c]pyridin-1-yl)phenyl) piperazine-1-carboxylate (0.41 g, 0.57 mmol), HCl-dioxane (15 mL) and concentrated hydrochloric acid (S mL) were added into a single-necked flask in sequence. The mixture was heated to 80° C. and the reaction was allowed to proceed for 8 h. The reaction solution was concentrated, and the concentrated reaction solution was treated with aqueous ammonia and concentrated to afford 0.20 g of a crude product as a yellow solid.

### Step 10: Synthesis of N-(4-(4-amino-1-(4-(-4-(1-(4-(2,4-dioxotetrahydropyrimidin-1(2H)-yl)-3-methylphenyl)piperidin-4-yl)methyl)piperazin-1-yl)phenyl)-2-oxo-1,2-dihydro-3H-imidazo[4,5-c]pyridin-3-yl)benzyl)-5-fluoro-2-methoxybenzamide

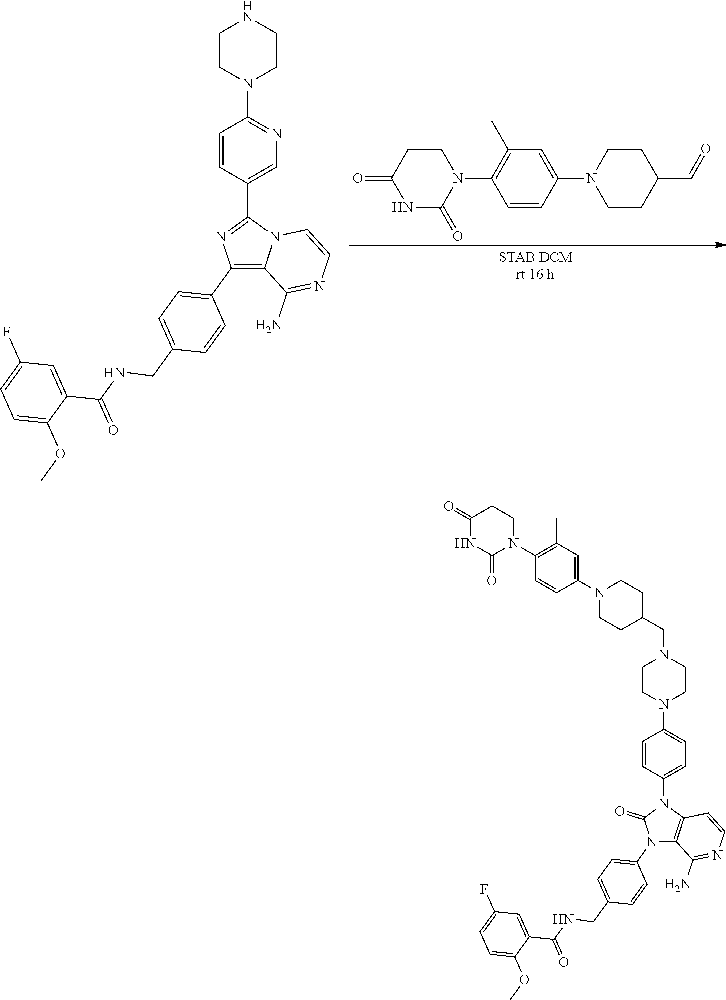 [SMILES: COc1ccc(F)cc1C(=O)NCc1ccc(-c2nc(-c3ccc(N4CCNCC4)nc3)n3ccnc(N)c23)cc1 ; Cc1cc(N2CCC(C=O)CC2)ccc1N1CCC(=O)NC1=O ; COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)cc3)c3ccnc(N)c32)cc1]

At room temperature, N-(4-(4-amino-2-oxo-1-(4-(piperazin-1-yl)phenyl)-1,2-dihydro-3H-imidazo[4,5-c]pyridin-3-yl)benzyl)-5-fluoro-2-methoxybenzamide (0.20 g, 0.35 mmol), 1-(4-(2,4-dioxotetrahydropyrimidin-1(2H)-yl)-3-methylphenyl)piperidine-4-carbaldehyde (0.13 g, 0.42 mmol), sodium triacetoxyborohydride (0.11 g, 0.53 mmol) and DCM (20 mL) were added into a single-necked flask in sequence and the reaction was allowed to proceed at room temperature for 16 h. The reaction solution was concentrated and purified by column chromatography (MeOH/DCM system) to afford 0.14 g of a yellow solid with a yield of 50%. ¹H NMR (400 MHz, DMSO-d₆) δ 10.27 (s, 1H), 8.94 (t, J=6.2 Hz, 1H), 7.74 (d, J=5.5 Hz, 1H), 7.57-7.47 (m, 5H), 7.39-7.31 (m, 3H), 7.20 (dd, J=9.2, 4.3 Hz, 1H), 7.07 (dd, J=22.0, 8.6 Hz, 3H), 6.84-6.75 (m, 2H), 6.41 (d, J=5.5 Hz, 1H), 4.83 (s, 2H), 4.61 (d, J=6.1 Hz, 2H), 3.74-3.62 (m, 3H), 3.25-3.21 (m, 4H), 2.78-2.62 (m, 5H), 2.24 (d, J=7.1 Hz, 2H), 2.12 (s, 3H), 1.85-1.72 (m, 3H), 1.22 (d, J=11.9 Hz, 2H). MS (ESI) m/z: 867.6 [M+H]⁺.

### Example 36: Synthesis of N-(4-(8-amino-3-(4-(4-(4-(2,4-dioxotetrahydropyrimidin-1(2H)-yl)-3-ethylphenyl) piperidin-4-yl)methyl)piperazin-1-yl)-3-ethylphenyl)imidazo[1,5-a]pyrazin-1-yl)benzyl)-5-fluoro-2-methoxybenzamide (Compound I-49)

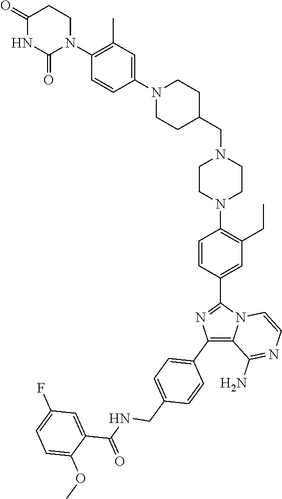 [SMILES: CCc1cc(-c2nc(-c3ccc(CNC(=O)c4cc(F)ccc4OC)cc3)c3c(N)nccn23)ccc1N1CCN(CC2CCN(c3ccc(N4CCC(=O)NC4=O)c(C)c3)CC2)CC1]

The synthesis method of N-(4-(8-amino-3-(3-ethyl-4-(piperazin-1-yl)phenyl)imidazo[1,5-a]pyrazin-1-yl)benzyl)-5-fluoro-2-methoxybenzamide follows Example 20.

### Step 1: Synthesis of N-(4-(8-amino-3-(4-(4-(4-(2,4-dioxotetrahydropyrimidin-1(2H)-yl)-3-ethylphenyl) piperidin-4-yl)methyl)piperazin-1-yl)-3-ethylphenyl)imidazo[1,5-a]pyrazin-1-yl)benzyl)-5-fluoro-2-methoxybenzamide

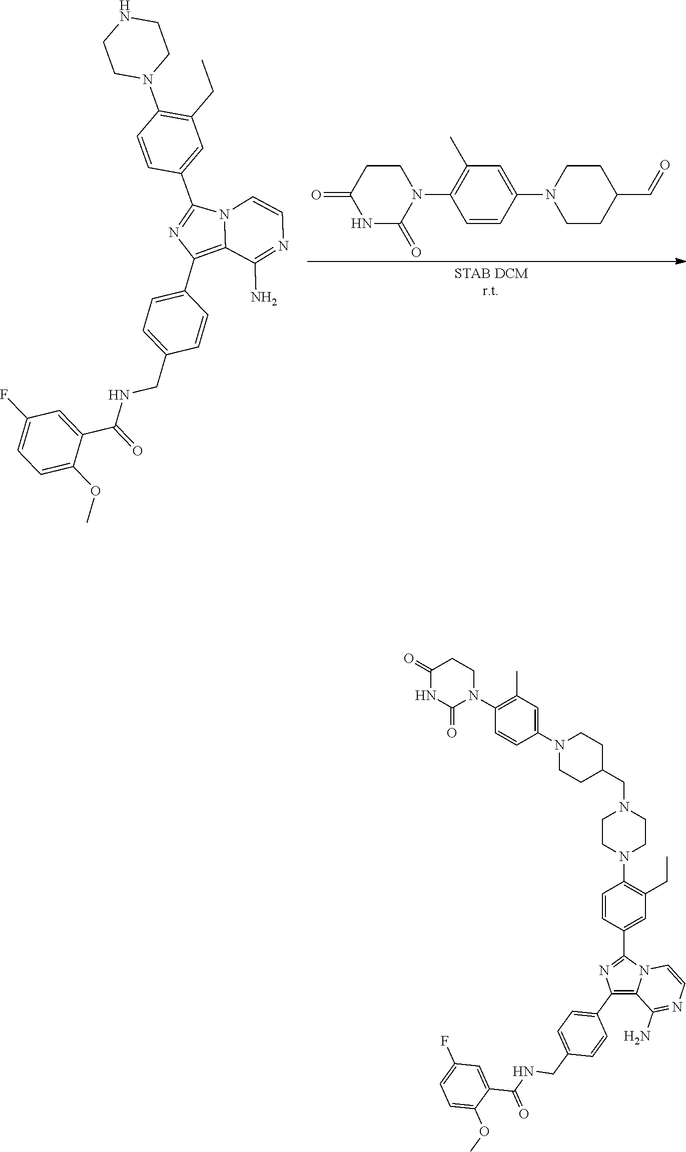

At room temperature, 1-(4-(2,4-dioxotetrahydropyrimidin-1(2H)-yl)-3-methylphenyl)piperidine-4-carbaldehyde (0.05 g, 0.17 mmol), N-(4-(8-amino-3-(3-ethyl-4-(piperazin-1-yl)phenyl)imidazo[1,5-a]pyrazin-1-yl)benzyl)-5-fluoro-2-methoxybenzamide (0.08 g, 0.13 mmol), sodium triacetoxyborohydride (0.04 g, 0.20 mmol) and dichloromethane (10 mL) were added into a single-necked flask in sequence. The reaction was allowed to proceed at room temperature for 2 h under nitrogen protection before termination. The reaction solution was concentrated and purified by column chromatography (MeOH/DCM=1/10) to afford 0.019 g of a yellow solid with a yield of 16%. MS (ESI) m/z: 879.89 [M+H]⁺.

### Example 37: Synthesis of N-(4-(8-amino-3-(4-(1-(1-(4-(2,4-dioxotetrahydropyrimidin-1(2H)-yl)-3-methylphenyl)piperidin-4-yl)methyl)-1,2,3,6-tetrahydropyridin-4-yl)phenyl)imidazo[1,5-a]pyrazin-1-yl)benzyl)-5-fluoro-2-methoxybenzamide (Compound I-50)

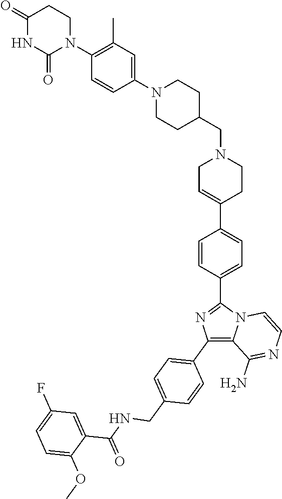 [SMILES: COc1ccc(F)cc1C(=O)NCc1ccc(-c2nc(-c3ccc(C4=CCN(CC5CCN(c6ccc(N7CCC(=O)NC7=O)c(C)c6)CC5)CC4)cc3)n3ccnc(N)c23)cc1]

### Step 1 and Step 2: Synthesis of tert-butyl 4-(4-(8-amino-1-(4-((5-fluoro-2-methoxybenzamido)methyl)phenyl)imidazo[1,5-a]pyrazin-3-yl)phenyl)-3,6-dihydropyridine-1(2H)-carboxylate

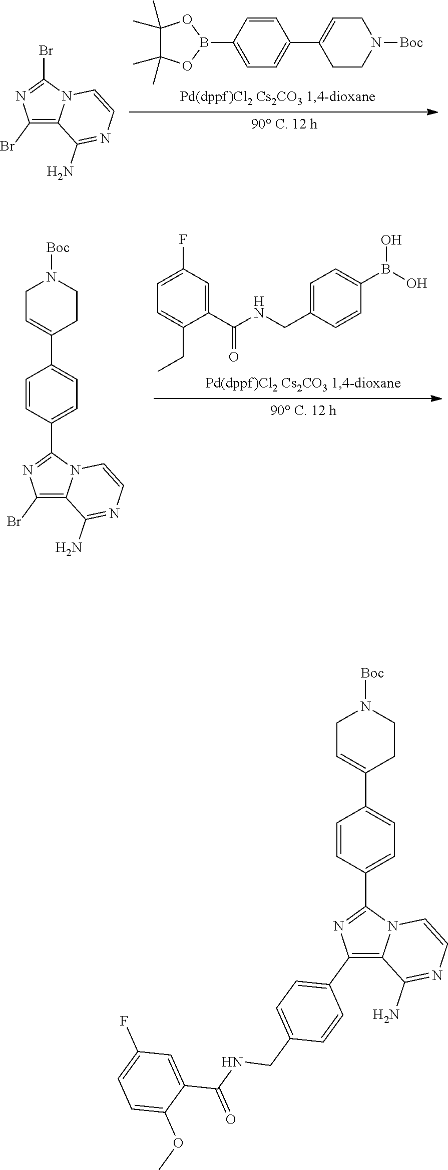 [SMILES: Nc1nccn2c(Br)nc(Br)c12 ; CC(C)(C)OC(=O)N1CC=C(c2ccc(B3OC(C)(C)C(C)(C)O3)cc2)CC1 ; CC(C)(C)OC(=O)N1CC=C(c2ccc(-c3nc(Br)c4c(N)nccn34)cc2)CC1 ; CCc1ccc(F)cc1C(=O)NCc1ccc(B(O)O)cc1 ; COc1ccc(F)cc1C(=O)NCc1ccc(-c2nc(-c3ccc(C4=CCN(C(=O)OC(C)(C)C)CC4)cc3)n3ccnc(N)c23)cc1]

At room temperature, 1,3-dibromoimidazo[1,5-a]pyrazin-8-amine (0.06 g, 0.20 mmol), tert-butyl 4-(4-(4,4,5,5-tetramethyl-1,3,2-dioxaborolan-2-yl)phenyl)-3,6-dihydropyridine-1(2H)-carboxylate (0.08 g, 0.22 mmol), cesium carbonate (0.13 g, 0.40 mmol), Pd(dppf)Cl₂ (0.02 g, 0.04 mmol), 1,4-dioxane (10 mL), ethylene glycol monomethyl ether (5 mL) and H₂O (1 mL) were added into a single-necked flask in sequence. Under nitrogen protection, the mixture was heated to 90° C. and the reaction was allowed to proceed for 12 h. The reaction solution was cooled to room temperature, and (4-((5-fluoro-2-methoxybenzamido)methyl)phenyl)boronic acid (0.06 g, 0.20 mmol) and Pd(dppf)Cl₂ (0.02 g, 0.04 mmol) were added. Under nitrogen protection, the mixture was heated to 90° C. and the reaction was allowed to proceed for another 12 h. The reaction solution was cooled to room temperature and filtered, and the filtrate was concentrated and purified by column chromatography (MeOH/DCM system) to afford 0.04 g of a yellow solid with a yield of 31%.

### Step 3: Synthesis of N-(4-(8-amino-3-(4-(1,2,3,6-tetrahydropyridin-4-yl)phenyl)imidazo[1,5-a]pyrazin-1-yl)benzyl)-5-fluoro-2-methoxybenzamide

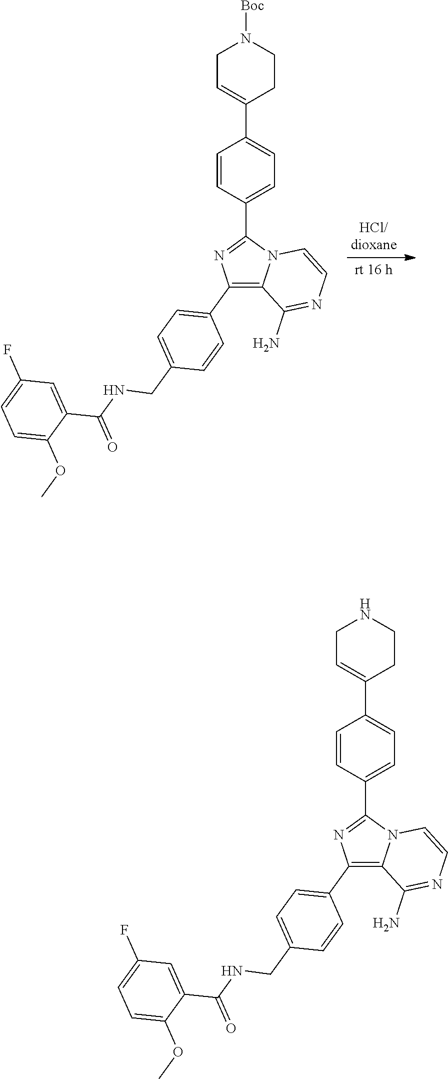 [SMILES: COc1ccc(F)cc1C(=O)NCc1ccc(-c2nc(-c3ccc(C4=CCN(C(=O)OC(C)(C)C)CC4)cc3)n3ccnc(N)c23)cc1 ; COc1ccc(F)cc1C(=O)NCc1ccc(-c2nc(-c3ccc(C4=CCNCC4)cc3)n3ccnc(N)c23)cc1]

At room temperature, tert-butyl 4-(4-(8-amino-1-(4-((5-fluoro-2-methoxybenzamido)methyl)phenyl)imidazo[1,5-a]pyrazin-3-yl)phenyl)-3,6-dihydropyridine-1(2H)-carboxylate (0.04 g, 0.06 mmol), HCl-dioxane (5 mL) and DCM (5 mL) were added into a single-necked flask in sequence and the reaction was allowed to proceed for 16 h. The reaction solution was concentrated, and the concentrated reaction solution was treated with aqueous ammonia and concentrated to afford 0.03 g of a crude product as a yellow solid.

### Step 4: Synthesis of N-(4-(8-amino-3-(4-(1-(1-(4-(2,4-dioxotetrahydropyrimidin-1(2H)-yl)-3-methylphenyl)piperidin-4-yl)methyl)-1,2,3,6-tetrahydropyridin-4-yl)phenyl)imidazo[1,5-a]pyrazin-1-yl)benzyl)-5-fluoro-2-methoxybenzamide

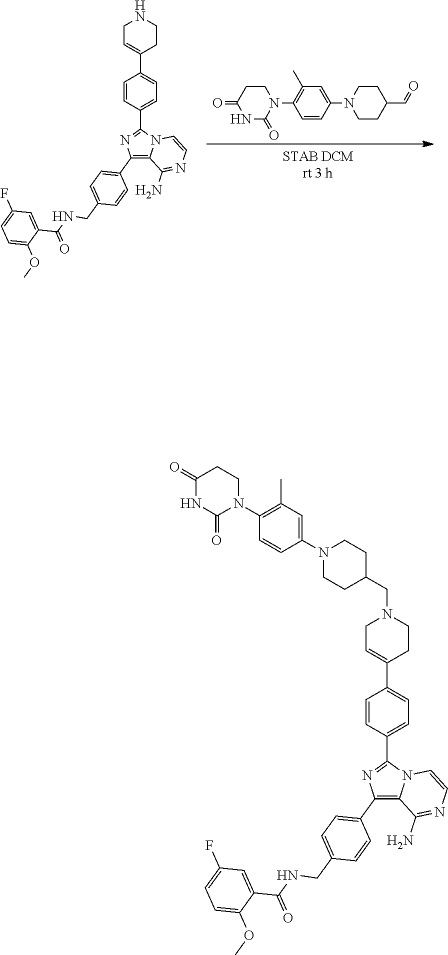 [SMILES: COc1ccc(F)cc1C(=O)NCc1ccc(-c2nc(-c3ccc(C4=CCNCC4)cc3)n3ccnc(N)c23)cc1 ; Cc1cc(N2CCC(C=O)CC2)ccc1N1CCC(=O)NC1=O ; COc1ccc(F)cc1C(=O)NCc1ccc(-c2nc(-c3ccc(C4=CCN(CC5CCN(c6ccc(N7CCC(=O)NC7=O)c(C)c6)CC5)CC4)cc3)n3ccnc(N)c23)cc1]

At room temperature, N-(4-(8-amino-3-(4-(1,2,3,6-tetrahydropyridin-4-yl)phenyl)imidazo[1,5-a]pyrazin-1-yl)benzyl)-5-fluoro-2-methoxybenzamide (0.03 g, 0.06 mmol), 1-(4-(2,4-dioxotetrahydropyrimidin-1(2H)-yl)-3-methylphenyl)piperidine-4-carbaldehyde (0.02 g, 0.07 mmol), sodium triacetoxyborohydride (0.02 g, 0.09 mmol) and DCM (20 mL) were added into a single-necked flask in sequence and the reaction was allowed to proceed at room temperature for 3 h. The reaction solution was concentrated and purified by column chromatography (MeOH/DCM system) to afford 0.008 g of a yellow solid with a yield of 16%. MS (ESI) m/z: 848.9 [M+H]⁺.

### Example 38: Synthesis of N-(4-(8-amino-3-(4-(4-(4-(2,4-dioxotetrahydropyrimidin-1(2H)-yl)-3-methoxyphenyl) piperidin-4-yl)methyl)piperazin-1-yl)-3-methylphenyl)imidazo[1,5-a]pyrazin-1-yl)benzyl)-5-fluoro-2-methoxybenzamide (Compound I-51)

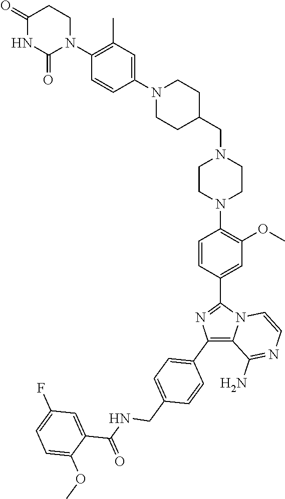 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)c(OC)c3)n3ccnc(N)c23)cc1]

The synthesis method of N-(4-(8-amino-3-(3-methoxy-4-(piperazin-1-yl)phenyl)imidazo[1,5-a]pyrazin-1-yl)benzyl)-5-fluoro-2-methoxybenzamide follows Example 20.

### Step 1: Synthesis of N-(4-(8-amino-3-(4-(4-(4-(2,4-dioxotetrahydropyrimidin-1(2H)-yl)-3-ethylphenyl) piperidin-4-yl)methyl)piperazin-1-yl)-3-methoxyphenyl)imidazo[1,5-a]pyrazin-1-yl)benzyl)-5-fluoro-2-methoxybenzamide

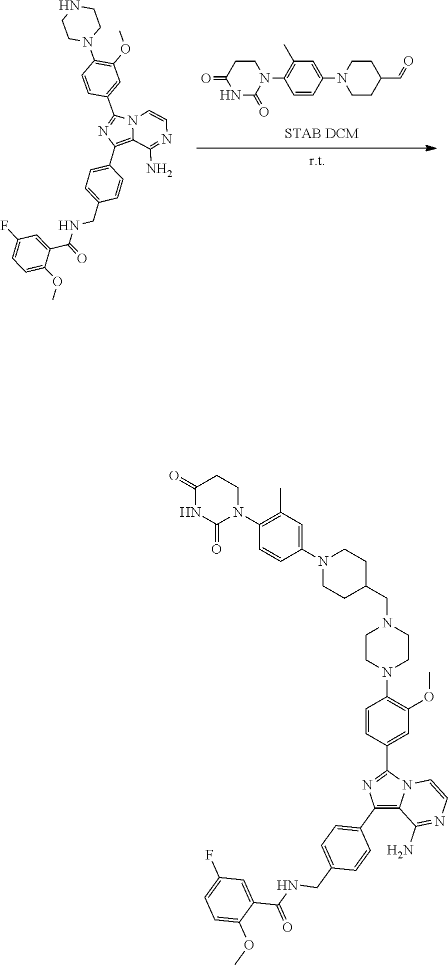 [SMILES: COc1ccc(F)cc1C(=O)NCc1ccc(-c2nc(-c3ccc(N4CCNCC4)c(OC)c3)n3ccnc(N)c23)cc1 ; Cc1cc(N2CCC(C=O)CC2)ccc1N1CCC(=O)NC1=O ; COc1ccc(F)cc1C(=O)NCc1ccc(-c2nc(-c3ccc(N4CCN(CC5CCN(c6ccc(N7CCC(=O)NC7=O)c(C)c6)CC5)CC4)c(OC)c3)n3ccnc(N)c23)cc1]

At room temperature, 1-(4-(2,4-dioxotetrahydropyrimidin-1(2H)-yl)-3-methylphenyl)piperidine-4-carbaldehyde (0.02 g, 0.04 mmol), N-(4-(8-amino-3-(3-methoxy-4-(piperazin-1-yl)phenyl)imidazo[1,5-a]pyrazin-1-yl)benzyl)-5-fluoro-2-methoxybenzamide (0.02 g, 0.03 mmol), sodium triacetoxyborohydride (0.01 g, 0.05 mmol) and dichloromethane (10 mL) were added into a single-necked flask in sequence. The reaction was allowed to proceed at room temperature for 2 h under nitrogen protection before termination. The reaction solution was concentrated and purified on a thick preparative plate (MeOH/DCM=1/10) to afford 0.007 g of a white solid with a yield of 24%. MS (EST) m/z: 881.62 [M+H]⁺.

### Example 39: Synthesis of N-(4-(4-amino-1-(6-(4-((1-(4-(2,4-dioxotetrahydropyrimidin-1(2H)-yl)-3-methylphenyl)piperidin-4-yl)methyl)piperazin-1-yl)pyridin-3-yl)-2-oxo-1,2-dihydro-3H-imidazo[4,5-c]pyridin-3-yl)benzyl)-5-fluoro-2-methoxybenzamide (Compound I-52)

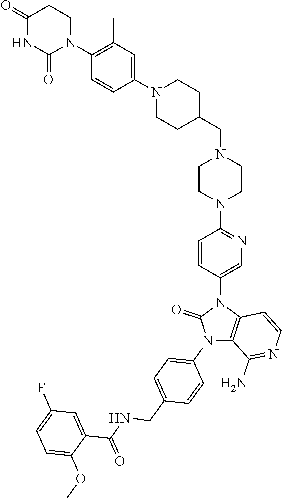 [SMILES: COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)nc3)c3ccnc(N)c32)cc1]

The synthesis method of N-(4-(4-amino-2-oxo-1-(6-(piperazin-1-yl)pyridin-3-yl)-1,2-dihydro-3H-imidazo[4,5-c]pyridin-3-yl)benzyl)-5-fluoro-2-methoxybenzamide follows Example 35.

### Step 1: Synthesis of N-(4-(4-amino-1-(6-(4-((1-(4-(2,4-dioxotetrahydropyrimidin-1(2H)-yl)-3-methylphenyl)piperidin-4-yl)methyl)piperazin-1-yl)pyridin-3-yl)-2-oxo-1,2-dihydro-3H-imidazo[4,5-c]pyridin-3-yl)benzyl)-5-fluoro-2-methoxybenzamide

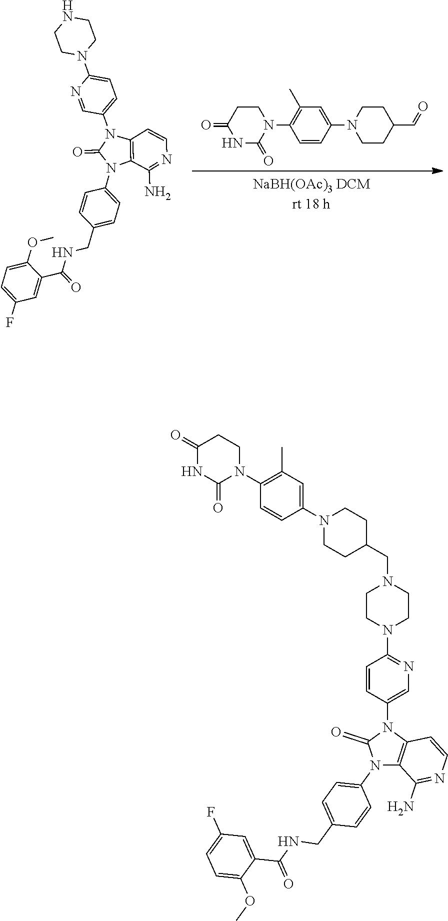 [SMILES: COc1ccc(F)cc1C(=O)NCc1ccc(-n2c(=O)n(-c3ccc(N4CCNCC4)nc3)c3ccnc(N)c32)cc1 ; Cc1cc(N2CCC(C=O)CC2)ccc1N1CCC(=O)NC1=O ; COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)nc3)c3ccnc(N)c32)cc1]

At room temperature, N-(4-(4-amino-2-oxo-1-(6-(piperazin-1-yl)pyridin-3-yl)-1,2-dihydro-3H-imidazo[4,5-c]pyridin-3-yl)benzyl)-5-fluoro-2-methoxybenzamide (0.06 g, 0.10 mmol), 1-(4-(2,4-dioxotetrahydropyrimidin-1(2H)-yl)-3-methylphenyl)piperidine-4-carbaldehyde (0.04 g, 0.12 mmol), sodium triacetoxyborohydride (0.03 g, 0.15 mmol) and DCM (20 mL) were added into a single-necked flask in sequence and the reaction was allowed to proceed at room temperature for 16 h. The reaction solution was concentrated and purified by column chromatography (MeOH/DCM system) to afford 0.03 g of a yellow solid with a yield of 31%. MS (ESI) m/z: 868.6 [M+H]⁺.

### Example 40: Synthesis of N-(4-(8-amino-3-(4-(6-(1-(4-(2,4-dioxotetrahydropyrimidin-1(2H)-yl)-3-methylphenyl)piperidin-4-yl)methyl)-3,6-diazabicyclo[3.1.1]heptane-3-yl)phenyl)imidazo[1,5-a]pyrazin-1-yl)benzyl)-5-fluoro-2-methoxybenzamide (Compound I-53)

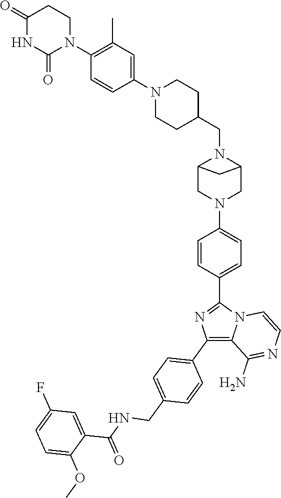 [SMILES: COc1ccc(F)cc1C(=O)NCc1ccc(-c2nc(-c3ccc(N4CC5CC(C4)N5CC4CCN(c5ccc(N6CCC(=O)NC6=O)c(C)c5)CC4)cc3)n3ccnc(N)c23)cc1]

The synthesis method of N-(4-(3-(4-(3,6-diazabicyclo[3.1.1]heptane-3-yl)phenyl)-8-aminoimidazo[1,5-a]pyrazin-1-yl)benzyl)-5-fluoro-2-methoxybenzamide follows Example 20.

### Step 1: Synthesis of N-(4-(8-amino-3-(4-(6-(1-(4-(2,4-dioxotetrahydropyrimidin-1(2H)-yl)-3-methylphenyl)piperidin-4-yl)methyl)-3,6-diazabicyclo[3.1.1]heptane-3-yl)phenyl)imidazo[1,5-a]pyrazin-1-yl)benzyl)-5-fluoro-2-methoxybenzamide

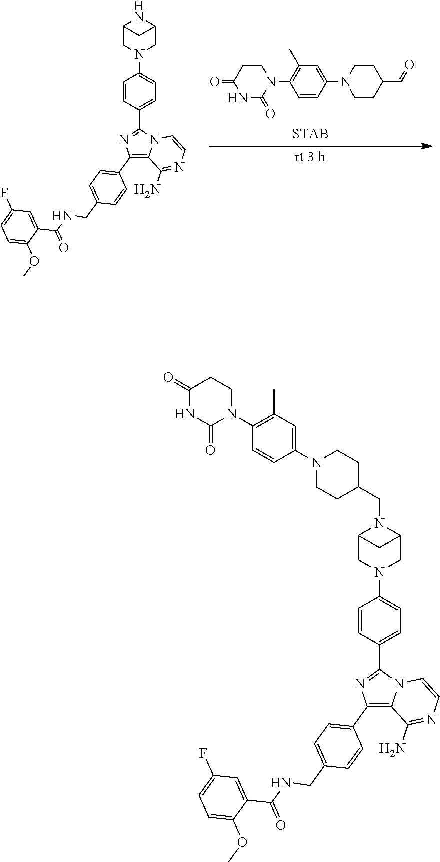 [SMILES: COc1ccc(F)cc1C(=O)NCc1ccc(-c2nc(-c3ccc(N4CC5CC(C4)N5)cc3)n3ccnc(N)c23)cc1 ; Cc1cc(N2CCC(C=O)CC2)ccc1N1CCC(=O)NC1=O ; COc1ccc(F)cc1C(=O)NCc1ccc(-c2nc(-c3ccc(N4CC5CC(C4)N5CC4CCN(c5ccc(N6CCC(=O)NC6=O)c(C)c5)CC4)cc3)n3ccnc(N)c23)cc1]

At room temperature, N-(4-(3-(4-(3,6-diazabicyclo[3.1.1]heptane-3-yl)phenyl)-8-aminoimidazo[1,5-a]pyrazin-1-yl)benzyl)-5-fluoro-2-methoxybenzamide (0.07 g, 0.12 mmol), 1-(4-(2,4-dioxotetrahydropyrimidin-1(2H)-yl)-3-methylphenyl)piperidine-4-carbaldehyde (0.05 g, 0.14 mmol), sodium triacetoxyborohydride (0.04 g, 0.18 mmol) and DCM (20 mL) were added into a single-necked flask in sequence and the reaction was allowed to proceed at room temperature for 16 h. The reaction solution was poured into a saturated aqueous sodium carbonate solution and extracted twice with DCM, the organic phases were combined, dried, and filtered, and the filtrate was concentrated and purified by column chromatography (MeOH/DCM system) to afford 0.02 g of a yellow solid with a yield of 19%. MS (ESI) m/z: 863.4 [M+H]⁺.

### Example 41: Synthesis of 5-(4-((1-(5-(4-amino-3-(4-phenoxyphenyl)-1H-pyrazolo[3,4-d]pyrimidin-1-yl)pyridin-2-yl) piperidin-4-yl)methyl)piperazin-1-yl)-2-(2,6-dioxopiperidin-3-yl) isoindoline-1,3-dione (Compound II-37)

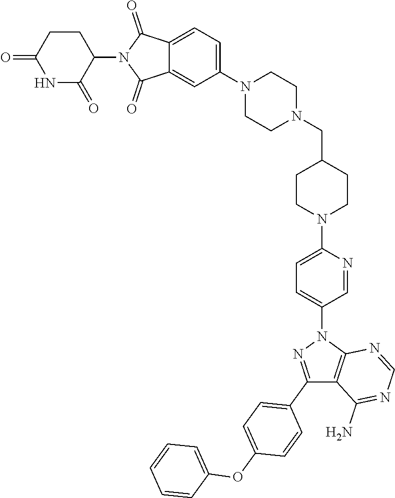 [SMILES: Nc1ncnc2c1c(-c1ccc(Oc3ccccc3)cc1)nn2-c1ccc(N2CCC(CN3CCN(c4ccc5c(c4)C(=O)N(C4CCC(=O)NC4=O)C5=O)CC3)CC2)nc1]

### Step 1: Synthesis of 5-bromo-2-(4-(dimethoxymethyl) piperidin-1-yl)pyridine

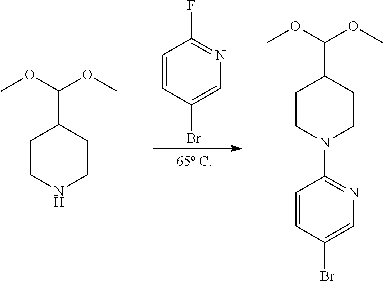

At room temperature, 2-fluoro-5-bromopyridine (3.52 g, 19.89 mmol), 4-(dimethoxymethyl)piperidine (3.80 g, 23.86 mmol), DMSO (60 mL) and DIEA (7.71 g, 59.66 mmol) were added into a single-necked flask in sequence. Under nitrogen protection, the mixture was heated to 65° C. and the reaction was allowed to proceed for 15 h before termination. The reaction solution was diluted with EA and washed with water and saturated brine, and the organic phase was dried over anhydrous Na₂SO₄, filtered, concentrated and purified by column chromatography (EA/PE system) to afford 4.50 g of a white solid. The total yield of the two steps was 73%. MS (ESI) m/z: 315.3 [M+H]⁺.

### Step 2: Synthesis of (E)-N,N-dimethyl-N-(3-(4-phenoxyphenyl)-1H-pyrazolo[3,4-d]pyrimidin-4-yl) carboxamide

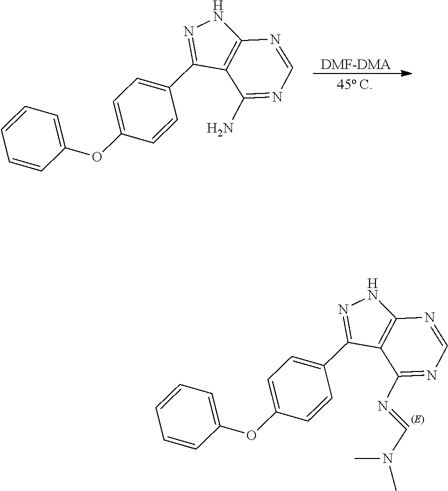

At room temperature, ibrutinib intermediate N-2 (2.00 g, 6.59 mmol) and DMF-DMA (30 mL) were added into a single-necked flask in sequence. Under nitrogen protection, the mixture was heated to 45° C. and the reaction was allowed to proceed for 3 h before termination. The reaction solution was concentrated and purified by column chromatography (MeOH/DCM system) to afford 2.64 g of a white solid. MS (ESI) m/z: 359.5 [M+H]⁺.

### Step 3: Synthesis of 1-(6-(4-(dimethoxymethyl) piperidin-1-yl)pyridin-3-yl)-3-(4-phenoxyphenyl)-1-H-pyrazolo[3,4-d]pyrimidine-4-amine

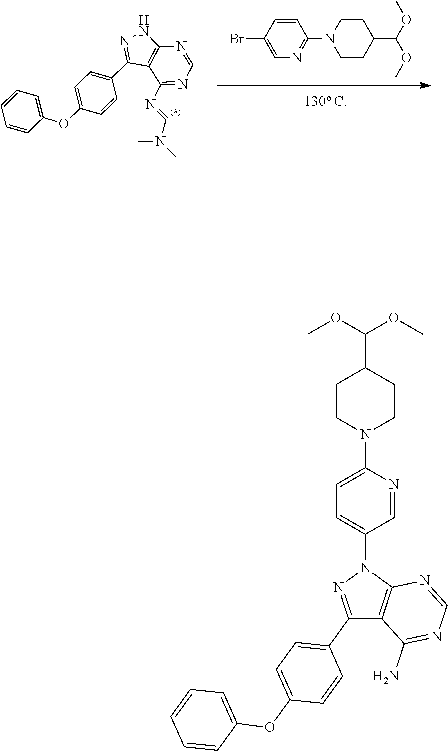 [SMILES: CN(C)/C=N/c1ncnc2[nH]nc(-c3ccc(Oc4ccccc4)cc3)c12 ; COC(OC)C1CCN(c2ccc(Br)cn2)CC1 ; COC(OC)C1CCN(c2ccc(-n3nc(-c4ccc(Oc5ccccc5)cc4)c4c(N)ncnc43)cn2)CC1]

At room temperature, (E)-N,N-dimethyl-N-(3-(4-phenoxyphenyl)-1H-pyrazolo[3,4-d]pyrimidin-4-yl) carboxamide (0.30 g, 0.84 mmol), 5-bromo-2-(4-(dimethoxymethyl) piperidin-1-yl)pyridine (0.53 g, 1.67 mmol), trans-N¹,N²-dimethylcyclohexane-1,2-diamine (0.06 g, 0.42 mmol), Cs₂CO₃ (0.82 g, 2.51 mmol), CuI (0.08 g, 0.42 mmol) and DMSO (20 mL) were added into a single-necked flask in sequence. Under nitrogen protection, the mixture was heated to 130° C. and the reaction was allowed to proceed for 16 h before termination. The reaction solution was diluted with water and extracted with EA, and the organic phase was dried over anhydrous Na₂SO₄, filtered, concentrated and purified by column chromatography (EA/PE system) to afford 0.15 g of a yellow solid. The total yield of the two steps was 33%, ¹H NMR (400 MHz, DMSO-d₆) δ 8.72 (d, J=2.8 Hz, 1H), 8.32 (s, 1H), 8.12 (dd, J=9.2, 2.8 Hz, 1H), 7.78-7.70 (m, 2H), 7.48-7.41 (m, 2H), 7.23-7.12 (m, 5H), 7.00 (d, J=9.3 Hz, 1H), 4.37 (d, J=12.9 Hz, 2H), 4.08 (d, J=6.9 Hz, 1H), 3.27 (s, 6H), 2.85-2.78 (m, 2H), 1.90-1.83 (m, 1H), 1.75-1.67 (m, 2H), 1.28-1.19 (m, 2H). MS (ESI) m/z: 538.6 [M+H]⁺.

### Step 4: Synthesis of 1-(5-(4-amino-3-(4-phenoxyphenyl)-1H-pyrazolo[3,4-d]pyrimidin-1-yl)pyridin-2-yl)piperidine-4-carboxaldehyde

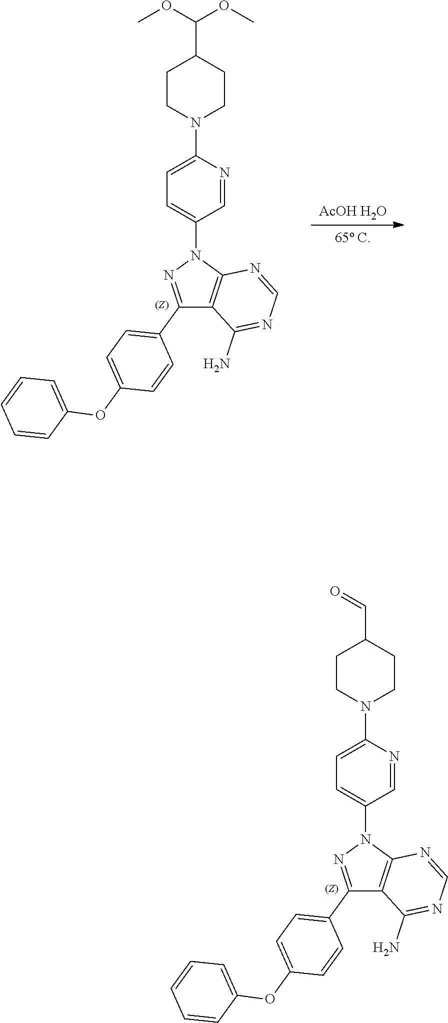 [SMILES: "AcOH H2O" ; COC(OC)C1CCN(c2ccc(-n3nc(-c4ccc(Oc5ccccc5)cc4)c4c(N)ncnc43)cn2)CC1 ; Nc1ncnc2c1c(-c1ccc(Oc3ccccc3)cc1)nn2-c1ccc(N2CCC(C=O)CC2)nc1]

At room temperature, 1-(6-(4-(dimethoxymethyl) piperidin-1-yl)pyridin-3-yl)-3-(4-phenoxyphenyl)-1-A-pyrazolo[3,4-d]pyrimidine-4-amine (0.15 g, 0.28 mmol), AcOH (10 mL) and H₂O (10 mL) were added into a single-necked flask in sequence. Under nitrogen protection, the mixture was heated to 65° C. and the reaction was allowed to proceed for 16 h before termination. The reaction solution was concentrated to dryness to afford 0.13 g of a yellow solid with a yield of 95%. MS (ESI) m/z: 492.5 [M+H]⁺.

### Step 5: Synthesis of 5-(4-((1-(5-(4-amino-3-(4-phenoxyphenyl)-1H-pyrazolo[3,4-d]pyrimidin-1-yl)pyridin-2-yl) piperidin-4-yl)methyl)piperazin-1-yl)-2-(2,6-dioxopiperidin-3-yl) isoindoline-1,3-dione

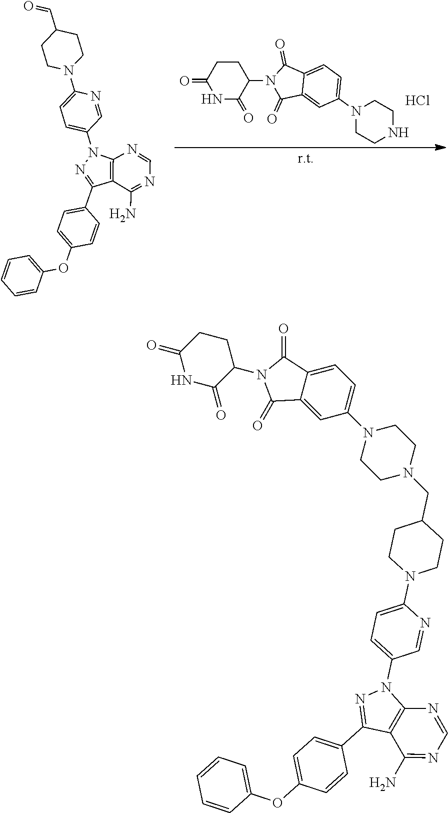 [SMILES: Nc1ncnc2c1c(-c1ccc(Oc3ccccc3)cc1)nn2-c1ccc(N2CCC(C=O)CC2)nc1 ; O=C1CCC(N2C(=O)c3ccc(N4CCNCC4)cc3C2=O)C(=O)N1 ; Cl ; Nc1ncnc2c1c(-c1ccc(Oc3ccccc3)cc1)nn2-c1ccc(N2CCC(CN3CCN(c4ccc5c(c4)C(=O)N(C4CCC(=O)NC4=O)C5=O)CC3)CC2)nc1]

At room temperature, 1-(5-(4-amino-3-(4-phenoxyphenyl)-1H-pyrazolo[3,4-d]pyrimidin-1-yl)pyridin-2-yl)piperidine-4-carboxaldehyde (0.13 g, 0.26 mmol), 2-(2,6-dioxopiperidin-3-yl)-5-(piperazin-1-yl) isoindoline-1,3-dione hydrochloride (0.12 g, 0.32 mmol), DCM (20 mL) and NaBH (OAc)₃ (0.11 g, 0.53 mmol) were added into a single-necked flask in sequence. The reaction was allowed to proceed at room temperature for 4 h under nitrogen protection before termination. The reaction was quenched, and the reaction solution was concentrated and purified by column chromatography (MeOH/DCM system) to afford 0.14 g of a yellow solid with a yield of 65%, 1H NMR (400 MHz, DMSO-d₆) δ 8.72 (d, J=2.8 Hz, 1H), 8.31 (s, 1H), 8.11 (dd, J=9.1, 2.7 Hz, 1H), 7.77-7.71 (m, 2H), 7.67 (d, J=8.5 Hz, 1H), 7.47-7.41 (m, 2H), 7.34 (d, J=2.2 Hz, 1H), 7.25 (dd, J=8.7, 2.3 Hz, 1H), 7.22-7.12 (m, 5H), 6.99 (d, J=9.3 Hz, 1H), 5.07 (dd, J=12.9, 5.4 Hz, 1H), 4.34 (d, J=12.7 Hz, 2H), 3.49-3.41 (m, 6H), 3.33-3.25 (m, 2H), 2.93-2.80 (m, 3H), 2.63-2.52 (m, 2H), 2.49-2.46 (m, 2H), 2.25-2.16 (m, 2H), 2.04-1.98 (m, 1H), 1.85-1.75 (m, 3H), 1.19-1.09 (m, 2H). MS (ESI) m/z: 819.2 [M+H]⁺.

### Example 42: Synthesis of 5-amino-1-(6-(4-((1-(2-(2,6-dioxopiperidin-3-yl)-3-oxoisoindolin-5-yl) piperidin-4-yl)methyl)piperazin-1-yl)pyridin-3-yl)-4-(((5-fluoro-2-methoxybenzamido)methyl)phenyl)-1H-pyrazole-4-carboxamide (Compound II-34)

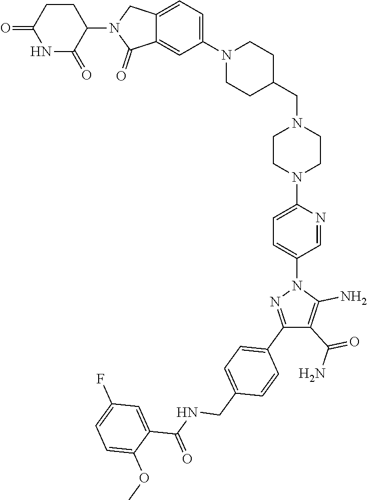 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)nc3)c(N)c2C(N)=O)cc1]

The synthesis method of 5-amino-3-(4-((5-fluoro-2-methoxybenzamido)methyl)phenyl)-1-(6-(piperazin-1-yl)pyridin-3-yl)-1H-pyrazole-4-carboxamide follows Example 28.

### Step 1:5-amino-1-(6-(4-((1-(2-(2,6-dioxopiperidin-3-yl)-3-oxoisoindolin-5-yl) piperidin-4-yl)methyl)piperazin-1-yl)pyridin-3-yl)-4-(((5-fluoro-2-methoxybenzamido)methyl)phenyl)-1H-pyrazole-4-carboxamide

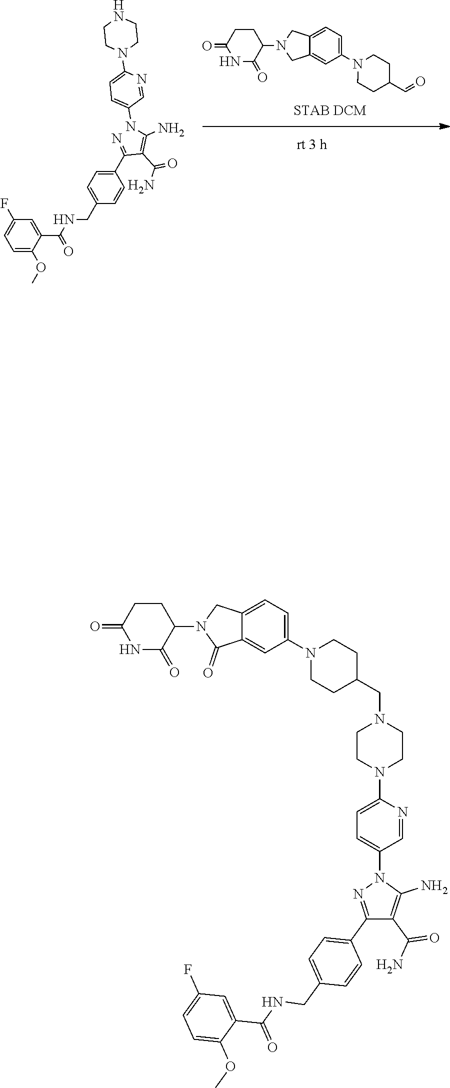 [SMILES: COc1ccc(F)cc1C(=O)NCc1ccc(-c2nn(-c3ccc(N4CCNCC4)nc3)c(N)c2C(N)=O)cc1 ; O=CC1CCN(c2ccc3c(c2)CN(C2CCC(=O)NC2=O)C3)CC1 ; COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)nc3)c(N)c2C(N)=O)cc1]

At room temperature, 5-amino-3-(4-((5-fluoro-2-methoxybenzamido)methyl)phenyl)-1-(6-(piperazin-1-yl)pyridin-3-yl)-1H-pyrazole-4-carboxamide (0.20 g, 0.37 mmol), 1-(2-(2,6-dioxopiperidin-3-yl)-3-oxoisoindolin-5-yl)piperidine-4-carboxaldehyde (0.16 g, 0.44 mmol), sodium triacetoxyborohydride (0.12 g, 0.55 mmol) and DCM (20 mL) were added into a single-necked flask in sequence and the reaction was allowed to proceed at room temperature for 3 h. The reaction solution was concentrated and subjected to column chromatography (MeOH/DCM system) to afford 0.13 g of a white solid with a yield of 40%, ¹H NMR (400 MHz, DMSO-d₆) δ 10.96 (s, 1H), 8.87 (d, J=6.2 Hz, 1H), 8.28 (s, 1H), 7.68 (dd, J=9.0, 2.7 Hz, 1H), 7.57-7.48 (m, 4H), 7.44 (d, J=7.9 Hz, 1H), 7.41-7.30 (m, 1H), 7.19 (dd, J=9.1, 4.3 Hz, 1H), 7.05 (d, J=8.4 Hz, 2H), 6.97 (d, J=9.1 Hz, 1H), 6.36 (s, 1H), 5.05 (dd, J=13.3, 5.1 Hz, 1H), 4.56 (d, J=6.1 Hz, 2H), 4.33 (d, J=16.8 Hz, 1H), 4.20 (d, J=16.8 Hz, 1H), 3.95-3.80 (m, 4H), 3.59-3.55 (m, 3H), 3.38 (s, 2H), 3.17 (d, J=4.8 Hz, 2H), 2.97-2.74 (m, 3H), 2.59 (d, J=14.9 Hz, 1H), 2.49-2.45 (m, 3H), 2.21 (d, J=6.6 Hz, 2H), 1.96 (d, J=11.9 Hz, 1H), 1.82 (d, J=11.7 Hz, 3H), 1.21 (d, J=12.6 Hz, 2H). MS (ESI) m/z: 884.6 [M+H]⁺.

### Example 43: Synthesis of 5-amino-1-(4-(1-(2,6-dioxopiperidin-3-yl)-3-oxoisoindolin-5-yl) piperidin-4-yl)methyl)piperazin-1-yl)phenyl)-3-(4-(5-fluoro-2-methoxybenzamidomethyl)phenyl)-1H-pyrazole-4-carboxamide (Compound II-10)

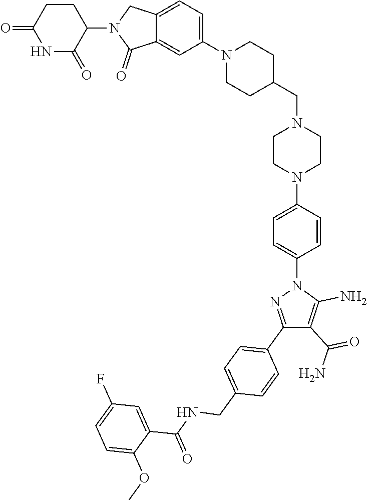 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)cc3)c(N)c2C(N)=O)cc1]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

### Step 1: Synthesis of 5-amino-1-(4-(1-(2,6-dioxopiperidin-3-yl)-3-oxoisoindolin-5-yl) piperidin-4-yl)methyl)piperazin-1-yl)phenyl)-3-(4-(5-fluoro-2-methoxybenzamidomethyl)phenyl)-1H-pyrazole-4-carboxamide

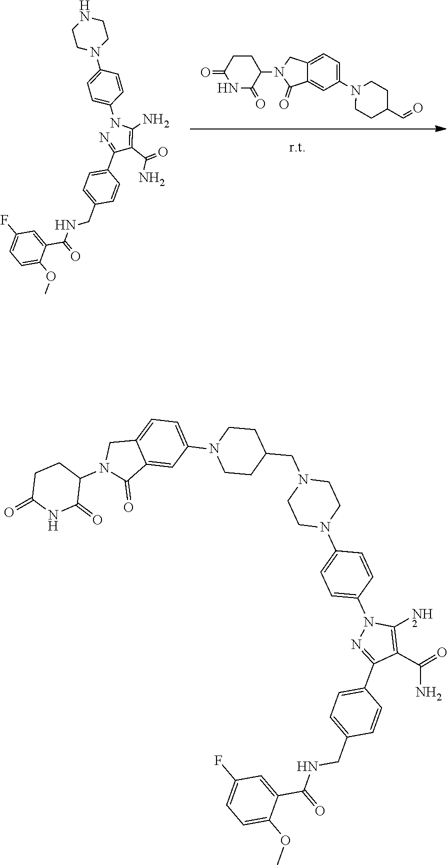 [SMILES: COc1ccc(F)cc1C(=O)NCc1ccc(-c2nn(-c3ccc(N4CCNCC4)cc3)c(N)c2C(N)=O)cc1 ; O=CC1CCN(c2ccc3c(c2)C(=O)N(C2CCC(=O)NC2=O)C3)CC1 ; COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)cc3)c(N)c2C(N)=O)cc1]

At room temperature, 5-amino-3-(4-(5-fluoro-2-methoxybenzamidomethyl)phenyl)-1-(4-(piperazin-1-yl)phenyl)-1H-pyrazole-4-carboxamide (0.04 g, 0.07 mmol), 1-(2-(2,6-dioxopiperidin-3-yl)-3-oxoisoindolin-5-yl)piperidine-4-carboxaldehyde (0.04 g, 0.10 mmol), sodium triacetoxyborohydride (0.02 g, 0.11 mmol) and DCM (10 mL) were added into a single-necked flask in sequence. The reaction was allowed to proceed at room temperature for 2 h under nitrogen protection before termination. The reaction solution was concentrated and purified by column chromatography (MeOH/DCM system) to afford 0.03 g of a white solid with a yield of 45%, 1H NMR (400 MHz, DMSO-d₆) δ 8.87 (s, 1H), 7.71-7.30 (m, 8H), 7.13 (m, 5H), 6.29 (s, 1H), 5.04 (m, 1H), 4.56 (m, 2H), 4.38-4.12 (m, 2H), 3.89 (m, 4H), 3.15 (m 8H), 3.00-2.75 (m, 3H), 2.22 (m, 5H), 1.85 (m, 5H), 1.21 (m, 3H). MS (ESI) m/z: 883.86 [M+H]⁺.

### Example 44: Synthesis of N-(4-(4-amino-1-(6-((2S)-4-((1-(2-(2,6-dioxopiperidin-3-yl)-1-oxoisoindol-5-yl) piperidin-4-yl)methyl)-2-methylpiperazin-1-yl)pyridin-3-yl)-1H-pyrazolo[3,4-d]pyrimidin-3-yl)benzyl)-5-fluoro-2-methoxybenzamide (Compound II-38)

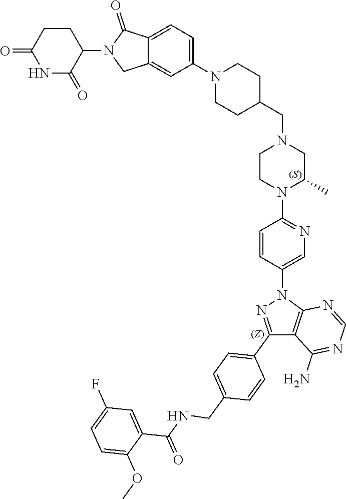

The synthesis method of (S)—N-(4-(4-amino-1-(6-(2-methylpiperazin-1-yl)pyridin-3-yl)-1H-pyrazolo[3,4-d]pyrimidin-3-yl)benzyl)-5-fluoro-2-methoxybenzamide follows Example 1.

### Step 1: Synthesis of N-(4-(4-amino-1-(6-((28)-4-((1-(2-(2,6-dioxopiperidin-3-yl)-1-oxoisoindol-5-yl) piperidin-4-yl)methyl)-2-methylpiperazin-1-yl)pyridin-3-yl)-1H-pyrazolo[3,4-d]pyrimidin-3-yl)benzyl)-5-fluoro-2-methoxybenzamide

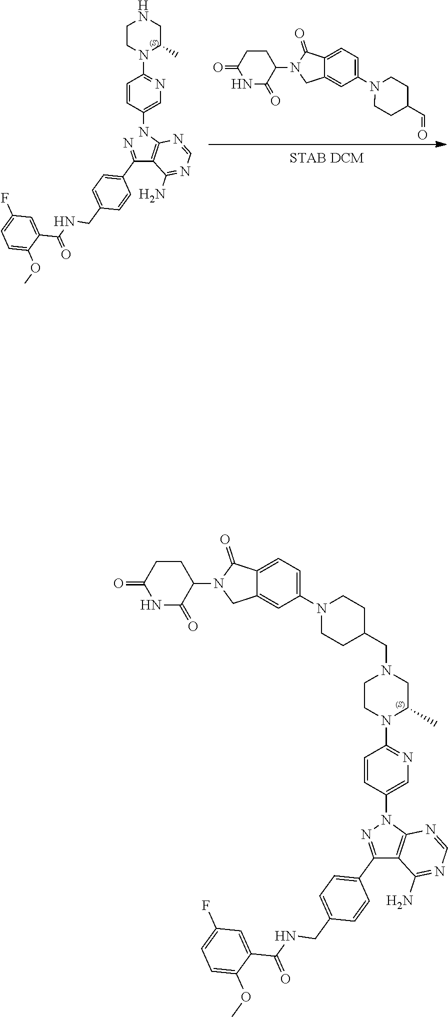

At room temperature, (S)—N-(4-(4-amino-1-(6-(2-methylpiperazin-1-yl)pyridin-3-yl)-1H-pyrazolo[3,4-d]pyrimidin-3-yl)benzyl)-5-fluoro-2-methoxybenzamide (0.08 g, 0.14 mmol), 1-(2-(2,6-dioxopiperidin-3-yl)-1-oxoisoindol-5-yl)piperidine-4-carbaldehyde (0.06 g, 0.17 mmol), sodium triacetoxyborohydride (0.04 g, 0.21 mmol) and DCM (20 mL) were added into a single-necked flask in sequence and the reaction was allowed to proceed at room temperature for 1 b. The reaction solution was concentrated and subjected to column chromatography (MeOH/DCM system) to afford 0.006 g of a white solid with a yield of 5%. MS (ESI) m/z: 907.8 [M+H]⁺.

### Example 45: Synthesis of 5-amino-1-(4-(1-((1-(2-(2,6-dioxopiperidin-3-yl)-3-oxoisoindolin-5-yl) piperidin-4-yl)methyl)piperidin-4-yl)phenyl)-3-(4-(((5-fluoro-2-methoxybenzamido)methyl)phenyl)-1H-pyrazole-4-carboxamide (Compound II-39)

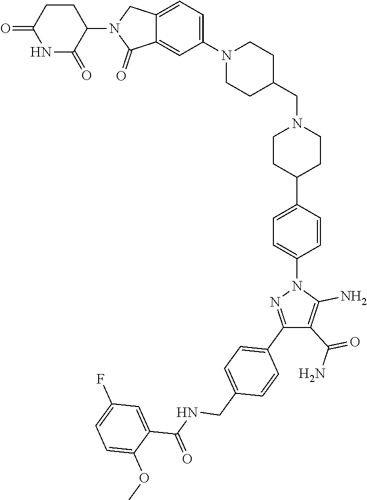 [SMILES: COc1ccc(F)cc1C(=O)NCc1ccc(-c2nn(-c3ccc(C4CCN(CC5CCN(c6ccc7c(c6)C(=O)N(C6CCC(=O)NC6=O)C7)CC5)CC4)cc3)c(N)c2C(N)=O)cc1]

The synthesis method of 5-amino-3-(4-((5-fluoro-2-methoxybenzamido)methyl)phenyl)-1-(4-(piperidin-4-yl)phenyl)-1H-pyrazole-4-carboxamide follows Example 28.

### Step 1:5-amino-1-(4-(1-((1-(2-(2,6-dioxopiperidin-3-yl)-3-oxoisoindolin-5-yl) piperidin-4-yl)methyl)piperidin-4-yl)phenyl)-3-(4-(((5-fluoro-2-methoxybenzamido)methyl)phenyl)-1H-pyrazole-4-carboxamide

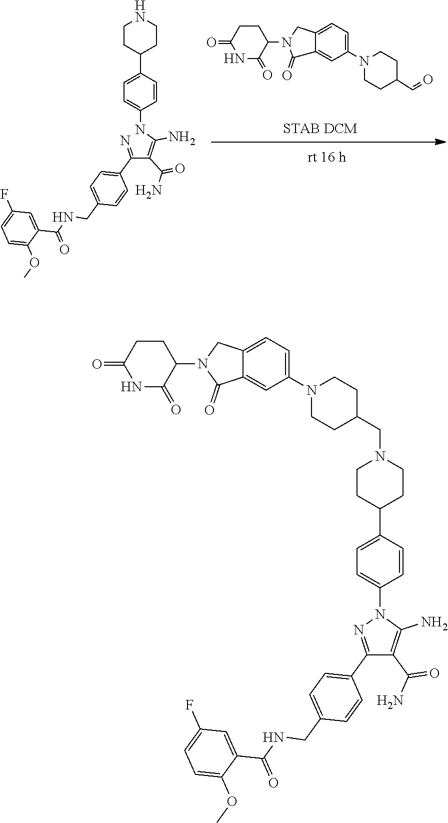 [SMILES: COc1ccc(F)cc1C(=O)NCc1ccc(-c2nn(-c3ccc(C4CCNCC4)cc3)c(N)c2C(N)=O)cc1 ; O=CC1CCN(c2ccc3c(c2)C(=O)N(C2CCC(=O)NC2=O)C3)CC1 ; COc1ccc(F)cc1C(=O)NCc1ccc(-c2nn(-c3ccc(C4CCN(CC5CCN(c6ccc7c(c6)C(=O)N(C6CCC(=O)NC6=O)C7)CC5)CC4)cc3)c(N)c2C(N)=O)cc1]

At room temperature, 5-amino-3-(4-((5-fluoro-2-methoxybenzamido)methyl)phenyl)-1-(4-(piperidin-4-yl)phenyl)-1H-pyrazole-4-carboxamide (0.10 g, 0.18 mmol), 1-(2-(2,6-dioxopiperidin-3-yl)-3-oxoisoindolin-5-yl)piperidine-4-carboxaldehyde (0.08 g, 0.22 mmol), sodium triacetoxyborohydride (0.06 g, 0.28 mmol) and DCM (20 mL) were added into a single-necked flask in sequence and the reaction was allowed to proceed at room temperature for 16 h. The reaction solution was concentrated and subjected to column chromatography (MeOH/DCM system) to afford 0.09 g of a yellow solid with a yield of 58%. ¹H NMR (400 MHz, DMSO-d₆) δ 10.95 (s, 1H), 8.86 (t, J=6.1 Hz, 1H), 7.62-7.31 (m, 10H), 7.23-7.11 (m, 2H), 7.05 (d, J=8.3 Hz, 2H), 6.45 (s, 1H), 5.05 (dd, J=13.3, 5.1 Hz, 1H), 4.56 (d, 2H), 4.33 (d, J=16.8 Hz, 1H), 4.20 (d, J=16.8 Hz, 1H), 4.00-3.74 (m, 5H), 3.40 (s, 1H), 3.18 (s, 1H), 3.09-2.74 (m, 5H), 2.63-2.55 (m, 2H), 2.24 (d, J=7.7 Hz, 1H), 2.08 (s, 3H), 2.00-1.93 (m, 3H), 1.86-1.66 (m, 6H), 1.21 (d, J=11.8 Hz, 2H). MS (ESI) m/z: 882.9 [M+H]⁺.

### Example 46: Synthesis of N-(4-(4-amino-1-(4-(-4-((1-(2-(2,6-dioxopiperidin-3-yl)-1-oxoisoindol-5-yl) piperidin-4-yl)methyl)piperazin-1-yl)phenyl)-2-oxo-1,2-dihydro-3H-imidazo[4,5-c]pyridin-3-yl)benzyl)-5-fluoro-2-methoxybenzamide (Compound II-24)

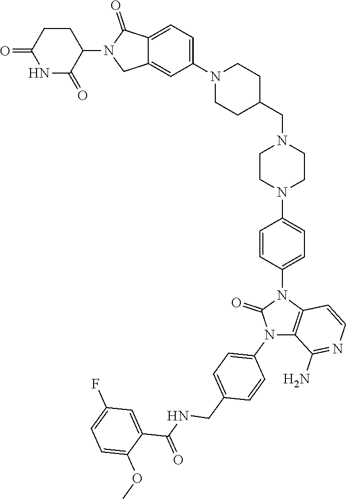 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)cc3)c3ccnc(N)c32)cc1]

The synthesis method of N-(4-(4-amino-2-oxo-1-(4-(piperazin-1-yl)phenyl)-1,2-dihydro-3H-imidazo[4,5-c]pyridin-3-yl)benzyl)-5-fluoro-2-methoxybenzamide follows Example 35.

### Step 1: Synthesis of N-(4-(4-amino-1-(4-(-4-((1-(2-(2,6-dioxopiperidin-3-yl)-1-oxoisoindol-5-yl) piperidin-4-yl)methyl)piperazin-1-yl)phenyl)-2-oxo-1,2-dihydro-3H-imidazo[4,5-c]pyridin-3-yl)benzyl)-5-fluoro-2-methoxybenzamide

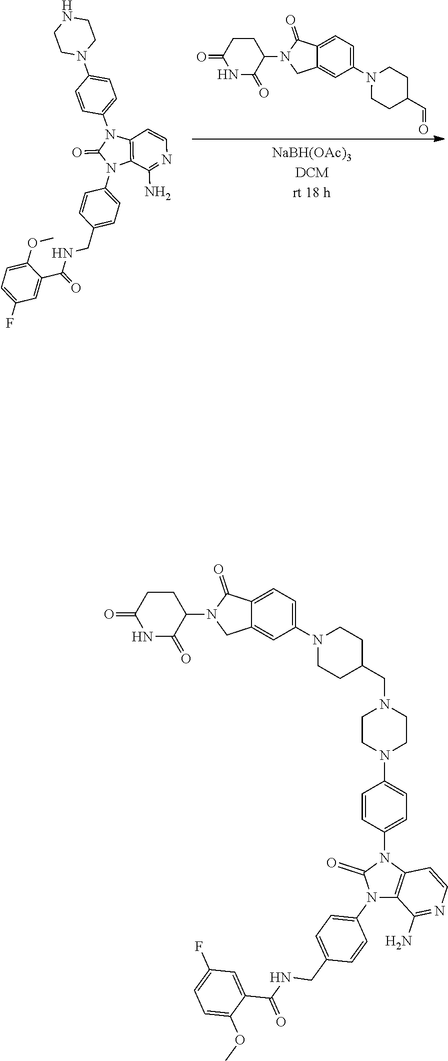 [SMILES: COc1ccc(F)cc1C(=O)NCc1ccc(-n2c(=O)n(-c3ccc(N4CCNCC4)cc3)c3ccnc(N)c32)cc1 ; O=CC1CCN(c2ccc3c(c2)CN(C2CCC(=O)NC2=O)C3=O)CC1 ; COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)cc3)c3ccnc(N)c32)cc1]

At room temperature, N-(4-(4-amino-2-oxo-1-(4-(piperazin-1-yl)phenyl)-1,2-dihydro-3H-imidazo[4,5-c]pyridin-3-yl)benzyl)-5-fluoro-2-methoxybenzamide (0.06 g, 0.10 mmol), 1-(2-(2,6-dioxopiperidin-3-yl)-1-oxoisoindolin-5-yl)piperidine-4-carboxaldehyde (0.04 g, 0.12 mmol), sodium triacetoxyborohydride (0.03 g, 0.15 mmol) and DCM (20 mL) were added into a single-necked flask in sequence and the reaction was allowed to proceed at room temperature for 16 h. The reaction solution was concentrated and purified by column chromatography (MeOH/DCM system) to afford 0.03 g of a yellow solid with a yield of 31%. MS (ESI) m/z: 907.6 [M+H]⁺.

### Example 47: Synthesis of N-(4-(4-amino-1-(6-(4-((1-(2-(2,6-dioxopiperidin-3-yl)-1-oxoisoindol-5-yl) piperidin-4-yl)methyl)piperazin-1-yl)pyridin-3-yl)-1H-pyrazolo[4,3-c]pyridin-3-yl)benzyl)-5-fluoro-2-methoxybenzamide (Compound II-12)

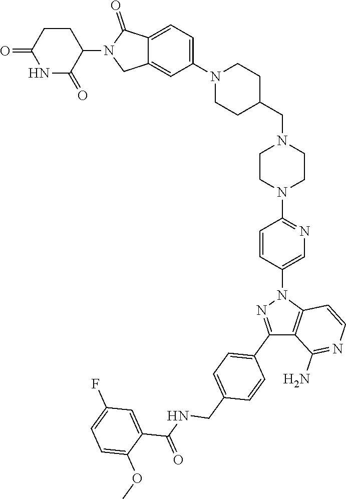 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ccnc(N)c23)cc1]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

### Step 1 Synthesis of (N-(4-aminopyrazolo[4,3-c]pyridin-3-yl)benzyl)-5-fluoro-2-methoxybenzamide

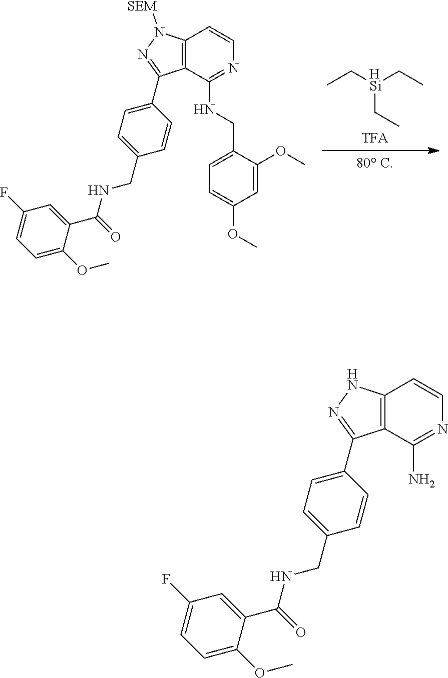

At room temperature, N-(4-(2,4-dimethoxybenzyl)amino)-1-(2-(trimethylsilyl) ethoxy)methyl-1H-pyrazolo[4,3-c]pyridin-3-yl)benzyl)-5-fluoro-2-methoxybenzamide (2.94 g, 4.38 mmol), trifluoroacetic acid (50 mL) and triethylsilane (5 mL) were added into a single-necked flask in sequence. The reaction was allowed to proceed at 80° C. for 1 h under nitrogen protection before termination. The reaction solution was cooled to room temperature, concentrated, and dissolved with a small amount of methanol, and the pH was adjusted to alkaline with ammonia water. The alkaline solution was diluted with water. The filter cake was collected by filtration and dried to afford 2.34 g of a crude product as a white solid.

### Step 2. Synthesis of tert-butyl 4-(5-(4-amino-3-(4-((5-fluoro-2-methoxybenzamido)methyl)phenyl)-1H-pyrazolo[4,3-c]pyridin-1-yl)pyridin-2-yl)piperazine-1-carboxylate

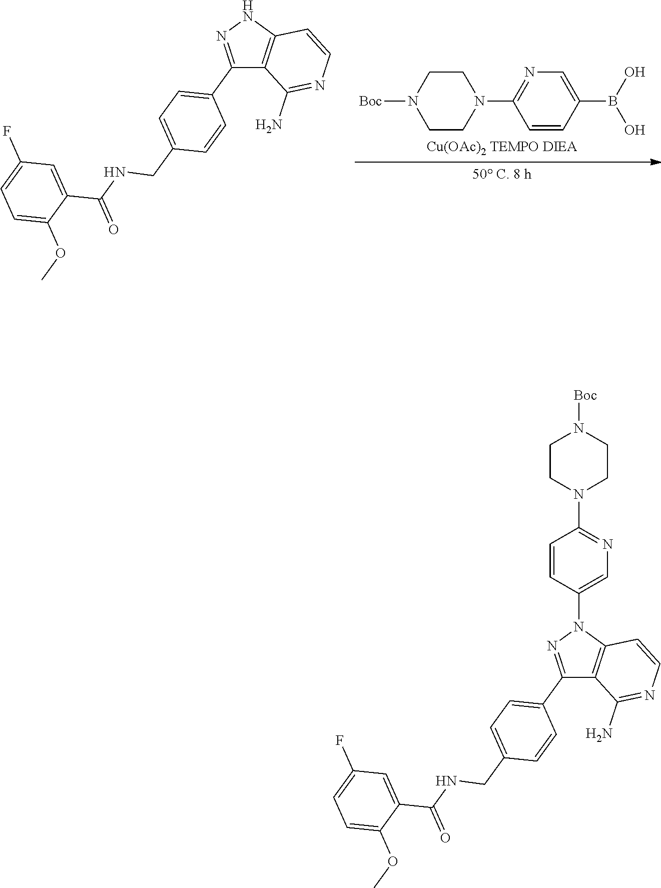 [SMILES: COc1ccc(F)cc1C(=O)NCc1ccc(-c2n[nH]c3ccnc(N)c23)cc1 ; CC(C)(C)OC(=O)N1CCN(c2ccc(B(O)O)cn2)CC1 ; COc1ccc(F)cc1C(=O)NCc1ccc(-c2nn(-c3ccc(N4CCN(C(=O)OC(C)(C)C)CC4)nc3)c3ccnc(N)c23)cc1]

At room temperature, (N-(4-aminopyrazolo[4,3-c]pyridin-3-yl)benzyl)-5-fluoro-2-methoxybenzamide (0.18 g, 0.46 mmol), (6-(4-(tert-butoxycarbonyl) piperazin-1-yl)pyridin-3-yl) boronic acid (0.17 g, 0.55 mmol), copper acetate (0.18 g, 0.92 mmol), TEMPO (0.07 g, 0.46 mmol), DIEA (0.18 g, 1.38 mmol), molecular sieves (4A) (0.1 g) and DMSO (20 mL) were added into a single-necked flask in sequence. The reaction was allowed to proceed at 50° C. for 8 h under oxygen atmosphere before termination. The reaction solution was cooled to room temperature, diluted with water, and extracted with EA, and the organic phase was dried over anhydrous Na₂SO₄, filtered, concentrated and purified by column chromatography (MeOH/DCM system) to afford 0.11 g of a black solid.

### Step 3: Synthesis of N-(4-(4-amino-1-(6-(piperazin-1-yl)pyridin-3-yl)-1H-pyrazolo[4,3-c]pyridin-3-yl)benzyl)-5-fluoro-2-methoxybenzamide

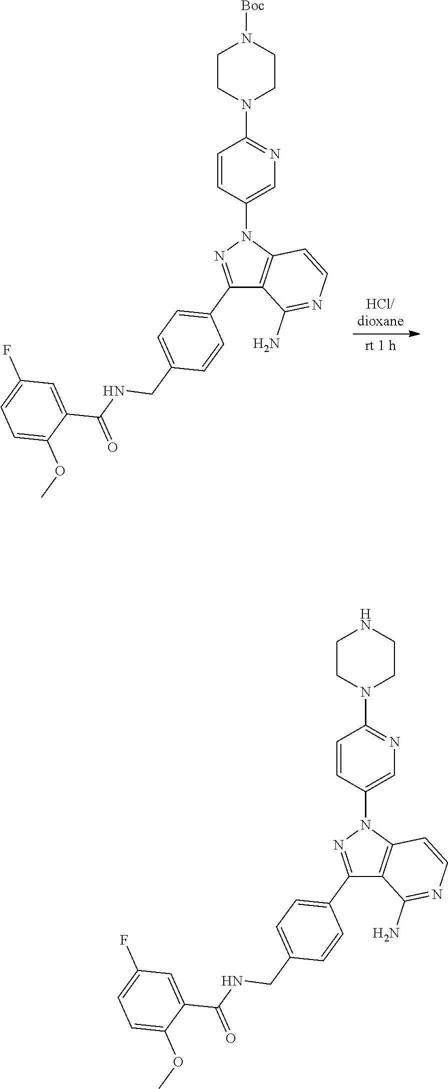 [SMILES: COc1ccc(F)cc1C(=O)NCc1ccc(-c2nn(-c3ccc(N4CCN(C(=O)OC(C)(C)C)CC4)nc3)c3ccnc(N)c23)cc1 ; COc1ccc(F)cc1C(=O)NCc1ccc(-c2nn(-c3ccc(N4CCNCC4)nc3)c3ccnc(N)c23)cc1]

At room temperature, tert-butyl 4-(5-(4-amino-3-(4-((5-fluoro-2-methoxybenzamido)methyl)phenyl)-1H-pyrazolo[4,3-c]pyridin-1-yl)pyridin-2-yl) piperazine-1-carboxylate (0.11 g, 0.17 mmol), hydrogen chloride (in 1,4-dioxane, 5 mL) and dichloromethane (10 mL) were added into a single-necked flask in sequence. The reaction was allowed to proceed at room temperature for 1 h under nitrogen protection before termination. The reaction solution was concentrated and diluted with a small amount of methanol, and the pH was adjusted to alkaline with ammonia water. The alkaline solution was concentrated to afford 0.09 g of a crude product as a brown solid.

### Step 4: Synthesis of N-(4-(4-amino-1-(6-(4-((1-(2-(2,6-dioxopiperidin-3-yl)-1-oxoisoindol-5-yl) piperidin-4-yl)methyl)piperazin-1-yl)pyridin-3-yl)-1H-pyrazolo[4,3-c]pyridin-3-yl)benzyl)-5-fluoro-2-methoxybenzamide

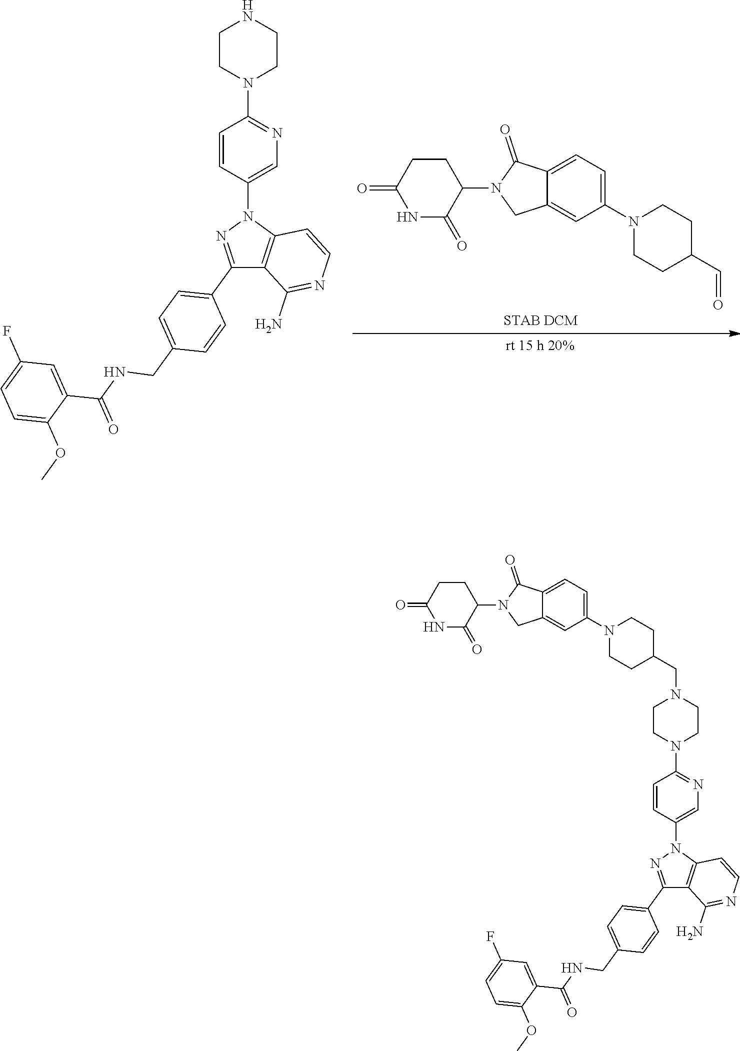 [SMILES: COc1ccc(F)cc1C(=O)NCc1ccc(-c2nn(-c3ccc(N4CCNCC4)nc3)c3ccnc(N)c23)cc1 ; O=CC1CCN(c2ccc3c(c2)CN(C2CCC(=O)NC2=O)C3=O)CC1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ccnc(N)c23)cc1]

At room temperature, N-(4-(4-amino-1-(6-(piperazin-1-yl)pyridin-3-yl)-1H-pyrazolo[4,3-c]pyridin-3-yl)benzyl)-5-fluoro-2-methoxybenzamide (0.09 g, 0.16 mmol), 1-(2-(2,6-dioxopiperidin-3-yl)-1-oxoisoindolin-5-yl)piperidine-4-carboxaldehyde (0.05 g, 0.13 mmol), sodium triacetoxyborohydride (0.04 g, 0.20 mmol) and dichloromethane (15 mL) were added into a single-necked flask in sequence. The reaction was allowed to proceed at room temperature for 16 h under nitrogen protection before termination. The reaction solution was concentrated and purified on a thick preparative plate to afford 0.003 g of a yellow solid with a yield of 20%. MS (ESI) m/z: 891.9 [M+H]⁺.

### Example 48: Synthesis of 1-(4-(4-((4-(5-(8-amino-1-(4-phenoxyphenyl)imidazo[1,5-a]pyrazin-3-yl)pyridin-2-yl) piperazin-1-yl)methyl)piperidin-1-yl)-2-methylphenyl)dihydropyrimidine-2,4(1H,3H)-dione (Compound I-54)

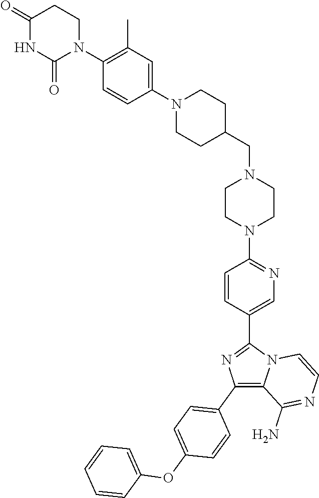 [SMILES: Cc1cc(N2CCC(CN3CCN(c4ccc(-c5nc(-c6ccc(Oc7ccccc7)cc6)c6c(N)nccn56)cn4)CC3)CC2)ccc1N1CCC(=O)NC1=O]

The synthesis method of N-(4-amino-1-(6-(piperazin-1-yl)pyridin-3-yl)-1H-pyrazolo[3,4-d]pyrimidin-3-yl)-2,3-difluorobenzyl)-5-fluoro-2-methoxybenzamide follows Example 1.

### Step 1 and Step 2: Synthesis of tert-butyl 4-(5-(8-amino-1-(4-phenoxyphenyl)imidazo[1,5-a]pyrazin-3-yl)pyridin-2-yl) piperazine-1-carboxylate

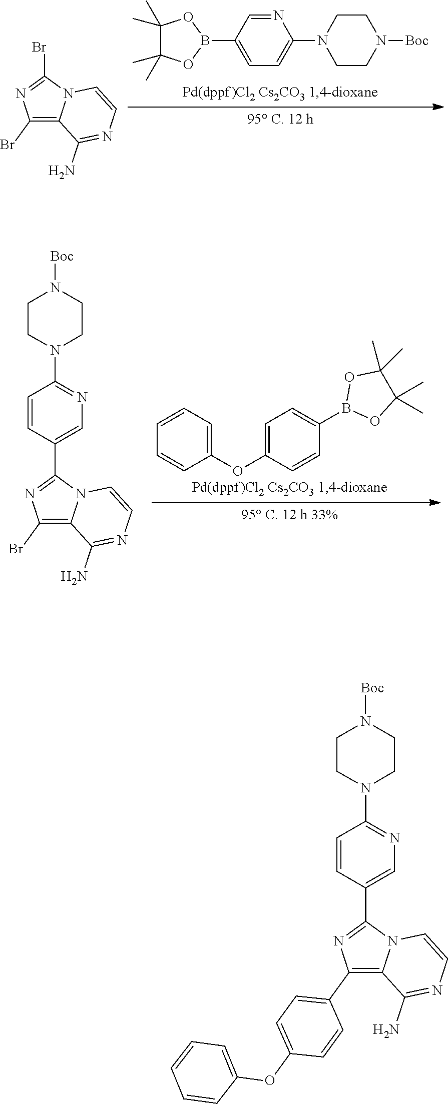

At room temperature, 1,3-dibromoimidazo[1,5-a]pyrazin-8-amine (1.00 g, 3.42 mmol), tert-butyl 4-(5-(4,4,5,5-tetramethyl-1,3,2-dioxaborolan-2-yl)pyridin-2-yl) piperazine-1-carboxylate (1.60 g, 4.11 mmol), cesium carbonate (1.11 g, 3.42 mmol), sodium carbonate (0.36 g, 3.42 mmol), Pd(dppf)Cl₂ (0.25 g, 0.34 mmol), 1,4-dioxane (30 mL), ethylene glycol monomethyl ether (10 mL) and H₂O (2 mL) were added into a single-necked flask in sequence. Under nitrogen protection, the mixture was heated to 90° C. and the reaction was allowed to proceed for 12 h. The reaction solution was cooled to room temperature, and 4,4,5,5-tetramethyl-2-(4-phenoxyphenyl)-1,3,2-dioxaborolane (1.22 g, 4.11 mmol) and Pd(dppf)Cl₂ (0.25 g, 0.34 mmol) were added. Under nitrogen protection, the mixture was heated to 90° C. and the reaction was allowed to proceed for another 12 h. The reaction solution was cooled to room temperature and filtered, and the filtrate was concentrated and purified by column chromatography (MeOH/DCM system) to afford 0.55 g of a yellow solid with a yield of 33%.

### Step 3: Synthesis of 1-(4-phenoxyphenyl)-3-(6-(piperazin-1-yl)pyridin-3-yl) imidazo[1,5-a]pyrazin-8-amine

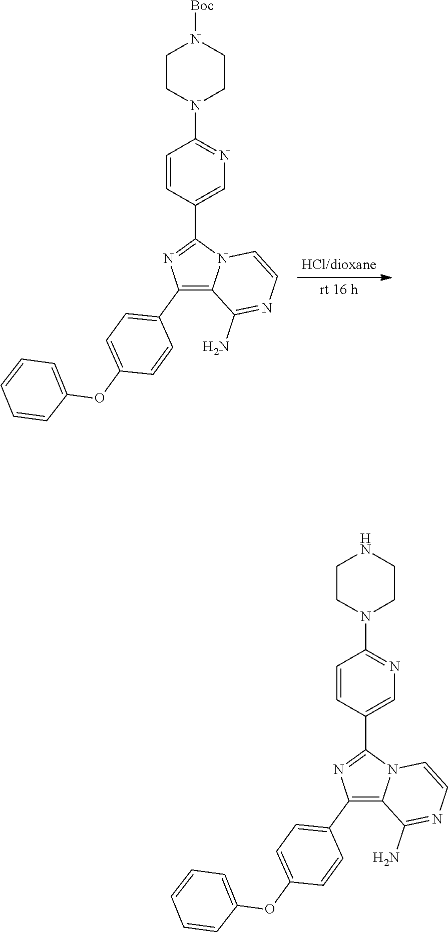 [SMILES: CC(C)(C)OC(=O)N1CCN(c2ccc(-c3nc(-c4ccc(Oc5ccccc5)cc4)c4c(N)nccn34)cn2)CC1 ; Nc1nccn2c(-c3ccc(N4CCNCC4)nc3)nc(-c3ccc(Oc4ccccc4)cc3)c12]

At room temperature, tert-butyl 4-(5-(8-amino-1-(4-phenoxyphenyl)imidazo[1,5-a]pyrazin-3-yl)pyridin-2-yl) piperazine-1-carboxylate (0.55 g, 0.98 mmol), HCl-dioxane (5 mL) and DCM (5 mL) were added into a single-necked flask in sequence and the reaction was allowed to proceed for 16 h. The reaction solution was concentrated, and the concentrated reaction solution was treated with aqueous ammonia and concentrated to afford 0.45 g of a crude product as a yellow solid.

### Step 4: Synthesis of 1-(4-(4-((4-(5-(8-amino-1-(4-phenoxyphenyl)imidazo[1,5-a]pyrazin-3-yl)pyridin-2-yl) piperazin-1-yl)methyl)piperidin-1-yl)-2-methylphenyl)dihydropyrimidine-2,4(1H,3H)-dione

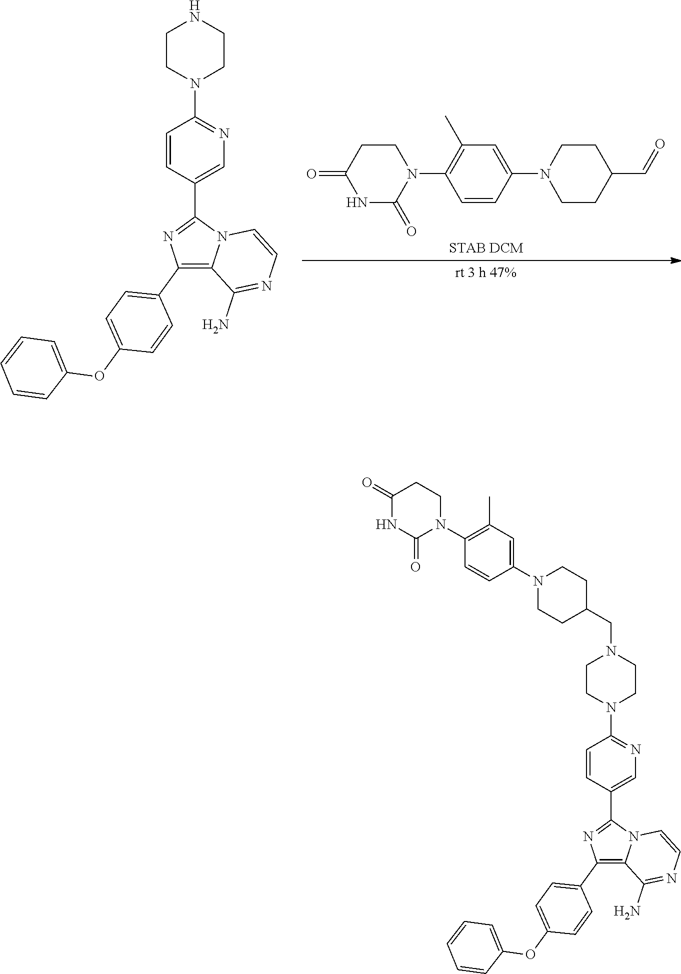 [SMILES: Nc1nccn2c(-c3ccc(N4CCNCC4)nc3)nc(-c3ccc(Oc4ccccc4)cc3)c12 ; Cc1cc(N2CCC(C=O)CC2)ccc1N1CCC(=O)NC1=O ; Cc1cc(N2CCC(CN3CCN(c4ccc(-c5nc(-c6ccc(Oc7ccccc7)cc6)c6c(N)nccn56)cn4)CC3)CC2)ccc1N1CCC(=O)NC1=O]

At room temperature, 1-(4-phenoxyphenyl)-3-(6-(piperazin-1-yl)pyridin-3-yl) imidazo[1,5-a]pyrazin-8-amine (0.45 g, 0.98 mmol), 1-(4-(2,4-dioxotetrahydropyrimidin-1(2H)-yl)-3-methylphenyl)piperidine-4-carbaldehyde (0.37 g, 1.20 mmol), sodium triacetoxyborohydride (0.31 g, 1.47 mmol) and DCM (20 mL) were added into a single-necked flask in sequence and the reaction was allowed to proceed at room temperature for 3 h. The reaction solution was concentrated and purified by column chromatography (MeOH/DCM system) to afford 0.35 g of a yellow solid with a yield of 47%. MS (ESI) m/z: 763.7 [M+H]⁺.

### Example 49: Synthesis of 1-(4-(4-(4-amino-3-(4-phenoxyphenyl)-1H-pyrazolo[3,4-d]pyrimidin-1-yl)phenyl) piperazin-1-yl)methyl)piperidin-1-yl)-2-methylphenyldihydropyrimidine-2,4(1H,3H)-dione (Compound I-55)

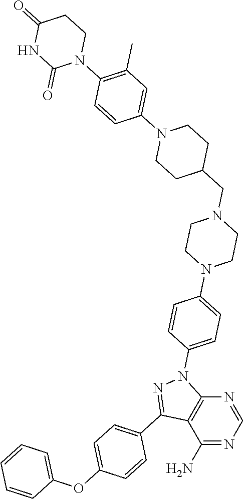 [SMILES: Cc1cc(N2CCC(CN3CCN(c4ccc(-n5nc(-c6ccc(Oc7ccccc7)cc6)c6c(N)ncnc65)cc4)CC3)CC2)ccc1N1CCC(=O)NC1=O]

### Step 1: Synthesis of tert-butyl 4-(4-(4-amino-3-(4-phenoxyphenyl)-1H-pyrazolo[3,4-d]pyrimidin-1-yl)phenyl) piperazine-1-carboxylate

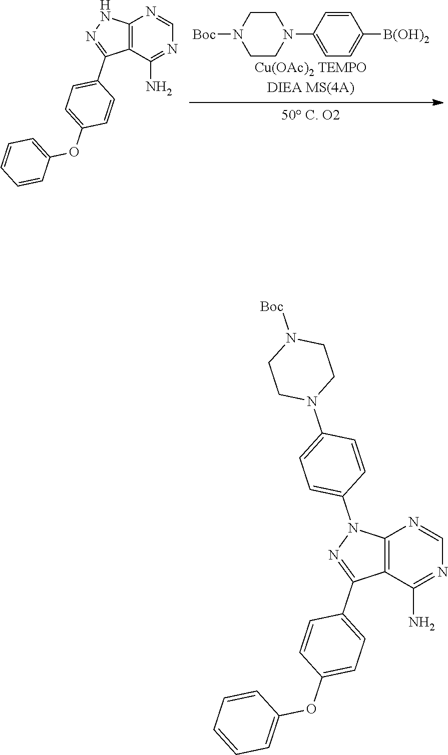 [SMILES: Nc1ncnc2[nH]nc(-c3ccc(Oc4ccccc4)cc3)c12 ; CC(C)(C)OC(=O)N1CCN(c2ccc(B(O)O)cc2)CC1 ; CC(C)(C)OC(=O)N1CCN(c2ccc(-n3nc(-c4ccc(Oc5ccccc5)cc4)c4c(N)ncnc43)cc2)CC1]

At room temperature, ibrutinib intermediate N-2 (1.0 g, 3.30 mmol), (4-(4-(tert-butoxycarbonyl) piperazin-1-yl)phenylboronic acid (1.21 g, 3.96 mmol), anhydrous copper acetate (0.66 g, 3.30 mmol), 2,2,6,6-tetramethylpiperidin-1-oxyl (0.52 g, 3.30 mmol), N,N-diisopropylethylamine (1.28 g, 9.89 mmol), molecular sieves (4A, 0.30 g) and DMSO (30 mL) were added into a single-necked flask in sequence. The reaction was allowed to proceed at 50° C. for 16 h under oxygen atmosphere before termination. The reaction solution was diluted with water and extracted with EA, and the organic phase was dried over anhydrous Na₂SO₄, filtered, concentrated and purified by column chromatography (EA/PE system) to afford 0.42 g of a yellow solid with a yield of 23%.

### Step 2: Synthesis of 3-(4-phenoxyphenyl)-1-(4-(piperazin-1-yl)phenyl)-1H-pyrazolo[3,4-d]pyrimidin-4-amine

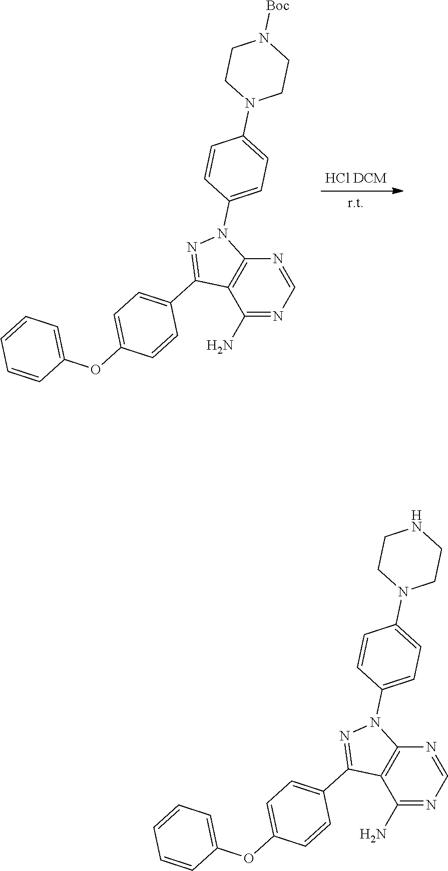 [SMILES: CC(C)(C)OC(=O)N1CCN(c2ccc(-n3nc(-c4ccc(Oc5ccccc5)cc4)c4c(N)ncnc43)cc2)CC1 ; Nc1ncnc2c1c(-c1ccc(Oc3ccccc3)cc1)nn2-c1ccc(N2CCNCC2)cc1]

At room temperature, tert-butyl 4-(4-(4-amino-3-(4-phenoxyphenyl)-1H-pyrazolo[3,4-d]pyrimidin-1-yl)phenyl) piperazine-1-carboxylate (0.42 g, 0.91 mmol), hydrogen chloride (in 1,4-dioxane, 5 mL) and dichloromethane (10 mL) were added into a single-necked flask in sequence. The reaction was allowed to proceed at room temperature for 1 h under nitrogen protection before termination. The reaction solution was concentrated and diluted with a small amount of methanol, and the pH was adjusted to alkaline with ammonia water. The alkaline solution was concentrated to afford 0.40 g of a crude product as a brown solid.

### Step 3: Synthesis of 1-(4-(4-(4-amino-3-(4-phenoxyphenyl)-1H-pyrazolo[3,4-d]pyrimidin-1-yl)phenyl) piperazin-1-yl)methyl)piperidin-1-yl)-2-methylphenyldihydropyrimidine-2,4(1H,3H)-dione

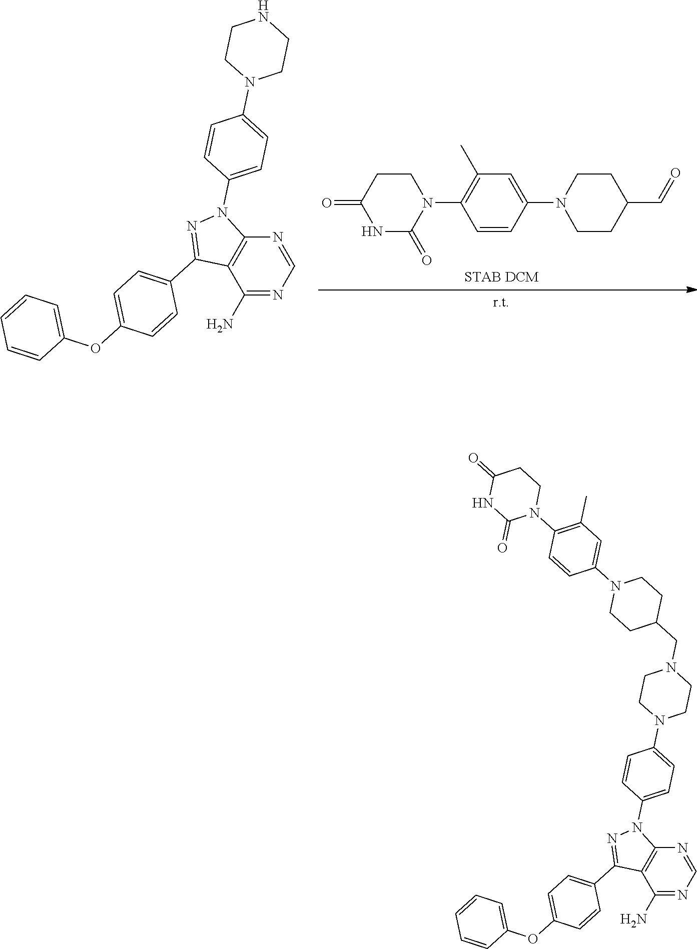 [SMILES: Nc1ncnc2c1c(-c1ccc(Oc3ccccc3)cc1)nn2-c1ccc(N2CCNCC2)cc1 ; Cc1cc(N2CCC(C=O)CC2)ccc1N1CCC(=O)NC1=O ; Cc1cc(N2CCC(CN3CCN(c4ccc(-n5nc(-c6ccc(Oc7ccccc7)cc6)c6c(N)ncnc65)cc4)CC3)CC2)ccc1N1CCC(=O)NC1=O]

At room temperature, 1-(4-(2,4-dioxotetrahydropyrimidin-1(2H)-yl)-3-methylphenyl)piperidine-4-carbaldehyde (0.37 g, 1.18 mmol), 3-(4-phenoxyphenyl)-1-(4-(piperazin-1-yl)phenyl)-1H-pyrazolo[3,4-d]pyrimidin-4-amine (0.40 g, 0.91 mmol), sodium triacetoxyborohydride (0.30 g, 1.37 mmol) and dichloromethane (20 mL) were added into a single-necked flask in sequence. The reaction was allowed to proceed at room temperature for 2 h under nitrogen protection before termination. The reaction solution was concentrated and purified by column chromatography (MeOH/DCM=1/10) to afford 0.29 g of a white solid with a yield of 61%, ¹H NMR (400 MHz, DMSO-d₆) δ 8.34 (s, 1H), 7.97 (d, J=8.6 Hz, 2H), 7.75 (d, J=8.5 Hz, 2H), 7.45 (t, J=7.7 Hz, 2H), 7.31-6.98 (m, 8H), 6.92-6.69 (m, 2H), 3.76-3.61 (m, 3H), 3.46 (m, 1H), 3.20 (m, 4H), 2.76-2.62 (m, 4H), 2.52 (m, 3H), 2.23 (m, 2H), 2.12 (s, 3H), 1.88-1.62 (m, 4H), 1.18 (m, 2H). MS (ESI) m/z: 763.86 [M+H]⁺.

### Example 50: Synthesis of 3-(5-(4-(((1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexyl)methyl)piperazin-1-yl)-1-oxoisoindolin-2-yl)piperidine-2,6-dione (Compound III-19)

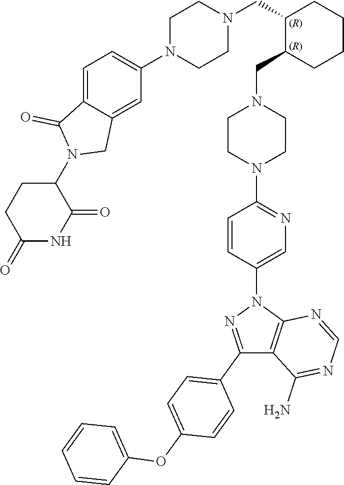

### Step 1: Synthesis of tert-butyl 4-(5-(4-amino-3-(4-phenoxyphenyl)-1H-pyrazolo[3,4-d]pyrimidin-1-yl)pyridin-2-yl) piperazine-1-carboxylate

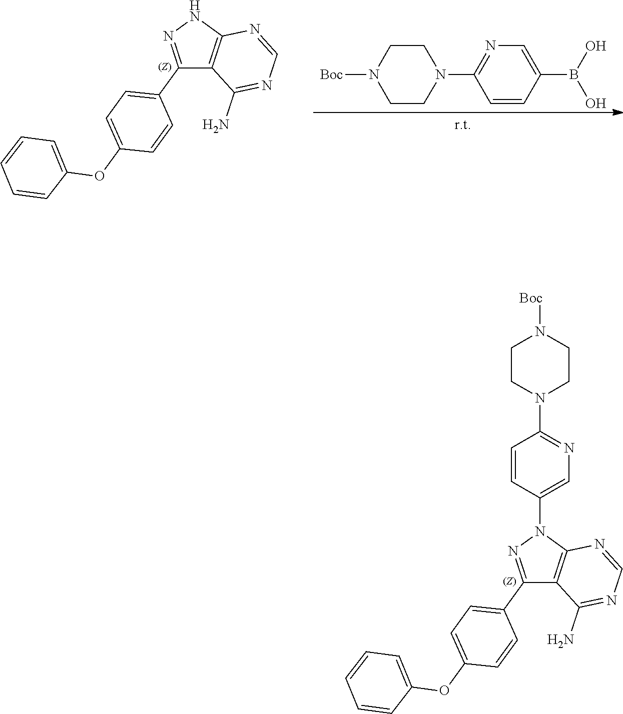 [SMILES: Nc1ncnc2[nH]nc(-c3ccc(Oc4ccccc4)cc3)c12 ; CC(C)(C)OC(=O)N1CCN(c2ccc(B(O)O)cn2)CC1 ; CC(C)(C)OC(=O)N1CCN(c2ccc(-n3nc(-c4ccc(Oc5ccccc5)cc4)c4c(N)ncnc43)cn2)CC1]

At room temperature, ibrutinib intermediate N-2 (2.00 g, 6.59 mmol), (6-(4-(tert-butoxycarbonyl) piperazin-1-yl)pyridin-3-yl) boronic acid (2.23 g, 7.25 mmol), Cu(OAc)₂ (0.66 g, 3.30 mmol), TEMPO (1.13 g, 7.25 mmol), DIEA (3.41 g, 26.37 mmol), DMSO (40 mL) and MS (4A) (2.00 g) were added into a single-necked flask in sequence, and the reaction was allowed to proceed at 50° C. for 18 h under oxygen protection before termination. The reaction solution was diluted with water and extracted with EA. The organic phase was dried over anhydrous Na₂SO₄, filtered, concentrated and purified by column chromatography (EA/PE system) to afford 4.50 g of a brown solid. MS (ESI) m/z: 565.6 [M+H]⁺.

### Step 2: Synthesis of 3-(4-phenoxyphenyl)-1-(6-(piperazin-1-yl)pyridin-3-yl)-1H-pyrazolo[3,4-d]pyrimidine-4-amine

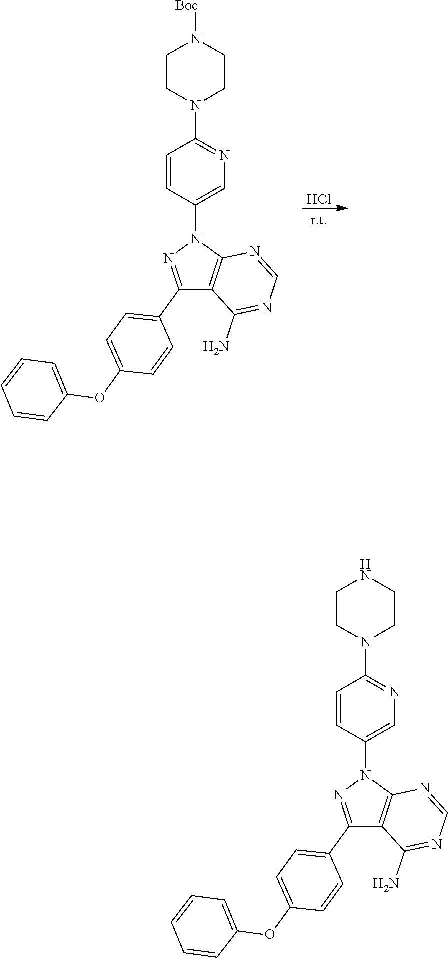 [SMILES: CC(C)(C)OC(=O)N1CCN(c2ccc(-n3nc(-c4ccc(Oc5ccccc5)cc4)c4c(N)ncnc43)cn2)CC1 ; Cl ; Nc1ncnc2c1c(-c1ccc(Oc3ccccc3)cc1)nn2-c1ccc(N2CCNCC2)nc1]

At room temperature, tert-butyl 4-(5-(4-amino-3-(4-phenoxyphenyl)-1H-pyrazolo[3,4-d]pyrimidin-1-yl)pyridin-2-yl) piperazine-1-carboxylate (3.72 g, 6.59 mmol), DCM (20 mL) and HCl/Dioxane (20 mL) were added into a single-necked flask in sequence. The reaction was allowed to proceed at room temperature for 1.0 h under nitrogen protection before termination. The reaction was quenched, the layers were separated, and the aqueous layer was extracted with DCM. The organic phases were combined, dried over anhydrous Na₂SO₄, filtered, and concentrated to afford 2.73 g of a crude product as a brown solid. The total yield of the two steps was 89%. MS (ESI) m/z: 465.5 [M+H]⁺.

### Step 3: Synthesis of (1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexane-1-carbaldehyde

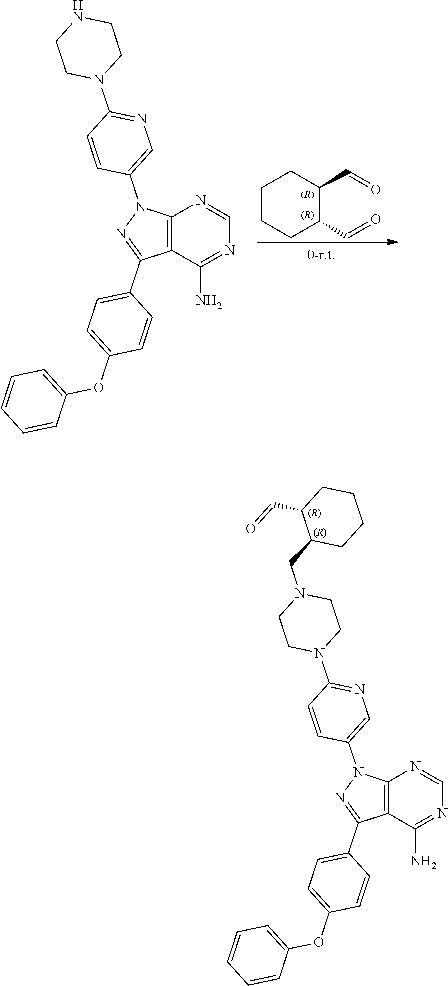

At 0° C., 3-(4-phenoxyphenyl)-1-(6-(piperazin-1-yl)pyridin-3-yl)-1H-pyrazolo[3,4-d]pyrimidine-4-amine (0.50 g, 1.08 mmol), (1R,2R)-cyclohexane-1,2-dicarbaldehyde (1.51 g, 10.76 mmol) and DCM (20 mL) were added to a single-necked flask in sequence, and NaBH(OAc)₃ (0.23 g, 1.08 mmol) was added slowly. Under nitrogen protection, the mixture was warmed to room temperature and the reaction was allowed to proceed for 2 h before termination. The reaction was quenched, the layers were separated, and extraction was performed with DCM. The organic phases were combined, dried over anhydrous Na₂SO₄, filtered, concentrated and purified by column chromatography (MeOH/DCM system) to afford 0.17 g of a yellow solid with a yield of 27%. MS (ESI) m/z: 589.7 [M+H]⁺.

### Step 4:3-(5-(4-(((1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexyl)methyl)piperazin-1-yl)-1-oxoisoindolin-2-yl)piperidine-2,6-dione

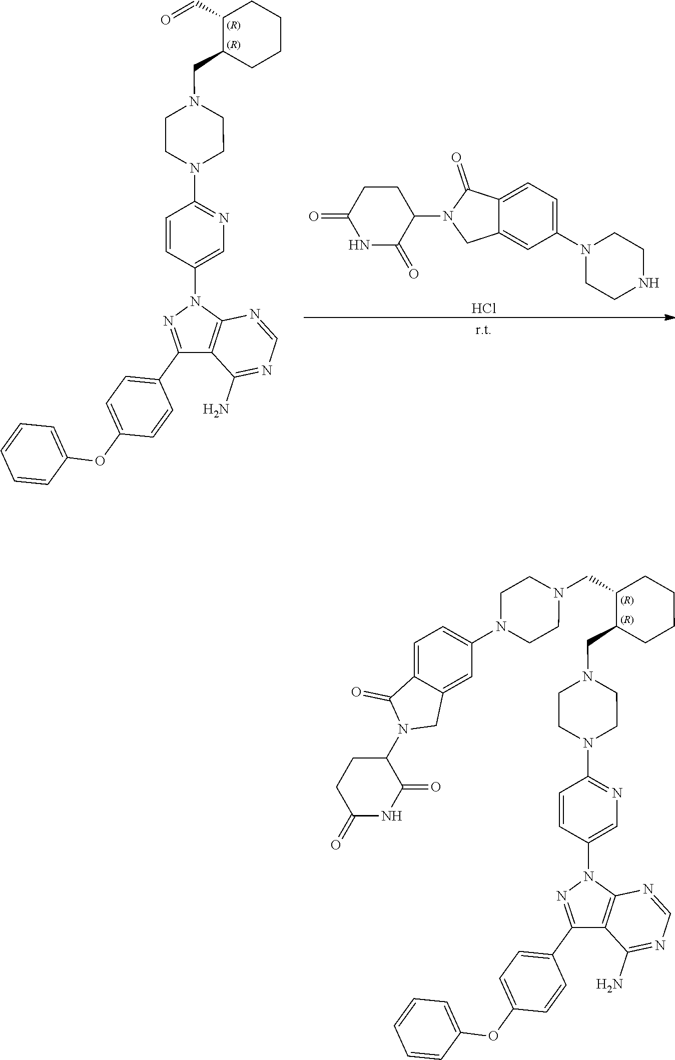

At room temperature, (1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexane-1-carbaldehyde (0.08 g, 0.14 mmol), 3-(1-oxo-5-(piperazin-1-yl) isoindolin-2-yl)piperidine-2,6-dione hydrochloride (0.06 g, 0.14 mmol), DCM (20 mL) and tetraisopropyl titanate (0.21 g, 0.72 mmol) were added to a single-necked flask in sequence. The reaction was allowed to proceed at room temperature for 14 h under nitrogen protection, and NaBH (OAc)₃ (0.06 g, 0.29 mmol) was added. The reaction was allowed to proceed at room temperature for 2 h under nitrogen protection before termination. The reaction was quenched, the reaction solution was filtered and rinsed, and the washings were combined, concentrated and purified by column chromatography (MeOH/DCM system) to afford 0.015 g of a white solid with a yield of 12%, ¹H NMR (400 MHz, DMSO-d₆) δ 8.76 (s, 1H), 8.32 (s, 1H), 8.16 (s, 1H), 7.89-7.67 (m, 3H), 7.60-7.38 (m, 4H), 7.28-6.98 (m, 6H), 5.09-5.00 (m, 1H), 4.37-4.13 (m, 3H), 3.59-3.50 (m, 6H), 3.00-2.81 (m, 2H), 2.44-2.33 (m, 4H), 2.16-1.84 (m, 6H), 1.66-1.42 (m, 5H), 1.21-1.17 (m, 6H), 1.11-0.77 (m, 4H). MS (ESI) m/z: 901.9 [M+H]⁺.

### Example 51: Synthesis of 1-(4-(4-(((1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexyl)methyl)piperazin-1-yl)-2-methylphenyl)dihydropyrimidine-2,4(1H,3H)-dione (Compound III-20)

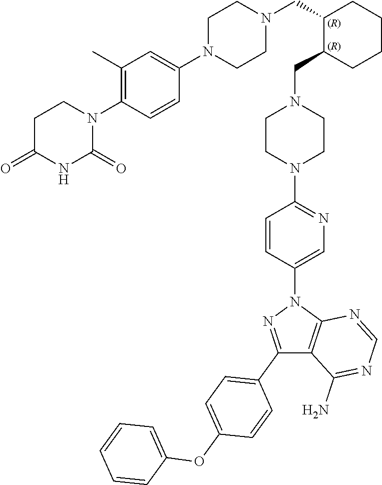

The synthesis method of (1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexane-1-carbaldehyde follows Example 50.

### Step 1:1-(4-(4-(((1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexyl)methyl)piperazin-1-yl)-2-methylphenyl)dihydropyrimidine-2,4(1H,3H)-dione

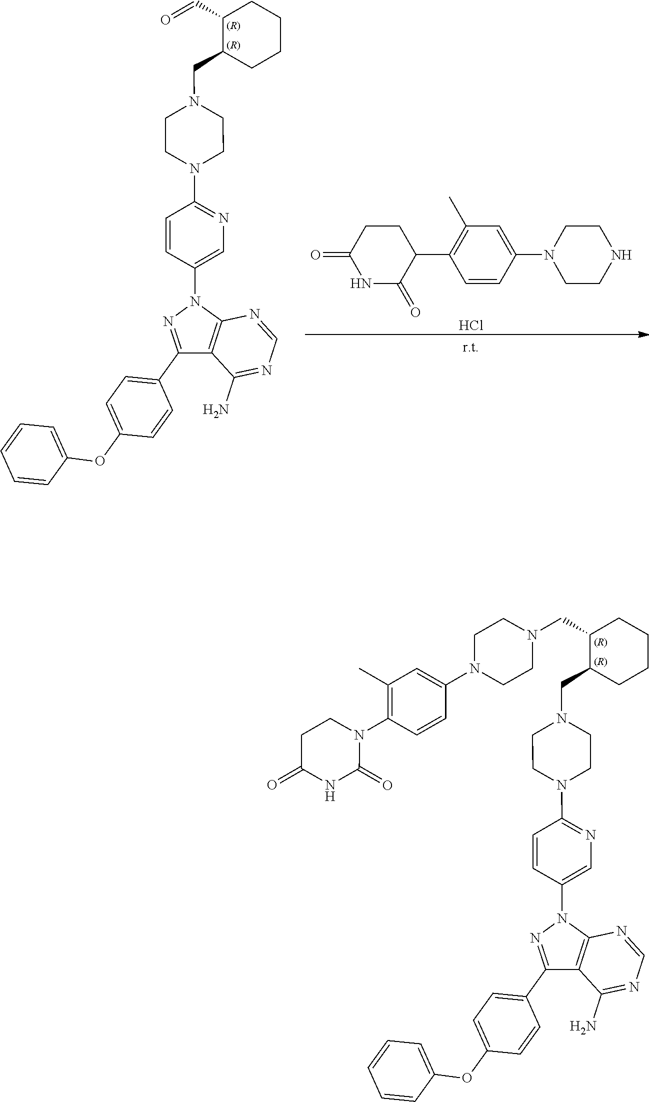

At room temperature, (1R,2R)-2-((4-(5-(4-amino-3-(4-phenoxyphenyl)-1H-pyrazolo[3,4-d]pyrimidin-1-yl)pyridin-2-yl) piperazin-1-yl)methyl)cyclohexane-1-carbaldehyde (0.08 g, 0.14 mmol), 1-(2-methyl-4-(piperazin-1-yl)phenyl)dihydropyrimidine-2,4-(1H,3H)-dione hydrochloride (0.05 g, 0.14 mmol), DCM (20 mL) and tetraisopropyl titanate (0.21 g, 0.72 mmol) were added to a single-necked flask in sequence. The reaction was allowed to proceed at room temperature for 14 h under nitrogen protection, and NaBH (OAc)₃ (0.06 g, 0.29 mmol) was added. The reaction was allowed to proceed at room temperature for 2 h under nitrogen protection before termination. The reaction was quenched, the reaction solution was filtered and rinsed, and the washings were combined, concentrated and purified by column chromatography (MeOH/DCM system) to afford 0.018 g of a white solid with a yield of 15%, ¹H NMR (400 MHz, DMSO-d₆) δ 8.76 (s, 1H), 8.32 (s, 1H), 8.16 (d, J=9.1 Hz, 1H), 7.78-7.71 (m, 2H), 7.44 (t, J=7.8 Hz, 2H), 7.21-7.13 (m, 5H), 7.02 (dd, J=19.0, 8.9 Hz, 2H), 6.84-6.73 (m, 2H), 3.71-3.43 (m, 7H), 3.14-3.09 (m, 3H), 2.74-2.64 (m, 2H), 2.43-2.33 (m, 4H), 2.16-2.07 (m, 5H), 1.95-1.80 (m, 3H), 1.62-4.42 (m, 5H), 1.28-1.17 (m, 6H), 1.05-0.82 (m, 4H). MS (ESI) m/z: 861.8 [M+H]⁺.

### Experimental Example 1: Activity Test in Mino Cells

Cell culture: mino cells (culture conditions: RPMI1640 medium+15% FBS, 37° C., 5% CO₂). Cell plating: The cells subcultured into the logarithmic growth phase were collected and transferred into a centrifuge tube, and centrifuged at 800 rpm for 5 min. The cells were resuspended in complete medium and counted. The cells were diluted and adjusted to a final concentration of 200000 cells/mL. The cells were then seeded into a 96-well plate with 100 μL per well. Cell administration: Each group of drug solutions was added into the 96-well plate, 100 μL per well, n=3 for each drug concentration. Meanwhile, negative wells (n=6) and blank wells (n=3) were set up. The 96-well plate was incubated in the incubator for further 3 days. CCK8 reagent was added to the 96-well plate, 20 μL per well, and then the plate was placed in the incubator for further 4 h. The absorbance of the solution was measured at a wavelength of 450 nm using an enzyme reader. The proliferation inhibition rate was calculated and the half inhibitory concentration IC₅₀ value was calculated.

| Compound | Cell inhibition IC₅₀ (μM) |
|---|---|
| I-2 | 0.014 |
| I-3 | 0.016 |
| I-4 | 0.062 |
| I-5 | 0.084 |
| I-6 | 0.016 |
| I-7 | <0.050 |
| I-8 | <0.025 |
| I-9 | <0.025 |
| I-11 | <0.025 |
| I-12 | <0.025 |
| I-18 | <0.025 |
| I-19 | <0.025 |
| I-24 | <0.100 |
| I-30 | <0.050 |
| I-39 | <0.100 |
| I-40 | <0.025 |
| I-41 | <0.025 |
| I-42 | <0.025 |
| I-43 | <0.050 |
| I-44 | <0.025 |
| I-45 | <0.050 |
| I-46 | <0.100 |
| I-47 | <0.025 |
| I-48 | <0.025 |
| I-49 | <0.100 |
| I-50 | <0.025 |
| I-51 | <0.025 |
| I-52 | <0.050 |
| I-53 | <0.050 |
| I-54 | <0.050 |
| I-55 | <0.100 |
| II-1 | 0.004 |
| II-2 | 0.017 |
| II-3 | 0.017 |
| II-4 | 0.012 |
| II-5 | 0.009 |
| II-6 | 0.021 |
| II-7 | 0.018 |
| II-8 | 0.053 |
| II-10 | <0.025 |
| II-12 | <0.500 |
| II-24 | <0.050 |
| II-34 | <0.025 |
| II-37 | <0.025 |
| II-38 | <0.025 |
| II-39 | <0.025 |
| III-2 | — |
| III-19 | <0.100 |
| III-20 | <0.500 |

Conclusion: The compounds of the present invention have good inhibitory activity in mino cells.

### Experimental Example 2: BTK Protein Degradation Activity Test

Cell culture: Ramos cells (culture conditions: RPMI1640 medium+10% FBS, 37° C., 5% CO₂); DOHH-2 cells (culture conditions: RPMI1640 medium+10% FBS, 37° C., 5% CO₂); and mino cells (culture conditions: RPMI1640 medium+15% FBS, 37° C., 5% CO₂). The cells subcultured into the logarithmic growth phase were collected and transferred into a centrifuge tube, and centrifuged at 800 rpm for 5 min. The cells were resuspended in complete medium and counted, and the cell number was adjusted to 1×10⁶ cells/mL. The cells were then evenly plated into a six-well plate, 1 ml per well. Drug treatment: The diluted drug solution was added to the six-well plate, 1 ml per well. Meanwhile, negative control well was set up, in which 1 ml of empty culture medium containing an equal amount of DMSO was added. The six-well plate was incubated in the incubator for 6 h. Extraction of cell protein: A protein lysis buffer containing inhibitors (adding 20 μl of protease inhibitors and 20 μl of phosphatase inhibitors in 1 ml of the lysis buffer) was prepared in advance for later use. The suspended cells were transferred to a 2 ml centrifuge tube, and centrifuged at 2000 rpm for 5 min. The supernatant was discarded, and the cells were washed twice with PBS, and centrifuged at 2000 rpm for 5 min each time and the supernatant was discarded. 100 μl of the pre-cooled protein lysis buffer containing inhibitors was added and lysis was carried out on ice for 30 min, with shaking every 5-10 min. The lysate was centrifuged at 12000 rpm for 10 min in a pre-cooled 4° centrifuge, and 70 μl of the supernatant was transferred to a new centrifuge tube and stored for later use. Detection of protein concentration: Following the protocol of the BCA kit, the OD value of the sample in each well was detected by a microplate reader, and the protein concentration in the sample was calculated based on the standard curve. The volume of the lysate was adjusted to equalize the protein concentration across all samples. Protein denaturation: A protein loading buffer (5×) was added to the sample protein supernatant at a ratio of 1:4 and mixed thoroughly. After mixing evenly, the mixture was heated at 100° C. for 10 min and then stored in a refrigerator at −80° C. Sample loading and electrophoresis: The pre-prepared SDS-PAGE gel plate was mounted in the electrophoresis box, the electrophoresis buffer was added, and the comb was slowly and gently withdrawn before sample loading. The electrophoresis voltage was set to 150 V until the end. Wet transfer: After cessation of the electrophoresis, the region where the target protein molecular weight was located was determined based on the Maker and cut out with the comb. Meanwhile, a PVDF membrane of similar size was cut, which was soaked in methanol for 15 sec before use. A transfer buffer was poured into the tray, followed by sequential placement of sponge pads and filter papers in the prescribed order. The transfer current was set to 200 mA and the transfer time was set to 1 h. Blocking: A small hole was cut at the lower right corner of the PVDF membrane to record the front and back sides in contact with the gel, and the membrane was submerged in 5% BSA solution and incubated at low speed on a horizontal shaker at room temperature for 1 h. Antibody incubation: After blocking, the membrane was washed three times in TBST solution for 10 min each. The PVDF membrane was directly placed in a primary antibody incubation solution and a secondary antibody incubation solution for BTK in sequence for incubation. The incubation with the primary antibody incubation solution should be continued on the horizontal shaker at 4° C. for more than 12 h, and the incubation with the secondary antibody incubation solution should be continued on the horizontal shaker at room temperature for 1 h. After each incubation, the membrane was washed three times in TBST solution for 10 min each. Development: The A/B solutions of a developer were mixed in a ratio of 1:1 and used immediately after preparation. The development was performed by a developing instrument according to the procedure.

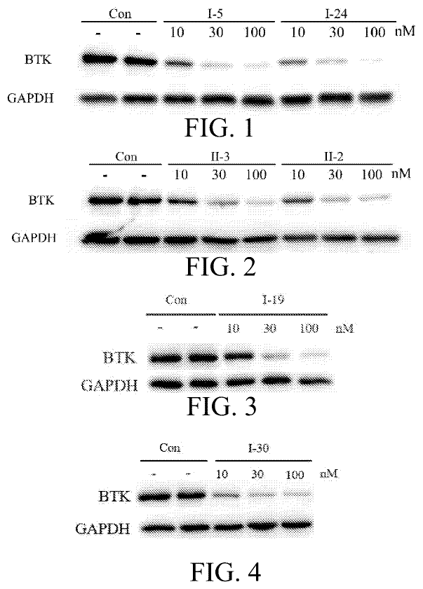

Conclusion: It can be seen from FIGS. 1-4 that the compounds of the present invention have good BTK protein degradation activity.

### Experimental Example 3: PK Test in Mice

Mice were weighed and dosed, and about 50 μL of whole blood was collected from the orbital venous plexus at 5 min, 0.25 h, 0.5 h, 1 h, 2 h, 4 h, 6 h, 8 h, and 24 h after administration, using EDTA-K₂ as anticoagulant. The blood samples were centrifuged at 4000 rpm for 10 min at 4° C., and 20 μL of the supernatant plasma sample was taken into an EP tube and immediately stored in a −80° C. refrigerator until analysis. 10 μL of the plasma sample was transferred to a 96-well plate and 250 μL of acetonitrile (containing internal standard) was added to precipitate proteins. The samples were centrifuged at 4000 rpm/min for 20 min in a 4° C. centrifuge. The supernatant was transferred and injected into LC MS/MS to detect the drug concentration. The data were processed by Thermo LC Quan software, and the standard curve was established using the internal standard method, with the theoretical standard curve concentration as the abscissa and the peak area ratio as the ordinate (test compound peak area/internal standard peak area). Sample concentrations were calculated using the standard curve. The pharmacokinetic parameters were calculated using WinNonlin 8.2 software.

| | I-2 | I-2 |
|---|---|---|
| PK parameters | IV-5 mg/kg | PO-20 mg/kg |
| Cₘₐₓ (ng/mL) | / | 8950 ± 339 |
| T_{1/2} (hr) | 5.35 ± 1.61 | 6.32 ± 3.49 |
| AUC₀₋ₜ (ng · hr/mL) | 42843 ± 5760 | 83466 ± 22231 |
| AUC_{0-∞} (ng · hr/mL) | 44454 ± 4614 | 92170 ± 33219 |
| MRT (hr) | 5.25 ± 0.17 | 6.46 ± 1.40 |
| Bioavialibility (%) | / | 51.8% |

Conclusion: The compounds of the present invention have good oral bioavailability and in vivo exposure, which is beneficial to the exertion of in vivo drug efficacy.

### Experimental Example 4: Brain Tissue Distribution Test in Mice

Mice were weighed and dosed, and about 50 μL of whole blood was collected from the orbital venous plexus at 5 min, 0.25 h, 0.5 h, 1 h, 2 h, 4 h, 6 h, 8 h, and 24 h after administration, using EDTA-K₂ as anticoagulant. At the same time, the animals were euthanized, and the brains were removed, and the brain tissue samples were stored in a −80° C. refrigerator until analysis. The blood samples were centrifuged at 4000 rpm for 10 min at 4° C., and 20 μL of the supernatant plasma sample was taken into an EP tube and immediately stored in a −80° C. refrigerator until analysis. 10 μL of the plasma sample was transferred to a 96-well plate and 250 μL of acetonitrile (containing internal standard) was added to precipitate proteins. The samples were centrifuged at 4000 rpm/min for 20 min in a 4° C. centrifuge. The supernatant was transferred and injected into LC MS/MS to detect the drug concentration. The brain tissue samples were weighed and homogenized with 4 times ice-cold PBS, and 50 μL of the brain tissue homogenate was transferred to a 96-well plate. 500 μL of acetonitrile (containing internal standard) was added to precipitate the protein. The samples were centrifuged at 4000 rpm/min for 20 min in a 4° C. centrifuge. 380 μL of the supernatant was transferred to a new 96-well plate and 380 μL of 0.1% formic acid in water was added and mixed thoroughly. 10 μL of the sample was taken and injected into LC MS/MS to detect the drug concentration. The data were processed by Thermo LC Quan software, and the standard curve was established using the internal standard method, with the theoretical standard curve concentration as the abscissa and the peak area ratio as the ordinate (test compound peak area/internal standard peak area). Sample concentrations were calculated using the standard curve. The pharmacokinetic parameters were calculated using WinNonlin 8.2 software.

| | I-18 | I-18 |
|---|---|---|
| | IV-10 | IV-10 |
| PK parameters | mg/kg-plasma | mg/kg-brain |
| T½ (hr) | 4.44 ± 1.38 | 9.39 ± 3.04 |
| AUC0-t (ng · hr/mL) | 39111 ± 5080 | 2273 ± 126 |
| AUC0-∞ (ng · hr/mL) | 39829 ± 4461 | 2708 ± 149 |
| MRT (hr) | 4.88 ± 0.14 | 7.17 ± 0.59 |
| B/P Ratio_{AUC (brain)/AUC (plasma)} | — | 6.86% ± 0.01 |

Conclusion: The compounds of the present invention have good brain permeability, which is beneficial to the exertion of drug efficacy in the brain.

### Experimental Example 5: Activity Test in Mino Cell Xenograft Model

Mino cell culture (culture conditions: RPMI1640 medium+15% FBS, 37° C., 5% CO₂). The cells subcultured into the logarithmic growth phase were collected and transferred into a centrifuge tube, and centrifuged at 800 rpm for 5 min. On the day of inoculation, the cell density was adjusted to 4*10⁸ cells/mL (culture medium: matrix gel=1:1).

Cell inoculation: The inoculation site was selected at the right shoulder blade of the experimental animal, and the inoculation density is 4*10⁷ cells/100 μL/animal.

Animal information: NOD SCID, female, 6-8 W.

Grouped administration: When the average tumor volume reached about 400 mm³, the animals were randomly grouped according to tumor volume, with the coefficient of variation (CV) of tumor volumes not exceeding ⅓ of the mean volume. The day of grouping was defined as DO, and the administration began on the day of grouping. The body weight of the mice was measured before administration, and the administration was based on the body weight on that day.

Experimental observation and data collection: After cell inoculation, the effect of the tumor on the normal behavior of the animals was routinely monitored every week. Specific contents include the activity, food and water intake, weight gain or loss, eyes, fur and other abnormalities of the experimental animals. After the start of administration, the mice were weighed and dosed daily, and tumor volume was measured twice a week. The tumor volume was calculated as follows: tumor volume (mm³)=0.52×tumor long diameter (mm)×tumor short diameter (mm)². At the end of the experiment, the following indicators were analyzed: tumor growth curve, mouse body weight curve, tumor weight, uniform photography of the excised tumors arranged according to groups, and calculation of tumor volume inhibition rate (TGI).

TGI_{TV} (Relative tumor volume inhibition rate) calculation formula:

$$\begin{matrix}{RTV}_{n} = {V}_{nt} + {V}_{n 0} \\ TGI = ( 1 - mean {RTV}_{treat} + mean {RTV}_{vehicle} ) \times 100 \%\end{matrix}$$

Vₙₜ: Tumor volume of the mouse numbered n on day t;
Vₙ₀: Tumor volume of the mouse numbered n on day 0;
RTVₙ: Relative tumor volume of the mouse numbered n on day t;
mean RTVₜᵣₑₐₜ: RTV mean of the administration group;
mean RTV_{vehicle}: RTV mean of the Vehicle group;

The experimental results such as tumor volume and mouse body weight of each group of animals are expressed as mean: standard error of the mean (Mean═SEM). P<0.05 was considered to be significantly different.

| | Tumor volume | | |
|---|---|---|---|
| Group | $\frac{Mean \pm SD}{( {mm}^{3} )} TGI \% \frac{P value}{( VS Model )}$ | | |
| Model | 4916.74 ± 1109.12 | / | / |
| Ibrutinib-50 mg/kg | 3018.11 ± 1293.65 | 40.73% | 0.0033 |
| I-2-30 mg/kg | 955.81 ± 343.68 | 80.75% | <0.0001 |
| I-40-30 mg/kg | 1146.34 ± 435.82 | 76.65% | <0.0001 |

Note:
Date: Mean ± SD;
** P < 0.01, *** P < 0.001 vs Model group.

The compounds of the present invention have significant inhibitory effects in the lymphoma Mino cell xenograft model, with stronger inhibitory activity compared to the positive drug ibrutinib.

### Experimental Example 6: Activity Test in REC-1 (BTK C481S) Cell Xenograft Model

REC-1 (BTK C481S) cell culture (culture conditions: RPMI1640 medium+10% FBS, 37° C., 5% CO₂). The cells subcultured into the logarithmic growth phase were collected and transferred into a centrifuge tube, and centrifuged at 1300 rpm for 3 min. On the day of inoculation, the cell density was adjusted to 5*10⁷ cells/mL.

Cell inoculation: The inoculation site was selected at the right shoulder blade of the experimental animal, and the inoculation density was 5*10⁶ cells+matrix gel/200 μL/animal.

Experimental animals: BALB/c-Nude, female, 6-8 W.

Grouped administration: When the average tumor volume reached 80-120 mm³, the animals were randomly grouped according to tumor volume, with the coefficient of variation (CV) of tumor volumes not exceeding ⅓ of the mean volume. The day of grouping was defined as D0, and the administration began on the day of grouping. The body weight of the mice was measured before administration, and the administration was based on the body weight on that day.

Experimental observation and data collection: After cell inoculation, the effect of the tumor on the normal behavior of the animals was routinely monitored every week. Specific contents include the activity, food and water intake, weight gain or loss, eyes, fur and other abnormalities of the experimental animals. After the start of administration, the mice were weighed and dosed daily, and tumor volume was measured twice a week. The tumor volume was calculated as follows: tumor volume (mm³)=0.52×tumor long diameter (mm)×tumor short diameter (mm)². At the end of the experiment, the following indicators were analyzed: tumor growth curve, mouse body weight curve, tumor weight, uniform photography of the excised tumors arranged according to groups, and calculation of tumor volume inhibition rate (TGI).

TGI_{TV} (Relative tumor volume inhibition rate) calculation formula:

$$\frac{Mean \pm SD}{( {mm}^{3} )} TGI \% \frac{P value}{( VS Model )}$$

Vₙₜ: Tumor volume of the mouse numbered n on day t;
Vₙ₀: Tumor volume of the mouse numbered n on day 0;
RTVₙ: Relative tumor volume of the mouse numbered n on day t;
mean RTVₜᵣₑₐₜ: RTV mean of the administration group;
mean RTV_{vehicle}: RTV mean of the Vehicle group;
The experimental results such as tumor volume and mouse body weight of each group of animals are expressed as mean±standard error of the mean (Mean±SEM). T test was used for comparison between the two groups. One-way ANOVA was used for comparisons among three or more groups. If there was a significant difference in the F value, multiple comparisons should be performed after the ANOVA analysis. p<—0.05 was considered to be significantly different.

| Group | Tumor weight (g)^{a} | T/C^{b} (%) | p value^{c} |
|---|---|---|---|
| Vehicle | 2.893 ± 0.417 | | — |
| I-2 15 mg/kg | 0.345 ± 0.082 | 11.93 | 0.008 |
| I-2 30 mg/kg | 0.369 ± 0.043 | 12.76 | 0.009 |

Note:
^{a}Mean ± SEM
^{b}Tumor growth inhibition was calculated by T_{weight}/C_{weight};
^{c}The p values were analyzed using One-way ANOVA based on the tumor weights of different groups with the Vehicle group as the control;

Conclusion: The compounds of the present invention have significant inhibitory effects in the lymphoma BTK C481S drug-resistant xenograft model and can completely inhibit the growth of tumor cells.

1. A compound as shown in formula (I), or an isomer or pharmaceutically acceptable salt thereof: 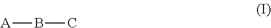
wherein,
A is 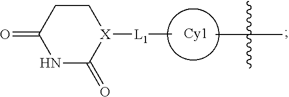
X is selected from N and CH;
Cy1 is selected from 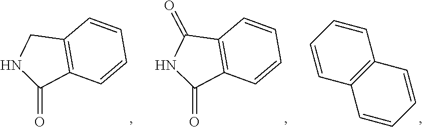
and phenyl, wherein the 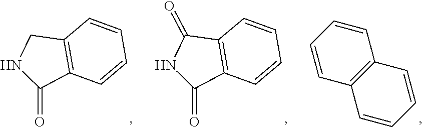
and phenyl are unsubstituted or each independently substituted with 1-3 Rₐ₁;
each Rₐ₁ is independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
B is -L2-Cy2-L3-(Cy3)p-L4-Cy4-L5-(Cy5)q-;
p and q are independently 0 or 1;
C is -L6-Cy6-L7-Cy7-L8-Cy8;
L1, L2, L3, L4, L5, L6, L7 and L8 are independently selected from a bond, —(CH₂)ₙ—(NH)ₘ—, —O—, —S—, —NR₂, —CR₃₁R₃₂, —C(O)—, —C(O)O—, —C(O)NR₄—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
Cy3 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₃, a 4-6 membered heterocyclyl group substituted with 1-3 Rₐ₃, a 4-6 membered cycloalkenyl group substituted with 1-3 Rₐ₃, and a 4-6 membered heterocycloalkenyl group substituted with 1-3 Rₐ₃, wherein the heteroatoms of the 4-6 membered heterocyclyl group and the 4-6 membered heterocycloalkenyl group are selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
Cy5 is a 4-6 membered cycloalkyl group, a 4-6 membered heterocyclyl group, a 4-6 membered heterocycloalkenyl group, a 4-6 membered cycloalkyl group substituted with 1-3 Rₐ₅, a 4-6 membered heterocyclyl group substituted with 1-3 Rₐ₅, a 4-6 membered heterocycloalkenyl group substituted with 1-3 Rₐ₅, or 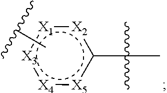
X₁ is N or CR_{b1};
X₂ is N or CR_{b2};
X₃ is N or CR_{b3};
X₄ is N or CR_{b4};
X₅ is N or CR_{b5};
R_{b1}, R_{b2}, R_{b3}, R_{b4} and Rₐ₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered heterocycloalkenyl group, a 9-10 membered heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₅, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₅, and a 5-6 membered heteroaryl group substituted with 1-3 Rₐ₅, wherein the heteroatom of the 5-6 membered heteroaryl group is selected from O, N and S; and
each of Rₐ₂, Rₐ₃, Rₐ₄, Rₐ₅, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

2. The compound according to claim 1, or the isomer or pharmaceutically acceptable salt thereof, having a structure as shown in general formula (II): 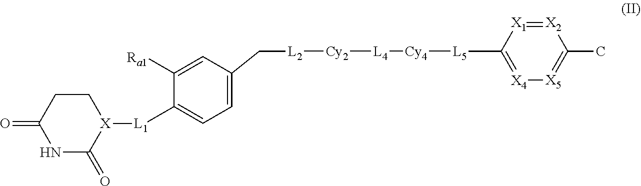
wherein,
X is selected from N and CH;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —CR₃₁R₃₂, —C(O)O—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₂ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
X₁ is N or CR_{b1};
X₂ is N or CR_{b2};
X₄ is N or CR_{b4},
X₅ is N or CR_{b5};
R_{b1}, R_{b2}, R_{b4}, and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, Cie alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered fused heterocycloalkenyl group, a 9-10 membered fused heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₅, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₅, wherein the heteroatom of the 5-6 membered heteroaryl group is selected from O, N and S; and
each of Rₐ₂, Rₐ₄, Rₐ₆, Rₐ₇, and Raz is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

3. The compound according to claim 2, or the isomer or pharmaceutically acceptable salt thereof,
X is selected from N and CH;
Rₐ₁ is selected from hydrogen, halogen, hydroxy, amino, C₁₋₆ alkyl, halogenated C₁₋₆ alkyl, C₁₋₆ alkoxy, halogenated C₁₋₆ alkoxy, and hydroxy C₁₋₆ alkyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —C(O)O—, and —C₁₋₆ alkyl-NR₅—C(O)—;
n and m are independently any integer from zero to three;
R₂, R₄, and R₅ are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 4-12 membered cycloalkyl group substituted with 1-3 Rₐ₄, and a 4-12 membered heterocyclyl group substituted with 1-3 Rₐ₄, wherein the heteroatom of the 4-12 membered heterocyclyl group is selected from O, N and S;
X₁ is N or CR_{b1};
X₂ is N or CR_{b2};
X₄ is N or CR_{b4};
X₅ is N or CR_{b5};
R_{b1}, R_{b2}, R_{b3}, R_{b4}, and R_{b5} are independently selected from hydrogen, halogen, hydroxy, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, and (C₁₋₆ alkyl)₂ amino;
Cy6 is selected from a 5-6 membered heteroaryl group, a 5-6 membered heterocycloalkenyl group, a 9-10 membered heterocycloalkenyl group, a 9-10 membered heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₅, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

4. The compound according to claim 3, or the isomer or pharmaceutically acceptable salt thereof, having a structure as shown in general formula (III): 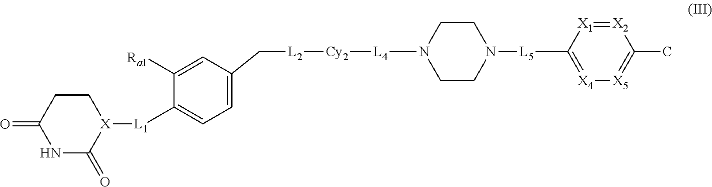
X is selected from N and CH;
Rₐ₁ is selected from hydrogen and methyl;
C is -L6-Cy6-L7-Cy7-L8-Cy8;
L1, L2, L4, L5, L6, L7 and L8 are independently selected from a bond, —CH₂—, and —CH₂—NH—C(O)—;
Cy2 is a 5-6 membered nitrogen-containing heterocyclyl group, preferably 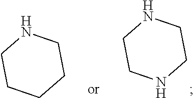 [SMILES: C1CCNCC1 ; C1CNCCN1]
X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen and F;
Cy6 is selected from a 5-6 membered heteroaryl group substituted with 1-3 Rₐ₆, and a 9-10 membered fused heteroaryl group substituted with 1-3 Rₐ₆, wherein the 5-6 membered heteroaryl group and the 9-10 membered fused heteroaryl group are preferably 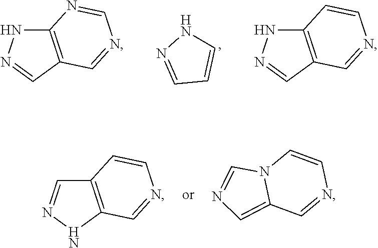 [SMILES: c1ncc2cn[nH]c2n1 ; c1cn[nH]c1 ; c1cc2[nH]ncc2cn1 ; c1cc2cn[nH]c2cn1 ; c1cn2cncc2cn1]
preferably 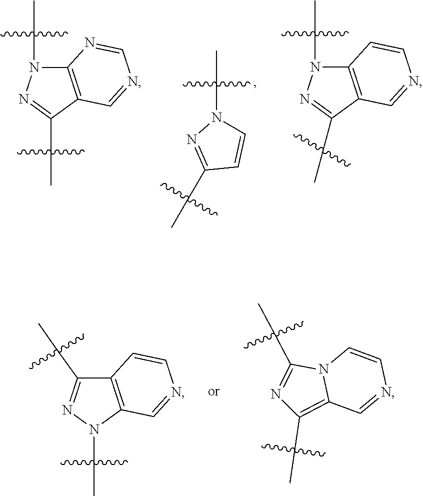 [SMILES: *c1nn(*)c2ncncc12 ; *c1ccn(*)n1 ; *c1nn(*)c2ccncc12 ; *c1nn(*)c2cnccc12 ; *c1nc(*)n2ccncc12]
more preferably 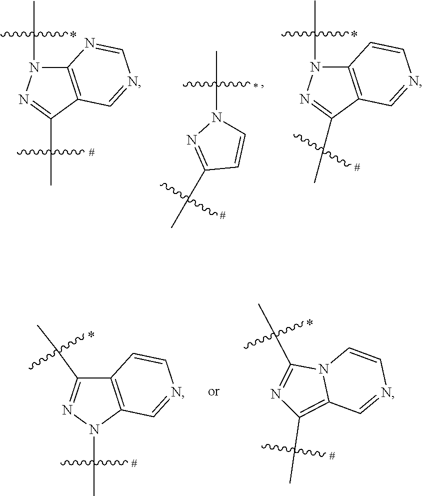 [SMILES: *c1nn(*)c2ncncc12 ; *c1ccn(*)n1 ; *c1nn(*)c2ccncc12 ; *c1nn(*)c2cnccc12 ; *c1nc(*)n2ccncc12]
wherein the * end is connected to L6, and the #end is connected to L7;
each Rₐ₆ is independently selected from hydrogen, amino, and aminocarbonyl;
Cy7 is selected from phenyl and phenyl substituted with 1-3 Rₐ₇;
each Raz is independently selected from hydrogen and methyl;
Cy8 is phenyl substituted with 1-3 Rₐ₈; and
each Rₐ₈ is independently selected from hydrogen, F, and methoxy.

5. (canceled)

6. (canceled)

7. The compound according to claim 1, or the isomer or pharmaceutically acceptable salt thereof, having a structure as shown in general formula (IV), 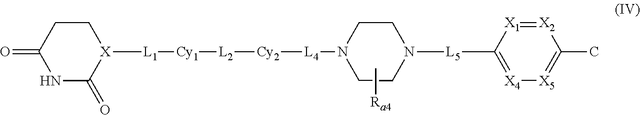
X is selected from N and CH;
Cy1 is selected from 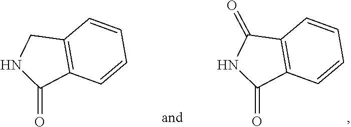 [SMILES: O=C1NCc2ccccc21 ; O=C1NC(=O)c2ccccc21]
wherein the 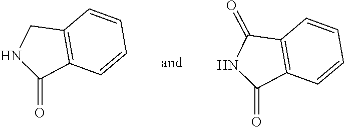 [SMILES: O=C1NCc2ccccc21 ; O=C1NC(=O)c2ccccc21]
are unsubstituted or each independently substituted with 1-3 Rₐ₁;
each Rₐ₁ is independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl); amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
C is -L6-Cy6-L7-Cy7-L8-Cy8;
L1, L2, L4, L5, L6, L7 and L8 are independently selected from a bond, —(CH₂)ₙ—(NH)ₘ—, —O—, —S—, —NR₂, —CR₃₁R₃₂, —C(O)O—, —C(O)NR₄—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₂ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
X₁ is N or CR_{b1};
X₂ is N or CR_{b2};
X₄ is N or CR_{b4};
X₅ is N or CR_{b5};
R_{b1}, R_{b2}, R_{b4}, and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₂ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
Cy6 is selected from a 5-6 membered heteroaryl group, a 5-6 membered heterocycloalkenyl group, a 9-10 membered heterocycloalkenyl group, a 9-10 membered heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₅, wherein the heteroatom of the 5-6 membered heteroaryl group is selected from O, N and S; and
each of Rₐ₂, Rₐ₄, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl.

8. (canceled)

9. (canceled)

10. The compound according to claim 2, or the isomer or pharmaceutically acceptable salt thereof, 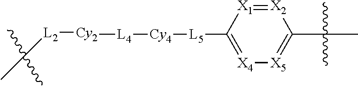
is selected from 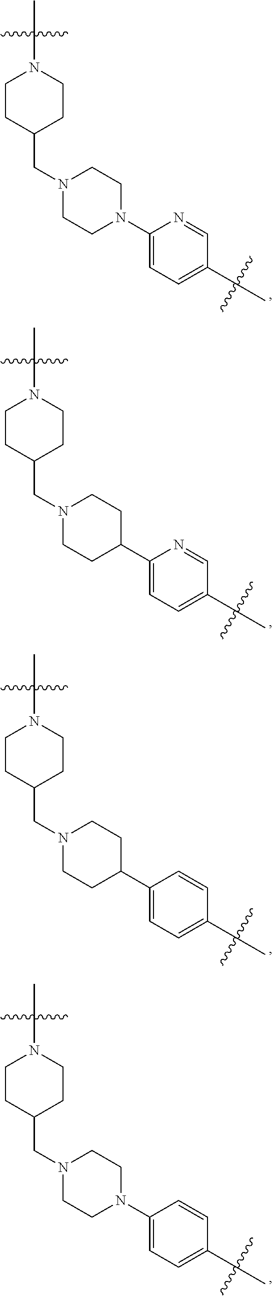 [SMILES: *c1ccc(N2CCN(CC3CCN(*)CC3)CC2)nc1 ; *c1ccc(C2CCN(CC3CCN(*)CC3)CC2)nc1 ; *c1ccc(C2CCN(CC3CCN(*)CC3)CC2)cc1 ; *c1ccc(N2CCN(CC3CCN(*)CC3)CC2)cc1] 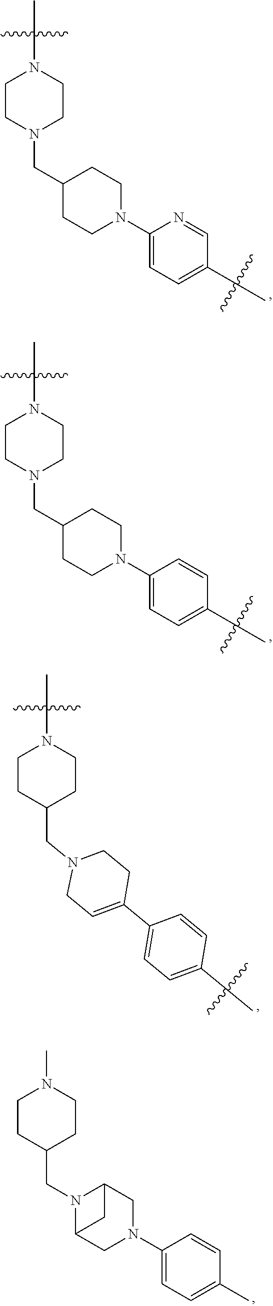 [SMILES: *c1ccc(N2CCC(CN3CCN(*)CC3)CC2)nc1 ; *c1ccc(N2CCC(CN3CCN(*)CC3)CC2)cc1 ; *c1ccc(C2=CCN(CC3CCN(*)CC3)CC2)cc1 ; Cc1ccc(N2CC3CC(C2)N3CC2CCN(C)CC2)cc1] 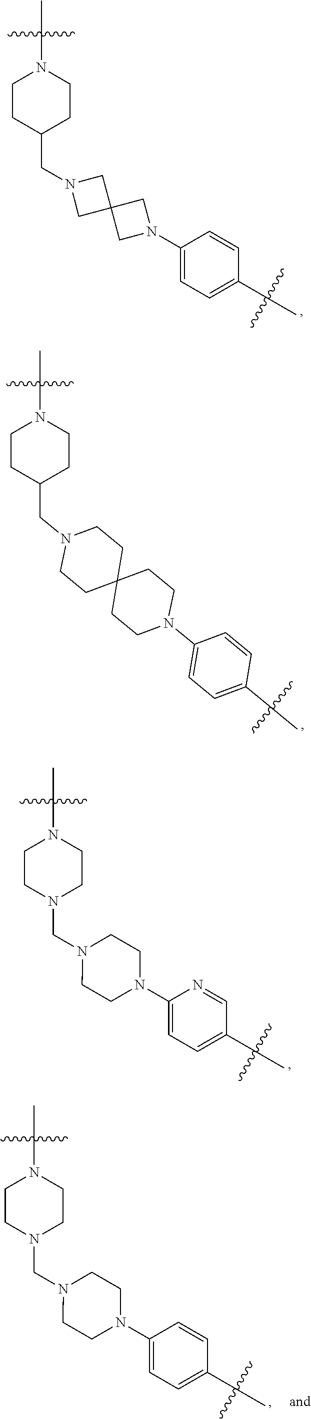 [SMILES: *c1ccc(N2CC3(CN(CC4CCN(*)CC4)C3)C2)cc1 ; *c1ccc(N2CCC3(CCN(CC4CCN(*)CC4)CC3)CC2)cc1 ; *c1ccc(N2CCN(CN3CCN(*)CC3)CC2)nc1 ; *c1ccc(N2CCN(CN3CCN(*)CC3)CC2)cc1] 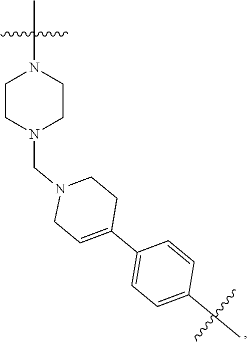 [SMILES: *c1ccc(C2=CCN(CN3CCN(*)CC3)CC2)cc1]
wherein the corresponding Cy2 group is unsubstituted or substituted with 1-3 Rₐ₂, the corresponding Cy4 group is unsubstituted or substituted with 1-3 Rₐ₄, and the corresponding X₁, X₂, X₄, and X₅ are unsubstituted or substituted with the corresponding R_{b1}, R_{b2}, R_{b4}, and Rₐ₈ respectively; and each of Rₐ₂, Rₐ₄, R_{b1}, R_{b2}, R_{b4}, and R_{b5} is at occurrence, independently selected from hydrogen and F.

11. The compound according to claim 7, or the isomer or pharmaceutically acceptable salt thereof, 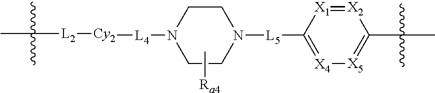
is selected from 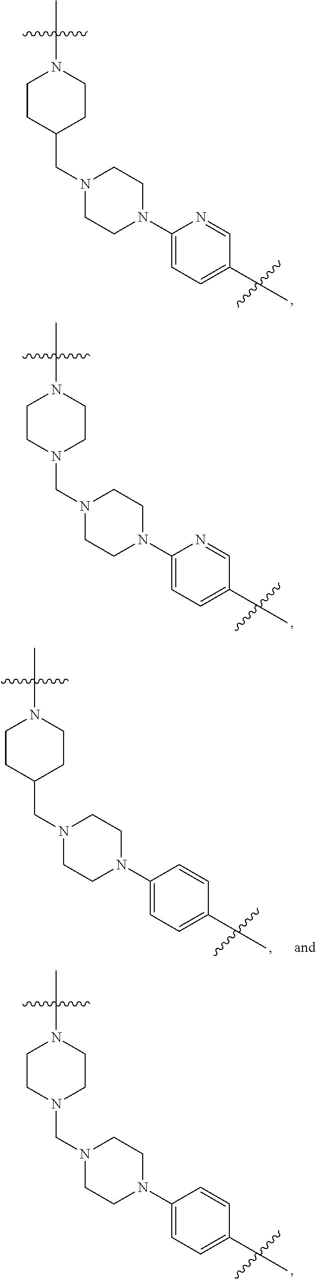 [SMILES: *c1ccc(N2CCN(CC3CCN(*)CC3)CC2)nc1 ; *c1ccc(N2CCN(CN3CCN(*)CC3)CC2)nc1 ; *c1ccc(N2CCN(CC3CCN(*)CC3)CC2)cc1 ; *c1ccc(N2CCN(CN3CCN(*)CC3)CC2)cc1]
wherein the corresponding Cy2 group is unsubstituted or substituted with 1-3 Rₐ₂, and the corresponding X₁, X₂, X₄, and X₅ are unsubstituted or substituted with the corresponding R_{b1}, R_{b2}, R_{b4}, and Rₐ₈ respectively; and each of Rₐ₂, Rₐ₄, R_{b1}, R_{b2}, R_{b4}, and R_{b5} is at occurrence, independently selected from hydrogen and F.

12. The compound according to any one of claim 1, or the isomer or pharmaceutically acceptable salt thereof,
C is -Cy6-L7-Cy7-L8-Cy8;
Cy6 is selected from a 5-6 membered heteroaryl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the 5-6 membered heteroaryl group, the 9-10 membered fused heterocycloalkenyl group, and the 9-10 membered fused heteroaryl group are preferably 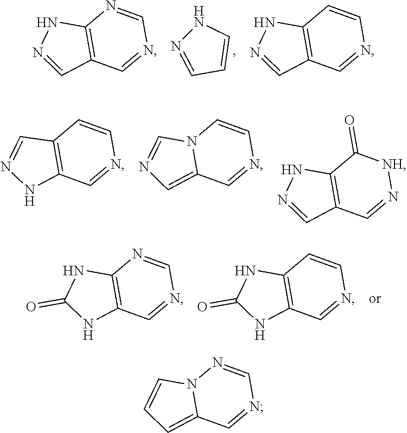
each Rₐ₆ is independently selected from hydrogen, amino, and aminocarbonyl;
-L7-Cy7-L8-Cy8 is selected from 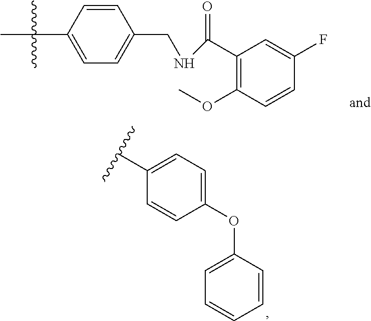 [SMILES: *c1ccc(CNC(=O)c2cc(F)ccc2OC)cc1 ; *c1ccc(Oc2ccccc2)cc1]
wherein the corresponding Cy7 group is unsubstituted or substituted with 1-3 Rₐ₇; and
Rₐ₇ is at each occurrence, independently selected from hydrogen, methyl, and F.

13. The compound according to claim 1, or the isomer or pharmaceutically acceptable salt thereof, having a structure as shown in general formula (V), 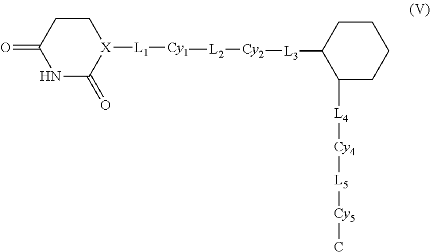
X is selected from N and CH;
Cy1 is selected from 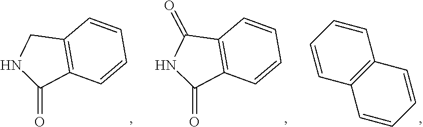
and phenyl, wherein the 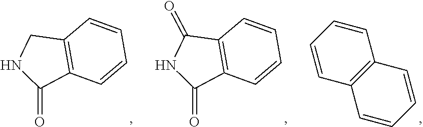
and phenyl are unsubstituted or each independently substituted with 1-3 Rₐ₁;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
C is -L6-Cy6-L7-Cy7-L8-Cy8;
L1, L2, L3, L4, L5, L6, L7 and L8 are independently selected from a bond, —(CH₂)ₙ—(NH)ₘ—, ~O—, —S—, —NR₂, —CR₃₁R₃₂, —C(O)—, —C(O)O—, —C(O)NR₄—, and —C₁₋₆ alkyl-NR₅—C(O)—;
n and m are independently any integer from zero to six;
R₂, R₃₁, R₃₂, R₄ and R₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl, wherein the C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, and halogenated C₁₋₆ alkoxy;
Cy2 is selected from a 4-6 membered cycloalkyl group, a 4-6 membered heterocyclyl group, a 4-6 membered cycloalkenyl group, a 4-6 membered heterocycloalkenyl group, a 4-6 membered cycloalkyl group substituted with 1-3 Rₐ₂, a 4-6 membered heterocyclyl group substituted with 1-3 Rₐ₂, a 4-6 membered cycloalkenyl group substituted with 1-3 Rₐ₂, and a 4-6 membered heterocycloalkenyl group substituted with 1-3 Rₐ₂, wherein the heteroatoms of the 4-6 membered heterocyclyl group and the 4-6 membered heterocycloalkenyl group are selected from O, N and S;
Cy4 is selected from a 4-12 membered cycloalkyl group, a 4-12 membered heterocyclyl group, a 4-12 membered cycloalkenyl group, a 4-12 membered heterocycloalkenyl group, a 4-12 membered cycloalkyl group substituted with 1-3 Rₐ₄, a 4-12 membered heterocyclyl group substituted with 1-3 Rₐ₄, a 4-12 membered cycloalkenyl group substituted with 1-3 Rₐ₄, and a 4-12 membered heterocycloalkenyl group substituted with 1-3 Rₐ₄, wherein the heteroatoms of the 4-12 membered heterocyclyl group and the 4-12 membered heterocycloalkenyl group are selected from O, N and S;
Cy5 is a 4-6 membered cycloalkyl group, a 4-6 membered heterocyclyl group, a 4-6 membered heterocycloalkenyl group, a 4-6 membered cycloalkyl group substituted with 1-3 Rₐ₅, a 4-6 membered heterocyclyl group substituted with 1-3 Rₐ₅, a 4-6 membered heterocycloalkenyl group substituted with 1-3 Rₐ₅, or 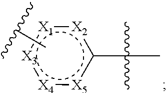
X₁ is N or CR_{b1};
X₂ is N or CR_{b2};
X₃ is N or CR_{b3};
X₄ is N or CR_{b4};
X₅ is N or CR_{b5};
R_{b1}, R_{b2}, R_{b3}, R_{b4} and R_{b5} are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl), aminocarbonyl;
Cy6 is selected from a 5-6 membered heteroaryl group, a 5-6 membered heterocycloalkenyl group, a 9-10 membered heterocycloalkenyl group, a 9-10 membered heteroaryl group, a 5-6 membered heteroaryl group substituted with 1-3 Rₐ₆, a 5-6 membered heterocycloalkenyl group substituted with 1-3 Rₐ₆, a 9-10 membered fused heterocycloalkenyl group substituted with 1-3 Rₐ₆, and a 9-10 membered fused heteroaryl group substituted with 1-3 Rₐ₆, wherein the heteroatoms of the 5-6 membered heteroaryl group, the 5-6 membered heterocycloalkenyl group, the 9-10 membered fused heterocycloalkenyl group, and the 9-10 membered fused heteroaryl group are selected from O, N and S;
Cy7 is selected from phenyl, a 5-6 membered heteroaryl group, phenyl substituted with 1-3 Rₐ₇, and a 5-6 membered heteroaryl group substituted with 1-3 Rₐ₇, wherein the heteroatom of the 5-6 membered heteroaryl group is selected from O, N and S;
Cy8 is selected from phenyl, a 5-6 membered heteroaryl group, phenyl substituted with 1-3 Rₐ₈, and a 5-6 membered heteroaryl group substituted with 1-3 Rₐ₅, wherein the heteroatom of the 5-6 membered heteroaryl group is selected from O, N and S; and
each of Rₐ₂, Rₐ₃, Rₐ₄, Rₐ₅, Rₐ₆, Rₐ₇, and Rₐ₈ is at occurrence, independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₂ alkynyl, C₁₋₆ alkylcarbonyl, aminocarbonyl, C₁₋₆ alkylaminocarbonyl, and (C₁₋₆ alkyl)₂ aminocarbonyl.

14. (canceled)

15. (canceled)

16. The compound according to claim 2, or the isomer or pharmaceutically acceptable salt thereof, having a structure as shown in general formula (VI), 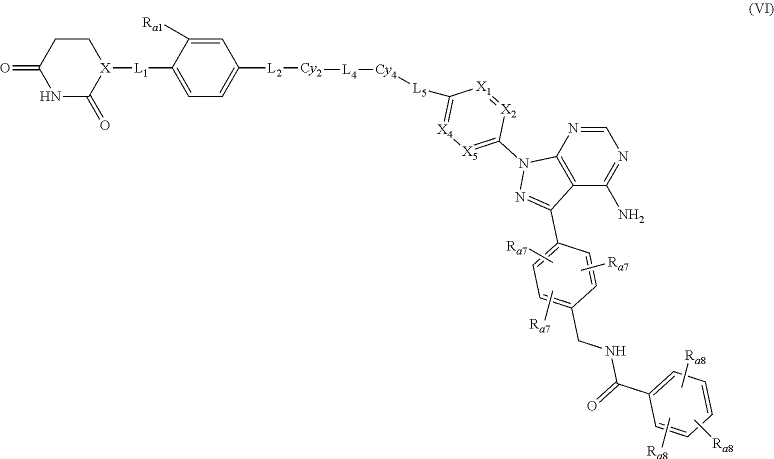
X is selected from N and CH;
L1 is a bond;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
L2, L4 and L5 are independently selected from a bond, —(CH₂)ₙ—(NH)ₘ—, —O—, and —S—;
n and m are independently any integer from zero to three;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 3-8 membered heterocycloalkenyl group, a 4-12 membered cycloalkyl group substituted with 1-3 Rₐ₄, a 4-12 membered heterocyclyl group substituted with 1-3 Rₐ₄, and a 3-8 membered heterocycloalkenyl group substituted with 1-3 Rₐ₄, wherein the heteroatoms of the 4-12 membered heterocyclyl group and the 3-8 membered heterocycloalkenyl group are selected from O, N and S;
each of Raz and Rₐ₄ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl); amino, C₁₋₆ alkylcarbonyl, aminocarbonyl, C₁₋₆ alkylaminocarbonyl, and (C₁₋₆ alkyl)₂ aminocarbonyl;
X₁ is N or CR_{b1};
X₂ is N or CR_{b2};
X₄ is N or CR_{b4};
X₅ is N or CR_{b5};
R_{b1}, R_{b2}, R_{b4}, and Rₐ₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl; and
each of Rₐ₇ and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

17. (canceled)

18. The compound according to claim 2, or the isomer or pharmaceutically acceptable salt thereof, having a structure as shown in general formula (VII), 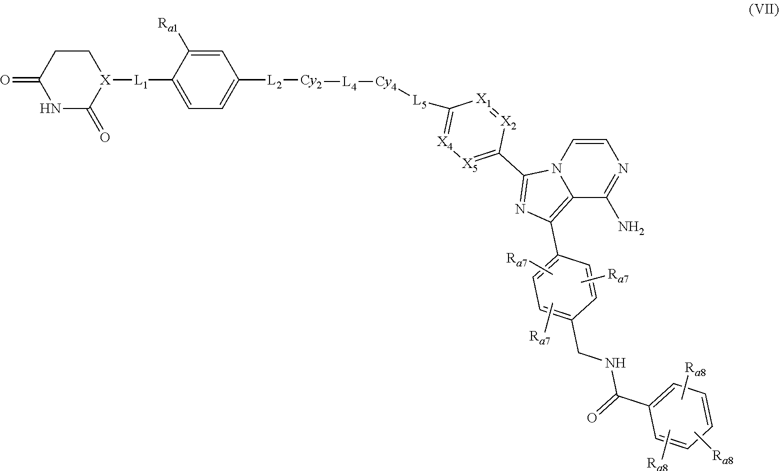
X is selected from N and CH;
L1 is a bond;
Rₐ₁ is selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₈ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl;
L2, L4 and L5 are independently selected from a bond, —(CH₂)ₙ—(NH)ₘ—, —O—, and —S—;
n and m are independently any integer from zero to three;
Cy2 is selected from a 4-6 membered cycloalkyl group, a 4-6 membered heterocyclyl group, a 4-6 membered cycloalkyl group substituted with 1-3 Rₐ₂, and a 4-6 membered heterocyclyl group substituted with 1-3 Rₐ₂, wherein the heteroatom of the 4-6 membered heterocyclyl group is selected from O, N and S;
Cy4 is selected from a 4-12 membered cycloalkyl group, a 4-12 membered heterocyclyl group, a 3-8 membered heterocycloalkenyl group, a 4-12 membered cycloalkyl group substituted with 1-3 Rₐ₄, a 4-12 membered heterocyclyl group substituted with 1-3 Rₐ₄, and a 3-8 membered heterocycloalkenyl group substituted with 1-3 Rₐ₄, wherein the heteroatoms of the 4-12 membered heterocyclyl group and the 3-8 membered heterocycloalkenyl group are selected from O, N and S;
each of Rₐ₂ and Rₐ₄ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl;
X₁ is N or CR_{b1};
X₂ is N or CR_{b2};
X₄ is N or CR_{b4};
X₅ is N or CR_{b5};
R_{b1}, R_{b2}, R_{b4}, and Rₐ₅ are independently selected from hydrogen, halogen, hydroxy, carboxyl, amino, cyano, nitro, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₂₋₂ alkenyl, C₂₋₈ alkynyl, C₁₋₆ alkylcarbonyl, aminocarbonyl, C₁₋₆ alkylaminocarbonyl, and (C₁₋₆ alkyl)₂ aminocarbonyl; and
each of Rₐ₇ and Rₐ₈ is at occurrence, independently selected from hydrogen, halogen, amino, C₁₋₆ alkyl, halogenated C₁₋₆ alkyl, C₁₋₆ alkoxy, halogenated C₁₋₆ alkoxy, hydroxy C₁₋₆ alkyl, C₁₋₆ alkylamino, (C₁₋₆ alkyl)₂ amino, C₁₋₆ alkylcarbonyl, aminocarbonyl, C₁₋₆ alkylaminocarbonyl, and (C₁₋₆ alkyl)₂ aminocarbonyl.

19. The compound according to claim 18, or the isomer or pharmaceutically acceptable salt thereof,
X is selected from N and CH;
L1 is a bond;
Rₐ₁ is selected from hydrogen and methyl;
L2, L4 and L5 are independently selected from a bond and —CH₂—;
Cy2 is a 5-6 membered nitrogen-containing heterocyclyl group or a 5-6 membered nitrogen-containing heterocyclyl group substituted with 1-3 Rₐ₂, wherein the 5-6 membered nitrogen-containing heterocyclyl group is preferably 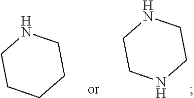 [SMILES: C1CCNCC1 ; C1CNCCN1]
Cy4 is a 5-6 membered nitrogen-containing heterocyclyl group, a 5-6 membered nitrogen-containing heterocycloalkenyl group, a 7-11 membered nitrogen-containing spiro heterocyclyl group or a 6-12 membered nitrogen-containing bridged heterocyclyl group, a 5-6 membered nitrogen-containing heterocyclyl group substituted with 1-3 Rₐ₄, a 5-6 membered nitrogen-containing heterocycloalkenyl group substituted with 1-3 Rₐ₄, a 7-11 membered nitrogen-containing spiro heterocyclyl group substituted with 1-3 Rₐ₄, or a 6-12 membered nitrogen-containing bridged heterocyclyl group substituted with 1-3 Rₐ₄, wherein the 5-6 membered nitrogen-containing heterocyclyl group is preferably 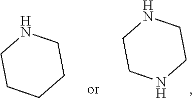 [SMILES: C1CCNCC1 ; C1CNCCN1]
the 5-6 membered nitrogen-containing heterocycloalkenyl group is preferably 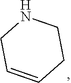
the 6-12 membered nitrogen-containing bridged heterocyclyl group is preferably 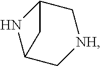
and the 7-11 membered nitrogen-containing spiro heterocyclyl group is preferably 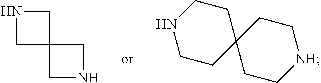 [SMILES: C1NCC12CNC2 ; C1CC2(CCN1)CCNCC2]
each of Rₐ₂ and Rₐ₄ is at occurrence, independently selected from hydrogen and methyl;
X₁ is N or CR_{b1};
X₂ is CR_{b2};
X₄ is CR_{b4};
X₅ is CR_{b5};
R_{b1}, R_{b2}, R_{b4}, and R_{b5} are independently selected from hydrogen, F, ethyl, and methoxy;
each Rₐ₇ is independently selected from hydrogen, methyl, and F; and
each Rₐ₈ is independently selected from hydrogen, F, and methoxy.

20. The compound according to any one of claim 1, or the isomer or pharmaceutically acceptable salt thereof,
C is

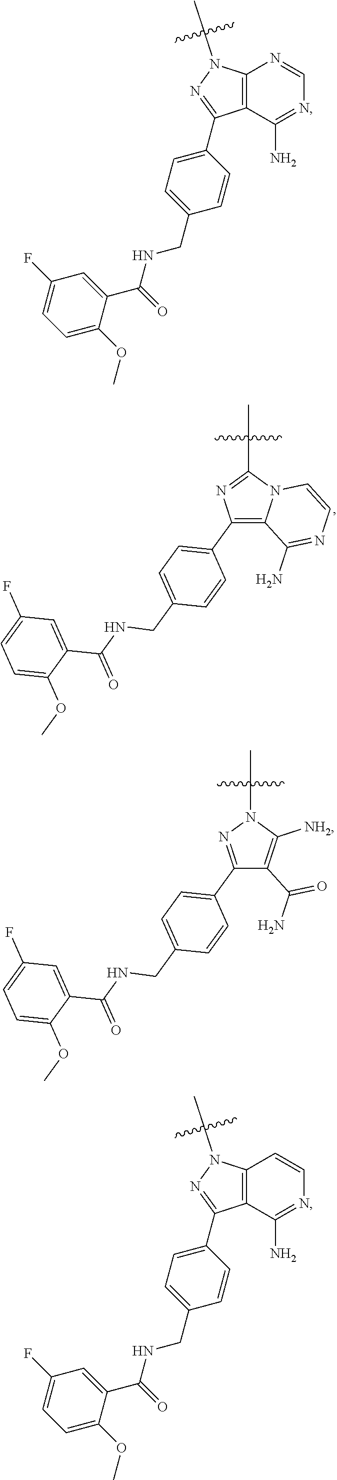 [SMILES: *n1nc(-c2ccc(CNC(=O)c3cc(F)ccc3OC)cc2)c2c(N)ncnc21 ; *c1nc(-c2ccc(CNC(=O)c3cc(F)ccc3OC)cc2)c2c(N)nccn12 ; *n1nc(-c2ccc(CNC(=O)c3cc(F)ccc3OC)cc2)c(C(N)=O)c1N ; *n1nc(-c2ccc(CNC(=O)c3cc(F)ccc3OC)cc2)c2c(N)nccc21]

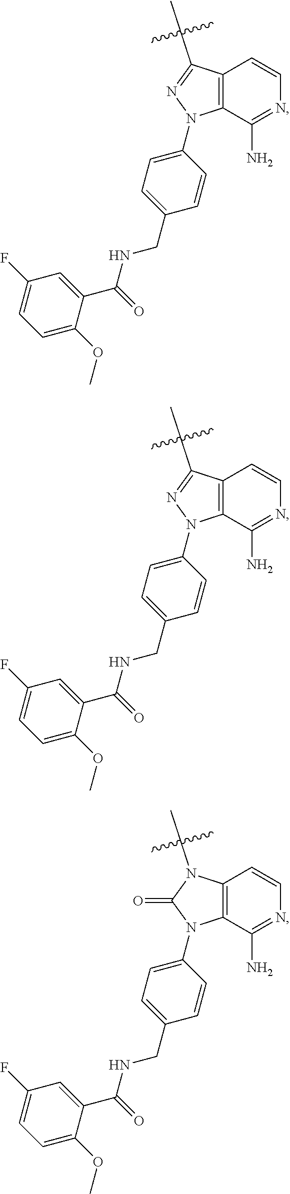 [SMILES: *c1nn(-c2ccc(CNC(=O)c3cc(F)ccc3OC)cc2)c2c(N)nccc12 ; *c1nn(-c2ccc(CNC(=O)c3cc(F)ccc3OC)cc2)c2c(N)nccc12 ; *n1c(=O)n(-c2ccc(CNC(=O)c3cc(F)ccc3OC)cc2)c2c(N)nccc21]

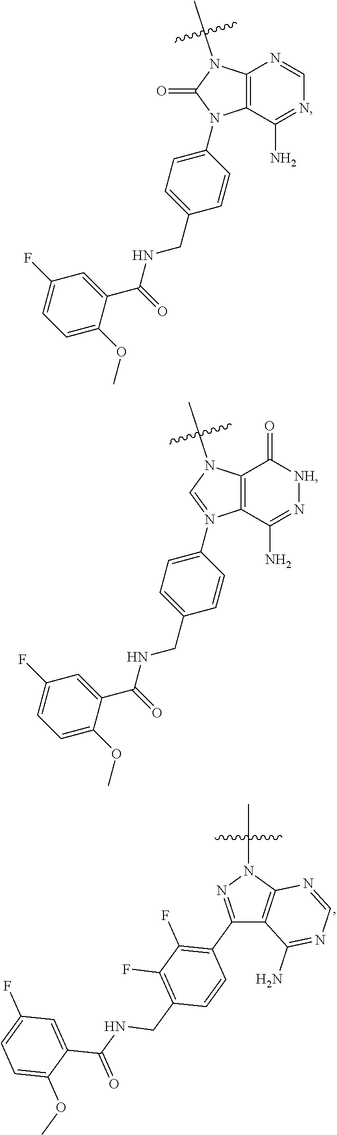

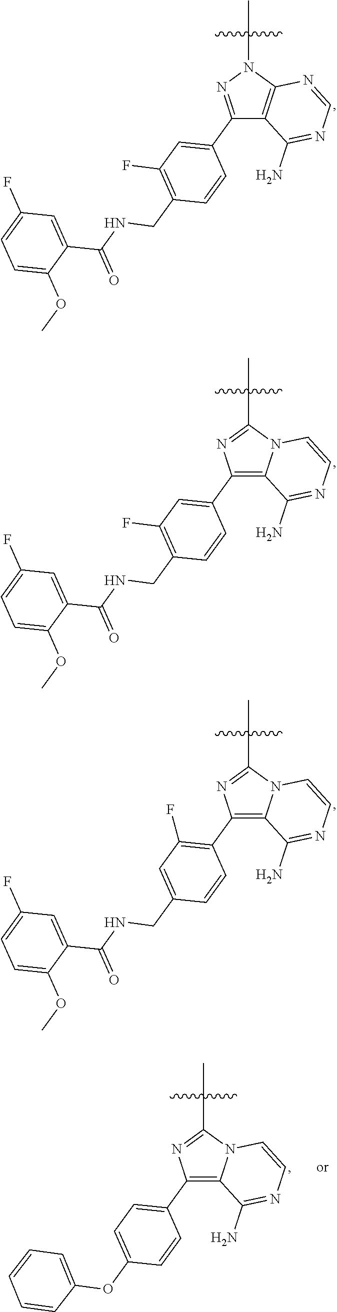 [SMILES: *n1nc(-c2ccc(CNC(=O)c3cc(F)ccc3OC)c(F)c2)c2c(N)ncnc21 ; *c1nc(-c2ccc(CNC(=O)c3cc(F)ccc3OC)c(F)c2)c2c(N)nccn12 ; *c1nc(-c2ccc(CNC(=O)c3cc(F)ccc3OC)cc2F)c2c(N)nccn12 ; *c1nc(-c2ccc(Oc3ccccc3)cc2)c2c(N)nccn12]

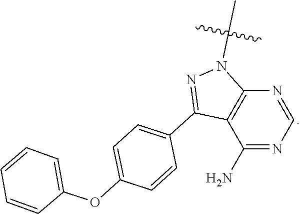 [SMILES: *n1nc(-c2ccc(Oc3ccccc3)cc2)c2c(N)ncnc21]

21. The compound according to claim 2, or the isomer or pharmaceutically acceptable salt thereof,

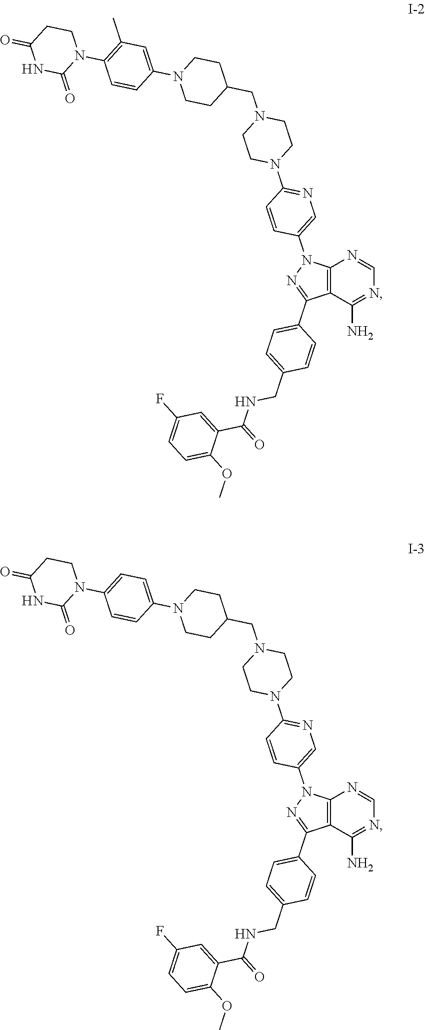 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1 ; COc1ccc(F)cc1C(=O)NCc1ccc(-c2nn(-c3ccc(N4CCN(CC5CCN(c6ccc(N7CCC(=O)NC7=O)cc6)CC5)CC4)nc3)c3ncnc(N)c23)cc1]

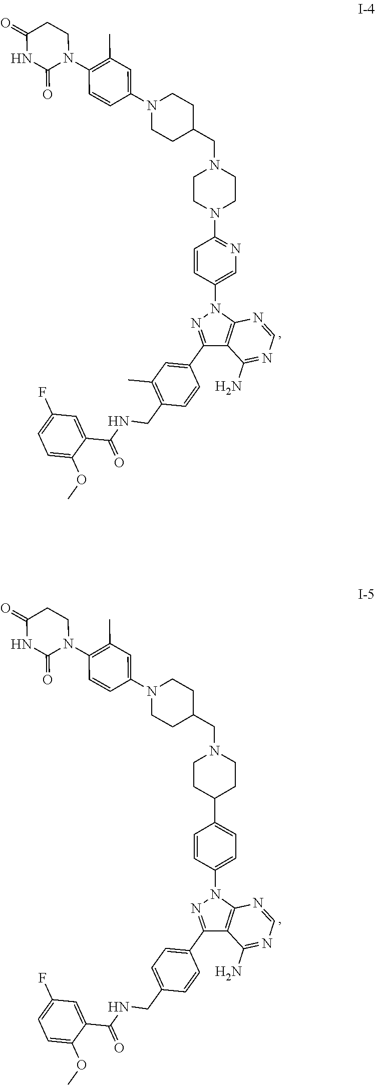 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)cc1C ; COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1]

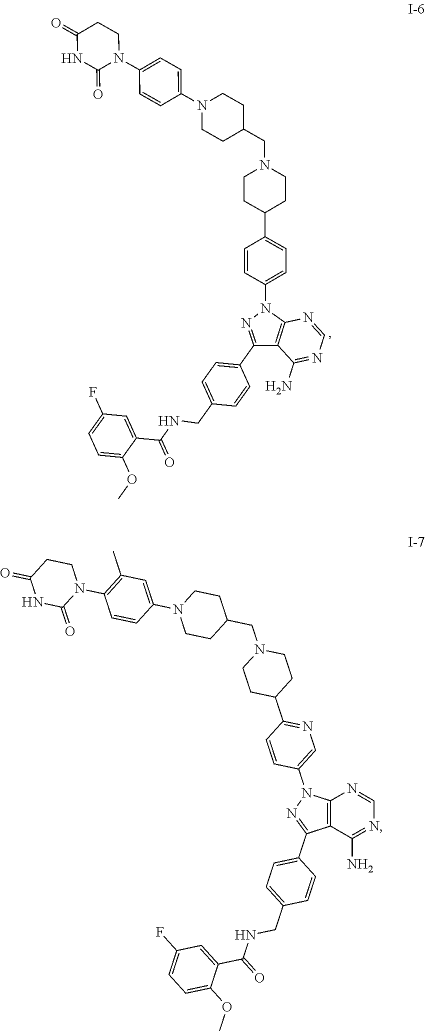 [SMILES: COc1ccc(F)cc1C(=O)NCc1ccc(-c2nn(-c3ccc(C4CCN(CC5CCN(c6ccc(N7CCC(=O)NC7=O)cc6)CC5)CC4)cc3)c3ncnc(N)c23)cc1 ; COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c3ncnc(N)c23)cc1]

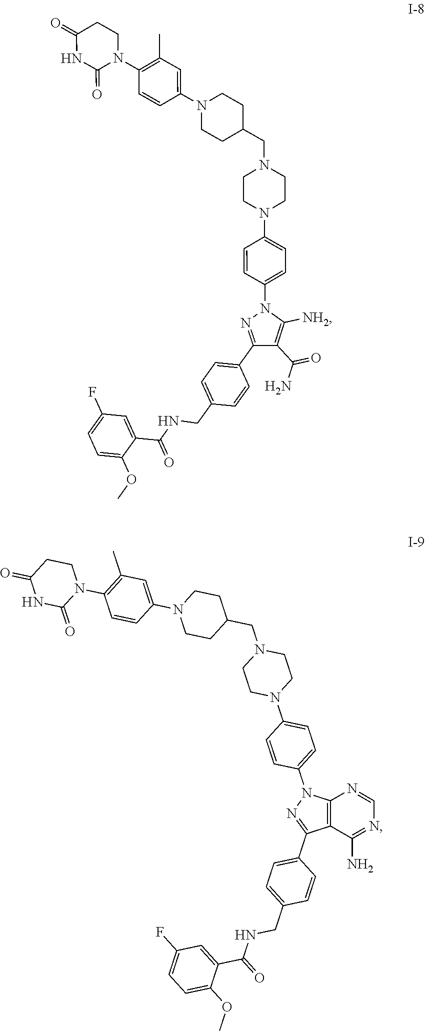 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c(N)c2C(N)=O)cc1 ; COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ncnc(N)c23)cc1]

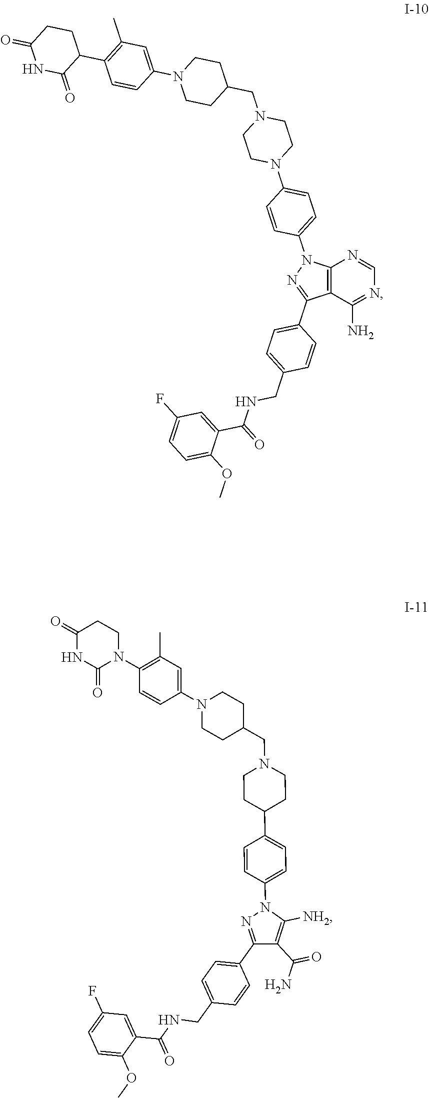 [SMILES: COc1ccc(F)cc1C(=O)NCc1ccc(-c2nn(-c3ccc(N4CCN(CC5CCN(c6ccc(C7CCC(=O)NC7=O)c(C)c6)CC5)CC4)cc3)c3ncnc(N)c23)cc1 ; COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c(N)c2C(N)=O)cc1]

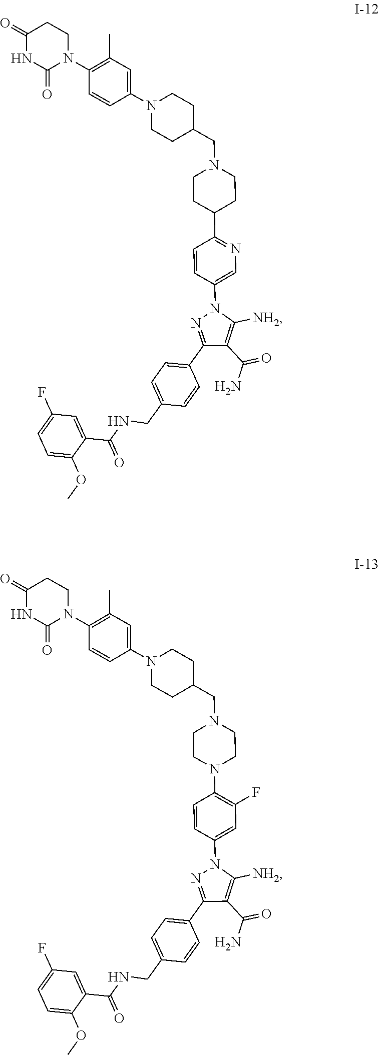 [SMILES: COc1ccc(F)cc1C(=O)NCc1ccc(-c2nn(-c3ccc(C4CCN(CC5CCN(c6ccc(N7CCC(=O)NC7=O)c(C)c6)CC5)CC4)nc3)c(N)c2C(N)=O)cc1 ; COc1ccc(F)cc1C(=O)NCc1ccc(-c2nn(-c3ccc(N4CCN(CC5CCN(c6ccc(N7CCC(=O)NC7=O)c(C)c6)CC5)CC4)c(F)c3)c(N)c2C(N)=O)cc1]

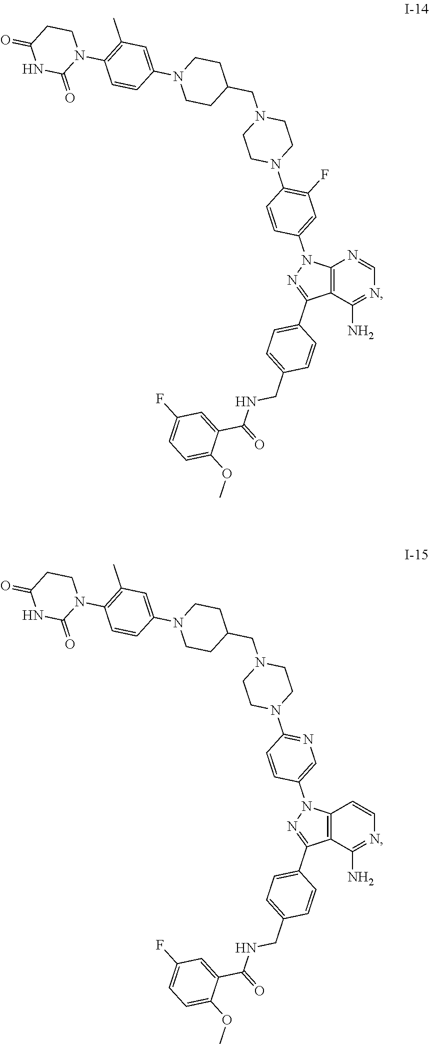 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)c(F)c3)c3ncnc(N)c23)cc1 ; COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ccnc(N)c23)cc1]

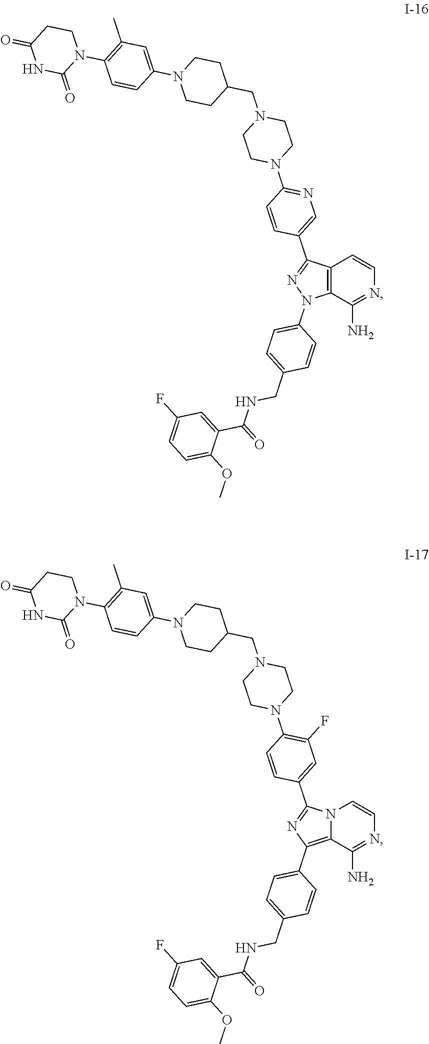 [SMILES: COc1ccc(F)cc1C(=O)NCc1ccc(-n2nc(-c3ccc(N4CCN(CC5CCN(c6ccc(N7CCC(=O)NC7=O)c(C)c6)CC5)CC4)nc3)c3ccnc(N)c32)cc1 ; COc1ccc(F)cc1C(=O)NCc1ccc(-c2nc(-c3ccc(N4CCN(CC5CCN(c6ccc(N7CCC(=O)NC7=O)c(C)c6)CC5)CC4)c(F)c3)n3ccnc(N)c23)cc1]

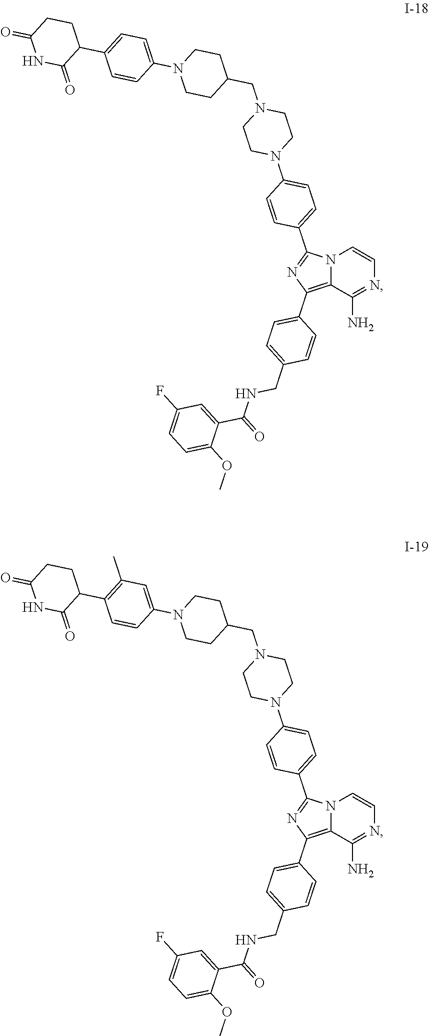 [SMILES: COc1ccc(F)cc1C(=O)NCc1ccc(-c2nc(-c3ccc(N4CCN(CC5CCN(c6ccc(C7CCC(=O)NC7=O)cc6)CC5)CC4)cc3)n3ccnc(N)c23)cc1 ; COc1ccc(F)cc1C(=O)NCc1ccc(-c2nc(-c3ccc(N4CCN(CC5CCN(c6ccc(C7CCC(=O)NC7=O)c(C)c6)CC5)CC4)cc3)n3ccnc(N)c23)cc1]

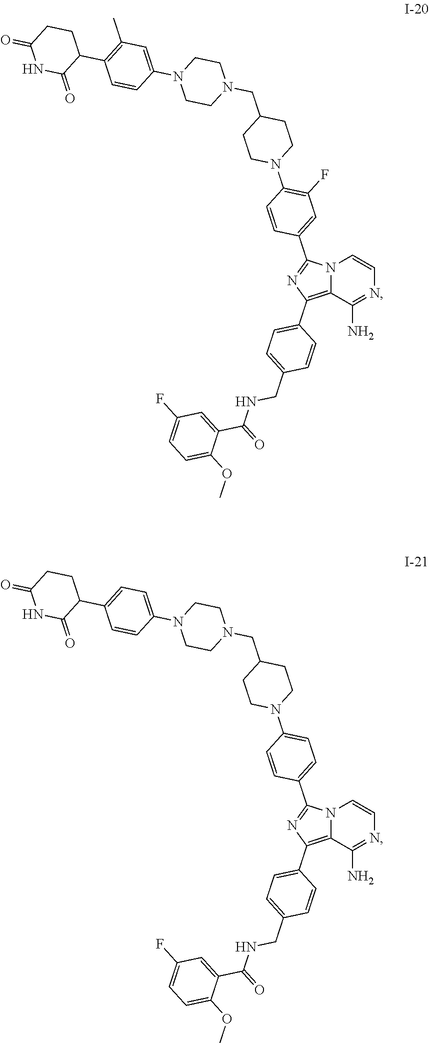 [SMILES: COc1ccc(F)cc1C(=O)NCc1ccc(-c2nc(-c3ccc(N4CCC(CN5CCN(c6ccc(C7CCC(=O)NC7=O)c(C)c6)CC5)CC4)c(F)c3)n3ccnc(N)c23)cc1 ; COc1ccc(F)cc1C(=O)NCc1ccc(-c2nc(-c3ccc(N4CCC(CN5CCN(c6ccc(C7CCC(=O)NC7=O)cc6)CC5)CC4)cc3)n3ccnc(N)c23)cc1]

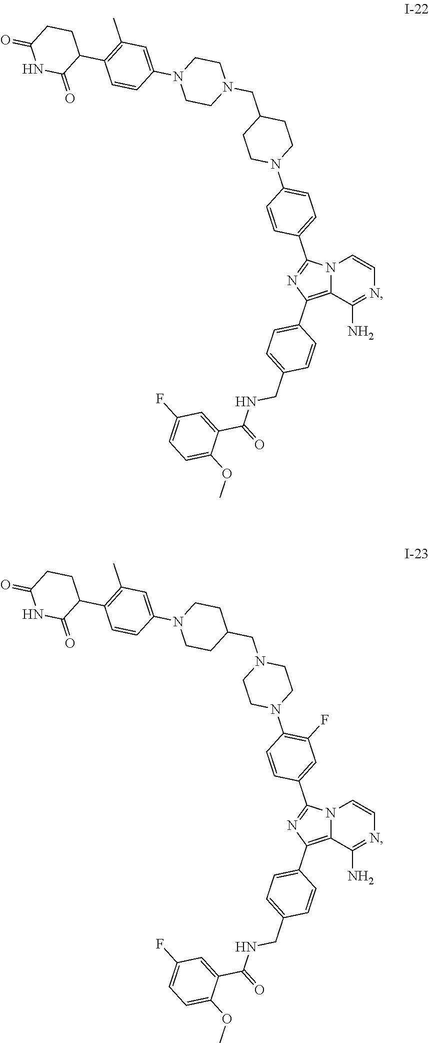 [SMILES: COc1ccc(F)cc1C(=O)NCc1ccc(-c2nc(-c3ccc(N4CCC(CN5CCN(c6ccc(C7CCC(=O)NC7=O)c(C)c6)CC5)CC4)cc3)n3ccnc(N)c23)cc1 ; COc1ccc(F)cc1C(=O)NCc1ccc(-c2nc(-c3ccc(N4CCN(CC5CCN(c6ccc(C7CCC(=O)NC7=O)c(C)c6)CC5)CC4)c(F)c3)n3ccnc(N)c23)cc1]

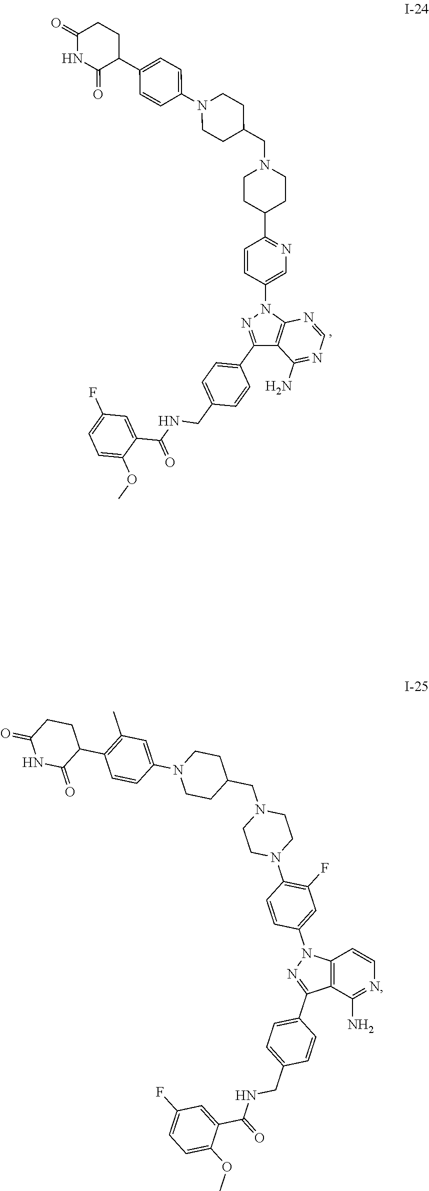 [SMILES: COc1ccc(F)cc1C(=O)NCc1ccc(-c2nn(-c3ccc(C4CCN(CC5CCN(c6ccc(C7CCC(=O)NC7=O)cc6)CC5)CC4)nc3)c3ncnc(N)c23)cc1 ; COc1ccc(F)cc1C(=O)NCc1ccc(-c2nn(-c3ccc(N4CCN(CC5CCN(c6ccc(C7CCC(=O)NC7=O)c(C)c6)CC5)CC4)c(F)c3)c3ccnc(N)c23)cc1]

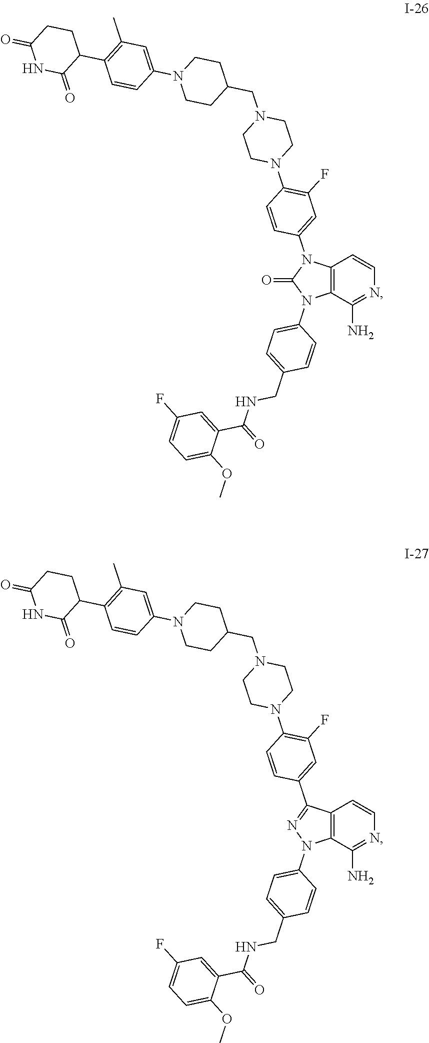 [SMILES: COc1ccc(F)cc1C(=O)NCc1ccc(-n2c(=O)n(-c3ccc(N4CCN(CC5CCN(c6ccc(C7CCC(=O)NC7=O)c(C)c6)CC5)CC4)c(F)c3)c3ccnc(N)c32)cc1 ; COc1ccc(F)cc1C(=O)NCc1ccc(-n2nc(-c3ccc(N4CCN(CC5CCN(c6ccc(C7CCC(=O)NC7=O)c(C)c6)CC5)CC4)c(F)c3)c3ccnc(N)c32)cc1]

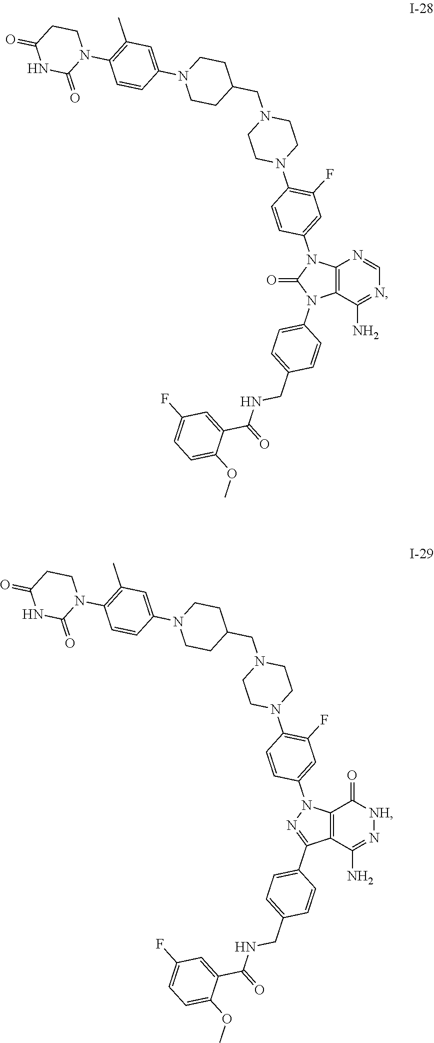 [SMILES: COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)c(F)c3)c3ncnc(N)c32)cc1 ; COc1ccc(F)cc1C(=O)NCc1ccc(-c2nn(-c3ccc(N4CCN(CC5CCN(c6ccc(N7CCC(=O)NC7=O)c(C)c6)CC5)CC4)c(F)c3)c3c(=O)[nH]nc(N)c23)cc1]

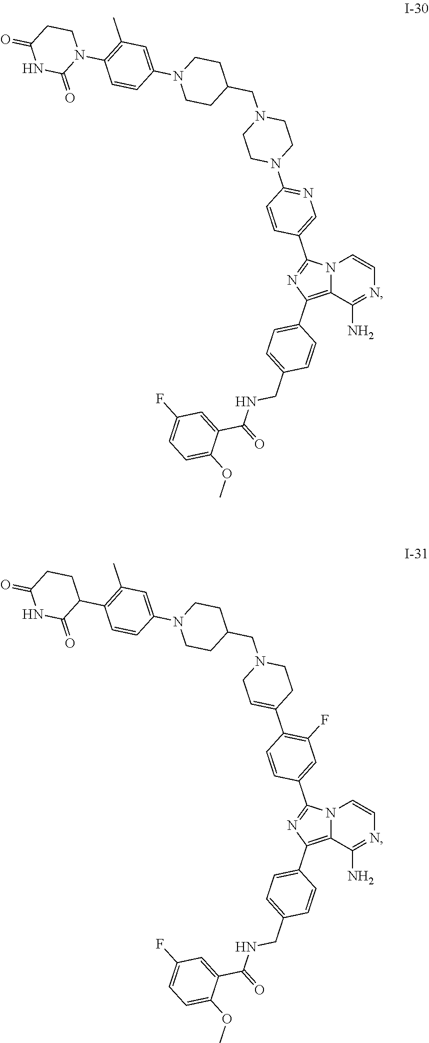 [SMILES: COc1ccc(F)cc1C(=O)NCc1ccc(-c2nc(-c3ccc(N4CCN(CC5CCN(c6ccc(N7CCC(=O)NC7=O)c(C)c6)CC5)CC4)nc3)n3ccnc(N)c23)cc1 ; COc1ccc(F)cc1C(=O)NCc1ccc(-c2nc(-c3ccc(C4=CCN(CC5CCN(c6ccc(C7CCC(=O)NC7=O)c(C)c6)CC5)CC4)c(F)c3)n3ccnc(N)c23)cc1]

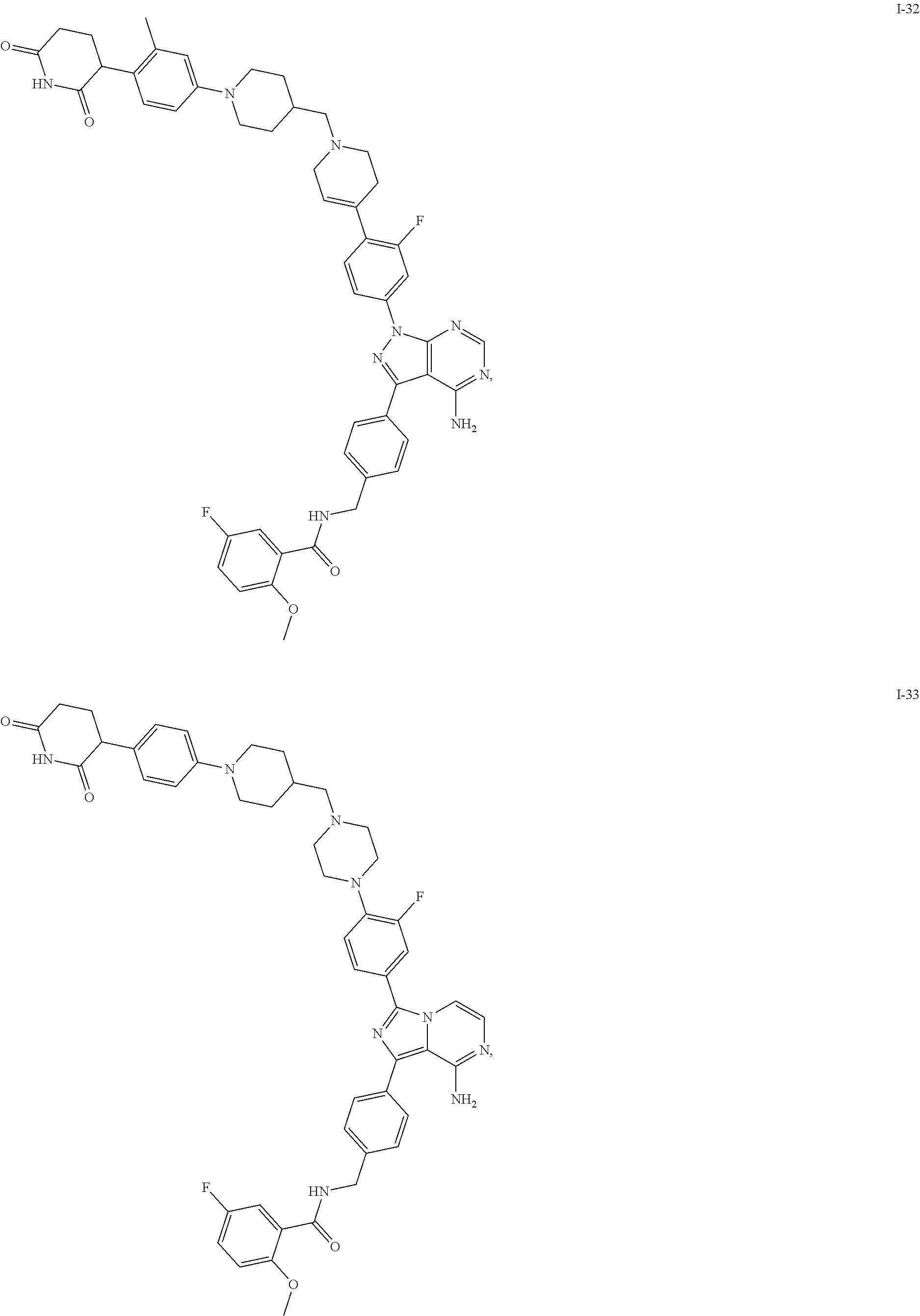 [SMILES: COc1ccc(F)cc1C(=O)NCc1ccc(-c2nn(-c3ccc(C4=CCN(CC5CCN(c6ccc(C7CCC(=O)NC7=O)c(C)c6)CC5)CC4)c(F)c3)c3ncnc(N)c23)cc1 ; COc1ccc(F)cc1C(=O)NCc1ccc(-c2nc(-c3ccc(N4CCN(CC5CCN(c6ccc(C7CCC(=O)NC7=O)cc6)CC5)CC4)c(F)c3)n3ccnc(N)c23)cc1]

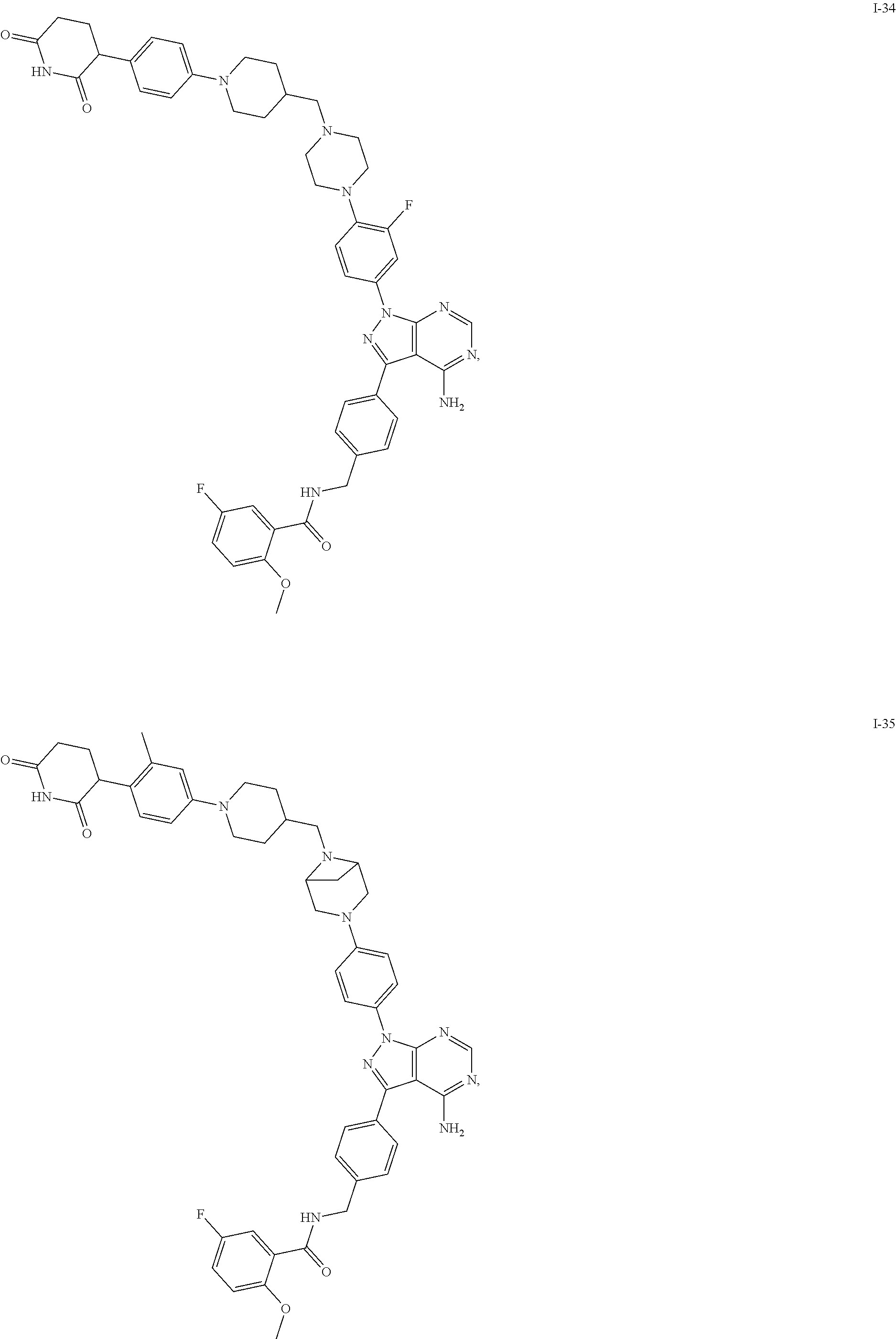 [SMILES: COc1ccc(F)cc1C(=O)NCc1ccc(-c2nn(-c3ccc(N4CCN(CC5CCN(c6ccc(C7CCC(=O)NC7=O)cc6)CC5)CC4)c(F)c3)c3ncnc(N)c23)cc1 ; COc1ccc(F)cc1C(=O)NCc1ccc(-c2nn(-c3ccc(N4CC5CC(C4)N5CC4CCN(c5ccc(C6CCC(=O)NC6=O)c(C)c5)CC4)cc3)c3ncnc(N)c23)cc1]

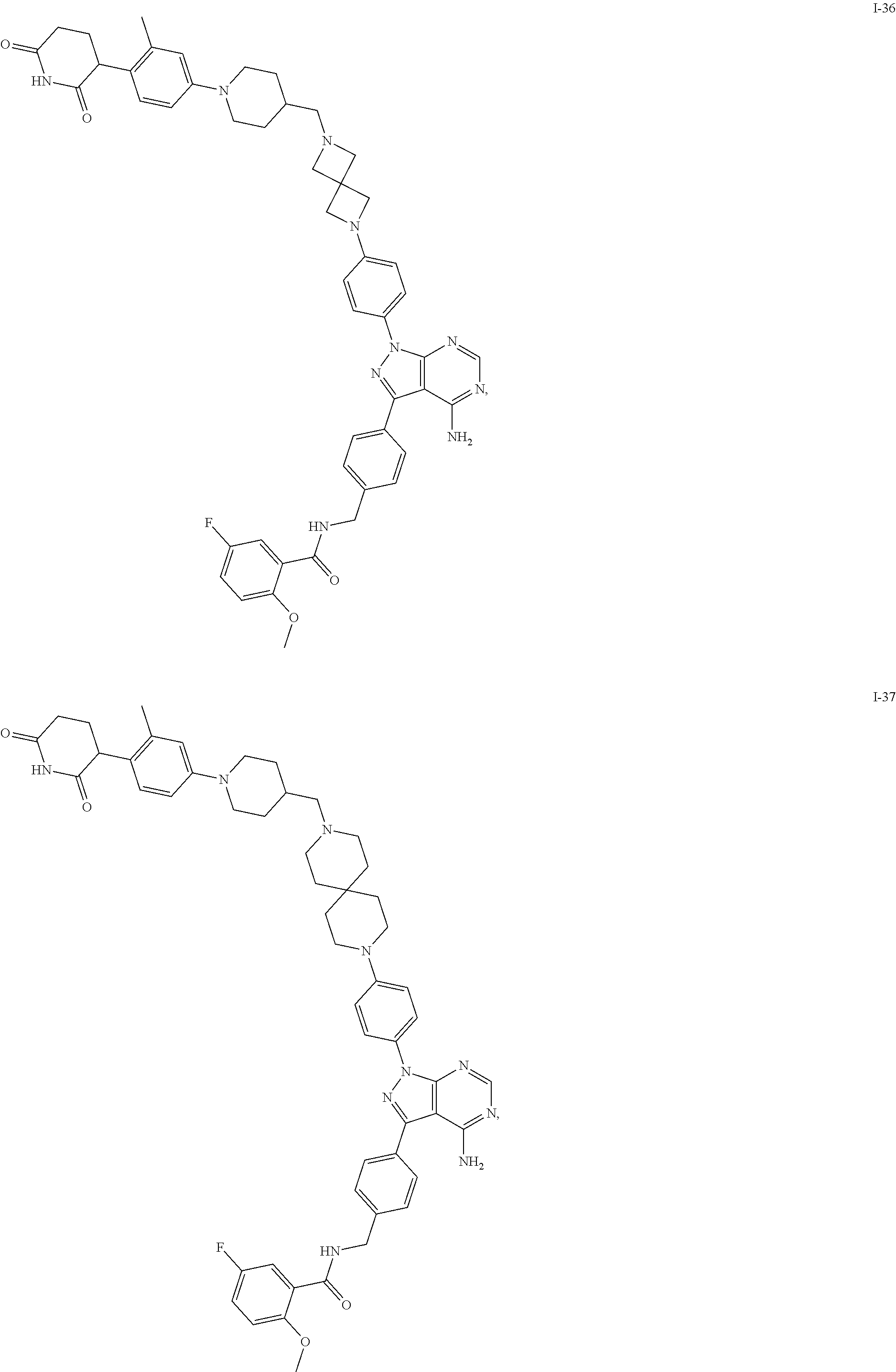 [SMILES: COc1ccc(F)cc1C(=O)NCc1ccc(-c2nn(-c3ccc(N4CC5(CN(CC6CCN(c7ccc(C8CCC(=O)NC8=O)c(C)c7)CC6)C5)C4)cc3)c3ncnc(N)c23)cc1 ; COc1ccc(F)cc1C(=O)NCc1ccc(-c2nn(-c3ccc(N4CCC5(CCN(CC6CCN(c7ccc(C8CCC(=O)NC8=O)c(C)c7)CC6)CC5)CC4)cc3)c3ncnc(N)c23)cc1]

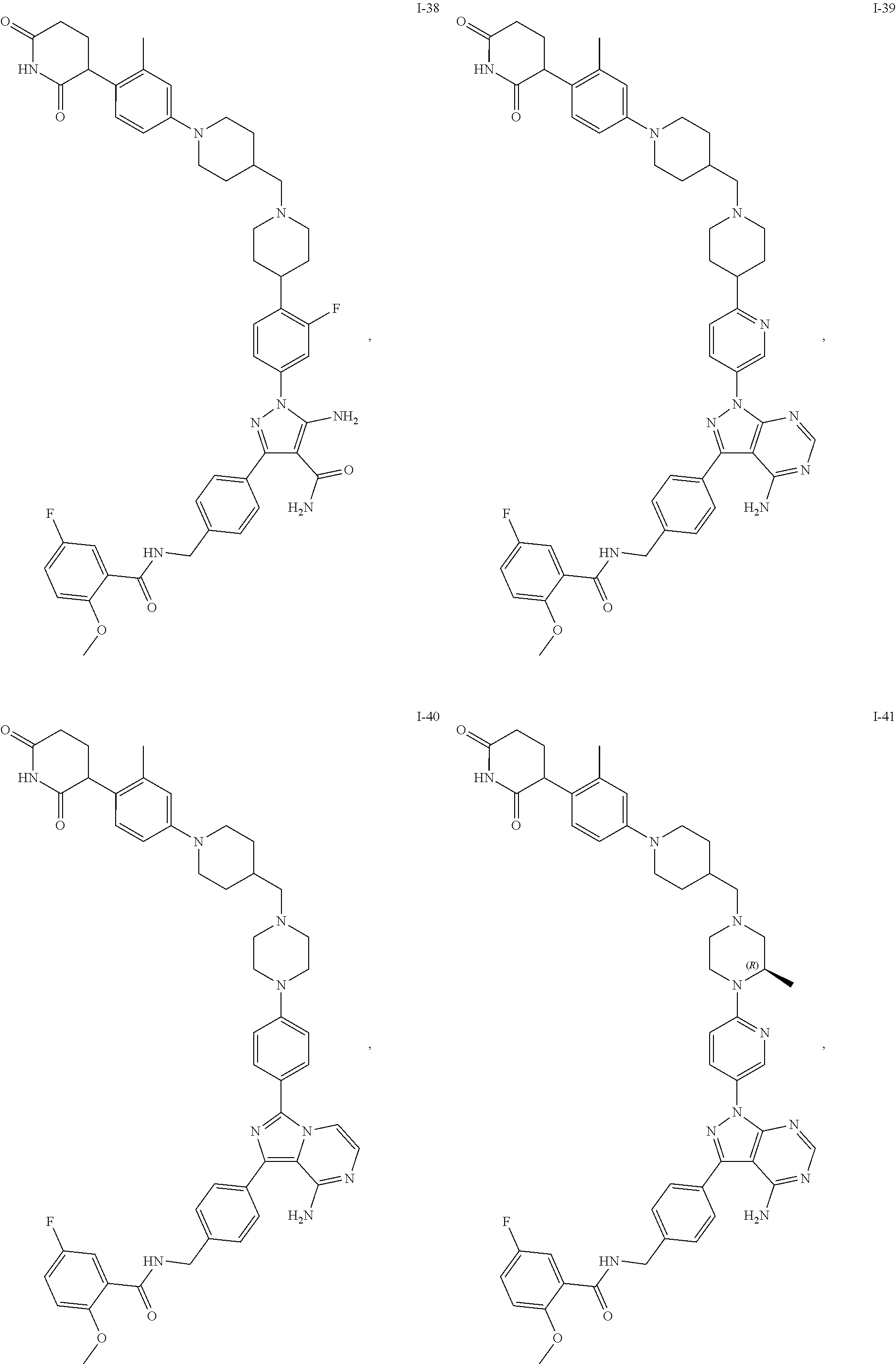

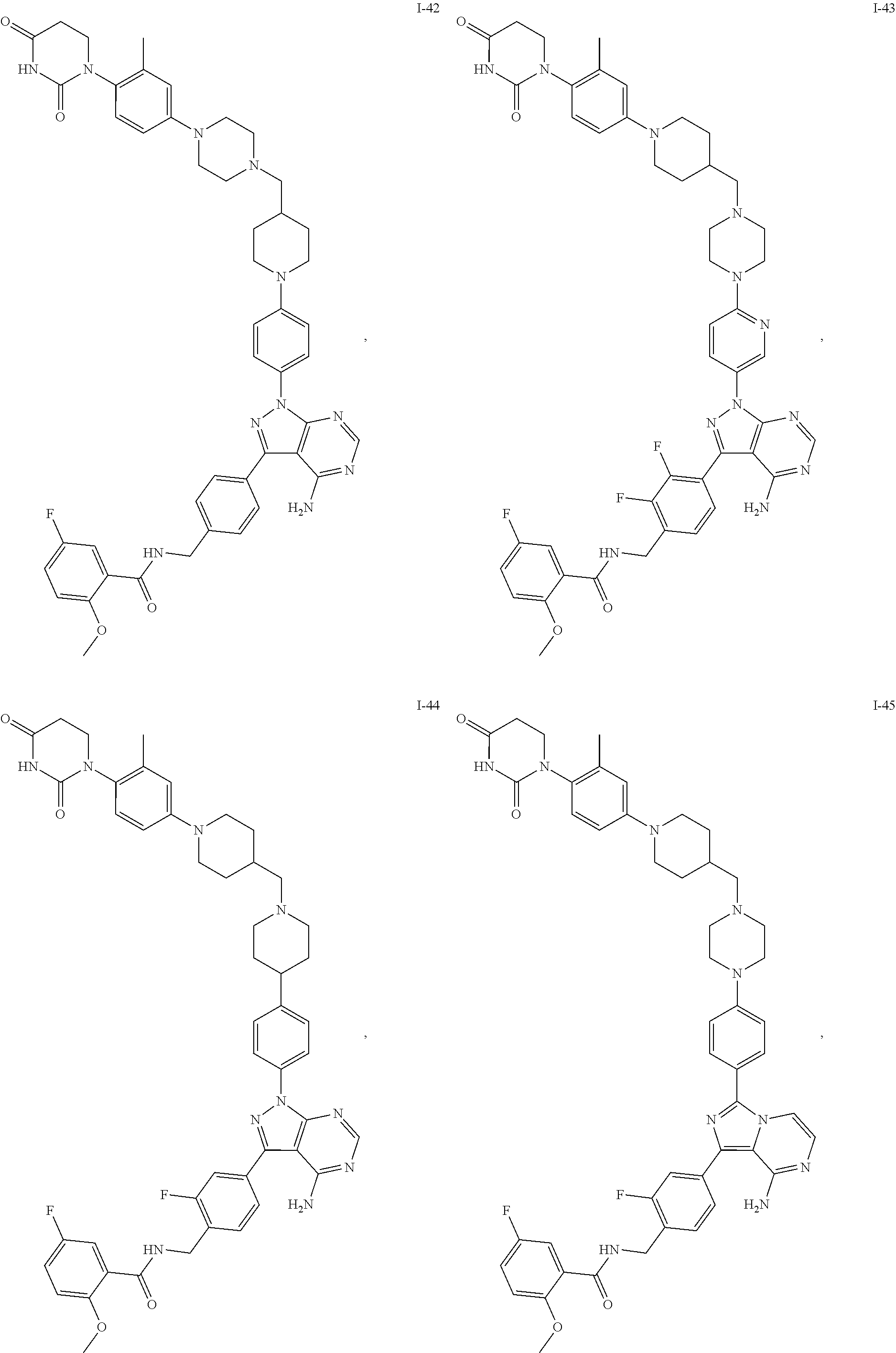 [SMILES: COc1ccc(F)cc1C(=O)NCc1ccc(-c2nn(-c3ccc(N4CCC(CN5CCN(c6ccc(N7CCC(=O)NC7=O)c(C)c6)CC5)CC4)cc3)c3ncnc(N)c23)cc1 ; COc1ccc(F)cc1C(=O)NCc1ccc(-c2nn(-c3ccc(N4CCN(CC5CCN(c6ccc(N7CCC(=O)NC7=O)c(C)c6)CC5)CC4)nc3)c3ncnc(N)c23)c(F)c1F ; COc1ccc(F)cc1C(=O)NCc1ccc(-c2nn(-c3ccc(C4CCN(CC5CCN(c6ccc(N7CCC(=O)NC7=O)c(C)c6)CC5)CC4)cc3)c3ncnc(N)c23)cc1F ; COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)cc1F]

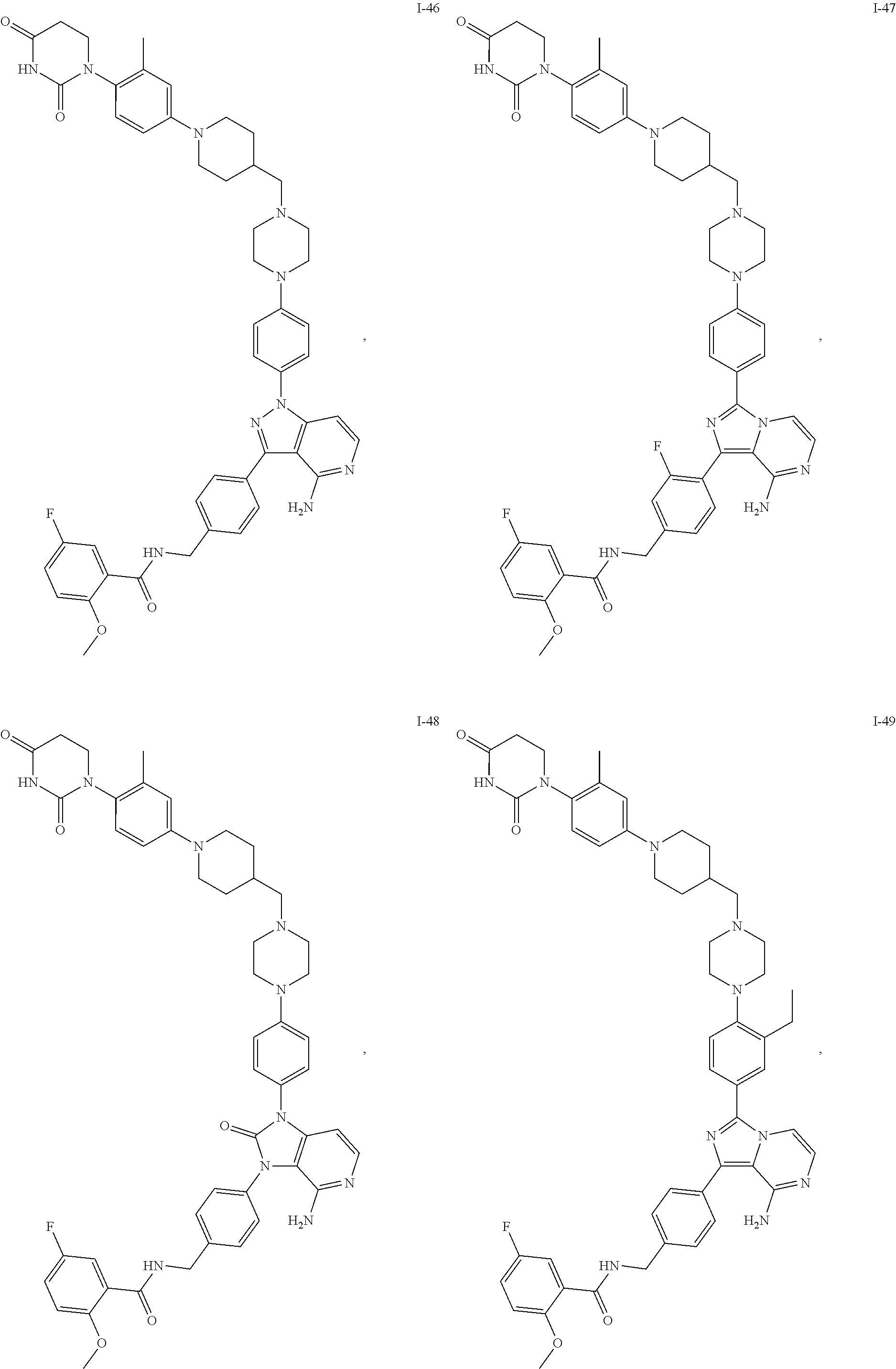 [SMILES: COc1ccc(F)cc1C(=O)NCc1ccc(-c2nn(-c3ccc(N4CCN(CC5CCN(c6ccc(N7CCC(=O)NC7=O)c(C)c6)CC5)CC4)cc3)c3ccnc(N)c23)cc1 ; COc1ccc(F)cc1C(=O)NCc1ccc(-c2nc(-c3ccc(N4CCN(CC5CCN(c6ccc(N7CCC(=O)NC7=O)c(C)c6)CC5)CC4)cc3)n3ccnc(N)c23)c(F)c1 ; COc1ccc(F)cc1C(=O)NCc1ccc(-n2c(=O)n(-c3ccc(N4CCN(CC5CCN(c6ccc(N7CCC(=O)NC7=O)c(C)c6)CC5)CC4)cc3)c3ccnc(N)c32)cc1 ; CCc1cc(-c2nc(-c3ccc(CNC(=O)c4cc(F)ccc4OC)cc3)c3c(N)nccn23)ccc1N1CCN(CC2CCN(c3ccc(N4CCC(=O)NC4=O)c(C)c3)CC2)CC1]

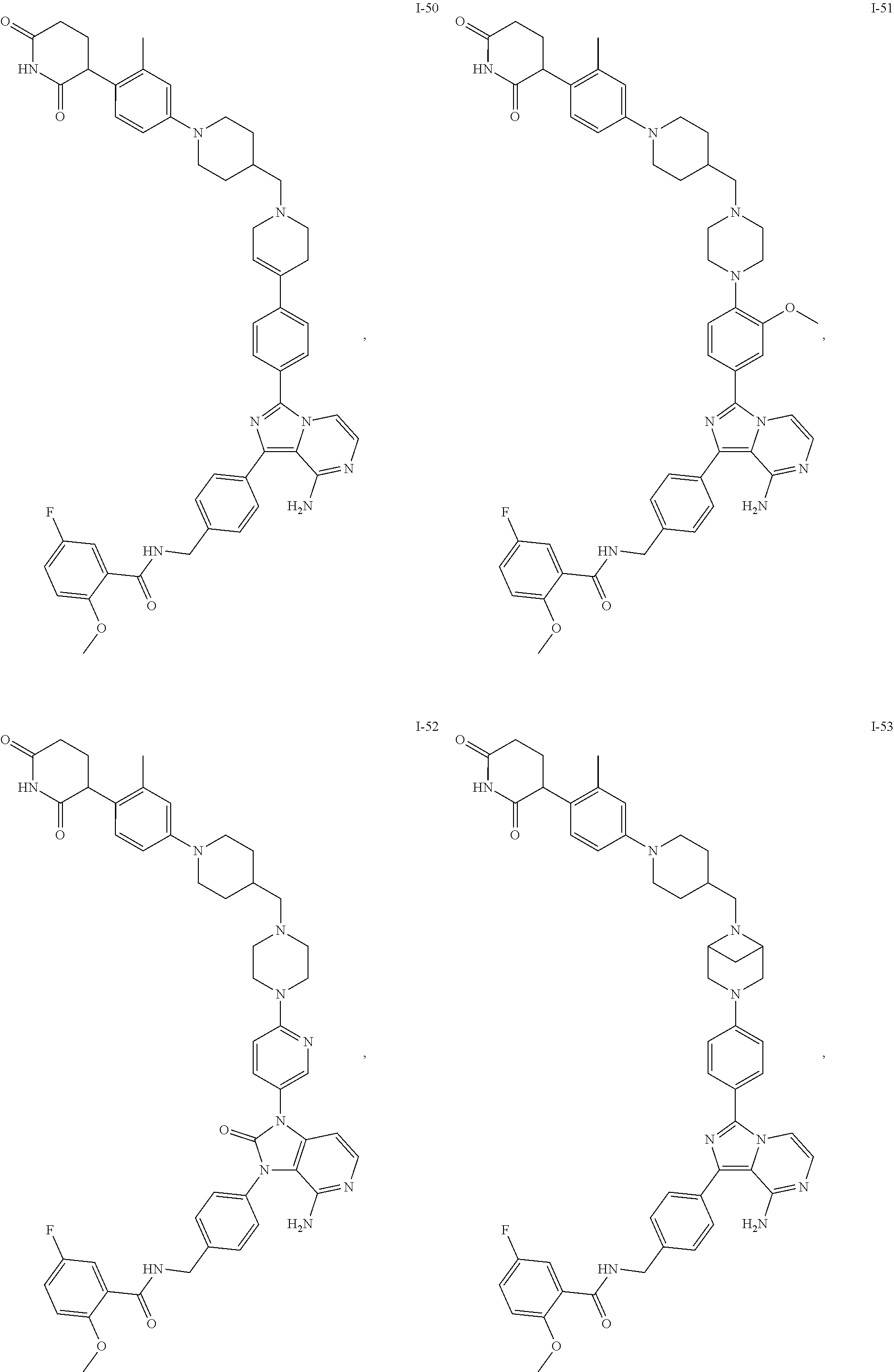 [SMILES: COc1ccc(F)cc1C(=O)NCc1ccc(-c2nc(-c3ccc(C4=CCN(CC5CCN(c6ccc(C7CCC(=O)NC7=O)c(C)c6)CC5)CC4)cc3)n3ccnc(N)c23)cc1 ; COc1ccc(F)cc1C(=O)NCc1ccc(-c2nc(-c3ccc(N4CCN(CC5CCN(c6ccc(C7CCC(=O)NC7=O)c(C)c6)CC5)CC4)c(OC)c3)n3ccnc(N)c23)cc1 ; COc1ccc(F)cc1C(=O)NCc1ccc(-n2c(=O)n(-c3ccc(N4CCN(CC5CCN(c6ccc(C7CCC(=O)NC7=O)c(C)c6)CC5)CC4)nc3)c3ccnc(N)c32)cc1 ; COc1ccc(F)cc1C(=O)NCc1ccc(-c2nc(-c3ccc(N4CC5CC(C4)N5CC4CCN(c5ccc(C6CCC(=O)NC6=O)c(C)c5)CC4)cc3)n3ccnc(N)c23)cc1]

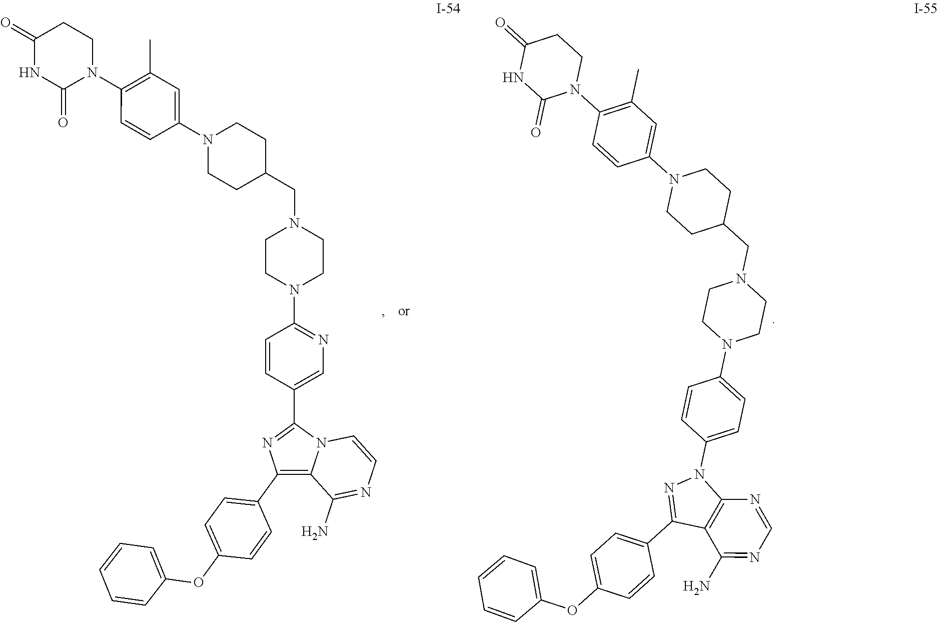 [SMILES: Cc1cc(N2CCC(CN3CCN(c4ccc(-c5nc(-c6ccc(Oc7ccccc7)cc6)c6c(N)nccn56)cn4)CC3)CC2)ccc1N1CCC(=O)NC1=O ; Cc1cc(N2CCC(CN3CCN(c4ccc(-n5nc(-c6ccc(Oc7ccccc7)cc6)c6c(N)ncnc65)cc4)CC3)CC2)ccc1N1CCC(=O)NC1=O]

22. The compound according to claim 7, or the isomer or pharmaceutically acceptable salt thereof,

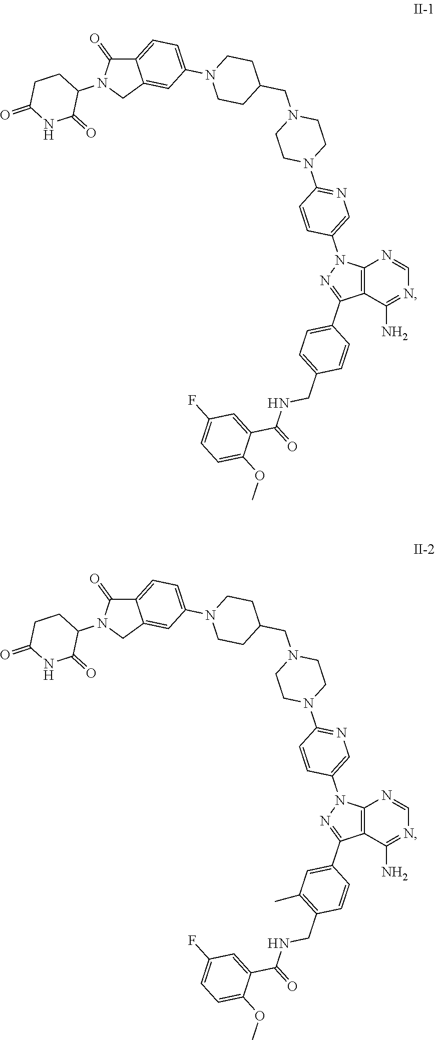 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1C]

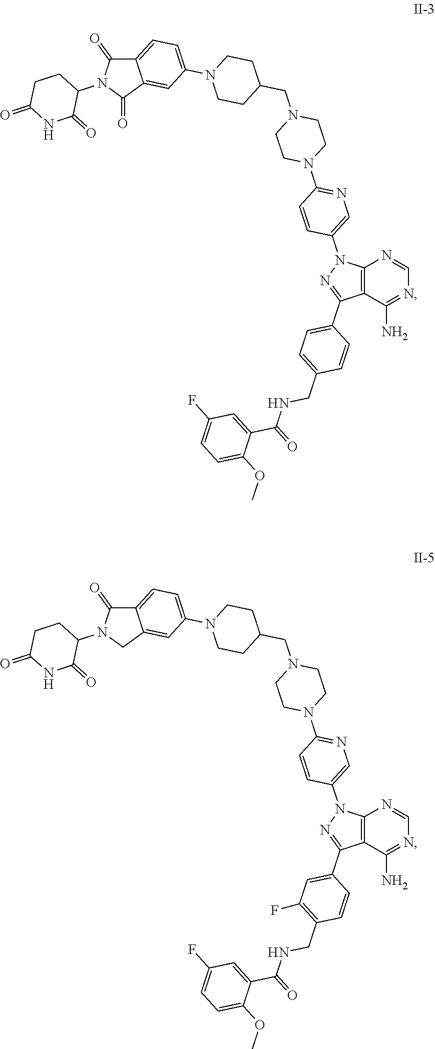 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ncnc(N)c23)cc1F]

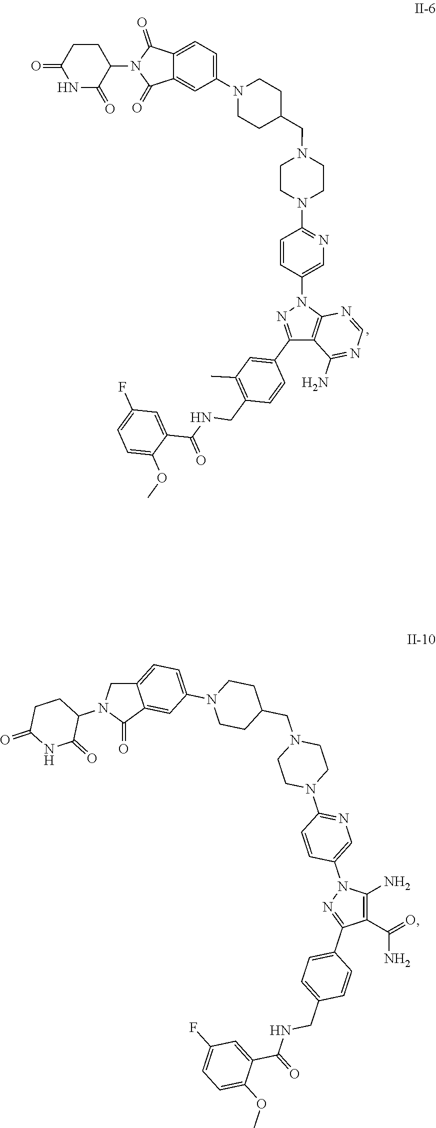 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7=O)CC5)CC4)nc3)c3ncnc(N)c23)cc1C ; COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)nc3)c(N)c2C(N)=O)cc1]

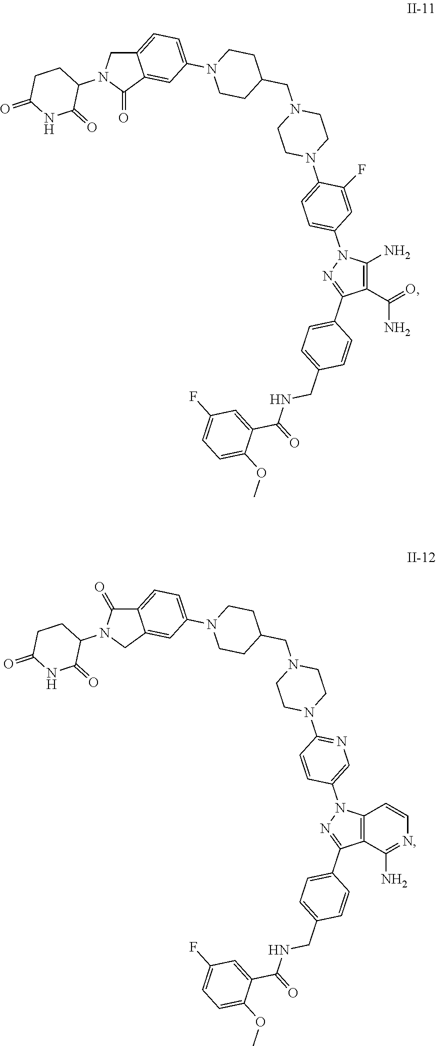 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)c(F)c3)c(N)c2C(N)=O)cc1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ccnc(N)c23)cc1]

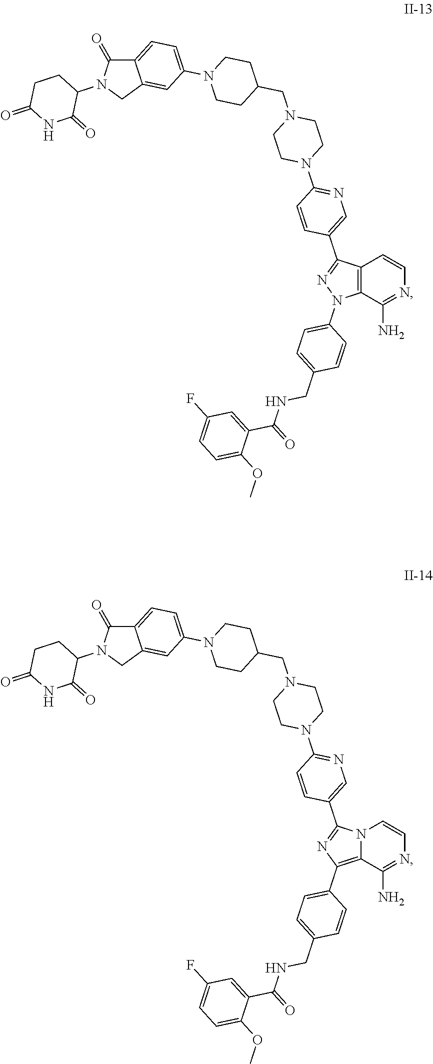 [SMILES: COc1ccc(F)cc1C(=O)NCc1ccc(-n2nc(-c3ccc(N4CCN(CC5CCN(c6ccc7c(c6)CN(C6CCC(=O)NC6=O)C7=O)CC5)CC4)nc3)c3ccnc(N)c32)cc1 ; COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)nc3)n3ccnc(N)c23)cc1]

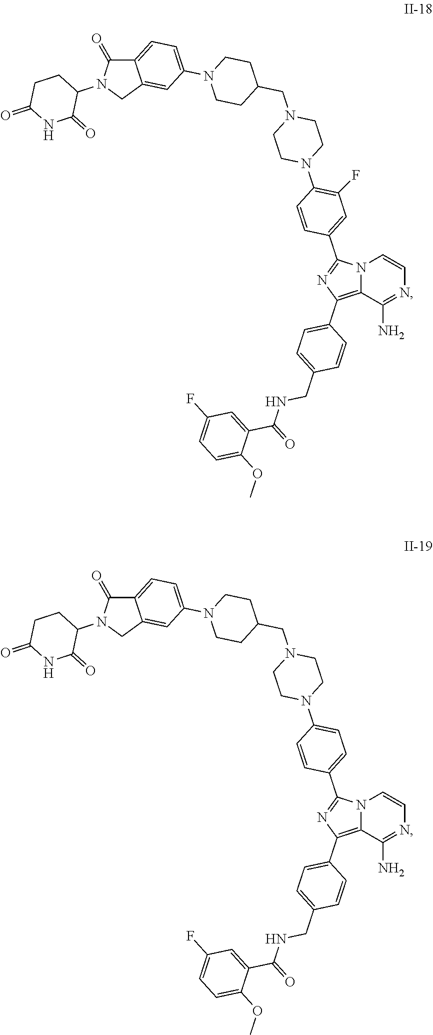 [SMILES: COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)c(F)c3)n3ccnc(N)c23)cc1 ; COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)cc3)n3ccnc(N)c23)cc1]

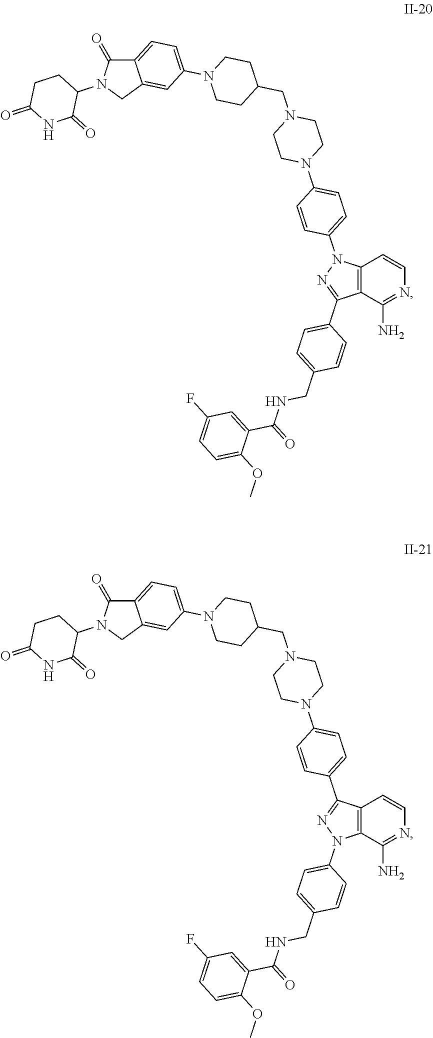 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)cc3)c3ccnc(N)c23)cc1 ; COc1ccc(F)cc1C(=O)NCc1ccc(-n2nc(-c3ccc(N4CCN(CC5CCN(c6ccc7c(c6)CN(C6CCC(=O)NC6=O)C7=O)CC5)CC4)cc3)c3ccnc(N)c32)cc1]

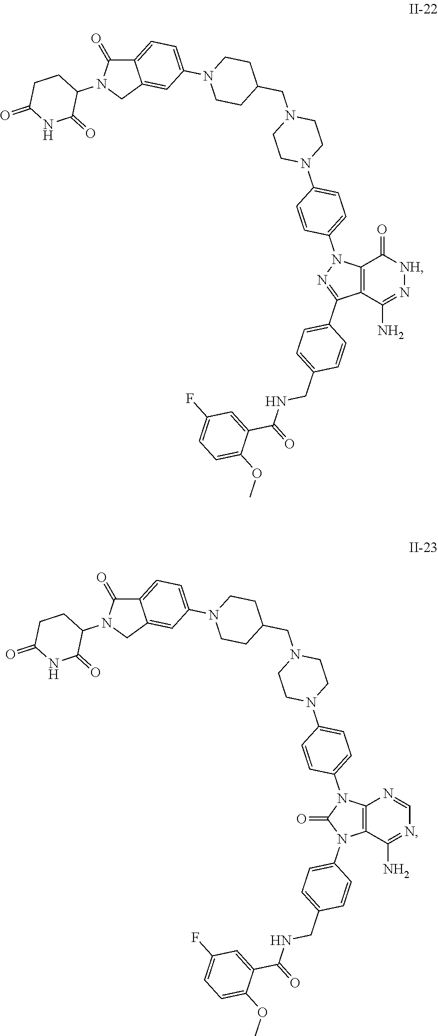 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)cc3)c3c(=O)[nH]nc(N)c23)cc1 ; COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)cc3)c3ncnc(N)c32)cc1]

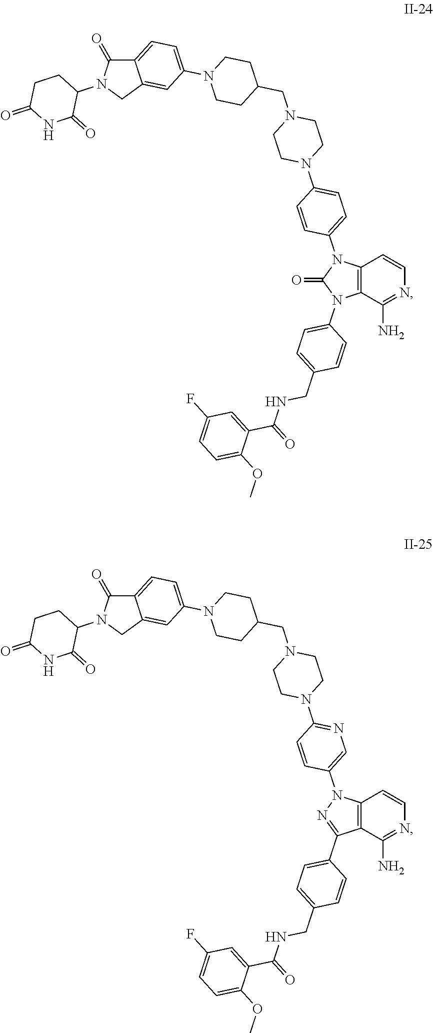 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)cc3)c3ccnc(N)c32)cc1 ; COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3ccnc(N)c23)cc1]

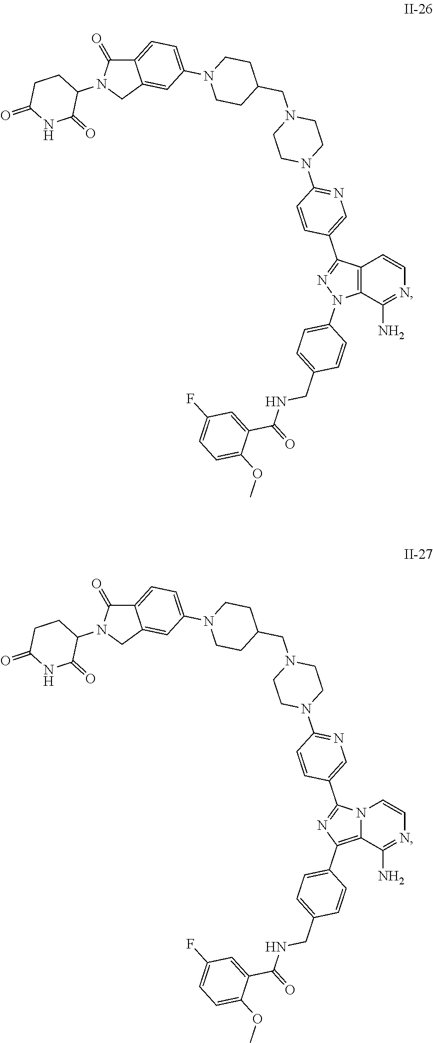 [SMILES: COc1ccc(F)cc1C(=O)NCc1ccc(-n2nc(-c3ccc(N4CCN(CC5CCN(c6ccc7c(c6)CN(C6CCC(=O)NC6=O)C7=O)CC5)CC4)nc3)c3ccnc(N)c32)cc1 ; COc1ccc(F)cc1C(=O)NCc1ccc(-c2nc(-c3ccc(N4CCN(CC5CCN(c6ccc7c(c6)CN(C6CCC(=O)NC6=O)C7=O)CC5)CC4)nc3)n3ccnc(N)c23)cc1]

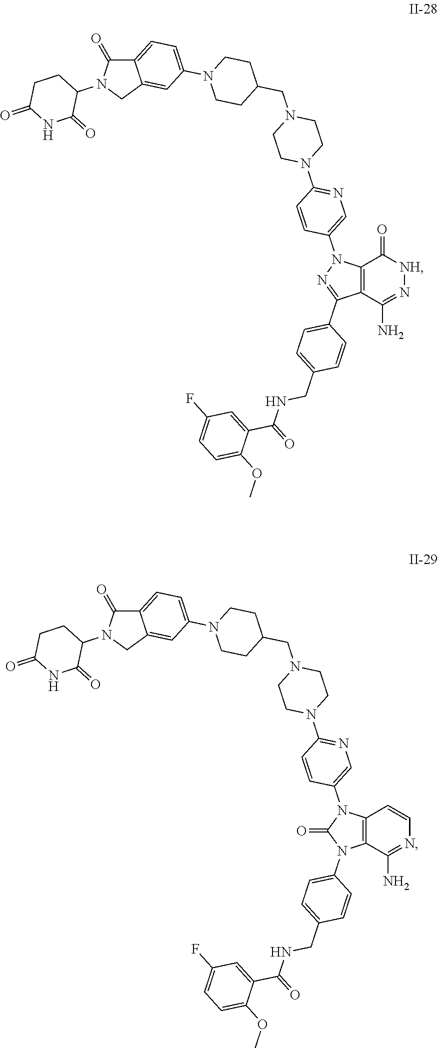 [SMILES: COc1ccc(F)cc1C(=O)NCc1ccc(-c2nn(-c3ccc(N4CCN(CC5CCN(c6ccc7c(c6)CN(C6CCC(=O)NC6=O)C7=O)CC5)CC4)nc3)c3c(=O)[nH]nc(N)c23)cc1 ; COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)nc3)c3ccnc(N)c32)cc1]

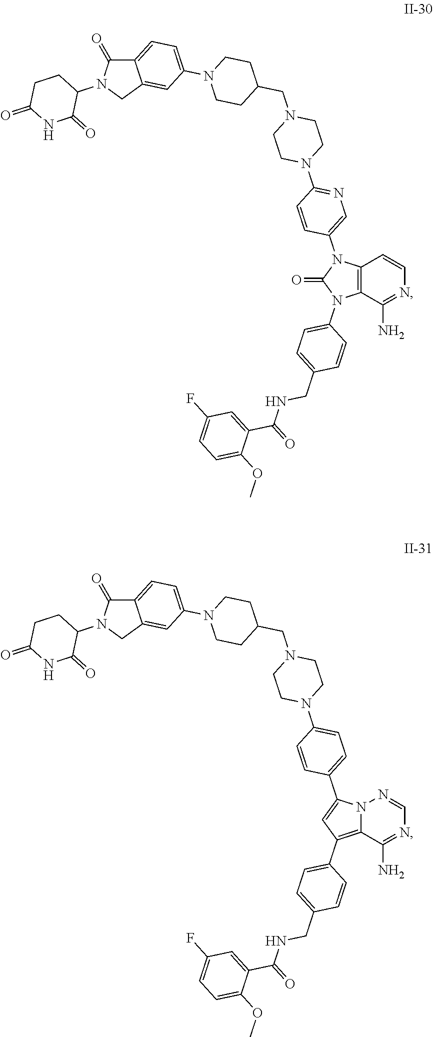 [SMILES: COc1ccc(F)cc1C(=O)NCc1ccc(-n2c(=O)n(-c3ccc(N4CCN(CC5CCN(c6ccc7c(c6)CN(C6CCC(=O)NC6=O)C7=O)CC5)CC4)nc3)c3ccnc(N)c32)cc1 ; COc1ccc(F)cc1C(=O)NCc1ccc(-c2cc(-c3ccc(N4CCN(CC5CCN(c6ccc7c(c6)CN(C6CCC(=O)NC6=O)C7=O)CC5)CC4)cc3)n3ncnc(N)c23)cc1]

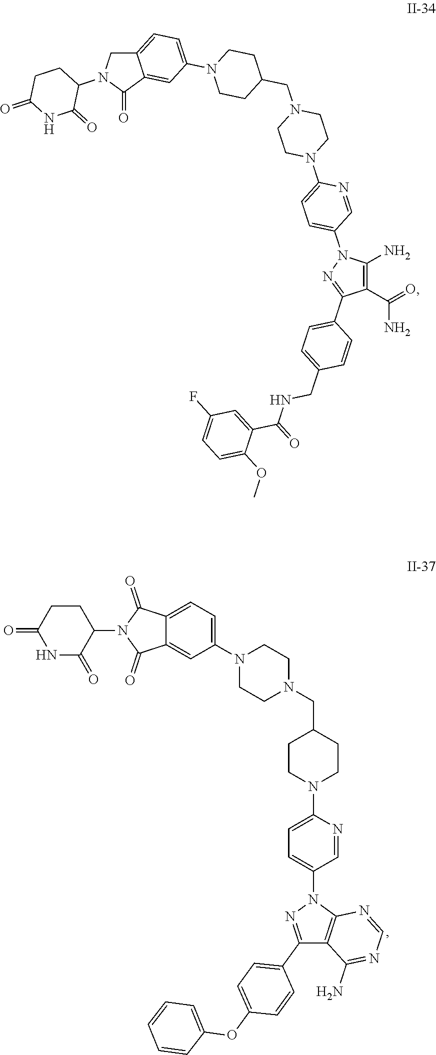 [SMILES: COc1ccc(F)cc1C(=O)NCc1ccc(-c2nn(-c3ccc(N4CCN(CC5CCN(c6ccc7c(c6)C(=O)N(C6CCC(=O)NC6=O)C7)CC5)CC4)nc3)c(N)c2C(N)=O)cc1 ; Nc1ncnc2c1c(-c1ccc(Oc3ccccc3)cc1)nn2-c1ccc(N2CCC(CN3CCN(c4ccc5c(c4)C(=O)N(C4CCC(=O)NC4=O)C5=O)CC3)CC2)nc1]

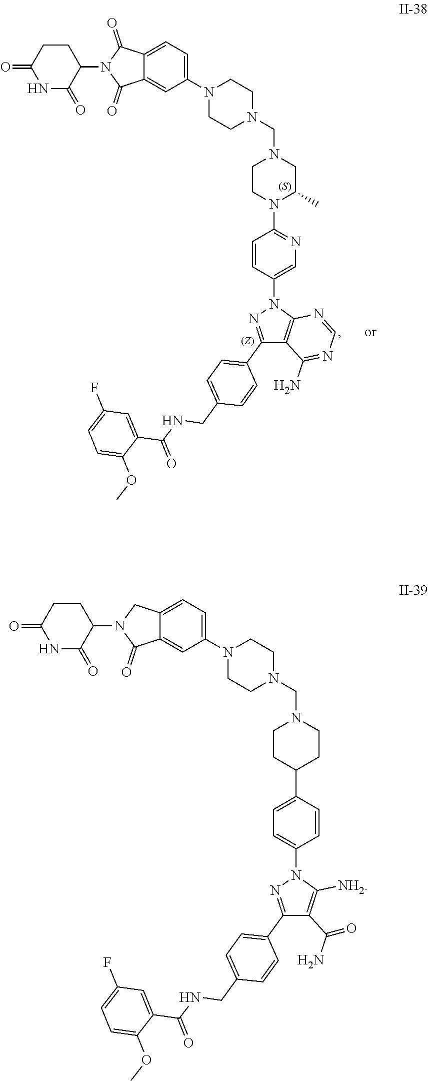

23. The compound according to claim 13, or the isomer or pharmaceutically acceptable salt thereof, selected from the following structures:

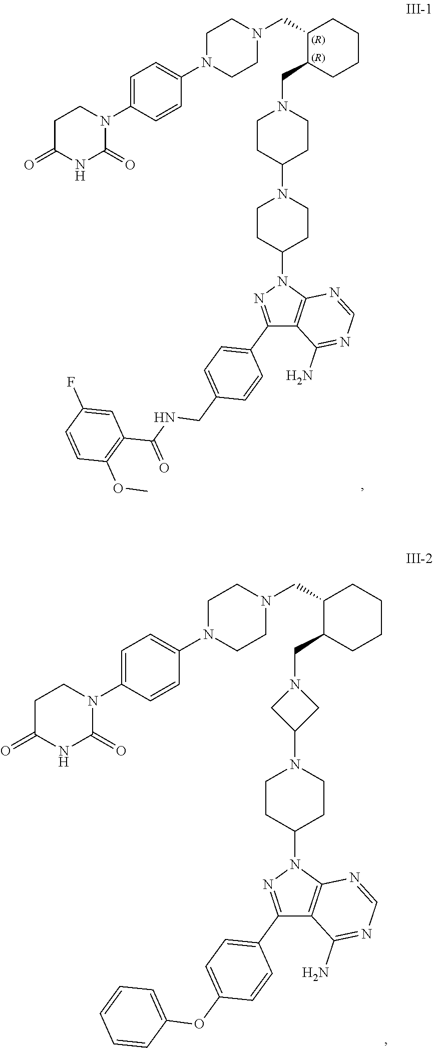

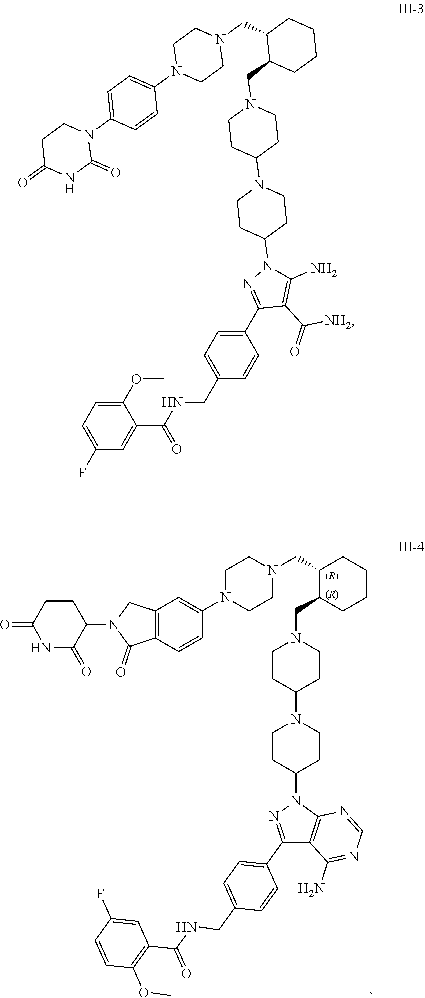

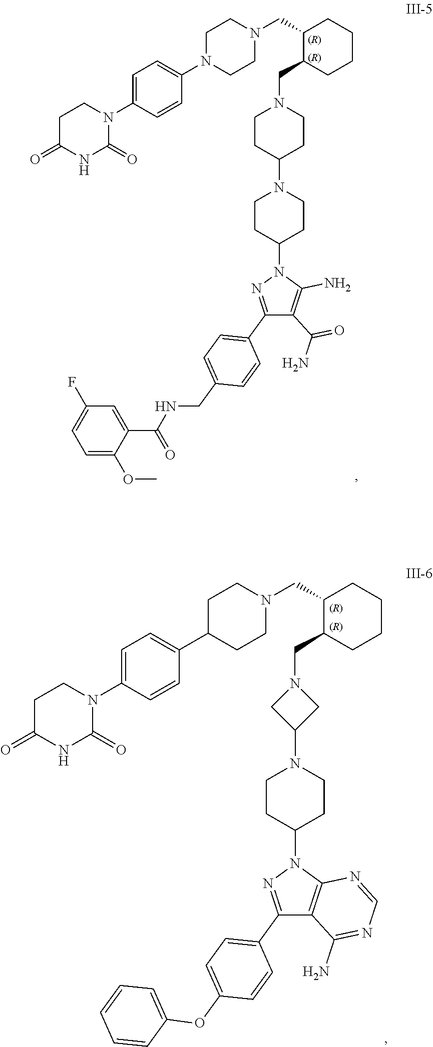

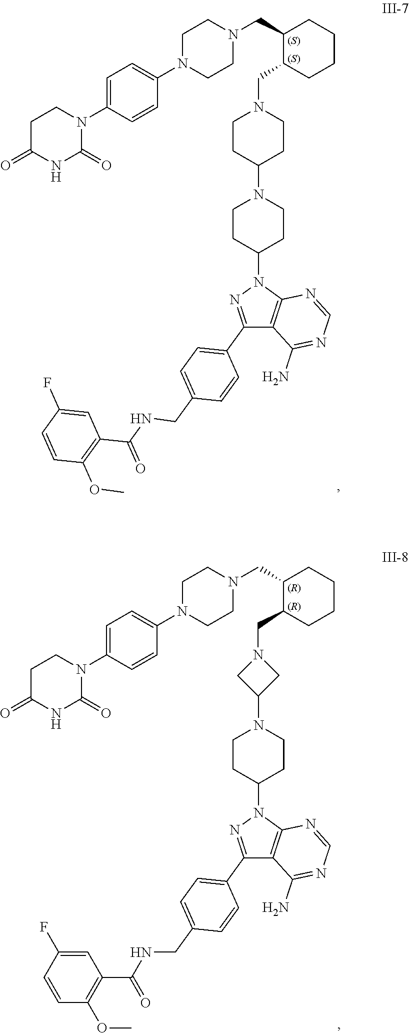

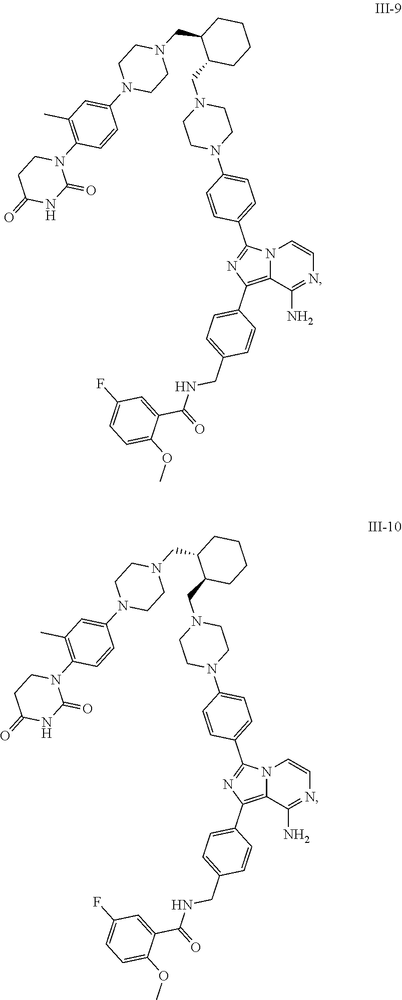 [SMILES: COc1ccc(F)cc1C(=O)NCc1ccc(-c2nc(-c3ccc(N4CCN(C[C@H]5CCCC[C@@H]5CN5CCN(c6ccc(N7CCC(=O)NC7=O)c(C)c6)CC5)CC4)cc3)n3ccnc(N)c23)cc1 ; COc1ccc(F)cc1C(=O)NCc1ccc(-c2nc(-c3ccc(N4CCN(C[C@@H]5CCCC[C@H]5CN5CCN(c6ccc(N7CCC(=O)NC7=O)c(C)c6)CC5)CC4)cc3)n3ccnc(N)c23)cc1]

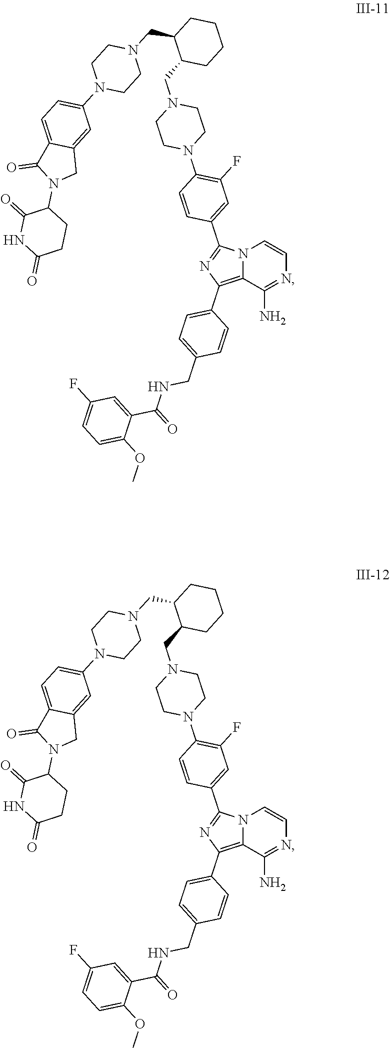 [SMILES: COc1ccc(F)cc1C(=O)NCc1ccc(-c2nc(-c3ccc(N4CCN(C[C@H]5CCCC[C@@H]5CN5CCN(c6ccc7c(c6)CN(C6CCC(=O)NC6=O)C7=O)CC5)CC4)c(F)c3)n3ccnc(N)c23)cc1 ; COc1ccc(F)cc1C(=O)NCc1ccc(-c2nc(-c3ccc(N4CCN(C[C@@H]5CCCC[C@H]5CN5CCN(c6ccc7c(c6)CN(C6CCC(=O)NC6=O)C7=O)CC5)CC4)c(F)c3)n3ccnc(N)c23)cc1]

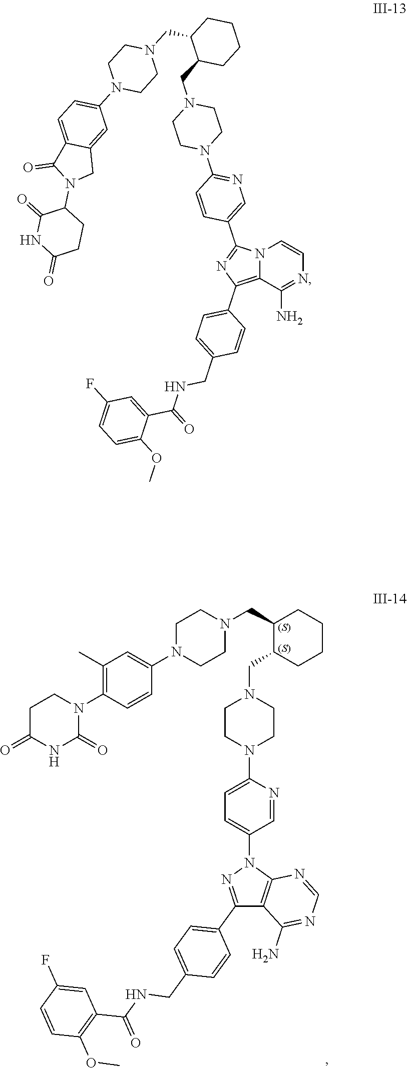

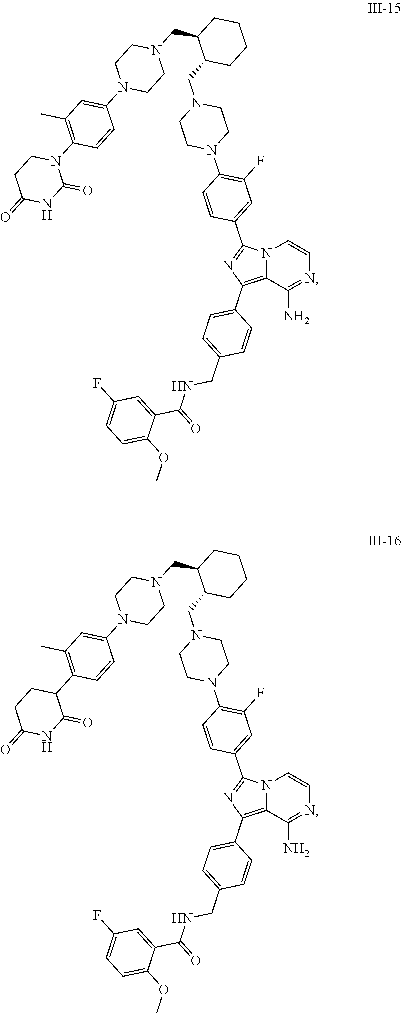 [SMILES: COc1ccc(F)cc1C(=O)NCc1ccc(-c2nc(-c3ccc(N4CCN(C[C@H]5CCCC[C@@H]5CN5CCN(c6ccc(N7CCC(=O)NC7=O)c(C)c6)CC5)CC4)c(F)c3)n3ccnc(N)c23)cc1 ; COc1ccc(F)cc1C(=O)NCc1ccc(-c2nc(-c3ccc(N4CCN(C[C@H]5CCCC[C@@H]5CN5CCN(c6ccc(C7CCC(=O)NC7=O)c(C)c6)CC5)CC4)c(F)c3)n3ccnc(N)c23)cc1]

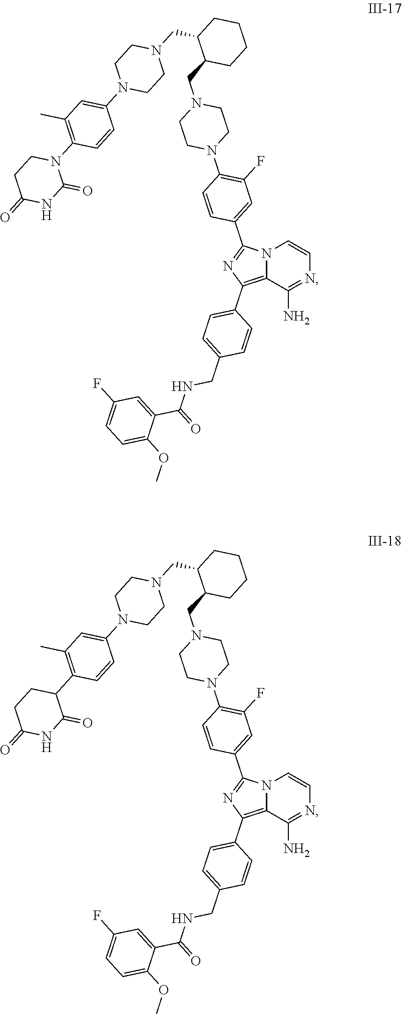 [SMILES: COc1ccc(F)cc1C(=O)NCc1ccc(-c2nc(-c3ccc(N4CCN(C[C@@H]5CCCC[C@H]5CN5CCN(c6ccc(N7CCC(=O)NC7=O)c(C)c6)CC5)CC4)c(F)c3)n3ccnc(N)c23)cc1 ; COc1ccc(F)cc1C(=O)NCc1ccc(-c2nc(-c3ccc(N4CCN(C[C@@H]5CCCC[C@H]5CN5CCN(c6ccc(C7CCC(=O)NC7=O)c(C)c6)CC5)CC4)c(F)c3)n3ccnc(N)c23)cc1]

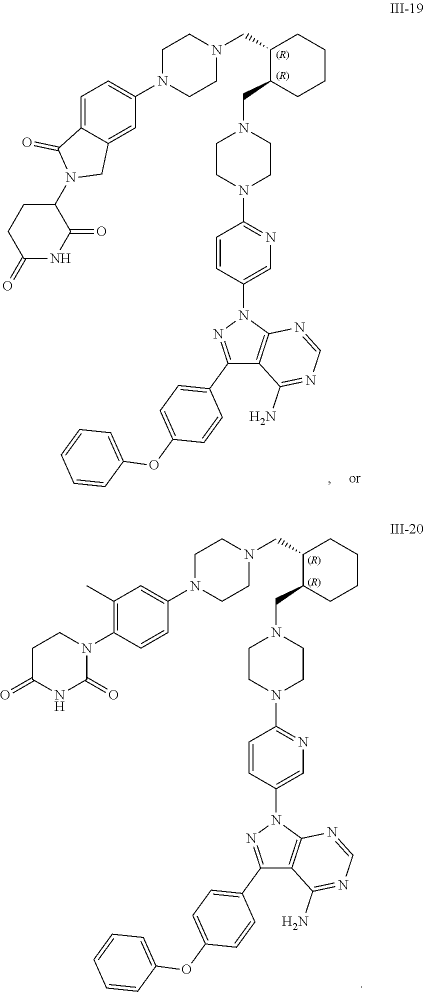

24. A pharmaceutical composition, comprising the compound according to any one of claim 1, or the isomer or pharmaceutically acceptable salt thereof, and pharmaceutically acceptable carriers.

25. A method for treating diseases related to the inhibition or degradation of BTK, comprising administering a therapeutically and/or prophylactically effective amount of the compound according to claim 1, the isomer or the pharmaceutically acceptable salt thereof to a subject in need thereof.

26. The method according to claim 25, wherein the disease is selected from autoimmune diseases, inflammatory diseases, or cancers.

27. The method according to claim 26, wherein the disease is selected from B cell malignancy, B cell lymphoma, diffuse large B cell lymphoma, chronic lymphocytic leukemia, small lymphocytic lymphoma, marginal zone lymphoma, non-Hodgkin's lymphoma, mantle cell lymphoma, follicular lymphoma, hairy cell leukemia, B-cell non-Hodgkin's lymphoma, Waldenstrom's macroglobulinemia, multiple myeloma, bone cancer, bone metastase, arthritis, multiple sclerosis, osteoporosis, irritable bowel syndrome, inflammatory bowel disease, Crohn's disease, lupus, Sjögren's syndrome, chronic graft-versus-host disease and a disorder associated with renal transplantation.
